(19) United States
(12) Patent Application Publication
(10) Pub. No.: US 2009/0307790 A1
(43) Pub. Date: Dec. 10, 2009

(54) ISOLATION OF THE T-COMPLEX DISTORTERS AND APPLICATIONS THEREOF

(75) Inventors: Bernhard Herrmann, Berlin (DE); Hermann Bauer, Berlin (DE)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: MAX-PLANCK-GESELLSCHAFT ZUR FORDERUNG DER WISSENSCHAFTEN E.V., 80539 Munchen (DE)

(21) Appl. No.: 11/997,992

(22) PCT Filed: Aug. 12, 2006
(86) PCT No.: PCT/EP2006/007977

§ 371 Date: Jul. 29, 2008

(30) Foreign Application Priority Data
Aug. 12, 2005 (EP) 05017651.0

(51) Int. Cl. A01K 67/027; C12Q 1/68; C12P 21/00; C12N 5/06; C12N 15/63; G01N 33/00; C07H 21/00; C12N 15/85

(52) U.S. Cl. 800/14; 435/6; 435/69.1; 435/325; 435/320.1; 436/94; 536/23.1; 800/13; 800/19; 800/20; 800/21

(57) ABSTRACT

The present invention relates to a method for producing a transgenic non human male animal, preferably a mammal, fish, bird or insect, wherein the transgene(s) confer(s) a change in the transmission ratio of (a) genetic trait(s) to the offspring of said non human male animal, preferably mammal, fish, bird or insect to a non-Mendelian ratio, said method comprising introducing (a) a first nucleic acid molecule encoding an expression product with a Responder function into a chromosome of a non-human germ cell, (fertilized) egg cell, embryonic cell or a cell derived therefrom, of the same species as the transgenic male to be prepared, said chromosome containing or conferring said genetic trait(s), thereby linking on said chromosome said Responder function to the genetic trait(s); and (b) at least one second nucleic acid molecule encoding an expression product with a Distorter function into (a) chromosome(s) of a non-human germ cell, (fertilized) egg cell, embryonic cell or a cell derived therefrom, of the same species as the transgenic male to be prepared, wherein said expression product with a Distorter function is a factor involved in G protein signaling, wherein said first nucleic acid molecule encoding an expression product with Responder function and said at least one second nucleic acid molecule encoding an expression product with a Distorter function are introduced into the same or different chromosomes.

The present invention relates to a method for producing a transgenic non human male animal, preferably a mammal, fish, bird or insect, wherein the transgene(s) confer(s) a change in the transmission ratio of (a) genetic trait(s) to the offspring of said non human male animal, preferably mammal, fish, bird or insect to a non-Mendelian ratio, said method comprising introducing (a) a first nucleic acid molecule encoding an expression product with a Responder function into a chromosome of a non-human germ cell, (fertilized) egg cell, embryonic cell or a cell derived therefrom, of the same species as the transgenic male to be prepared, said chromosome containing or conferring said genetic trait(s), thereby linking on said chromosome said Responder function to the genetic trait(s); and (b) at least one second nucleic acid molecule encoding an expression product with a Distorter function into (a) chromosome(s) of a non-human germ cell, (fertilized) egg cell, embryonic cell or a cell derived therefrom, of the same species as the transgenic male to be prepared, wherein said expression product with a Distorter function is a factor involved in G protein signaling, wherein said first nucleic acid molecule encoding an expression product with Responder function and said at least one second nucleic acid molecule encoding an expression product with a Distorter function are introduced into the same or different chromosomes.

Furthermore, the invention relates to a method for producing a transgenic non human male animal, preferably a mammal, fish, bird or insect, wherein the transgene(s) confer(s) a change in the transmission ratio of (a) genetic trait(s) to the offspring of said non human male animal, preferably mammal, fish, bird or insect to a non-Mendelian ratio, said method comprising introducing (a) a first nucleic acid molecule encoding an expression product with a Responder function into a chromosome of a non-human germ cell, (fertilized) egg cell, embryonic cell or a cell derived therefrom, of the same species as the transgenic male to be prepared, said chromosome containing or conferring said genetic trait(s), thereby linking on said chromosome said Responder function to the genetic trait(s); and (b) at least one second nucleic acid molecule encoding an expression product directed against the Distorter function into (a) chromosome(s) of a non-human germ cell, (fertilized) egg cell, embryonic cell or a cell derived therefrom, of the same species as the transgenic male to be prepared, wherein said Distorter function is an expression product which is encoded by a nucleic acid molecule encoding an expression product with a Distorter function, wherein said expression product with a Distorter function is a factor involved in G protein signaling; and/or (c) a second nucleic acid molecule for inactivation of the Distorter function by homologous recombination, wherein said second nucleic acid molecule for inactivation of the Distorter function by homologous recombination is at least partially identical to said nucleic acid molecule encoding an expression product with a Distorter function, wherein said first nucleic acid molecule encoding an expression product with a Responder function and said at least one second nucleic acid molecule encoding an expression product directed against the Distorter function and/or said second nucleic acid molecule for inactivation of the Distorter function by homologous recombination are introduced into the same or different chromosomes, thereby partially or completely inactivating the Distorter function.

In this specification, a number of documents are cited. The disclosure content of these documents including manufacturers' manuals is herewith incorporated by reference in its entirety.

The mouse t-complex, a region of approximately 12 cM genetic distance on the proximal part of chromosome 17, contains several loci acting in concert to produce a phenomenon called transmission ratio distortion (TRD). The latter designation indicates the fact that the so-called t-haplotype form of this chromosomal region has a selective advantage over the wild type form in that it is transmitted to the offspring at non-Mendelian ratios of up to 99%. This transmission at non-Mendelian ratio is achieved by the concerted action of at least five loci, the t complex Distorters Tcd1a and Tcd1b (D1a, D1b), Tcd2 (D2) and Tcd3 (D3), and the t complex responder, Tcr (R^{t})(Lyon 1984, Lyon et al 2000). More Distorters have been postulated (Silver and Remis 1987).

According to Lyon's model (Lyon 1986) which formally explains the genetic interactions of these loci, D1, D2 and D3 act strongly and harmfully on the wild type allele of the Responder and weakly on the t form of the Responder (R^{t}), leading to distortion in favor of R^{t}. R^{t} might protect sperm carrying it from this harmful action of the Distorters. The Distorters act in trans while the Responder acts in cis. This means that the chromosome, which contains R^{t} is transmitted at non-Mendelian ratio to the offspring. If D2 or all the Distorters are present, the chromosome containing R^{t} is transmitted at a frequency of more than 50% up to 99% to the offspring. If no Distorter or only D1 or D3 are present, however, the chromosome containing R^{t} is transmitted at less than 50% to the offspring (as low as 12%, “low” phenotype). The Distorters are only transmitted at ratios over 50% if they are tightly linked to R^{t}. The trans-acting and cis-acting properties of the Distorters and the Responder, respectively, have been demonstrated by the transmission ratio properties of so-called partial t-haplotypes, which carry only a subset of the above named loci.

Genetic mapping of molecular markers on partial t-haplotypes allowed a rough localization of D1a, D1b, D2, D3 and R^{t} to subregions of the T/t-complex and relative to these molecular markers (Lyon 1984); (Fox, Martin et al. 1985); (Herrmann, Bucan et al. 1986); (Silver and Remis 1987); (Bullard, Ticknor et al. 1992); (Lyon et al 2000). Only one locus, R^{t} could be mapped fairly precisely to a region of appr. 200 kb, the so-called T66B region (renamed later Leh66B; (Fox, Martin et al. 1985); (Schimenti, Vold et al. 1987); (Nadeau, Varnum et al. 1989); (Rosen, Bullard et al. 1990); (Bullard, Ticknor et al. 1992)). The genomic region T66B has been cloned molecularly and analyzed. A partial restriction map covering approximately 145 kb of it has been published ((Schimenti, Vold et al. 1987); (Rosen, Bullard et al. 1990); (Bullard, Ticknor et al. 1992)).

An extensive and careful search of this region for genes expressed during spermatogenesis led to the identification of a fusion gene expressed during spermiogenesis, the haploid phase of sperm development. Molecular and genetic analyses showed that the fusion gene encoding a mutant form of a novel protein kinase, Smok, represents Tcr (Herrmann, Koschorz et al. 1999). Transgene analyses demonstrated that Smok^{Tcr}, in combination with Tcd loci, distorts the transmission ratio of itself and preferably closely linked genetic traits. Co-segregation of a transgene construct encoding Smok^{Tcr} with the Y-chromosome resulted in sex ratio distortion (Herrmann, Koschorz et al. 1999).

t complex Distorters could only be mapped very roughly to large chromosomal subregions of several megabase each in size due to suppression of meiotic recombination between the t-haplotype and the wild type chromosome. Rare recombinants have occurred between these chromosomes allowing separation of the different loci, but molecular access to the Distorter loci is extremely difficult. Several attempts to isolate t-Distorters have been reported, though none of the candidates has been verified by genetic means (for review see (Schimenti 2000); (Lyon 2003), Systematic approaches using deletion mapping in the Tcd1 region and candidate gene isolation also has failed to identify a Distorter at the molecular level (Planchart, You et al. 2000); (Lyon, Schimenti et al. 2000).

In Schimenti et al. (2005) a BAC encoding several genes was used for testing of its potential to rescue a sterility phenotype in transgenic animals. Two genes encoded on the BAC were disclosed as candidates for expressing rescuing activity, among them Synj2. An additional test for distorter activity encoded on the BAC was negative, thus none of the genes was linked to the transmission distortion phenomenon. The fact that Synj is related to G-protein signalling is merely fortuitous, and the data excluded a relation of any gene encoded on the BAC to transmission ratio distortion. Thus, the authors were able to relate two genes of different nature to male fertility rather than to isolate a candidate distorter.

The combined teachings of the prior art thus did not provide any clue how the genetic elements responsible for the Distorter phenotype might be identified and, hence, did not disclose any means of applicability related to said genetic entity. A preferred goal would be the targeted transmission ratio distortion using, as a basis, the molecular entity of the Distorter(s). The technical problem underlying the present invention therefore was to overcome these long standing prior art difficulties and to provide such means.

The solution to said technical problem is achieved by providing the embodiments characterized in the claims

The present invention relates to a method for producing a transgenic non human male animal, preferably a mammal, fish, bird or insect, wherein the transgene(s) confer(s) a change in the transmission ratio of (a) genetic trait(s) to the offspring of said non human male animal, preferably mammal, fish, bird or insect to a non-Mendelian ratio, said method comprising introducing (a) a first nucleic acid molecule encoding an expression product with a Responder function into a chromosome of a non-human germ cell, (fertilized) egg cell, embryonic cell or a cell derived therefrom, of the same species as the transgenic male to be prepared, said chromosome containing or conferring said genetic trait(s), thereby linking on said chromosome said Responder function to the genetic trait(s); and (b) at least one second nucleic acid molecule encoding an expression product with a Distorter function into (a) chromosome(s) of a non-human germ cell, (fertilized) egg cell, embryonic cell or a cell derived therefrom, of the same species as the transgenic male to be prepared, wherein said expression product with a Distorter function is a factor involved in G protein signaling, wherein said first nucleic acid molecule encoding an expression product with Responder function and said at least one second nucleic acid molecule encoding an expression product with a Distorter function are introduced into the same or different chromosomes.

Furthermore, the invention relates to a method for producing a transgenic non human male animal, preferably a mammal, fish, bird or insect, wherein the transgene(s) confer(s) a change in the transmission ratio of (a) genetic trait(s) to the offspring of said non human male animal, preferably mammal, fish, bird or insect to a non-Mendelian ratio, said method comprising introducing (a) a first nucleic acid molecule encoding an expression product with a Responder function into a chromosome of a non-human germ cell, (fertilized) egg cell, embryonic cell or a cell derived therefrom, of the same species as the transgenic male to be prepared, said chromosome containing or conferring said genetic trait(s), thereby linking on said chromosome said Responder function to the genetic trait(s); and (b) at least one second nucleic acid molecule encoding an expression product directed against the Distorter function into (a) chromosome(s) of a non-human germ cell, (fertilized) egg cell, embryonic cell or a cell derived therefrom, of the same species as the transgenic male to be prepared, wherein said Distorter function is an expression product which is encoded by a nucleic acid molecule encoding an expression product with a Distorter function, wherein said expression product with a Distorter function is a factor involved in G protein signaling; and/or (c) a second nucleic acid molecule for inactivation of the Distorter function by homologous recombination, wherein said second nucleic acid molecule for inactivation of the Distorter function by homologous recombination is at least partially identical to said nucleic acid molecule encoding an expression product with a Distorter function, wherein said first nucleic acid molecule encoding an expression product with a Responder function and said at least one second nucleic acid molecule encoding an expression product directed against the Distorter function and/or said second nucleic acid molecule for inactivation of the Distorter function by homologous recombination are introduced into the same or different chromosomes, thereby partially or completely inactivating the Distorter function.

The term “genetic trait” relates to a heritable feature or characteristic of an organism encoded by (a) nucleic acid molecule(s) contained in its genome, comprising naturally occurring characteristics as well as (a) feature(s) encoded by (a) nucleic acid molecule(s) engineered in vitro, which has/have been introduced into its genome.

The term “confer a change in the transmission ratio of a genetic trait(s) to the offspring of said non human male animal, preferably mammal, fish, bird or insect to a non-Mendelian ratio” as used in accordance with the present invention refers to changing the transmission ratio of (a) genetic trait(s) from the parents to their offspring to ratios markedly deviating from the expected Mendelian ratio of 50% (equal transmission). “Markedly deviating” in connection with the present invention means that the ratios might be less than 50 percent, preferably less than 40%, more preferably less than 30%, even more preferably less than 20% and most preferably less than 10% (reduced transmission) or might be at least 60%, more preferably at least 70%, even more preferably at least 80% and most preferably at least 90% (enhanced transmission).

The term “said chromosome containing said genetic trait(s)” as used in connection with the present invention means that the genetic trait is part of the chromosome and will then be transmitted together with said chromosome through the germline.

The term “said chromosome conferring said genetic trait(s)” as used in connection with the present invention means that the entire chromosome is to be considered as the genetic trait therefore the transmission ratio of said genetic trait is also changed when the entire chromosome will be transmitted through the germline.

The term “Responder function” as used in connection with the present invention refers to the unique property of a Responder to distort the transmission ratio of itself and (a) closely linked genetic trait(s) to non-Mendelian ratios, i.e. a marked deviation from 50%.

The term “Distorter function” as used in connection with the present invention refers to the potency of (a) Distorter(s) to enhance or reduce the transmission ratio of a Responder and (a) closely linked genetic trait(s), wherein the transmission ratio of the Responder and (a) closely linked genetic trait(s) markedly deviates from the Mendelian ratio.

The term “nucleic acid molecule encoding an expression product with Distorter function/with Responder function” relates to nucleic acid molecules wherein the deduction of the amino acid sequence of the nucleic acid molecules used in connection with the method of the present invention allows the conclusion that the (poly)peptide is the expression product that contributes to the Distorter/Responder function. However, it is not excluded that the mRNA contributes to or triggers said Distorter/Responder function. Also, it is envisaged in accordance with the present invention that the expression level, stage of expression during spermatogenesis or the copy number of said nucleic acid molecule results in or contributes to the Distorter/Responder function. Therefore, in a preferred embodiment of the nucleic acid molecule used in connection with the method of the invention said expression product is an RNA or a (poly)peptide.

The term “(poly)peptide” as used in the present invention describes a group of molecules which comprise the group of peptides, as well as the group of polypeptides. The group of peptides is consisting of molecules with up to 30 amino acids, the group of polypeptides is consisting of molecules with more than 30 amino acids. Furthermore, the term “protein” as used in connection with the present invention is to be considered identical with the term “(poly)peptide”.

The terms “Distorter” or “Responder” therefore as used in connection with the present invention are to be considered in their broadest sense. Preferably, the terms refer to the (poly)peptide with Distorter or Responder function encoded by the corresponding nucleic acid molecules. Also, as mentioned above, said term might refer to the corresponding mRNAs or the nucleic acid molecule encoding the (poly)peptide with Distorter or Responder function.

Preferably, the nucleic acid molecule encoding a Distorter function is selected from the group consisting of SEQ ID NOs 1 and 2 (mouse wildtype Tagap1); SEQ ID NOs 3 to 6 (mouse Tagap^{t1}; Tagap^{t2}; Tagap^{t3}; Tagap^{t4} (Tcd1a)); SEQ ID NOs 7 and 8 (homo sapiens and Rattus Tagap); SEQ ID NOs 9 to 11 (mouse wildtype Fgd2 transcript variants); SEQ ID NOs 12 and 55 (mouse Fgd2^{t6/w5}; (Tcd2, transcript variants 1 and 1a)); SEQ ID NOs 13 and 14 (mouse and human Tiam2 (Tcd 1b)); SEQ ID NOs 31 to 33 (Bos taurus, Canis familiaris and Gallus gallus Tagap1); SEQ ID NOs 56 to 60 (Danio rerio, Macacca mulatta, Monodelphis domestica, Xenopus tropicalis and Pan troglodytes Tagap1), SEQ ID NOs 34 to 38 (mouse Fgd2^{t6/w5} (Tcd2; transcript variant 2); Bos Taurus, Canis familiaris and Rattus Fgd2; Rattus Fgd2 (splice variant)); SEQ ID NOs 61 to 66 (Macacca mulatto (3 transcript variants), Monodelphis domestica, Pan troglodytes and Homo sapiens Fgd2); SEQ ID NOs 47 to 50 (Bos taurus, Gallus gallus, Rattus and Canis familiaris Tiam2) and SEQ ID NOs 67 to 72 (Macacca mulatto (2 transcript variants), Monodelphis domestica (3 transcript variants) and Pan troglodytes Tiam2).

It is also preferred that the nucleic acid molecule encoding a Responder function is as shown in SEQ ID NO 15 or 16 (Smok^{Tcr}, Tcr, R^{t}).

The term “factors involved in G protein signaling” as used in connection with the present invention refers to any factor and preferably any protein that is a part of a G protein signaling cascade and in particular to members of the GTPase superfamily. For example, said members comprise trimeric G proteins and monomeric GTPases, and (poly)peptides triggering, controlling, modifying (a) signal pathway(s) involving small GTPases or regulated by (a) signal pathway(s) involving small GTPases.

The term “homologous recombination” refers to gene targeting of a nuclear gene locus of interest by integration of a nucleic acid molecule construct containing (a) genomic fragment(s) of said gene thereby altering the DNA sequence of said nuclear gene locus. It is preferred that by introducing said nucleic acid molecule construct into the nuclear gene locus the gene activity of the Distorter is down-regulated or abolished.

Within the meaning of the present invention, the term “directed against the Distorter function” means that the nucleic acid molecule or the expression product of said nucleic acid molecule or the antibody directed against the Distorter reduces or interferes with the activity of the expression product(s).

The term “thereby partially or completely inactivating the Distorter function” as used in connection with the present invention refers to interfering with the gene activity of the Distorter by destruction of the mRNA, inhibition of translation of the mRNA, inhibition of the protein or enzymatic activity, or by other mechanisms allowing down-regulation or abolishment of the gene or protein activity of the Distorter. For example, in any of the above interferences partial inactivation means inactivation of at least 50%, preferably of at least 60%, more preferably of at least 70%, even more preferably of at least 80%, even more preferably of at least 90%, even more preferably of at least 95% and most preferably 100% (complete inactivation) A number of methods and assays which are known to the person skilled in the art allowing measuring down-regulation of transcript or protein levels, inhibition of protein translation or down-regulation of protein activity (Sambrook J. 1989). The skilled person can devise an assay wherein for example the amount of Distorter transcripts in cells containing said Distorter and expressing a nucleic acid molecule directed against said Distorter is compared to cells containing said Distorter but not said nucleic acid molecule directed against said Distorter. Likewise, the protein expression level of cells expressing said Distorter can be compared to cells expressing in addition a nucleic acid molecule allowing down-regulation or abolishment of the gene or protein activity of the Distorter for instance by western blot analysis using an antibody binding to the protein product of said Distorter. The protein activity of the expression product of a Distorter allele which has been altered in vitro in order to interfere with the protein activity of said wild type Distorter product can be compared to the activity of said wild type Distorter protein using in vitro activity assays such as for example those known in the art devised for assaying the activity of GAP or GEF proteins on target GTPases, such as for example, the one shown herein below, the method comprising expression of the Distorter protein in vitro or in bacterial cells. Expression products derived from dominant negative alleles can be assayed in mixing experiments in the presence of the wild type protein for its ability to interfere with the activity of the wild type protein. Furthermore, the activity of constitutively active proteins can be compared to the activity of the wild type protein comprising relating the activity of either protein to the protein amounts used in the assay.

The term “partially identical”, as used herein, means in a first alternative that the genomic fragments used for integrating the nucleic acid molecule construct by homologous recombination are completely identical with the target nucleic acid molecule encoding an expression product with Distorter function. In this alternative the overall construct is partially identical because the target nucleic acid molecule encoding an expression product with Distorter function is not identical with the sequence in the construct used for the inactivation. Alternatively, even said genomic fragments may not be completely identical with the target nucleic acid molecule encoding an expression product with Distorter function but are sufficiently identical to allow recombination.

As outlined above and in other terms, the invention solves the recited technical problem by providing a reproducible method for changing the transmission ratio of genetic traits in non-human mammals, birds, fish or insects. In particular, as mentioned above, prior art methods failed to identify a Distorter which, however, is needed for carrying out the method of the present invention.

Several genes with respect to their role as a distorter were examined. This included analyses to determine the genomic position, expression analyses to compare the t-haplotype versus the wild type and detailed sequence analyses. From these experiments, only a subset of these genes turned out to be promising and these selected candidates were further analyzed functionally by establishing transgenic and knock out mouse lines.

In the prior art, all t-Distorter candidate genes which have been reported had been identified by the criteria that they were a) located within the t-complex region and b) play a role in sperm specific functions or are primarily expressed in sperm cells, such as Tctex1, Tctex2, Tcte2 and Tcp11 (Fraser and Dudley, 1999). This obvious assumption that t-Distorters are likely involved in sperm specific functions was used as an aid for preselection of likely candidates from the hundreds of genes located in the t-complex region, but turned out to be false. Additionally, it was known in the prior art that transmission ratio distortion relates to sperm motility and that the Responder relates to a Smok kinase (Herrmann et al, 1999), from which a person skilled in the art might have derived that factors involved in calcium signaling or cAMP signaling might be involved in the Distorter phenotype. However, the prior art did not give any clue whatsoever that factors involved in G protein signaling might function as a Distorter. The method of the present invention therefore for the first time makes use of nucleic acid molecules encoding such factors which are involved in G protein signaling for the above-indicated purpose.

The method of the present invention is based on the fact that in mouse the t-haplotype chromosome is transmitted at non-Mendelian ratio (significantly higher or lower than 50%) to the offspring. This phenomenon involves Tcr (Responder) and several Tcd (Distorter) loci, wherein the Tcd loci in the t-haplotype, that is the mutant forms, enhance the transmission ratio of Tcr. In the wild-type form, on the other hand, the Tcd+ loci reduce the transmission ratio of Tcr (the “low” phenotype). In the prior art, Tcd loci could not be localized precisely by chromosomal mapping due to recombination suppression between the t-haplotype and the wild type chromosome. Thus the coarse localization of Tcd loci to regions of several megabases in size each prevented the identification of t-Distorters. Even deletion mapping of Tcd1 did not allow identification of this factor (Lyon et al 2000); (Schimenti et al 2000).

Thus, these prior art difficulties had to be overcome in order to solve the ignorance of the molecular nature of a t-Distorter. The present invention not only makes use of Distorters which are factors involved in G protein signaling, but on top of this solved the problem which was in the prior art that the isolation of a Distorter could not be achieved. In the course of isolating a nucleic acid molecule which encodes one of the Distorters which can be used in connection with the present invention, in particular Tagap1, the inventors isolated a specific fusion gene, which showed similarity to FGF receptor oncogene partner (Fop) and is highly expressed in testis of wild type mice, but not of mice carrying the t-haplotype, suggesting that it is related to transmission ratio distortion. However, the inventors could demonstrate by gene targeting and genetic testing that this fusion gene did not show any Distorter activity. Nevertheless, the inventors did not turn to another candidate, but continued the study of this locus. Southern blot analysis indicated that this gene or a part thereof was present in a second locus on the chromosome, but there was no indication that that second gene or gene fragment was expressed. Rapid amplification of cDNA end (RACE) technology was attempted to complete the 5′-region of the second gene transcript, but these experiments produced the 5′ end sequence of the known fusion gene instead because this gene is highly expressed in testis. Only after several months of unsuccessful trials eventually a different 5′-end was identified, which then led to the isolation of Tagap1. The transcript of the missing locus, Tagap1, per se was only very weakly expressed in testis and did not hint to a Distorter function either. Only by completing the entire gene the inventors could recognize the missing (second) gene as a gene involved in G protein signalling. Gene targeting and genetic testing demonstrated that Tagap1 is able to alter the transmission ratio of a t-haplotype carrying Tcr. However, the targeted allele reduced the transmission ratio, in contrast to the teachings of the prior art, since Lyon had shown that a deletion of Tcd1 on the wild type chromosome enhanced the transmission ratio (Lyon 1992). Large efforts involving extensive genetic analyses and the production and analyses of transgenic lines had to be undertaken by the inventors to finally show that the t-loci of Tagap1 constitute a Distorter which enhances the transmission ratio of a t-haplotype and which must be different from the t-Distorter identified by Lyon by genetic means using the deletion chromosome T^{22H}.

From the above, the inventors hypothesized that further factors involved in G protein signaling might be involved in Distorter function as for example shown in Example 3 and could identify further factors in the cascade showing Distorter function. Hence, the present invention for the first time links G protein signalling to the phenomenon of transmission ratio distortion.

The method of the present invention comprises deriving an adult animal from said non human germ cell, fertilized egg cell, embryonic cell or cell derived therefrom containing the nucleic acid molecules as defined in the present invention. The fertilized egg cell or an embryo into which said embryonic cell has been introduced (thereby forming a chimera), or the fertilized egg or zygote derived from introducing the nucleus of the cell containing the nucleic acid molecules as defined in the present invention into an egg cell or zygote whose genome has been removed (thereby forming an embryo containing said the nucleic acid molecules as defined in the present invention in its genome), is/are transferred into a foster mother and the embryo let develop to term.

The offspring of the foster mother are then determined for the integration of the nucleic acid molecules as described in the present invention and the sex is determined by methods known to the person skilled in the art. Such methods comprise for example genotyping by PCR and further methods as also described below.

The male offspring can further be characterized by visual inspection of outer genitalia and also by detecting male specific markers. Said techniques are also known to the person skilled in the art.

All methods employed for deriving an adult animal are known in the prior art and further described, where applicable, in the specification of the present invention.

The transgenic non-human male, fish, bird or insect produced by the method of the present invention can be prepared in at least two alternative ways. The nucleic acid molecules as defined in the present invention can be introduced into the same non-human germ cell, (fertilized) egg cell, embryonic cell or a cell derived therefrom and the adult organism let develop as described above. Alternatively, the nucleic acid molecules as defined in the present invention can be introduced into different cells thereby producing two different adult organisms with the same methods as described above. The male and female of said two organisms are then crossed and the offspring is analyzed for the nucleic acid molecule as described in the present invention and the male is characterized as described above.

As mentioned, the techniques involved in animal breeding and animal crossing and the techniques involved in transgenesis are known to the person skilled in the art and, where applicable, are described in the present specification.

Alternatively, in the case that the investigator wishes to start at a different stage of accomplishing the invention the following alternative embodiments are set up.

According to one of these embodiments the invention relates to a method for producing a transgenic non human animal, preferably mammal, fish, bird or insect, said method comprising introducing (a) a first nucleic acid molecule encoding an expression product with a Responder function into a chromosome of a non-human germ cell, (fertilized) egg cell, embryonic cell or a cell derived therefrom, of the same species as the transgenic animal to be prepared, said chromosome containing or conferring said genetic trait(s), thereby linking on said chromosome said Responder function to the genetic trait(s); and (b) at least one second nucleic acid molecule encoding an expression product with a Distorter function into (a) chromosome(s) of a non-human germ cell, (fertilized) egg cell, embryonic cell or a cell derived therefrom, of the same species as the transgenic female to be prepared, wherein said expression product with a Distorter function is a factor involved in G protein signaling, wherein said first nucleic acid molecule encoding an expression product with Responder function and said at least one second nucleic acid molecule encoding an expression product with a Distorter function are introduced into the same or different chromosomes.

Alternatively, the invention envisages a method for producing a transgenic non human animal, preferably mammal, fish, bird or insect, said method comprising introducing (a) a first nucleic acid molecule encoding an expression product with a Responder function into a chromosome of a non-human germ cell, (fertilized) egg cell, embryonic cell or a cell derived therefrom, of the same species as the transgenic animal to be prepared, said chromosome containing or conferring said genetic trait(s), thereby linking on said chromosome said Responder function to the genetic trait(s); and (b) at least one second nucleic acid molecule encoding an expression product directed against the Distorter function into (a) chromosome(s) of a non-human germ cell, (fertilized) egg cell, embryonic cell or a cell derived therefrom, of the same species as the transgenic female to be prepared, wherein said Distorter function is an expression product which is encoded by a nucleic acid molecule encoding an expression product with a Distorter function, wherein said expression product with a Distorter function is a factor involved in G protein signaling; and/or (c) a second nucleic acid molecule for inactivation of the Distorter function by homologous recombination, wherein said second nucleic acid molecule for inactivation of the Distorter function by homologous recombination is at least partially identical to said nucleic acid molecule encoding an expression product with a Distorter function, wherein said first nucleic acid molecule encoding an expression product with a Responder function and said at least one second nucleic acid molecule encoding an expression product directed against the Distorter function and/or said second nucleic acid molecule for inactivation of the Distorter function by homologous recombination are introduced into the same or different chromosomes, thereby partially or completely inactivating the Distorter function.

The above animal may be male or female. In further embodiments, the invention envisages methods for the production of transgenic animals wherein only step (b) is carried out. Again, said animals are preferably mammals, birds, fish or insects.

From these animals the male animal of the main embodiments (if the outcome is not a male anyway) can be generated by the above embodiments.

Methods for the generation of transgenic mammal, fish, bird or insects are well known in the art and are described (for example in (DePamphilis 1993), (Chapman, Lawson et al. 2005)).

Other methods comprise the use of retroviral, in particular lentiviral particles carrying constructs engineered in vitro for the infection of cells, preferably egg cells, zygotes or early embryos, the integration of recombinant DNA constructs into embryonic stem cells and production of stem cell/embryo chimera, or the generation of egg cells or sperm cells from embryonic stem cells having integrated the recombinant DNA construct, by differentiation of said cells in vitro (Lever et al., 2004); (Hubner et al., 2003); (Geijsen et al., 2004).

A further method comprises recombinase mediated cassette exchange (RMCE) whereby a construct which is flanked by non-identical target sites, such as loxP and lox2272 sites, recognized by a site specific recombinase, such as Cre is exchanged by homologous recombination mediated by the recombinase for a fragment which is contained in a chromosome and which is flanked by said sites (such as loxP and lox2272 in this example), thereby integrating the construct into said chromosome (Pirottin, Grobet et al. 2005).

The method of the invention also comprises embodiments related to the cloning of transgenic animals. These embodiments include the steps of introducing the nucleic acid molecule as defined in the present invention, recombinant DNA molecule or vector comprising said nucleic acid molecule into the nucleus of a cell, preferably an embryonic cell, replacing the nucleus of an oocyte, a zygote or an early embryo with said nucleus comprising said nucleic acid molecule, recombinant DNA molecule or vector, transferring either said oocyte, zygote or early embryo into a foster mother or first in vitro or in vivo culturing said oocyte, zygote or early embryo and subsequently transferring the resulting embryo into a foster mother and allowing the embryo to develop to term; see, for example, (Wilmut, Schnieke et al. 1997).

A method for the production of a transgenic non-human animal, for example transgenic mouse, comprises introduction of a nucleic acid molecule or targeting vector into a germ cell, an embryonic cell, stem cell or an egg or a cell derived therefrom. Production of transgenic embryos and screening of those can be performed, e.g., as described (Joyner 1993). The DNA of the embryonal membranes of embryos can be analyzed using, e.g., Southern blots with an appropriate probe. A general method for making transgenic non-human animals is described in the art, see for example WO 94/24274. For making transgenic non-human organisms (which include homologously targeted non-human animals), embryonic stem cells (ES cells) are preferred. Murine ES cells, such as AB-1 line grown on mitotically inactive SNL76/7 cell feeder layers (McMahon and Bradley, Cell 62: 1073-1085 (1990)) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al., Nature 326: 292-295 (1987)), the D3 line (Doetschman et al., J. Embryol. Exp. Morph. 87: 2745 (1985)), the CCE line (Robertson et al., Nature 323: 445-448 (1986)), the AK-7 line (Zhuang et al., Cell 77: 875-884 (1994) which is incorporated by reference herein). The success of generating a mouse line from ES cells bearing a specific targeted mutation depends on the pluripotency of the ES cells (i.e., their ability, once injected into a host developing embryo, such as a blastocyst or morula, to participate in embryogenesis and contribute to the germ cells of the resulting animal). The blastocysts containing the injected ES cells are allowed to develop in the uteri of pseudopregnant nonhuman females and are born as chimeric mice. The resultant transgenic mice are chimeric for cells having either the recombinase or reporter loci and are backcrossed and screened for the presence of the correctly targeted transgene (s) by PCR or Southern blot analysis on tail biopsy DNA of offspring so as to identify transgenic mice heterozygous for either the recombinase or reporter locus/loci.

Methods for producing transgenic flies, such as Drosophila melanogaster are also described in the art, see for example U.S. Pat. No. 4,670,388, Brand & Perrimon, Development (1993) 118: 401-415; and Phelps & Brand, Methods (April 1998) 14: 367-379.

In a preferred embodiment of the method of the present invention said mammal is selected from the group consisting of Mus, Rattus, Bos, Sus and Ovis.

It is more preferred that Mus is Mus musculus, Rattus is Rattus norvegicus, Bos is Bos taurus, Sus is Sus scrofa f. domestica.

In another preferred embodiment of the method of the present invention said genetic trait is sex.

In further preferred embodiment of the method of the present invention said chromosome is an X or Y chromosome or a corresponding sex chromosome in birds (W, Z), fish or insect. Changing the transmission ratio in insects will have an important impact on the fight against insect pests. For example, by mixing a number of transgenic male Anopheles prepared in accordance with this invention with a naturally occurring Anopheles population, responsible for the spreading of malaria, the production of e.g. predominantly male offspring of the transgenic Anopheles is expected (see also explanation herein below). These male Anopheles will change the overall frequency of males in the population and thus lead to an overall mating problem in the Anopheles population which eventually will lead to an overall reduced amount of Anopheles. A corresponding strategy may be employed with, for example, locusts.

In a still further preferred embodiment of the method of the present invention said chromosome is an autosome.

In still a further preferred embodiment of the method of the present invention the factor involved in G protein signalling is a factor involved in Rho signalling.

The term “a factor involved in Rho signaling” as used in connection with the present invention refers to small G proteins of the Rho subfamily, as well as to molecules acting upstream or downstream of G proteins in terms of signal transduction. Such molecules comprise in particular members of the families of GEFs (guanine nucleotide exchange factors), which enhance the activity of small G proteins, GAPs (GTPase activating proteins) which act as negative regulators and GDIs (guanine nucleotide dissociation inhibitors) which also attenuate small G protein signaling (Schmidt and Hall, 2002); (Donovan et al., 2002); (DerMardirossian and Bokoch, 2005). Finally, the term refers to target molecules, influenced by small G proteins (Bishop and Hall, 2000).

In a further preferred embodiment of the method of the present invention said first nucleic acid molecule encoding an expression product with Responder function is selected from the group consisting of (a) a nucleic acid molecule comprising or consisting of the nucleic acid molecule as shown in SEQ ID No: 15 or 16 or a fragment thereof; (b) a nucleic acid molecule being an allelic variant or a homologue or orthologue of the nucleic acid molecule of (a); (c) a nucleic acid molecule hybridizing to a nucleic acid molecule complementary to the nucleic acid molecule of (a) or (b); and (d) a nucleic acid molecule which is related to the nucleic acid molecule of (a), (b) or (c) by the degeneration of the genetic code.

In further preferred embodiment of the method of the present invention said (at least one) second nucleic acid molecule encoding an expression product with a Distorter function is/are selected from the group consisting of (a) a nucleic acid molecule comprising or consisting of the nucleic acid molecule as shown in any one of SEQ ID NOs: 1 to 14, 31 to 33, 34 to 38, 47 to 72 or a fragment thereof, (b) a nucleic acid molecule being an allelic variant or a homologue or orthologue of the nucleic acid molecules of (a); (c) a nucleic acid molecule hybridizing to a nucleic acid molecule complementary to the nucleic acid molecule of (a) or (b); and (d) a nucleic acid molecule which is related to the nucleic acid molecule of (a), (b) or (c) by the degeneration of the genetic code.

The term “an allelic variant or homologue” as used in connection with the present invention refers to different wild type forms and t-alleles of the nucleic acid molecules.

The nucleic acid molecules can be further manipulated in vitro in order to achieve an optimized transmission ratio distortion effect and/or to adapt it to the specific requirements of the breeding scheme employed, thus further improving the selectability of genetic traits as further described below. A number of standard manipulations known in the field are taken into consideration, such as those resulting in the exchange of amino acids in the catalytic domain(s) which is the GAP domain in case of the GTPase activating proteins and the DH (Dbl-homology) domain in case of the guanine nucleotide exchange factors, overexpression or knock out mutagenesis of said nucleic acid molecules, construction of hypomorphic or hypermorphic (poly)peptides by mutagenesis, deletion or alteration of candidate modification sites on said (poly)peptide, deletion or alteration of binding sites for other (poly)peptides involved in the G protein signaling cascade (see for example (Dvorsky and Ahmadian, 2004)), synthesis of antisense RNA, siRNA, shRNA, N-terminal or C-terminal truncations, introduction of frame shifts, which alter part of the amino acid sequence of the protein, etc., resulting either in null, hypomorphic, constitutively active, antimorphic or dominant negative alleles. It is also envisaged that a distortion of the transmission ratio can be achieved with several, if not all, manipulated forms of the nucleic acid molecules described above. Thus, a manipulated allele affecting the transmission ratio most effectively will have to be identified empirically for each gene by employing activity assays in vitro and in cell culture systems such as NIH-3T3 cells and transgenic animal systems.

The term “orthologue” as used in connection with the present invention refers to genes present in different organisms and which have the same function.

The term “fragment” as used in connection with the method of the present invention relates to the fact that said fragment retains the Responder/Distorter function.

If fragments, allelic variants, homologues or orthologues of a specifically identified sequence conferring responder or Distorter function are referred to throughout this specification, it is understood that these fragments etc. retain or essentially retain the Responder or Distorter function. “Essentially retain” means in accordance with these embodiments that at least 70% of the function are retained, preferably at least 80% such as at least 90%.

The term “hybridizing” as used in connection with the present invention and as used in the description of the present invention, preferably refers to “hybridizing under stringent conditions”, and is well known to the skilled artisan and corresponds to conditions of high stringency. Appropriate stringent hybridization conditions for each nucleic acid sequence may be established by a person skilled in the art on well-known parameters such as temperature, composition of the nucleic acid molecules, salt conditions etc.; see, for example, (Sambrook J. 1989) (Hames 1985), see in particular the chapter “Hybridization Strategy” by Britten & Davidson, 3 to 15. Stringent hybridization conditions are, for example, conditions comprising overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 micrograms/ml denatured, sheared salmon sperm DNA, followed by washing the filters in 0.1×SSC at about 65°. Other stringent hybridization conditions are for example 0.2×SSC (0.03 M NaCl, 0.003M sodium citrate, pH 7) at 65° C. In addition, to achieve even higher stringency, washes performed following stringent hybridization can be done at higher salt concentrations (e.g. 5×SSC). Note that variations in the above conditions may be accomplished through the inclusion and/or substitution of alternate blocking reagents used to suppress background in hybridization experiments. Typical blocking reagents include, but are not limited to, Denhardt's reagent, BLOTTO, heparin, denatured salmon sperm DNA, and commercially available proprietary formulations. The inclusion of specific blocking reagents may require modification of the hybridization conditions described above, due to problems with compatibility. Also contemplated are nucleic acid molecules encoding an interaction partner of a biomolecule, wherein the interaction partner is capable of modulating the activity said biomolecule and wherein the nucleic acid molecules hybridize to the nucleic acid molecule encoding the biomolecule at even lower stringency hybridization conditions. Changes in the stringency of hybridization and signal detection are, for example, accomplished through the manipulation of formamide concentration (lower percentages of formamide result in lowered stringency); salt conditions, or temperature. For example, lower stringency conditions include an overnight incubation at 37 degree C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include, but are not limited to, Denhardt's reagent, BLOTTO, heparin, denatured salmon sperm DNA, and commercially available proprietary formulations. The inclusion of specific blocking reagents may require modification of the hybridization conditions described above, due to problems with compatibility.

As shown above, it is preferred that the method of the present invention be carried out by using the nucleic acid molecules encoding a Distorter function described in SEQ ID NOs: 1 to 14, 31 to 33, 34 to 38, 47 to 72. These sequences relate to wildtype Tagap1 (SEQ ID NO: 1 and 2), the t-alleles of Tagap1, Tagap1^{t1 to t4} (SEQ ID NOs: 3 to 6), the homo sapiens Tagap (SEQ ID NO: 7), the Rattus Tagap (SEQ ID NO: 8), three transcript variants of wildtype mouse Fgd2 (SEQ ID NOs: 9 to 11), three t-alleles of Fgd2 (SEQ ID NOs: 12, 34 and 55), the mouse and homo sapiens Tiam 2 (SEQ ID NOs 13 and 14), the Bos taurus, Canis familiaris and Gallus gallus Tagap (SEQ ID NOs 31 to 33), the Danio rerio, Macacca mulatta, Monodelphis domestica, Xenopus tropicalis and Pan troglodytes Tagap1 (SEQ ID NOs 56 to 60); Bos Taurus, Canis familiaris and Rattus Fgd2; Rattus Fgd2 (splice variant) (SEQ ID NOs 35 to 38), the Macacca mulatta (3 transcript variants), Monodelphis domestica and Pan troglodytes Fgd2 (SEQ ID NOs 61 to 66), Bos taurus, Gallus gallus, Rattus and Canis familiaris Tiam2 (SEQ ID NOs 47 to 50) and Macacca mulatta (2 transcript variants), Monodelphis domestica (3 transcript variants) and Pan troglodytes Tiam2 (SEQ ID NOs 67 to 72).

In another preferred embodiment of the method of the present invention said at least one second nucleic acid molecule encoding an expression product with a Distorter function is/are selected from the group consisting of (a) a nucleic acid molecule comprising or consisting of the nucleic acid molecule as shown in any one of SEQ ID NOs 1 to 12, 31 to 38 or 55 to 66 or a fragment thereof (b) a nucleic acid molecule being an allelic variant or a homologue or orthologue of the nucleic acid molecule of (a); (c) a nucleic acid molecule hybridizing to a nucleic acid molecule complementary to the nucleic acid molecule of (a) or (b); and (d) a nucleic acid molecule which is related to the nucleic acid molecule of (a), (b) or (c) by the degeneration of the genetic code, thereby enhancing said transmission ratio of said genetic trait(s).

In a further preferred embodiment of the method of the present invention said at least one second nucleic acid molecule encoding an expression product with a Distorter function is/are selected from the group consisting of (a) a nucleic acid molecule comprising or consisting of the nucleic acid molecule as shown in of SEQ ID NO: 13 or 14, 47 to 50 or 67 to 72 or a fragment thereof; (b) a nucleic acid molecule being an allelic variant or a homologue or orthologue of the nucleic acid molecule of (a); (c) a nucleic acid molecule hybridizing to a nucleic acid molecule complementary to the nucleic acid molecule of (a) or (b); and (d) a nucleic acid molecule which is related to the nucleic acid molecule of (a), (b) or (c) by the degeneration of the genetic code, thereby reducing said transmission ratio of said genetic trait(s).

In another preferred embodiment of the method of the present invention said nucleic acid molecule encoding an expression product with a Distorter function is selected from the group consisting of (a) a nucleic acid molecule comprising or consisting of the nucleic acid molecule as shown in any one of SEQ ID NOs: 1 to 12, 31 to 38 or 55 to 66 or a fragment thereof; (b) a nucleic acid molecule being an allelic variant or a homologue or orthologue of the nucleic acid molecule of (a); (c) a nucleic acid molecule hybridizing to a nucleic acid molecule complementary to the nucleic acid molecule of (a) or (b); and (d) a nucleic acid molecule which is related to the nucleic acid molecule of (a), (b) or (c) by the degeneration of the genetic code, thereby reducing said transmission ratio of said genetic trait(s).

In still another preferred embodiment of the method of the present invention said nucleic acid molecule encoding an expression product with a Distorter function is selected from the group consisting of (a) a nucleic acid molecule comprising or consisting of the nucleic acid molecule as shown in SEQ ID NOs 13 or 14, 47 to 50 or 66 to 72 or a fragment thereof; (b) a nucleic acid molecule being an allelic variant or a homologue or orthologue of the nucleic acid molecule of (a); (c) a nucleic acid molecule hybridizing to a nucleic acid molecule complementary to the nucleic acid molecule of (a) or (b); and (d) a nucleic acid molecule which is related to the nucleic acid molecule of (a), (b) or (c) by the degeneration of the genetic code, thereby enhancing said transmission ratio of said genetic trait(s).

In further preferred embodiment the method of the present invention further comprises crossing the transgenic non human male mammal, fish, bird or insect obtained by the method of the present invention with a non human female mammal, fish, bird or insect and analyzing the offspring of said cross for transmission of said genetic trait(s).

The above preferred embodiments of the method of the present invention are of particular interest for the applicability of the present invention. The genetic trait(s) of interest can be transmitted to the offspring either at an enhanced or at a reduced ratio with respect to the Mendelian ratio. In particular it is envisaged to use the methods of the present invention in the field of farm animal breeding as a tool for manipulating the transmission ratio of genetic traits. The most interesting trait in this respect is sex. The method of the present invention making use of the Distorter now allows an enhancement of the effect of the Responder, for example it will be possible to obtain strong selection for or against sperm carrying the Y chromosome. It is therefore envisaged that a transgene construct expressing the nucleic acid molecule encoding the Responder function and expressing at least one other nucleic acid molecule encoding (a) Distorter function(s) and/or products directed against the Distorter function be integrated on the Y-chromosome of the farm animal species. In one embodiment of the present invention action of the Distorter(s) would impair the sperm cells carrying the Responder, which would result in a preferential or exclusive transmission of the X-chromosome and thus generation of female offspring. In another embodiment of the present invention the action of the Distorter(s) and/or the product(s) directed against the Distorter function would impair all sperm cells, while the sperm cells carrying and expressing the Responder would be rescued. In that latter embodiment the Y chromosome would be preferentially or exclusively transmitted to the offspring resulting in the production of male offspring. Likewise, the construct expressing the Responder function and the construct(s) expressing at least one Distorter function and/or product(s) directed against the Distorter function could be integrated on the X chromosome and allow the generation of males preferentially or exclusively transmitting the Y chromosome or, in the latter example, wherein a high transmission ratio of the Responder construct is achieved, the X chromosome. It will depend on the design of the construct(s) (as taught above) expressing the Distorter function and/or product(s) directed against the Distorter function whether enhanced transmission or reduced transmission of the chromosome carrying the Responder construct will be achieved. For example, in the present invention it could be shown that inactivation of wild type Tagap1 and inactivation of wild type Fgd2 both resulted in a reduced transmission of Tcr, while the respective t-Distorters Tagap1^{Tcd1a} and Fgd2^{Tcd2} enhance the transmission of Tcr. In contrast, it is envisaged that loss of Tiam2 function enhances the transmission of Tcr, while a hypermorphic allele is envisaged to reduce the transmission ratio of Tcr. Thus, the teachings of the t-Distorters and of the mutations in the wild type gene provided by the present invention provide the knowledge how Distorter alleles need to be engineered to achieve enhancement or reduction of the transmission ratio of the chromosome carrying the Responder construct and the genetic trait(s) linked to it. Importantly, Distorters act additively or synergistically, thus the combinatorial use of several nucleic acids encoding Distorter function(s) and/or products directed against the Distorter function(s) is preferred in order to achieve an optimal effect. For example, it is envisaged that the combination of nucleic acid molecules encoding products directed against the function of Tagap1 and of Fgd2 and of a construct overexpressing Tiam2 achieves a strong effect with respect to selection against sperm carrying and expressing the Responder construct, since all three expression products singly should reduce the transmission ratio of the Responder.

It is furthermore envisaged that the constructs expressing the Distorter(s) and/or the constructs expressing an expression product directed against the Distorter function and/or the constructs for inactivation of the Distorter function can be integrated independently of the Responder construct on the same or on different chromosomes. Such a tool for preselection of sex in farm animals is most desirable for Bos taurus, a species for which specialized strains for milk or meat production have been derived.

Female offspring is needed for milk production whereas for meat production male offspring is preferred in this species. In most other farm animal species female offspring is most desired. Thus, preselection of sex is of general importance in farm animal breeding.

In another preferred embodiment of the method of the present invention said Responder function and/or said Distorter function is the mouse-t-complex Responder/Distorter function.

Although it is possible without undue burden to identify mutated or wild-type Distorters in animals other than the mouse on the basis of the genetic structure of the Distorter that is provided in accordance with the present invention, it is envisaged that the mouse t-complex Distorter may find applications, for example in breeding, also when introduced into other animals. Specific applications of the Distorter function are addressed herein below.

It is furthermore envisaged that generally the set of genes, Responder and one or more distorters, used to create a transgenic animal is chosen independently of the target animal species and can be applied in any combination allowed by the included sequence listing. For example, it is imaginable that the mouse responder is combined with one or more distorters of one or more different species to be transferred to an animal which belongs to none of said species.

For example, it is imaginable that the mouse responder is combined with one or more distorters of one or more different species to be transferred to an animal which belongs to none of said species. A more specific example would include the mouse responder gene combined with e.g. Macacca distorter genes to be transduced into Bos taurus or e.g. one Macacca distorter and a different Canis distorter to be transduced into Bos taurus.

In another preferred embodiment one or more distorters may be chosen from the sequences corresponding to the targeted animal. For example, the distorters belong to the species Bos taurus and are used to create a transgenic animal belonging to the same species.

In still another preferred embodiment of the method of the present invention said expression product directed against the Distorter function is an aptamer, a siRNA or shRNA or miRNA, a ribozyme, or an antisense nucleic acid molecule specifically hybridizing to said nucleic acid molecules encoding a factor involved in G protein signaling, or is an antibody, an antibody fragment or derivative thereof specific for the Distorter (poly)peptides as used in connection with the present invention.

It is envisaged that shRNA (small hairpin RNA) molecules expressed from a construct integrated into the genome can be used for degradation of RNA molecules (known as RNA interference) transcribed from (a) endogenous gene(s) encoding (a) Distorter(s) thereby partially or completely down-regulating the function of said Distorter(s). Likewise vectors comprising nucleic acid molecules encoding a miRNA (microRNA) can be utilized for inhibition of translation of the RNA encoding said Distorter(s) (Kim 2005). Constructs expressing aptamers can be utilized to inhibit protein-protein interaction such as between a Distorter protein and another (poly)peptide of the Distorter/Responder signaling cascade in order to interfere with the propagation of the signal thereby inhibiting said signal pathway. The person skilled in the art is able to design shRNA or miRNA constructs on the basis of the sequence of the mRNA of the gene the function of which shall be down-regulated. The efficacy of the constructs can easily be tested in cellular systems. Aptamers able to inhibit protein-protein interaction can be selected in vitro or in cellular system such as the yeast the method comprising assaying inhibition of protein-protein interaction as measured in the yeast two-hybrid assay (Schmidt, Diriong et al. 2002); (Kurtz, Esposito et al. 2003); (Cassiday and Maher 2003).

The term “antibody fragment or derivative thereof” relates to single chain antibodies, or fragments thereof, synthetic antibodies, antibody fragments, such as Fab, a F(ab₂)′, Fv fragments, single domain antibodies etc., or a chemically modified derivative of any of these. Derivatives include scFvs. Antibodies to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) (e.g. posttranslational and chemical modifications, such as glycosylation and phosphorylation) known in the art either alone or in combination. Methods for introducing such modifications in the DNA sequence underlying the amino acid sequence of an immunoglobulin chain are well known to the person skilled in the art; see, e.g., Sambrook et al.; Molecular Cloning: A Laboratory Manual; Cold Spring Harbor Laboratory Press, 2nd edition 1989 and 3rd edition 2001.

The term “antibody fragment or derivative thereof” particularly relates to (poly)peptide constructs comprising at least one CDR such as two, three and preferably all six CDRs of an antibody, e.g. in the scFv format. Framework regions of the antibody may also be replaced by unspecific non-antibody-related sequences.

Fragments or derivatives of the recited antibody molecules may also define (poly)peptides which are parts of the above antibody molecules and/or which are modified by chemical/biochemical or molecular biological methods. Corresponding methods are known in the art and described inter alia in laboratory manuals (see Sambrook et al., loc cit.; Gerhardt et al.; Methods for General and Molecular Bacteriology; ASM Press, 1994; Lefkovits; Immunology Methods Manual: The Comprehensive Sourcebook of Techniques; Academic Press, 1997; Golemis; Protein-Protein Interactions: A Molecular Cloning Manual; Cold Spring Harbor Laboratory Press, 2002; Antibodies, A Laboratory Manual, Ed Harlow and David Lane, Cold Spring Harbor Laboratory, 1988).

In a preferred embodiment of the method of the present invention said at least one second nucleic acid molecule encoding the expression product with a Distorter function is modified, thereby further reducing or further enhancing the Distorter function activity.

The term “further reducing or further enhancing the Distorter function activity” as used in connection with the method of the present invention refers to the fact that the method of the present invention can be further optimized by genetically manipulating the nucleic acid molecules encoding the Distorters. For example, dominant active or dominant negative alleles of (a) Distorter(s) may be designed and assayed in vitro for their ability to enhance or interfere with the activity of the wild type allele of said Distorter, followed by genetic testing in vivo in transgenic animals of the allele(s) which improve the activity of said Distorter or enhance down-regulation of said Distorter in vitro. Other alterations of the nucleic acid sequence resulting in changes of the polypeptide encoded by said Distorter gene may be introduced in vitro by exchanging nucleic acid molecules or by synthesizing genes or gene parts in vitro or by random mutagenesis, and (high-throughput) in vitro assays can be designed to measure the activity of the altered proteins or their ability to enhance or inhibit or interfere with (a) component(s) of the Distorter/Responder signal cascade.

In further preferred embodiment of the method of the present invention said first nucleic acid molecule encoding an expression product with a Responder function and said at least one second nucleic acid molecule encoding an expression product with Distorter function and/or said at least one second nucleic acid molecule encoding an expression product directed against the Distorter function and/or said second nucleic acid molecule for inactivation of the Distorter function by homologous recombination and a promoter controlling expression in spermatogenesis and/or spermiogenesis and/or a stop cassette are integrated in said X or Y chromosome or corresponding sex chromosome or in one of said autosomes in a reversible inactive state of expressibility. Preferably, said promoter is a heterologous promoter.

The term “reversible inactive state of expressibility” as used in connection with the method of the present invention refers to the possibilty to keep the above nucleic acid molecules in an inactive state of expressibility which can by genetic means be activated, as further described below.

In particular, it is envisaged that the construct(s) expressing the Distorter(s) and/or products directed against the Distorter function(s) reduce the transmission of the nucleic acid molecule encoding a Responder function to such a low ratio that the transmission of the Responder construct is almost or completely excluded. This would mean that one important component of the functional principle of the present invention is not passed on to the next generation by natural breeding. To circumvent this problem it is envisaged to introduce a construct containing the nucleic acid molecule encoding the Responder in an inactive state, for instance by inserting a transcription stop cassette between the promoter controlling expression of the Responder gene and the nucleic acid encoding the Responder, comprising flanking the stop cassette by loxP sites in same orientation. This construct would be inactive with respect to expression of the Responder and thus could be transmitted at Mendelian ratio to the offspring. Males producing sperm allowing preselection of (a) trait(s) are envisaged to be produced by breeding the male or female carrying the inactive Responder construct to a female or male carrying a construct expressing Cre recombinase prior to spermiogenesis. Activation of the Responder construct would then occur by excision of the transcription Stop cassette due to the action of the Cre recombinase during embryonic development or in germ cells. Other combinations of recombinase and specific recognition sites for the recombinase, or the use of other nucleic acid molecules instead of a stop cassette, or the inverse orientation of the nucleic acid encoding the Responder flanked by sites for site specific recombinases in inverse orientation, are also envisaged ways to achieve a reversible inactive state of expressibility.

It is also envisaged to achieve transmission of (a) construct(s) which is not transmitted through sperm cells by propagation of said construct(s) in females. This is particularly useful when the construct(s) is/are integrated on the X chromosome and/or (an) autosome(s). It is furthermore envisaged that the construct(s) will only be activated in sperm cells, in particular if promoter(s) are used which activate transcription specifically during spermatogenesis and/or spermiogenesis. Thus, it is envisaged that selection against transmission of said construct(s) will be restricted to transmission through sperm cells, while transmission through the female germ cells occurs normally.

The use of constructs designed for selection against a genetic trait such as male sex is rendered in some rare cases difficult by the fact that the transgene construct may not or hardly be transmitted to the offspring of the carrier male animal. In such cases it is envisaged to use sperm cells at random or after preselection of cells carrying the transgene construct in order to significantly enhance the likelihood for the production of offspring carrying said transgene construct. Selection can be effected, e.g., by cell sorting.

It is also envisaged to make use of in vitro fertilization since it has been shown that transmission ratio distortion in mouse does not occur during in vitro fertilization procedures; other methods are ICSI (intracellular sperm cell injection), (Horiuch, Emuta et al. 2002).

Furthermore, the present invention relates to a non human male or female animal, preferably mammal, fish, bird or insect, wherein said non human male or female animal, preferably mammal, fish, bird or insect is transgenic for the nucleic acid molecule encoding an expression product with a Distorter function and/or the nucleic acid molecule encoding an expression product directed against the Distorter function and/or the nucleic acid molecule for inactivation of the Distorter function by homologous recombination as defined in the present invention and optionally for the nucleic acid molecule encoding an expression product with a Responder function.

The present invention also relates to a pair of non human male and female animals, preferably mammals, fish, birds or insects, wherein at least one of the male and/or female is a transgenic non human mammal, fish, bird or insect as defined in the present invention.

Preferably, the nucleic acid molecule or part thereof encoding an expression product with a Responder function and/or the nucleic acid molecule or part thereof encoding an expression product with a Distorter function and/or the nucleic acid molecule or part thereof encoding an expression product directed against the Distorter function and/or the nucleic acid molecule or part thereof for inactivation of the Distorter function by homologous recombination as defined in the present invention is/are flanked by recombinase recognition sites.

It is also preferred that one of the pair has (only) the Responder stably integrated into the germline whereas the partner of the pair has (only) integrated the Distorter into the germline. Upon crossing the offspring will carry both the Responder and the Distorter in the germline. In this manner, male offspring may be selected that is described in accordance with the main embodiments of the invention.

It is further preferred that the above pair of non-human male and female animal, preferably mammal, fish, bird or insect has further stably integrated into its genomic DNA a nucleic acid molecule encoding a site specific DNA recombinase.

In the specific cases with low or no transmission of the transgene construct designed for selection against a genetic trait it is envisaged to use constructs, which are in an inactive state and therefore not selected against under standard breeding conditions, but can be activated after expression of a site specific recombinase. Using this method the (inactive) transgene construct can be transmitted at Mendelian rates. After expression of a site specific recombinase (such as Cre) from an inducible construct or by breeding of the male or female carrying the Cre gene in an active state to the female or male carrying said inactive transgene construct offspring can be generated which carries said transgene construct in an active state allowing selection against the sperm cells carrying said transgene construct. The latter offspring could then be utilized for the production of animals, which do not carry the undesired genetic trait.

Several methods can be utilized to keep a transgene construct in an inactive state, the most common being the use of a transcription stop cassette and/or a reporter gene inserted between the promoter driving expression of the transgene construct and the open reading frame (ORF) of the gene kept inactive by this method. It is envisaged that the stop cassette and/or reporter is flanked by recognition sequences for the site specific recombinase in direct repeat orientation allowing deletion of the stop cassette and/or reporter upon recombination, and subsequent expression of the ORF made active by this recombination event.

It is more preferred that in the pair of non human male and female animal, preferably mammal, fish, bird or insect of the present invention said DNA recombinase is Cre, wherein said recognition sites are loxP sites, or flp, wherein said recognition sites are FRT sites, or φc31, wherein said recognition sites are att sites.

It is also more preferred that in the pair of transgenic non human male and female animals, preferably mammals, fish, birds or insects of the present invention said DNA recombinase is controlled by regulatory elements that are active prior to spermiogenesis.

The present invention also relates to sperm obtainable from a male of the transgenic non-human animal, preferably mammal, fish, bird or insect of the present invention.

The present invention further relates to the use of the sperm of the present invention for the production of offspring.

The present invention also relates to the use of the nucleic acid molecule encoding an expression product with a Distorter function as defined in the present invention, for the identification of chemicals or biological compounds able to trigger the (premature) activation or inhibition of the Responder/Distorter signalling cascade.

The term “Responder/Distorter signalling cascade” as used above refers to any G protein signalling cascade, wherein at least one of the G proteins or other proteins in the cascade confers Responder/Distorter function.

Such compounds could be applicable as potent contraceptive since it is envisaged that the activation or inhibition (repression) of said signaling cascade may affect the motility of sperm, due to rapid exhaustion of their energy reserve, and/or by inhibiting sperm movement and/or by affecting the ability of sperm to fertilize ovulated eggs.

It is envisaged that the identification of said chemical or biological compounds could be achieved by standard screening technology using the activity of the wild type Distorter protein expressed in vitro or in cell culture cells as an assay. It is e.g. known that GTPase-activating proteins such as Tagap1 enhance the GTPase activity of target GTPases rendering them inactive, or that GEFs such as Fgd2 or Tiam2 exchange GDP for GTP in said GTPases rendering them active. Assay systems for the activity of GAPs and GEFs and GTPases and other proteins involved in G protein signaling are well known in the art (Balch 1995); (Der 2000) allowing an artisan to screen for compounds triggering or inhibiting said proteins in vitro or in cell culture systems. It is envisaged that the compounds are then tested for their effects on sperm motility in vitro and on their effect in preventing fertilization of egg cells by sperm in vivo.

The present invention further relates to the use of the nucleic acid molecule encoding an expression product with a Distorter function as defined in the present invention for the isolation of receptor molecules and/or other members of the Responder/Distorter signaling cascade to which said expression product may bind.

Furthermore, the nucleic acid molecule as defined in the method of the present invention or the expression product as defined in the method of the present invention can be used for the isolation of receptor molecules and/or other members of the Responder/Distorter signaling cascade to which said expression product which would be expected to be a (poly)peptide may bind. Said signal transducing molecules are envisaged to be preferably identified by immunoprecipitation of protein complexes involving the Distorter (poly)peptide and cloning of the corresponding genes encoding them, or by Two Hybrid Screening techniques in yeast employing standard technology. In particular, most preferably the Distorter gene or (poly)peptide may be used to isolate the membrane receptor of the signaling molecule which is envisaged to activate said Responder/Distorter signaling cascade. Said membrane receptor is envisaged to be most preferable as a target for the development of novel contraceptives.

The present invention also relates to a method for the detection of a nucleic acid molecule encoding an expression product with a Distorter function and/or a nucleic acid molecule encoding an expression product directed against the Distorter function and/or a nucleic acid molecule for inactivation of the Distorter function by homologous recombination as defined in the present invention in a non human male or female animal, preferably mammal, fish, bird or insect as defined in the present invention comprising identifying said nucleic acid molecule encoding an expression product with a Distorter function and/or said nucleic acid molecule encoding an expression product directed against the Distorter function and/or said nucleic acid molecule for inactivation of the Distorter function by homologous recombination in said non human male or female animal, preferably mammal, fish, bird or insect by polymerase chain reaction (PCR), gene (micro)array hybridization, single nucleotide polymorphism (SNP) analysis, and/or sequencing with primers hybridizing to said nucleic acid molecule.

The present invention also relates to a nucleic acid molecule encoding an expression product with a Distorter function, wherein said expression product with a Distorter function is a factor involved in G protein signaling, selected from the group consisting of: (a) a nucleic acid molecule comprising or consisting of the nucleic acid molecule of any one of SEQ ID NOs: 3 to 6 and 12 or a fragment thereof; (b) a nucleic acid molecule being an allelic variant or a homologue or orthologue of the nucleic acid molecule of (a); (c) a nucleic acid molecule which hybridizes under stringent conditions to the nucleic acid molecule of (a), wherein said nucleic acid molecule encodes a polypeptide which has (i) at the position corresponding to position 49 of SEQ ID NO: 17 an I (ii) at the position corresponding to position 144 of SEQ ID NO: 17 an L (iii) at the position corresponding to position 323 of SEQ ID NO: 17 a T and (iv) which terminates after position 442; (d) a nucleic acid molecule which hybridizes under stringent conditions to the nucleic acid molecule of (a), wherein said nucleic acid molecule encodes a polypeptide which has (i) at the position corresponding to position 49 of SEQ ID NO: 17 an I; (ii) at the position corresponding to position 137 of SEQ ID NO: 17 an E; (iii) at the position corresponding to position 207 of SEQ ID NO: 17 an F; (iv) at the position corresponding to position 301 of SEQ ID NO: 17 an M; (v) at the position corresponding to position 323 of SEQ ID NO: 17 an T; (vi) at the position corresponding to position 332 of SEQ ID NO: 17 a D; (vii) at the position corresponding to position 407-413 of SEQ ID NO: 17 an internal deletion; (viii) at the position corresponding to position 440 of SEQ ID NO: 17 an M; (ix) at the position corresponding to position 471 of SEQ ID NO: 17 an L; (x) at the position corresponding to position 552 of SEQ ID NO: 17 an I; (xi) at the position corresponding to position 596 of SEQ ID NO: 17 a K; (xii) at the position corresponding to position 607 of SEQ ID NO: 17 an R; (xiii) at the position corresponding to position 610 of SEQ ID NO: 17 an S; and (xiv) at the position corresponding to position 703 of SEQ ID NO: 17 a V; (e) a nucleic acid molecule which hybridizes under stringent conditions to the nucleic acid molecule of (a), wherein said nucleic acid molecule encodes a polypeptide which has (i) at the position corresponding to position 49 of SEQ ID NO: 17 an I; (ii) at the position corresponding to position 54 of SEQ ID NO: 17 a G; (iii) at the position corresponding to position 137 of SEQ ID NO: 17 an E; (iv) at the position corresponding to position 173 of SEQ ID NO: 17 a G; (v) at the position corresponding to position 207 of SEQ ID NO: 17 an F; (vi) at the position corresponding to position 301 of SEQ ID NO: 17 an M; (vii) at the position corresponding to position 323 of SEQ ID NO: 17 a T; (viii) at the position corresponding to position 332 of SEQ ID NO: 17 a D; (ix) at the position corresponding to position 407-413 of SEQ ID NO: 17 an internal deletion; (x) at the position corresponding to position 440 of SEQ ID NO: 17 an M; (xi) at the position corresponding to position 471 of SEQ ID NO: 17 an L; (xii) at the position corresponding to position 508 of SEQ ID NO: 17 an S; (xiii) at the position corresponding to position 552 of SEQ ID NO: 17 an I; (xiv) at the position corresponding to position 596 of SEQ ID NO: 17 a K; (xv) at the position corresponding to position 607 of SEQ ID NO: 17 an R; (xvi) at the position corresponding to position 610 of SEQ ID NO: 17 an S; and (xvii) at the position corresponding to position 703 of SEQ ID NO: 17 a V; (f) a nucleic acid molecule which hybridizes under stringent conditions to the nucleic acid molecule of (a), wherein said nucleic acid molecule encodes a polypeptide which has (i) at the position corresponding to position 49 of SEQ ID NO: 17 an I; (ii) at the position corresponding to position 137 of SEQ ID NO: 17 an E; (iii) at the position corresponding to position 207 of SEQ ID NO: 17 an F; (iv) at the position corresponding to position 301 of SEQ ID NO: 17 an M; (v) at the position corresponding to position 323 of SEQ ID NO: 17 a T; (vi) at the position corresponding to position 332 of SEQ ID NO: 17 a D; (vii) at the position corresponding to position 407-413 of SEQ ID NO: 17 an internal deletion; (viii) at the position corresponding to position 440 of SEQ ID NO: 17 an M; (ix) at the position corresponding to position 471 of SEQ ID NO: 17 an L; (x) at the position corresponding to position 530 of SEQ ID NO: 17 an E; (xi) at the position corresponding to position 552 of SEQ ID NO: 17 an I; (xii) at the position corresponding to position 573 of SEQ ID NO: 17 an R; (xiii) at the position corresponding to position 596 of SEQ ID NO: 17 a K; (xiv) at the position corresponding to position 607 of SEQ ID NO: 17 an R; (xv) at the position corresponding to position 610 of SEQ ID NO: 17 an S; and (xvi) at the position corresponding to position 703 of SEQ ID NO: 17 a V.

The nucleic acid molecule of the invention in any case retains the Distorter function. It has preferably a minimal length of at least 200 or 300 nucleotides. Such a molecule may also be used for example as a specific probe for hybridization reactions and would comprise at least one of the mutations of any one of SEQ ID NOs: 3 to 6. It is however also preferred that the nucleic acid molecules of the invention be significantly larger such as at least 500 or 1000 nucleotides. The nucleic acid molecules or fragments thereof of the invention may be fused to flanking sequences. In any case, the nucleic acid molecules of the invention may have a length of up to 500 nucleotides, 1000 nucleotides, 2000 nucleotides, 5000 nucleotides, 10000 nucleotides and in particular cases even up to 100000 nucleotides. When integrated into larger genomic regions, the nucleotides of the invention may have chromosomal length.

Additionally, the invention encompasses oligonucleotides/primers of a length of at least 8 and up to preferably 50 nucleotides, that are part of the above identified sequences or hybridize to the complementary strand thereof wherein said oligonucleotides/primers contain the sequence of at least one codon coding for any of the above-identified specific amino acid positions (or a complementary sequence thereof).

It is preferred that the nucleic acid molecule of the present invention is a DNA molecule.

It is furthermore preferred that said expression product is an RNA or a (poly)peptide.

The deduction of the amino acid sequence from the nucleic acid sequence of the invention allows the conclusion that the polypeptide is the expression product that contributes to the Responder/Distorter phenotype. However, it is not excluded that the mRNA contributes to said Responder/Distorter phenotype. Also, it is envisaged in accordance with the present invention that in certain embodiments the expression level, stage of expression during spermatogenesis or the copy number of said gene results in or contributes to the Distorter phenotype. Therefore, in a preferred embodiment of the nucleic acid molecule of the invention said expression product is an RNA or a (poly)peptide.

The present invention also relates to a recombinant DNA molecule comprising the nucleic acid molecule as defined above and a regulatory region being capable of controlling expression of said nucleic acid molecule.

It is further preferred that said regulatory region is a naturally occurring region or a genetically engineered derivative thereof.

It is further preferred that said regulatory region comprises or is a promoter.

The present invention also relates to a vector comprising the recombinant DNA molecule of the present invention.

The vector of the invention may simply be used for propagation of the genetic elements comprised therein. Advantageously, it is an expression vector and/or a targeting vector. Expression vectors such as Pichia pastoris derived vectors or vectors derived from viruses such as CMV, SV40, baculovirus or retroviruses, vaccinia virus, adeno-associated virus, herpes viruses, or bovine papilloma virus, may be used for delivery of the recombinant DNA molecule or vector of the invention into targeted cell population. Methods which are well known to those skilled in the art can be used to construct recombinant viral vectors; see, for example, the techniques described in Sambrook, loc. cit. and Ausubel, loc. cit. Alternatively, the recombinant DNA molecules and vectors of the invention can be reconstituted into liposomes for delivery to target cells.

It is preferred that the vector of the present invention comprises a heterologous promoter.

It is preferred according to one further embodiment that said vector comprises a heterologous promoter.

Said heterologous promoter not naturally operatively linked with the nucleic acid contributing to the Distorter function may be used to determine a certain time point of the onset of Distorter expression. This time point may be the same or a different one that is set when the natural Distorter transcription unit is employed. For example, said heterologous promoter may also be active in the early or late haploid phase of spermatogenesis.

It is more preferred that said heterologous promoter is controlling gene expression in spermatogenesis and/or in spermiogenesis.

It is even more preferred that said the heterologous promoter is the testis promoter of c-kit, ACE, Tcr or Smok.

The present invention also relates to a host cell or organism transformed or transfected with the nucleic acid molecule of the present invention, the recombinant DNA molecule of the present invention or the vector of the present invention.

The host cell can be any prokaryotic or eukaryotic cell, such as a bacterial, insect, fungal, plant, animal or human cell. Prokaryotic host cells will usually only be employed for the propagation of the nucleic acid molecule of the invention and sometimes for the production of the expression product. Suitable mammalian, fish or bird cell lines are well known or can easily be determined by the person skilled in the art and comprise COS cells, Hela cells, primary embryonic cell lines etc.

The term “transfected or transformed” is used herein in its broadest possible sense and also refers to techniques such as electroporation, infection or particle bombardment.

The present invention furthermore relates to a method of recombinantly producing an expression product as defined for the nucleic acid of the present invention comprising the steps of culturing the host cell of the present invention under conditions to cause expression of the protein and recovering said protein from the culture.

The method of the invention is most advantageously carried out along conventional protocols which have been described, for example, in Sambrook, loc. cit.

The present invention also relates to an expression product encoded by the nucleic acid molecule of the present invention or obtainable by the above method of recombinantly producing an expression product.

In accordance with the invention, said expression product may either be an mRNA or a polypeptide. Said expression product is, in accordance with the present invention, involved in the Responder/Distorter phenotype and contributes to the phenomenon of transmission ratio distortion.

In particular, the expression products relating to a (poly)peptide are preferred. This embodiment therefore comprises the (poly)peptides as shown in any of the sequences listed as SEQ ID NOs: 19 to 22, referring to mouse Tagap1^{t1-t4}, respectively, and SEQ ID NO: 28, relating to mouse Fgd2.

The conditions and characteristics described in the description of the present invention for the nucleic acid molecules as used in connection with the method of the present invention are to be considered also applicable to the conditions and characteristics described in the description of the present invention for the nucleic acid molecules of the invention, and vice versa.

The present invention further relates to a method for the identification of a nucleic acid molecule encoding an expression product with a Distorter function, comprising the steps of (a) isolating a nucleic acid molecule encoding a candidate expression product with a Distorter function from the mouse t-complex by means of genomic localization, wherein said nucleic acid molecule is involved in G protein signalling; and (b) testing the nucleic acid molecule isolated in step (a) for a change of the transmission ratio of the Responder or of a genetic trait linked to a Responder in an experimental non human animal, wherein when said transmission ratio is enhanced or reduced, said nucleic acid molecule isolated in (a) is a nucleic acid molecule encoding an expression product with Distorter function.

The above method is exemplified, inter alia, in Example 4 for Fgd2. The methods described in detail in Example 4 provide an ideal example how a Distorter can be identified and verified in vivo by genetic testing of a null allele.

The present invention relates in addition to a method for the identification of an expression product of a nucleic acid molecule encoding a Distorter, comprising the steps of (a) isolating an expression product of a nucleic acid molecule encoding a candidate Distorter by means of protein-protein interaction with a known Distorter derived from the mouse t-complex; and (b) testing the nucleic acid molecule encoding said expression product isolated in (a) for change of the transmission ratio of the Responder or of a genetic trait linked to a Responder in an experimental non human animal, wherein when said transmission ratio is enhanced or reduced, said expression product isolated in (a) is an expression product with Distorter function.

It is preferred that in step (b) of the above identification methods, hypomorphic or hypermorphic alleles of said nucleic acid molecule are used for testing for change of the transmission ratio.

It is envisaged that (a) (poly)peptide(s) binding to a known Distorter (poly)peptide is/are identified by co-immunoprecipitation of protein complexes involving the Distorter (poly)peptide, or by affinity chromatography purification of a protein binding to said Distorter polypeptide or a part thereof, or by other methods allowing purification and analysis of protein complexes such as mass spectrometry, and subsequent cloning of the corresponding genes encoding the proteins binding to said Distorter (poly)peptide, or by Two Hybrid Screening techniques in yeast employing standard technology (Chien, Bartel et al. 1991).

Genetic testing in transgenic animals for the ability of the Distorter candidate identified by the methods described above to enhance or reduce the transmission ratio of the Responder can be performed using, for example, hypomorphic or amorphic or hypermorphic alleles of said Distorter candidate, which are constructed for example by introduction of (a) nucleic acid molecule(s) expressing a shRNA directed against said Distorter candidate, or by targeting the nuclear gene locus of said Distorter candidate thereby inactivating the gene function, or by introducing a construct expressing the wild type Distorter candidate thereby increasing the dosage of the expression products of said Distorter candidate.

The figures show:

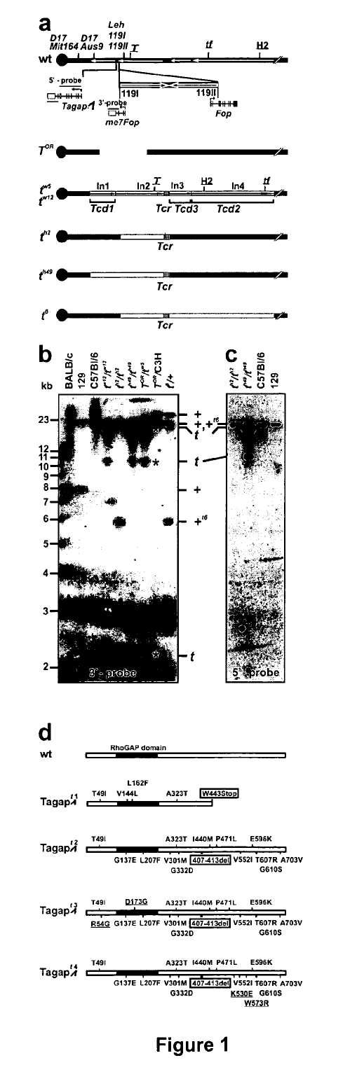

FIG. 1 Tagap1 is a candidate for Tcd1. a, Schematic map of the t-complex on chromosome 17. The approximate positions of Tagap1, Fop and the fusion gene me7Fop are indicated, gene maps are expanded. The centromere is indicated by a filled circle, wild-type chromatin by filled bars, t-chromatin by open bars, inversions (In1-In4) by arrows. The molecular and genetic markers, and the structure of partial t-haplotypes have been described previously (Lyon 1984). The approximate extent of the deletion in T^{OR} is indicated by a gap, positions of Tcds by brackets, Tcr by a hatched box. Maps are not to scale. b, Genomic mapping of me7Fop and Tagap1 to the Tcd1 region in t-haplotypes. The 3′-probe of me7Fop detects two wild-type bands, which are polymorphic in different strains and in t⁶, and three t-specific bands, which are absent from T^{OR} and not observed in t⁶, localizing both genes to the Tcd1 interval. c, Tagap1 maps to the Tcd1 region and is amplified in t-haplotypes. A Tagap1-specific 5′-probe detects two polymorphic fragments in t^{h49}/t^{h49} DNA equaling four-fold the signal intensity of the wild-type band, as determined by quantification. d, t-haplotypes encode four different classes of Tagap1 transcript. Schematic representation of representative cDNA clones isolated from testis of t^{h49}/t^{h49} mutant animals, in comparison to the wild-type gene. Mutations resulting in deletion of amino acid residues are boxed, mutations distinguishing products derived from different Tagap1 loci are underlined.

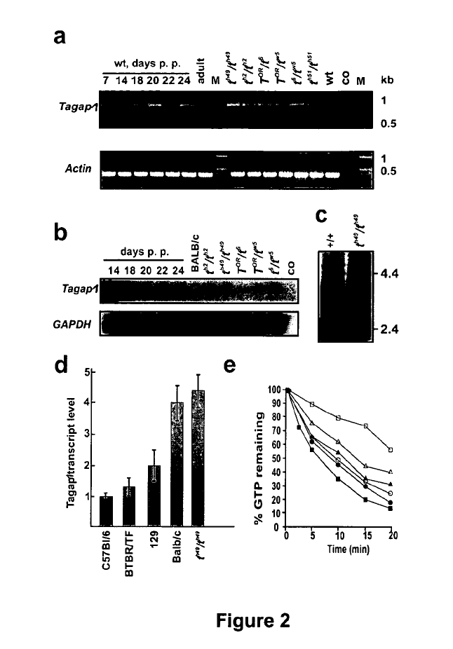

FIG. 2 Expression analysis and GAP-activity assays of Tagap1. a, Tagap1 is already expressed at early stages of spermatogenesis. RT-PCR analysis of RNA isolated from various postnatal (p.p.) testes, and from testes of adult wild type mice or males carrying various t-haplotypes; Actin served as control. b, RNAse protection assay confirming the RT-PCR data. c, Northern blot analysis of 8 μg poly(A+)RNA hybridized with the Tagap1-specific 5′-probe. The t-specific mRNA migrates faster than the wild type. d, Analysis of Tagap1 transcripts by quantitative PCR reveals up to four-fold higher levels of Tagap1 transcript in t^{h49} as compared to wild type strains. e, Tagap1 enhances the GTPase activity of RhoA. Squares, RhoA; triangles, cdc42; circles, Rac1; open symbols, reaction carried out without Tagap1; filled symbols, with Tagap1. Abbr.: +, wild type; p.p., days post-partum.

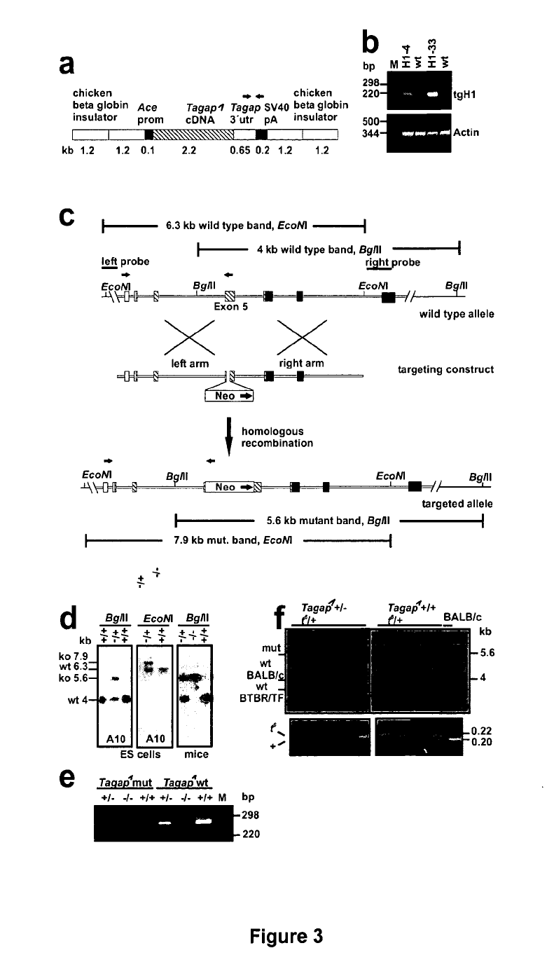

FIG. 3 Construction of gain- and loss-of-function alleles of Tagap1. a, Transgenic construct used for over-expression of wild type Tagap1. Black arrows indicate primers for genotyping. b, RT-PCR analysis of testis RNA verifying expression of the transgenic constructs. Actin served as positive control. Abbr.: H1-4, Tg(Tagap1)H1-4Bgh; H1-33, Tg(Tagap1)H1-33Bgh. c, Targeting of the Tagap1 gene. Introns are depicted as double lines, exons as boxes; coding regions are hatched, GAP-domain encoding regions filled. A PGKneo selection cassette was integrated into exon 5. Primers used for expression analysis in (e) are indicated by black arrows. d, Identification of the targeted allele Tagap1^{tm3Bgh} in clone A10. Genomic Southern blot analysis identifies the predicted size BgIII and EcoNI fragments, detected with the right and left probe, respectively, in the ES-cell clone A10. Right panel: genotyping of a heterozygous and a homozygous mutant male, confirming germ line transmission of the mutant allele. e, Genotyping of males used for testing the effect of the Tagap1 knock-out allele on the transmission ratio of the t⁶-haplotype. f, RT-PCR analysis of testis RNA from wild type (+/+), heterozygous (+/−) and homozygous (−/−) mutant animals with primers specific for the mutated (left part) and wild type allele (right part), demonstrating loss of the wild-type Tagap1 transcript in −/− animals.

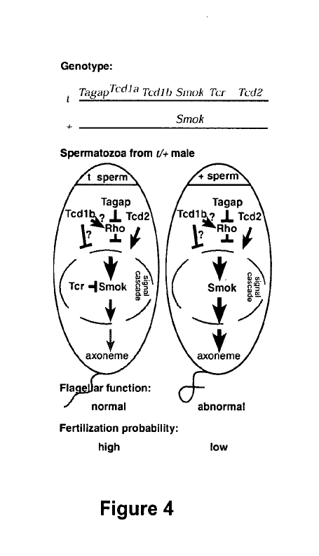

FIG. 4 Model of the role of Tcds and Tcr in transmission ratio distortion. t-haplotypes encode several Tcds (Tcd1^{Tagap1}, Tcd1b, Tcd2 are indicated, wild type alleles not shown) and Tcr acting upstream of Smok kinase controlling flagellar behaviour. Tagap1 is a negative regulator of a Rho family member, which inhibits Smok. Tagap1^{Tcd1a} (Tagap1) enhances down-regulation of Rho, resulting in up-regulation of Smok. Tcd1b is a hypomorphic or amorphic allele of an activator of Rho or of an inhibitor of Smok, further enhancing Smok activity epistatically to Tagap1. Likewise, Tcd2 further promotes up-regulation of Smok. All sperm (t and +) produced by t/+males are affected by Tcds, which act in trans. This negative effect of Tcds is counter-balanced by Tcr, which is restricted to t-sperm and thus rescues t-sperm only. This results in an advantage of t-sperm in fertilizing the eggs and promotes the transmission of the t-haplotype to the offspring. For details see text.

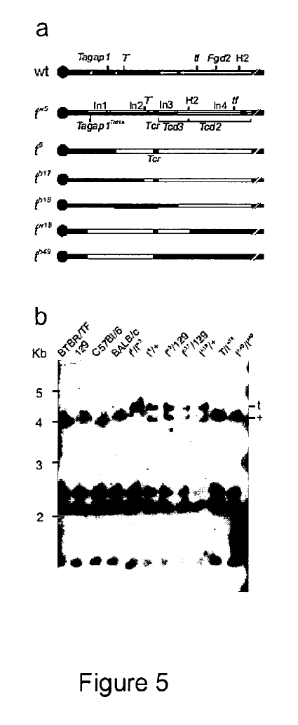

FIG. 5 Fgd2 maps to the Tcd2 region. a, Structure of a complete (t^{w5}) and various partial t haplotypes (t^{x}) used for mapping of Fgd2. The Tcd2 region is defined as the segment of t chromatin, which is present in t^{h18} and exchanged for wild type chromatin in t^{w18}. The centromere is shown as filled circle at the left, wild type chromatin is symbolized by filled bars, t chromatin by open bars and inversions (In1-In4) by arrows. Markers and t haplotypes have been described (Lyon 1984). wt, wild type. b, Southern blot analysis of genomic DNA digested with PstI, using a full length Fgd2 cDNA clone as probe, reveals a t-specific band of 4.5 kb, which occurs in t haplotypes carrying Tcd2, but not in t^{w18}, thus mapping Fgd2 to the Tcd2 region.

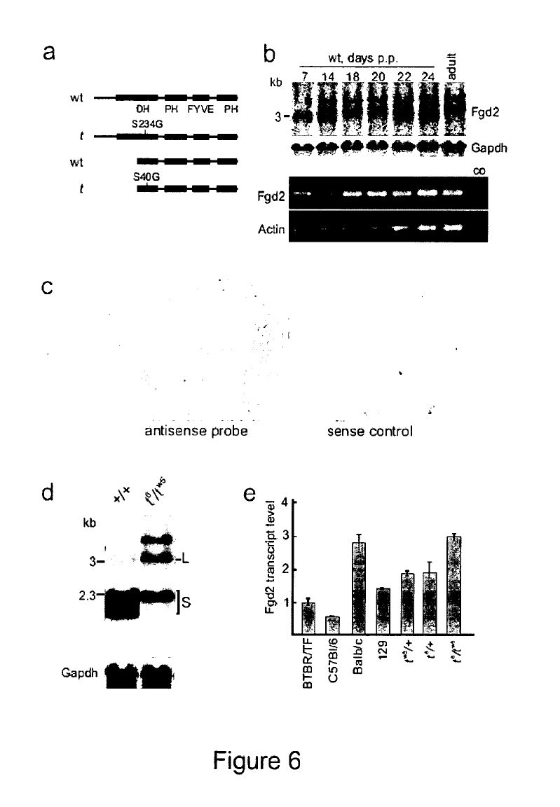

FIG. 6 Fgd2 expression from the t haplotype allele is strongly enhanced as compared to wild type alleles. a, Domain structure of Fgd2 proteins encoded by the long and the short transcript variants derived from this gene. The mutation S40G in transcript variant 2 is equivalent to the S234G mutation in transcript variant 1. b, Temporal expression profile of the long Fgd2 transcript in postnatal testes, representing the first cycle of spermatogenesis. Top panels: Northern blot, lower panels: RT-PCR-analysis. p.p., post partum. c, In situ hybridization of Fgd2 antisense or sense control transcripts to testis cryo-sections showing expression of Fgd2 in meiotic spermatocytes and round spermatids. d, Northern blot analysis of Fgd2 expression in testis derived from wild type (+/+) and t⁶/t^{w5} males demonstrates strongly enhanced expression of the long (L) and simultaneous strongly reduced expression of the short (S) Fgd2 transcript in t haplotypes. e, Quantitative RT-PCR analysis of the long Fgd2 testis transcript in various strains and t haplotypes, demonstrating up to 6-fold higher expression of Fgd2 in t haplotypes compared to wild type strains, which show considerable differences. +, wild type strain BTBR/TF; Gapdh, Gapdh loading control.

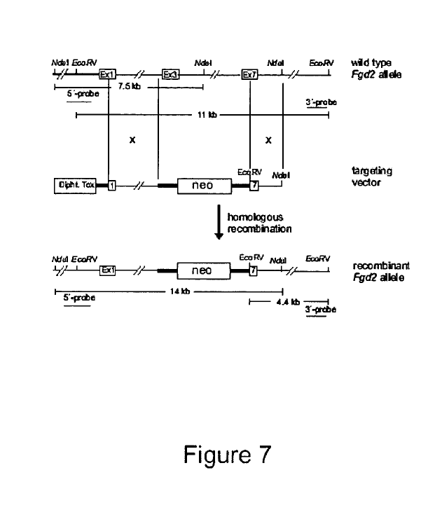

FIG. 7 Targeting of the mouse Fgd2 gene by homologous recombination. The targeting vector was constructed by ligation of the left and right homology arm, both derived by PCR amplification of genomic DNA, to the vector pDT/pGKneoflox 3xpA (see Methods). Out of 132 clones analyzed, 1 displayed the expected RFLP with the 5′-probe (7.5 kb NdeI fragment for the wild type allele and 14 kb for the targeted allele) and 3′-probe (11 kb EcoRV versus 4.4 kb for the wild type allele and mutant allele respectively) demonstrating successful targeting of the genomic locus by homologous recombination.

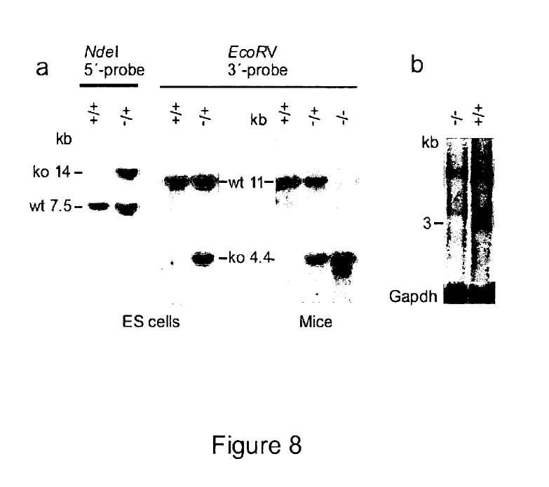

FIG. 8 Gene targeting of Fgd2 by homologous recombination. a, Southern blot analysis of DNA derived from targeted and control ES cells (left panel), and of mice (right panel) carrying the mutant allele (−), with the 5′- and 3′-probes. b, Northern blot analysis of testis RNA derived from wild type and mutant animals. The long Fgd2 transcript is not detected in homozygous mutant animals. Gapdh, Gapdh loading control; ko, band derived from targeted allele; wt, wild type fragment; +, wild type locus; −, mutant locus.

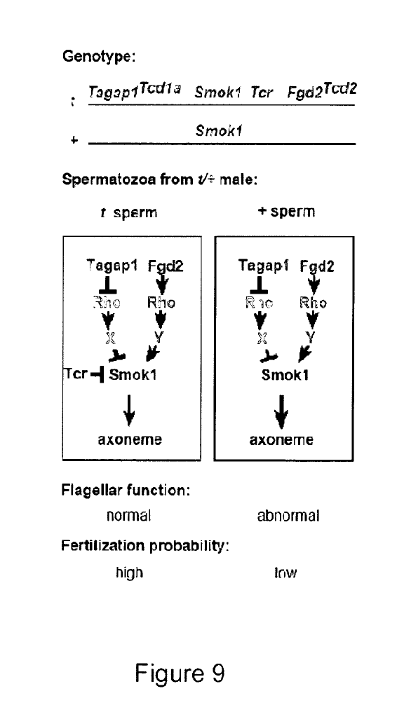

FIG. 9 Model of transmission ratio distortion. The t haplotype encodes several Distorters. Only two are shown for clarity, which are expressed in all sperm cells derived from a V+male and act on two opposing Rho signalling pathways regulating Smok1. Smok1 is thought to be involved in sperm motility control. Tagap1^{Tcd1a} and Fgd2^{Tcd2} represent hypermorphic alleles expressing strongly elevated gene activity as compared to the wild type. Enhanced down-regulation of the inhibitory pathway by Tagap1 and stronger up-regulation of the activating pathway by Fgd2 additively induce hyper-activation of Smok1 in all sperm, resulting in abnormal flagellar function and low fertilization probability. This harmful effect of the Distorters is rescued by the dominant-negative action of Tcr, which is restricted to t sperm, giving the latter an advantage in fertilizing the egg cells. Neither the Rho switch molecules nor their target effector proteins (X, Y) are known. Arrows symbolize activation, bars inhibition; green arrow, normal signalling; purple arrow, impaired signalling.

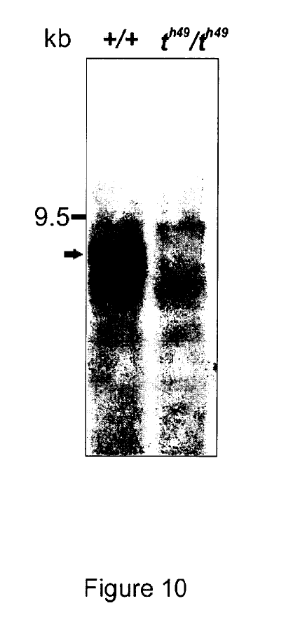

FIG. 10 Northern blot analysis of 8 μg poly(A+)RNA hybridized with a Tiam2 specific probe. The Tiam2 specific band, observed in wild type testis RNA is not detected in t^{h49}/t^{h49}, indicating, that the gene is not expressed in the t-haplotype.

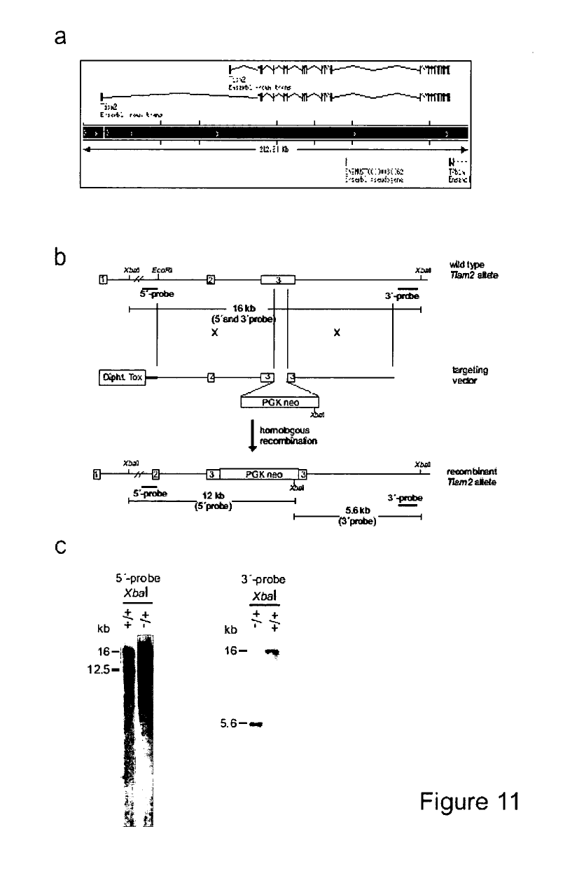

FIG. 11 Genomic Structure of Tiam2 and generation of a loss-of-function allele in embryonic stem cells. a, Transcripts annotated by the ensembl genome server (http://www.ensembl.org). b, Targeting of the Tiam2 gene. Introns are depicted as lines, exons as boxes. The targeting vector was constructed by ligation of the left and right homology arm, both derived by PCR amplification of genomic DNA, to the vector pDT/pGKnoflox 3xpA. The targeting event resulted in the integration of the PGK neo selection cassette into exon 3, thereby interrupting the open reading frame of the gene. c, Identification of the targeted Tiam2 allele. Genomic southern blot analyses identify XbaI fragments of the predicted size with the left (5′-) and right (3′-) probe respectively in ES cells.

The examples illustrate the invention.

### Material and Methods

### Mice and Genetics

### Tagap1

For mapping of Tagap1 on genomic DNA the following t-haplotypes were used: t^{w12}/t^{w12} t^{h2}/t^{h2}, t^{h49}/t^{h49}, t^{h51}/t^{h51}, T^{OR}/t^{w5}, T^{OR}/t⁶, t⁶/+. The t-haplotypes t^{w12}, t^{h49}, t^{h51} and t^{w5} carry Tcd1, while t⁶ and t^{h2}, which is derived from t⁶, lack Tcd1 activity. Southern blot analysis of KpnI digested genomic DNA was performed by standard procedures (Church and Gilbert 1984) using a fragment corresponding to position 942-3001 of the Tagap1 cDNA as 3′-probe, and position 124-942 as 5′-probe. Genotyping of mice for the transmission of t⁶ was done by PCR using primers for the marker Hba-4ps. Transgenic lines were generated in the inbred strain FVB/N by pronuclear injection of construct DNA using standard procedures. The Tagap1^{tm3Bgh} allele was generated in Balb/c ES-cells obtained by B. Ledermann (Basel Institute for Immunology) (Dinkel, Aicher et al. 1999). Also other ES cells known to the person skilled in the art, however, can be used. Transgenic and knock-out lines were back crossed several generations to the strain BTBR/TF-+tf/+tf before testing for Distorter activity.

### Fgd2

Fgd2 was mapped by Southern blot analysis of PstI digested genomic DNA derived from various complete and partial t haplotypes obtained by rare recombination between wild type and t haplotype chromosomes (see FIG. 5a), using a cDNA of Fgd2 as probe (position 170-2576 in Acc. Nr. AF017368, SEQ ID NO: 9). We derived an ES-cell line from the strain BTBR/TF-+tf/+tf and generated the targeted allele Fgd2^{tm4Bgh} by standard procedures (FIG. 8 a, b). A heterozygous Fgd2^{tm4Bgh}/+female was mated to a t^{h49}/t^{h49} male to generate male litter mates of the genotypes +/+; t^{h49}/+ and +/Fgd2^{tm4Bgh}; t^{h49}/+ for testing the effect of the targeted allele on the transmission ratio of the t haplotype (table 1). We genotyped mice for t^{h49} by Southern blot analysis of KpnI (or alternatively BamHI) digested genomic DNA using the 3′-fragment of Tagap1 as probe as described.

### Transcript Analysis

### Tagap1

The 5′ ends of me7Fop and Tagap1 were obtained by 5′-RACE using the GeneRacer kit (Invitrogen). Standard reverse transcription was performed with 1 microgram of total RNA using AMV-RT (Promega). RNAse protection assays were done using standard procedures (Gilman 1997). The Tagap1 probe was synthesized in vitro in the presence of [³²P] UTP from a fragment derived from the Tagap1 cDNA by PCR (sense 5′-GACTCCTAGGGTCAGAGTGTCATG-3′, antisense 5′-TGGGCTCCACATCTGGGTCATT-3′) cloned in pCRII TOPO (Invitrogen). The GAPDH control RNA was transcribed from the pTRI-GAPDH template (Ambion). Northern analysis was performed by standard techniques (Sambrook J. 1989) using 8 μg of single purified poly(A+)RNA using the Fast Track system (Invitrogen). Quantitative PCR analysis was carried out on an ABI PRISM 7900 HT SDS (Applied Biosystems) using the TaqMan probe 5′-ATCCTCTGCCTTAAAGGTCCTTCAACGGAA-3′ (5′ FAM and 3′ TAMRA labeled) and primers sense 5′-CCAGACCCATCCAGGACATC-3′ and antisense 5′-CTGGCAGCTTTCCTGAATATC-3′. As a reference, GAPDH expression was determined using the mouse GAPDH assay (Applied Biosystems).

### Fgd2

A plasmid cDNA library from testis RNA derived from a t⁶/t^{w5} male using the SuperScript plasmid cDNA cloning system (Life Technologies) was constructed and screened by colony filter hybridization using Fgd2 derived cDNAs as probes. We also obtained cDNAs encompassing the full coding sequence from t haplotypes and wild type by RT-PCR. We sequenced clones from both sources and analyzed the results using the Lasergene DNA Star package. We isolated total RNA using Trizol (Invitrogen). For quantitative real time PCR we used an ABI PRISM 7900 HT SDS (Applied Biosystems). As a reference gene, we analyzed Gapdh expression with the mouse GAPDH assay (Applied Biosystems). We isolated polyA+ RNA using the Fast Track system (Invitrogen) and performed northern blot analysis using the Ambion GlyMAX Northern kit. In situ hybridization on 10 μm cryostat sections was essentially performed as described (Brent, Schweitzer et al. 2003). We produced riboprobes by in vitro transcription from the TOPO pCRII vector containing a Fgd2 cDNA fragment obtained by PCR amplification. Digoxygenin-labeled probes were detected by phosphatase reaction of the substrate NBT/BCIP (Sigma).

### Gene Targeting and Transgene Constructs

### Tagap1

The Tagap1 targeting vector was constructed by ligation of the left and right arm, both derived by PCR amplification of genomic DNA, to the PGK-neo cassette. Culture, electroporation, selection, isolation of ES-cell clones, DNA preparation in 96 well plates and Southern blot analysis were done according to standard procedures (Ramirez-Solis, Davis et al. 1993). The transgenic construct Tg(Tagap1)H1Bgh consists of the Angiotensin Converting Enzyme (ACE) testis promoter and transcription start (extending from −91 to +17 bp), driving expression in elongating spermatids (Morita, Murata et al. 1993), followed by the complete ORF of wild-type Tagap1, its 3′UTR and the SV40 polyadenylation signal derived from the vector pCS2+ (Rupp, Snider et al. 1994), replacing the Tagap1 polyA-signal sequence. This transcription unit is flanked by tandem copies of the chicken-globin insulator (Chung, Whiteley et al. 1993). Transgenic animals were identified by PCR using the primers: sense 5′-AGGGCCCTTGGGGTCAGG-3′, antisense 1 5′-CTGTCAGTCTCCATTCCAATGAAG-3′ and antisense 2: 5′-CAGTTAGCTGGCAAATGCTGTC-3′. The wild-type band is 541 bp in length, the transgenic construct produces bands of 165 bp and 266 bp.

For gene targeting of the Fgd2 locus, a fragment containing the PGK-promoter driven neomycin resistance gene flanked by loxP sites was isolated from the vector pPGKneo FloxI (gift of Moises Mallo) by EcoRV/EcoRI digest and ligated into the EcoRV/EcoRI digested pDT Bluescript vector (provided by Achim Gossler), which contains the diphtheria toxin-A chain coding sequence under the control of RNA polymerase II promoter. pDT/pGKneoflox 3xpA was digested with NotI, filled in, cut XbaI and ligated to the left homology region, which was obtained by PCR amplification of genomic DNA using primers s: 5′-ACTAGTCTGCTTCTGGGGTAACT-3′ containing a SpeI site and as: 5′-ATAGGCCTGCTCCGTCT-3′ followed by digestion with SpeI. The obtained construct was digested EcoRV/SalI and the right homology region, obtained by PCR on genomic DNA with primers s (EcoRV): 5′-GATATCAAGAATCCCGCGGTACGAACTG-3′ and as (SalI): 5′-GTCGACGACAACGCCCGACATCATAGAG-3′ and cut with EcoRV and SalI was ligated into this vector. The resulting targeting vector was linearized by restriction digest with SalI. Establishment of a BTBR/TF-ES cell line, culture, electroporation, selection, isolation of ES-cell clones, DNA preparation in 96 well plates and Southern blot analysis was done according to standard procedures. The left probe (LP) and right probe (RP) for Southern blot analysis were generated by PCR with primers LPs 5′-ACAGGTCTCACGTAGCCGAATC-3′, LPas 5′-CGGGTGAAGCAGGTCTACCACA-3′ and RPs 5′-TGGATGCCGCTCAGTTGCTAAT-3′, RPas 5′-TGAAACTCAGTGTGTAGACCAG-3′ respectively.

### Fgd2

We isolated the left (3.9 kb) and right (2.6 kb) homology regions by PCR-amplification of genomic DNA derived from the strain BTBR/TF-+tf/+tf. Using restriction sites included in the oligonucleotides we ligated the homology regions to either side of a PGK-promoter/neo resistance gene/triple-pA cassette inserted in a pBluescript vector containing the diphtheria toxin-A chain gene (kindly provided by Achim Gossler), which thereby flanked the left homology region. An EcoRV restriction site creating a RFLP for genotyping of the targeted allele was introduced by ligation of the right homology region to the selection cassette. We linearized the resulting targeting vector with SalI. We established a BTBR/TF ES cell line, electroporated the targeting construct, selected and isolated ES cell clones, prepared DNA in 96-well plates, and performed Southern blot analysis according to standard procedures, using 5′- and 3′-probes obtained by PCR amplification of genomic DNA. The targeting event replaced exons 3 to 6 and part of exon 7 by the neo selection cassette, which also removed a genomic NdeI site, creating another RFLP for genotyping. We verified correct targeting of the locus and genotyped mice using EcoRV digested DNA hybridized with the 3′-probe, which detects an 11 kb fragment derived from the wild type and a 4.4 kb fragment derived from the targeted Fgd2 allele. The 5′-probe detects a 7.5 kb fragment in NdeI digested DNA derived from the wild type and a 14 kb fragment derived from the mutant Fgd2 allele. Out of 132 clones analyzed, 1 displayed the expected mutant fragments with both probes demonstrating successful targeting of the Fgd2 locus by homologous recombination.

Oligonucleotide sequences and PCR conditions for PCR experiments concerning the characterization of Fgd2 are listed in table 4.

### In Vitro GAP Assays

The catalytic domain of wild type Tagap1, small G proteins and the C-terminal polypeptide of Tagap1, the latter serving as negative control, were produced as GST-fusion proteins in E. coli BL21 using the pGEX vectors as described (Frangioni and Neel 1993)(Self and Hall 1995). For quantification of relative amount of proteins used, all preparations were adjusted relative to a BSA standard. GAP assays were performed in triplicate (G proteins at 6 nM; Tagap1: 15 nM) essentially as published (Self and Hall 1995).

### EXAMPLE 1

### Isolation of a Candidate Gene for Tcd1

We have used a positional cloning approach to identify a candidate for Tcd1, based on the following criteria: 1) The gene must be located in the genomic interval comprising Tcd1, 2) it must be expressed in testis, 3) show alterations in the t-haplotype form vs. the wild type, and 4) should encode a protein involved in signalling. The latter criterion was based on our proposal that Tcds encode components of signalling cascades acting upstream of Smok (Herrmann, Koschorz et al. 1999).

Since chromosomal rearrangements have a high potential of affecting gene function we started our search for Tcd candidates in the region D17Leh1191I, which marks the end of a large inverted duplication in the wild type chromosome (Herrmann, Barlow et al. 1987). Genomic fragments, spanning the duplication breakpoint were hybridised to a cDNA library and a gene, designated me7Fop, showing similarity with FGF receptor oncogene partner (Fop) was identified (FIG. 1a). Northern analysis showed that this gene is highly expressed in wild type testis, whereas no transcripts are detectable in testes from males carrying the Tcd1 region in the t-haplotype form (e.g. t^{h51}, t^{h49} or complete t-haplotypes). A detailed analysis of the gene structure showed that the 5′-region of me7Fop is derived from Fop, while most of the coding region and the 3′-untranslated sequence come from an unrelated gene.

The part of me7Fop, which is not derived from Fop occurs in a second locus on the wild type chromosome. This is shown by genomic mapping using the 3′-region of me7Fop as probe (FIG. 1b). In wild type genomes two bands are detectable, whereas t-haplotypes produce three polymorphic t-specific bands. Both wild type fragments are missing in the deletion chromosome T^{OR} since genomic DNA from a T^{OR}/t^{w5} animal shows only the t-specific bands. This data maps both gene fragments to the proximal t-haplotype.

The analysis of the partial t-haplotype t⁶ allows a more accurate assignment of both genes to the region harbouring Tcd1. The t-haplotype t⁶ and its derivative t^{h2} have lost Tcd1 activity by a recombination event in which the proximal portion of the t-haplotype has been exchanged for wild type DNA (Lyon 1984). Thus Tcd1 is located in the region, which is wild type in t⁶. The Southern blot analysis revealed no t-specific band in t⁶ or t^{h2}, but a wild type and a t⁶-specific band. Accordingly, both gene fragments map to the Tcd1 region (FIG. 1a). We mutated me7Fop by gene targeting and analysed its possible role as Distorter of the transmission ratio. Distorter activity was not observed excluding me7Fop as candidate for Tcd1.

We extended the analysis to the gene from which me7Fop was derived. 5′-RACE protocols and database searches were utilized to obtain a complete cDNA clone. Sequence analysis showed that it encodes a protein of 714 amino acid residues involving a domain with high similarity to GTPase-activating proteins (GAP) for Rho small G proteins (FIG. 1d).

In the course of our studies this gene appeared in public databases as T-cell activation Rho GTPase-activating protein (Tagap1, accession number NM_{—}145968). Genomic Southern blot analysis using a Tagap1-specific 5′-fragment of the cDNA as probe showed that wild type strains contain a single band, which is also present in t⁶/+ DNA. In contrast, genomic DNA derived from t-haplotypes showed two stronger polymorphic bands (FIG. 1c). This polymorphism confirms the mapping of Tagap1 to the Tcd1 region. Quantification of the wild-type and the t-specific signals revealed that the Tcd1-bearing t-haplotypes harbour four Tagap1 loci, while the wild-type genome contains a single complete Tagap1 gene. This result was confirmed by quantitative PCR on genomic DNA using Tagap1 specific primers. Hybridisation with the 3′-probe, which detects me7Fop and Tagap1, and quantification of the bands revealed two-fold higher signal intensity in genomic DNA from t^{h49}/t^{h49} mice compared to t^{h2}/t^{h2} mice or wild type strains.

In accordance with multiple genomic copies of Tagap1 on the t-haplotype, sequencing of Tagap1 cDNAs derived from testis of t^{h49}/t^{h49} males revealed several types of transcripts with multiple non-silent nucleotide changes compared to the wild type sequence (FIG. 1d). The major cDNA class (51 out of 74 clones analysed), derived from Tagap1^{t1} contains a transition of G to A at codon 433, turning a tryptophane (TGG) into a premature stop codon (TGA). This mutation truncates the predicted protein, leaving the N-terminal RhoGAP domain intact. Two additional mutations (V144L, L162F) were found in the RhoGAP domain of Tagap1^{t1}, and two (T49I, A323T) outside of this region. None of these alterations were found in transcripts derived from wild-type strains, nor in the partial t-haplotypes lacking Tcd1, in t⁶ and t^{h2}.

The remaining three t-specific Tagap1 cDNAs do not contain the W443X mutation. Instead, a number of non-silent point mutations and a 21 base pair deletion 3′ to the RhoGAP domain were detected in these clones (FIG. 1d). Two alterations in the RhoGAP domain (G137E, L270F) are shared by all three genes, while Tagap1^{t3} differs from Tagap1^{t2} and Tagap1^{t4} by one additional mutation (D173G) in this domain. Aside from shared alterations, these cDNAs also show differences distinguishing them from each other, suggesting that they are derived from three distinct Tagap1 genes, which arose by triplication of a single locus.

The combined genomic and cDNA sequence data demonstrate that t-haplotypes contain four Tagap1 loci in the Tcd1 region, while the wild type has two, one complete (Tagap1) and one altered gene (me7Fop), which has lost the GAP-domain due to a rearrangement. The fact that the t-alleles of Tagap1 are altered with respect to the wild type is consistent with the criteria for a Tcd candidate.

RNA expression analysis by RT-PCR and RNAse protection assays showed that Tagap1 is transcribed in the testis already at the earliest stage analysed, day 7 post partum (FIG. 2 a,b). Thus Tagap1 is expressed already in diploid spermatocytes, which may be conducive to distribution of the gene products to all sperm cells, since spermatids develop in a syncytium. Northern blot analysis using poly(A+) RNA suggested low level transcription in this organ (FIG. 2c). In situ hybridisation analysis on testis sections using a Tagap1 specific probe did not produce distinct signals.

The transcript detected in the t-haplotype shows a slightly faster migration compared to the wild type mRNA (FIG. 2c). The reason for this is unclear, as no major size differences (except for the 21-base deletion) were observed in any of the t-specific cDNA clones analysed. It is conceivable that poly-adenylation differences may account for the smaller transcript size. Shortening of the poly-A tail has been shown to accompany translational activation of some mRNAs during spermiogenesis (Kleene 1989). Whether or not the observed difference has a functional relevance, however, remains to be determined.

Tagap1 transcripts were detected in all organs examined by RT-PCR analysis, and Tagap1 ESTs have been reported in public databases from a large variety of tissues and organs, suggesting that the gene is ubiquitously expressed.

Quantitative RT-PCR showed that t-haplotypes express up to four fold higher levels of Tagap1 transcripts than wild-type strains (FIG. 2d). This finding suggests that sperm derived from t/+ males on different wild type backgrounds may produce substantially different levels of Tagap1 protein. This may have a profound effect on the transmission ratio of the t-haplotype, consistent with earlier reports that the genetic background has an important impact in transmission ratio distortion (Gummere, McCormick et al. 1986).

Finally we examined the specificity of the GAP-domain of wild type Tagap1 towards three “classical” small GTPases, RhoA, cdc42 and Rac1, which are, among several others, expressed in testis (Wennerberg and Der 2004). G proteins act as molecular switches, which transmit a signal in their active, GTP-bound form, whereas they become inactive after GTP hydrolysis. GAPs enhance the intrinsic GTPase activity of small G proteins, promoting their inactive state. Our data show that the GTPase activity of RhoA was strongly enhanced by the GAP domain of Tagap1, whereas the other family members were only mildly (Cdc42) or hardly (Rac1) stimulated, identifying RhoA as a possible in vivo target of Tagap1 (FIG. 2e).

### EXAMPLE 2

### Tagap1 Distorts the Transmission Ratio of t-Haplotypes

Since the genetic and molecular data suggested that the t-loci of Tagap1 might cause a gain-of-function phenotype, we tested whether over-expression of wild-type Tagap1 in elongating spermatids, from a transgene construct controlled by the testis-specific ACE promoter, would alter the transmission ratio of the partial t-haplotype t⁶ lacking Tcd1 (FIG. 1a, 3a) (Howard, Balogh et al. 1993). Two independent transgenic lines harbouring the construct and expressing the transcript were generated, crossed into males carrying t⁶ and analysed (FIG. 3b, Table 1). In both lines, a significant increase of the transmission of the t-haplotype to the offspring was observed, as compared to non-transgenic lifter mates (combined data: 88% vs. 80% t⁶ offspring, p<0.01). Thus, a dosage increase of wild type Tagap1 in testis phenocopied a t-complex Distorter, consistent with the idea that the t-loci of Tagap1 encode Tcd1 activity.

To create a loss-of-function allele of Tagap1, we disrupted the gene by inserting a selection cassette into exon 5, resulting in premature termination of the transcript (FIG. 3c,d,f). Accordingly, the translation product is predicted to be truncated upstream of the RhoGAP domain. This allele, termed Tagap^{tm3Bgh} (in accordance with standard nomenclature) was bred in trans to the partial t-haplotype t⁶. Litter mates carrying either the wild type allele or Tagap^{tm3Bgh} in trans to t⁶ were tested for transmission ratio distortion (FIG. 3e, Table 1). Complementary to the transgenic experiments, the transmission ratio of t⁶ from Tagap^{tm3Bgh}/+; t⁶/+ males was strongly reduced compared to the ratio obtained from Tagap1 +/+; t⁶/+ litter mates (69% vs. 84% t⁶ offspring; Table 1). Statistical analysis demonstrated that the difference is highly significant (p<0.001). Thus, the loss of function experiment confirmed the results of the gain-of-function experiment, directly demonstrating a role of Tagap1 in transmission ratio distortion.

Taken together, the genetic and molecular data strongly suggest that the t-haplotype loci of Tagap1 represent Tcd1. From the transgenic gain-of-function phenotype one would conclude that these loci act as dominant gain-of-function mutation. Consistent with this conclusion is the fact the Tagap1 gene is amplified in t-haplotypes and that the four loci together express up to fourfold more transcript in testis than wild type strains. The inactivation (in terms of GAP activity) of one of the originally two Tagap1 genes by a chromosomal rearrangement in the wild type may have had a selective advantage over the progenitor chromosome since it decreased the overall Tagap1 activity in t/+ heterozygotes, possibly “defending” the wild type chromosome better against the disadvantageous hyperactivity caused by the t-specific Tagap1 loci.

It has been shown that a large deletion of the wild type chromosome, T^{22H}, phenocopies Tcd1, which led to the suggestion that Tcd1 represents a hypomorphic or amorphic allele (Lyon 1992). In contrast, we demonstrated that a hypermorphic allele of Tagap1 phenocopies a t-complex Distorter located in the Tcd1 region. This discrepancy could be reconciled by recent data suggesting the existence of two separate loci, Tcd1a and Tcd1b, which are both lacking from T^{22H} (ref. (Lyon, Schimenti et al. 2000)). Moreover, the distortion of the t⁶ transmission ratio caused by the gain- and loss-of-function alleles of Tagap1 is considerably lower than that expected for Tcd1 encoded by the partial t-haplotype t^{h51} (8-15% for Tagap1 vs. >27% for t^{h51}; ref. (Lyon 1984)). Though some of this difference may be accounted for by variation in the genetic background or by a stronger effect of the Tagap1 loci in the t-haplotype, our data support the identification of two Tcd1 loci.

We suggest that the t-Tagap1 loci, in accordance with their map position on the chromosome, encode Tcd1a, and should be named Tagap1^{Tcd1a} (according to nomenclature rules). The contribution of each of the four loci and the exact mechanism by which they produce a hypermorph remains to be explored in detail.

In accordance with the finding of two Tcd1 loci and the data shown here, we predict that the second locus, Tcd1b, represents a hypomorphic or amorphic allele of a gene acting upstream of or epistatically to the G protein controlled by Tagap1.

### EXAMPLE 3

### Model of the Role of Tagap1 in Transmission Ratio Distortion

Whereas the applicant does not wish to be bound by any theory, the following model of the role of Tagap1 is envisaged.

We have previously proposed that the Tcds act upstream of Smok (Herrmann, Koschorz et al. 1999). According to our model, Smok activity is enhanced through the action of t-Distorters in all spermatozoa derived from t/+ males resulting in abnormal flagellar function. This negative effect of the t-Distorters is counterbalanced by Tcr, which is restricted to cells expressing the gene, thus rescuing t-sperm, while +-sperm remain dysfunctional. This would then lead to an advantage of the t-sperm in fertilising the eggs.

The findings presented here allow us to refine this model (FIG. 4): Tagap1 down-regulates a member of the Rho subfamily of small G proteins, which acts as negative regulator of Smok kinases. Tagap1^{Tcd1a} enhances down-regulation of this Rho GTPase, which leads to indirect up-regulation of Smok. We predict that the wild type allele of Tcd1b encodes either an activator of the Rho protein controlled by Tagap1, or an indirect inhibitor of Smok acting through another factor. If the t-haplotype allele Tcd1b represents a hypomorphic or amorphic allele, as suggested, this mutation would cause a reduction of the Tcd1b protein level indirectly resulting in up-regulation of Smok. Tcd1b would thus act additively or synergistically with and epistatically to Tagap1^{Tcd1a}. Tcd2 would further enhance this effect. Only Tcr expressing spermatozoa would be rescued from the motility defect caused by Tcds, resulting in preferential fertilisation of the eggs by t-sperm. Of course, at this point of analysis, other models are conceivable.

Tagap1 for the first time links Rho signalling to transmission ratio distortion. Previous reports have provided evidence for a role of Rho-GTPases in sperm motility, and the Rho binding protein Rhophilin and its interaction partner Ropporin are found in the flagellum (Hinsch, Habermann et al. 1993); (Nakamura, Fujita et al. 1999); (Fujita, Nakamura et al. 2000). Rhophilin is localised on the outer surface of the outer dense fibers of the sperm tail, directly opposing Ropporin, which is localised at the inner surface of the fibrous sheath (Fujita, Nakamura et al. 2000).

Rho-GTPases are well known for their essential role in cell motility and chemotaxis, which has been extensively studied in human neutrophils, fibroblasts and in the slime mould Dictyostelium discoideum (Van Haastert and Devreotes 2004). In these cell types Rho-GTPases control repeated extension of pseudopodia at the leading edge in response to a shallow gradient of signalling molecules, enabling the cell to move towards the stimulus. Whether these analogous functions of Rho-GTPases in cell motility in neutrophils and in sperm motility are part of a common mechanism, despite the fact that the former involves actin and myosin fibers while the latter involves microtubuli remains to be explored.

The identification of a t-complex-Distorter provides access to understanding the molecular principles of transmission ratio distortion and promotes the investigation of the role of Rho signalling in sperm motility.

### EXAMPLE 4

### Isolation of a Candidate Gene for Tcd2

The identification of Tagap1 as a distorter inspired us to search also for candidates of Tcd2 in the distal t haplotype region. We identified several genes encoding signalling molecules in this region, and analysed them with respect to our criteria for Tcd candidates outlined in Example 1. One candidate gene fulfilling these criteria was investigated in detail. It encodes Fgd2, a G-nucleotide exchange factor (GEF) for Rho small G proteins (Pasteris et Gorsky, 1999). GEFs promote the active state of small G-proteins by catalyzing the exchange of GDP for GTP (Schmidt and Hall, 2002).

According to its map position in the ENSEMBL genome database, Fgd2 should be located within the fourth inversion of the t complex, which contains Tcd2. We confirmed that Fgd2 indeed is located in the Tcd2 region by mapping it to partial t haplotypes, previously used to define the location of Tcd2 (FIG. 5a, b) (Lyon, 1984). Fgd2 encodes a protein with a N-terminal Dbl-homology (GEF) domain in tandem with a PH-domain, a FYVE domain and an additional C-terminal PH domain (FIG. 6a). The t form of the deduced Fgd2 protein differs from the published wild type sequence in a single amino acid residue. Serine 234 in the GEF domain was replaced in the t form by a glycine residue (S234G). Fgd2 is expressed in a number of organs (Pasteris and Gorsky, 1999). Expression in the testis is already detected at seven days post partum, corresponding to early meiotic stages of spermatogenesis, as revealed by northern- and RT-PCR analyses (FIG. 6b). In situ hybridization analysis of testis sections confirmed this result and furthermore showed that Fgd2 transcripts can be detected up to the round spermatid stage (FIG. 6c). This expression pattern may facilitate the distribution of Fgd2 products to all sperm cells, a prerequisite for a Distorter. Early expression during spermatogenesis was previously also shown for the Distorter Tagap1^{Tcd1a} (see Example 1).

Fgd2 also expresses a shorter (approximately 2.3 kb) transcript of variable size in wild type strains, from a promoter located within the gene (FIG. 6d). The shorter transcript encodes a N-terminally truncated protein lacking a substantial part of its DH domain (FIG. 6a). Since the GEF domain of this protein most likely is not functional, its role remains obscure. Northern blot analysis of testis RNA derived from wild type and t⁶/t^{w5} compound heterozygous mice showed that t haplotype mice express much higher levels of the long Fgd2 RNA than wild type mice (FIG. 6d). This observation was confirmed by qRT-PCR (FIG. 6e). We found that t⁶/t^{w5} males express up to 6-fold higher levels of Fgd2 RNA in the testis than wild type strains, which show various levels of transcripts. A similar result was recently obtained in the analysis of the Distorter Tagap1^{Tcd1a}, which was shown to represent a hypermorph. In Example 1 the high levels of Tagap1^{Tcd1a} transcripts were shown to be caused at least in part by amplification of the Tagap1 gene. This mechanism does not hold true for Fgd2. Instead, it seems that in t⁶/t^{w5} testis the level of the large Fgd2 transcript is highly increased on the expense of the smaller transcript, which is strongly reduced compared to wild type.

Tagap1, as Fgd2, also shows various levels of RNA expression in different wild type strains. These findings are in line with the observation of different penetrance of the TRD phenotype on various wild type strains (Gummere et al., 1986).

### EXAMPLE 5

### Fgd2 is a Candidate for Tcd2

The identification of Tagap1 as Distorter of the transmission ratio of t-haplotypes for the first time demonstrated an important role of small GTPases (preferentially of the Rho type) in transmission ratio distortion. This finding led us to suggest that other proteins involved in G protein signalling might also play a role in TRD. Therefore, the genomic region comprising the t-haplotype was searched with bioinformatics tools for genes encoding proteins of this group. Several were identified, among them Fgd2, encoding for a protein containing a Dbl homology domain and belonging to the GEF (guanine nucleotide exchange factor) family of proteins. Fgd2 is located in the distal portion of the t-haplotype, the In4 region. Fgd2 cDNA fragments were isolated by RT-PCR from testis of a male carrying the t-haplotypes t⁶/t^{w5}. Sequence analysis demonstrated a number of mutations in the t-specific transcript with respect to the wild type transcript suggesting Fgd2 as candidate for Tcd2. Only one amino acid mutation S234G was consistently found in the t-haplotypes.

On the basis of these data, involvement in G protein signalling, location in the t-haplotype region, expression in testis and modification of the coding region in the t-allele, Fgd2 was genetically analysed with respect to enhancement or reduction of the transmission ratio of the Tcr carrying t-haplotype t^{h49}, which lacks Tcd2. We engineered a targeted (loss-of-function) allele of Fgd2, Fgd2^{tm4Bgh}, in which the Dbl homology domain was inactivated, in embryonic stem cells as described (FIGS. 7, 8a, b). This allele was crossed into animals carrying t^{h49}. Males heterozygous for Fgd2^{tm4Bgh} and for t^{h49} were tested for the transmission ratio of t^{h49} to the offspring. Litter mates heterozygous for t^{h49} and wild type at the Fgd2 locus served as control. The breeding results demonstrate a significant reduction of the transmission ratio of t^{h49} from males carrying Fgd2^{tm4Bgh} as compared to litter mates of genotype t^{h49}/+; +/+. This result as shown in Table 2 (replaced by a more recent table including the data of former table 2 supplemented by additional data; the overall result is not altered) clearly demonstrates that Fgd2 is involved in transmission ratio distortion and a candidate for Tcd2.

Thus the teachings of Tagap1 led us to the discovery of another Distorter, Fgd2.

### EXAMPLE 6

### Model for Fgd2 in Transmission Ratio Distortion

The identification of Tcd2 as hypermorphic allele of the GEF encoding gene Fgd2 allows refining of our model of the molecular basis of transmission ratio distortion (FIG. 9). In Example 2 it was shown that over-expression of Tagap1, a GTPase activating protein and inhibitor of Rho small G proteins, increases the transmission rate of the t haplotype, while a loss-of-function allele has the opposite effect. It was shown that Fgd2, a GEF and activator of Rho GTPases acts in the same manner, that is a dosage increase enhances while a reduction of its activity lowers the t transmission rate. Thus, both Distorters act in parallel, while having opposing effects on their respective target Rho proteins. From these data we conclude that Fgd2 and Tagap1 must regulate different Rho targets. Therefore there are two signalling cascades exerting opposing effects on Smok1. One pathway, revealed by Tagap1, inhibits, the other, identified by Fgd2, activates Smok1. The hypermorph Tagap1^{Tcd1a} reduces inhibition, while Fgd2^{Tcd2} enhances activation of Smok1. In this manner both Distorter signalling cascades additively hyper-activate Smok1, followed by impairment of motility parameters in all sperm. Tcr is able to rescue this harmful effect of the Distorters, thus restoring normal flagellar function. Since the effect of Tcr is restricted to t sperm, the latter are able to out-compete the impaired wild type sperm in the race for eggs.

### EXAMPLE 7

### Tiam2 is a Candidate for Tcd1b

Another candidate for a t-Distorter was identified with bioinformatics tools in the Tcd1 subregion of the t-haplotype. This region contains another member of the family of Dbl homology domain proteins, Tiam2. Thus, this gene also belongs to the GEF family of proteins involved in G protein signalling. Primary characterization of the Tiam2 transcripts in t-haplotypes failed to identify a t-specific transcript in testis using the sensitive RT-PCR technology, suggesting that the t-allele of Tiam2 is not transcribed in testis. This finding was confirmed by Northern analysis, which failed to identify a transcript of the expected size in RNA derived from the testis of a t^{h49}/t^{h49} male, whereas a strong band was detected in testis RNA of a wild type control male (FIG. 10). Thus, the t-haplotype appears to carry a loss-of-function allele of Tiam2, strongly suggesting Tiam2 as candidate for a second Distorter in the Tcd1 region. According to our model of TRD (see example 3), Tiam2 is a candidate for Tcd1b.

Based on these results, we decided to functionally analyze Tiam2 with respect to its role in TRD using the same strategies as for Tagap1. For a loss-of function analysis, we engineered a mutant allele of Tiam2 in mouse embryonic stem cells (FIG. 11b). We constructed a targeting vector, which, after homologous recombination with the wild type locus results in an insertion of the PGKneo selection cassette in exon 3, thereby inactivating the gene (FIG. 11b). Out of approximately 750 clones analyzed, 2 were shown by southern blot analysis to have undergone the desired homologous recombination event. In XbaI digested genomic DNA of these clones, the 5′-probe detects a 16 kb fragment in wild type and a 12.5 kb probe in correctly targeted clones. Southern blot analysis using the 3′-probe detected a 16 kb band derived from the wild type and a 5.6 kb band originating from the mutant allele (FIG. 11c).

The correctly targeted clones were injected into blastocysts and chimerae were obtained. These chimerae are mated with mice heterozygous for the t-haplotype t⁶ (t⁶/+). In the next generation, the transmission rate of t⁶ from male offspring with the genotype t⁶/+; +/+will be compared with the t⁶ transmission rate of littermates heterozygous for the Tiam2 mutant allele (t⁶/+; +/−). A statistically significant difference of the transmission ratio between these two groups demonstrates that Tiam2 is a distorter of the transmission ratio.

In addition, as for Tagap1, we also will analyze Tiam2 function by transgenic overexpression of a wild type Tiam2 allele. We have isolated cDNAs from the Tiam2 gene, which are used to clone a transgenic construct. This construct consists of a testis specific ACE promoter (Howard et al., 1993) controlling expression of the full length Tiam2 open reading frame, and the rabbit beta-globin polyadenylation sequence. The transgenic construct will be injected into pronuclei of C57BL/6 derived oocytes to produce transgenic lines, which will be tested for expression of the transgenic construct in testis. Transgenic animals will be crossed to animals heterozygous for t⁶ (t⁶/+). Male littermates of the genotype t⁶/+; +/+ and t⁶/+; Tg(Tiam2)H2Bgh/0 will be tested for the transmission rate of t⁶. A statistically significant difference of the transmission ratio between these two groups of transgenic and non-transgenic littermates demonstrates that Tiam2 is a distorter of the transmission ratio.

### REFERENCES

Balch, W. E., Der, C. J., Hall, A. (editors) (1995). “Small GTPases and Their Regulators.” Methods in Enzymoloy 256.
Brent, A. E., Schweitzer, R. & Tabin, C. J. (2003) A somitic compartment of tendon progenitors. Cell 113, 235-48.
Bishop, A. L. and A. Hall (2000). “Rho GTPases and their effector proteins.” Biochem J 348 Pt 2: 241-55.
Bullard, D. C., C. Ticknor, et al. (1992). “Functional analysis of at complex responder locus transgene in mice.” Mamm Genome 3(10): 579-87.
Cassiday, L. A. and L. J. Maher, 3rd (2003). “Yeast genetic selections to optimize RNA decoys for transcription factor NF-kappa B.” Proc Natl Acad Sci USA 100(7): 3930-5.
Chapman, S. C., A. Lawson, et al. (2005). “Ubiquitous GFP expression in transgenic chickens using a lentiviral vector.” Development 132(5): 935-40.
Chien, C. T., P. L. Bartel, et al. (1991). “The two-hybrid system: a method to identify and clone genes for proteins that interact with a protein of interest.” Proc Natl Acad Sci USA 88(21): 9578-82.
Chung, J. H., M. Whiteley, et al. (1993). “A 5′ element of the chicken beta-globin domain serves as an insulator in human erythroid cells and protects against position effect in Drosophila.” Cell 74(3): 505-14.
Church, G. M. and W. Gilbert (1984). “Genomic sequencing.” Proc Natl Acad Sci USA 81(7): 1991-5.
DePamphilis, e. W. P. M. M. L. (1993). “Guide to techniques in mouse development.” Methods in Enzymology, Academic Press NY, 1993. Vol. 225.
Der, C. J., Hall, A. (2000). “Regulators and effectors of small GT Pases.” Methods in Enzymology. Vol. 325.
DerMardirossian, C. and G. M. Bokoch (2005). “GDIs: central regulatory molecules in Rho GTPase activation.” Trends Cell Biol 15(7): 356-63.
Dinkel, A., W. K. Aicher, et al. (1999). “Efficient generation of transgenic BALB/c mice using BALB/c embryonic stem cells.” J Immunol Methods 223(2): 255-60.
Donovan, S., K. M. Shannon, et al. (2002). “GTPase activating proteins: critical regulators of intracellular signaling.” Biochim Biophys Acta 1602(1): 23-45.
Dvorsky, R. and M. R. Ahmadian (2004). “Always look on the bright site of Rho: structural implications for a conserved intermolecular interface.” EMBO Rep 5(12): 1130-6.
Eddy, E. M., Toshimori, K. et al. (2003). Fibrous sheath of mammalian spermatozoa. FEBS Lett 334, 32-6.
Fox, H. S., G. R. Martin, et al. (1985). “Molecular probes define different regions of the mouse t complex.” Cell 40(1): 63-9.
Frangioni, J. V. and B. G. Neel (1993). “Solubilization and purification of enzymatically active glutathione S-transferase (pGEX) fusion proteins.” Anal Biochem 210(1): 179-87.
Fraser, L. R. and K. Dudley (1999). “New insights into the t-complex and control of sperm function.” Bioessays 21(4): 304-12.
Fujita, A., K. Nakamura, et al. (2000). “Ropporin, a sperm-specific binding protein of rhophilin, that is localized in the fibrous sheath of sperm flagella.” J Cell Sci 113 (Pt 1): 103-12.
Geijsen, N., M. Horoschak, et al. (2004). “Derivation of embryonic germ cells and male gametes from embryonic stem cells.” Nature 427(6970): 148-54.
Gilman, M. (1997). Ribonuclease Protection Assay, John Wiley & Sons, Inc., New York.
Gummere, G. R., P. J. McCormick, et al. (1986). “The influence of genetic background and the homologous chromosome 17 on t-haplotype transmission ratio distortion in mice.” Genetics 114(1): 235-45.
Hames, S. J. H. a. B. D. (1985). “Nucleic acid hybridization, a practical approach”. IRL Press, Oxford.
Herrmann, B., M. Bucan, et al. (1986). “Genetic analysis of the proximal portion of the mouse t complex: evidence for a second inversion within t haplotypes.” Cell 44(3): 469-76.
Herrmann, B. G., D. P. Barlow, et al. (1987). “A large inverted duplication allows homologous recombination between chromosomes heterozygous for the proximal t complex inversion.” Cell 48(5): 813-25.
Herrmann, B. G., B. Koschorz, et al. (1999). “A protein kinase encoded by the t complex responder gene causes non-mendelian inheritance.” Nature 402(6758): 141-6.
Hinsch, K. D., B. Habermann, et al. (1993). “ADP-ribosylation of Rho proteins inhibits sperm motility.” FEBS Lett 334(1): 32-6.
Horiuch, T., C. Emuta, et al. (2002). “Birth of normal calves after intracytoplasmic sperm injection of bovine oocytes: a methodological approach.” Theriogenology 57(3): 1013-24.
Howard, T., R. Balogh, et al. (1993). “Sperm-specific expression of angiotensin-converting enzyme (ACE) is mediated by a 91-base-pair promoter containing a CRE-like element.” Mol Cell Biol 13(1): 18-27.
Hubner, K., G. Fuhrmann, et al. (2003). “Derivation of oocytes from mouse embryonic stem cells.” Science 300(5623): 1251-6.
Joyner, A. L. E. (1993). “Gene Targeting, A Practical Approach.” Oxford University Press.
Kim, V. N. (2005). “MicroRNA biogenesis: coordinated cropping and dicing.” Nat Rev Mol Cell Biol 6(5): 376-85.
Kleene, K. C. (1989). “Poly(A) shortening accompanies the activation of translation of five mRNAs during spermiogenesis in the mouse.” Development 106(2): 367-73.
Kurtz, S. E., K. Esposito, et al. (2003). “Inhibition of an activated Ras protein with genetically selected peptide aptamers.” Biotechnol Bioeng 82(1): 38-46.
Lever, A. M., P. M. Strappe, et al. (2004). “Lentiviral vectors.” J Biomed Sci 11(4): 439-49.
Lyon, M. F. (1984). “Transmission ratio distortion in mouse t-haplotypes is due to multiple distorter genes acting on a responder locus.” Cell 37(2): 621-8.
Lyon, M. F. (1986). “Male sterility of the mouse t-complex is due to homozygosity of the distorter genes.” Cell 44(2): 357-63.
Lyon, M. F. (1992). “Deletion of mouse t-complex distorter-1 produces an effect like that of the t-form of the distorter.” Genet Res 59(1): 27-33.
Lyon, M. F. (2003). “Transmission ratio distortion in mice.” Annu Rev Genet 37: 393-408.
Lyon, M. F., J. C. Schimenti, et al. (2000). “Narrowing the critical regions for mouse t complex transmission ratio distortion factors by use of deletions.” Genetics 155(2): 793-801.
Morita, T., K. Murata, et al. (1993). “Mouse t haplotype-specific double insertion of B2 repetitive sequences in the Tcp-1 intron 7.” Mamm Genome 4(1): 58-9.
Nadeau, J. H., D. Varnum, et al. (1989). “Genetic evidence for two t complex tail interaction (tct) loci in t haplotypes.” Genetics 122(4): 895-903.
Nakamura, K., A. Fujita, et al. (1999). “Rhophilin, a small GTPase Rho-binding protein, is abundantly expressed in the mouse testis and localized in the principal piece of the sperm tail.” FEBS Lett 445(1): 9-13.
Pasteris, N. G and Gorsky, J. L. (1999). Isolation, characterization, and mapping of the mouse and human Fgd2 genes, faciogenital dysplasia (FDG1; Aarskog syndrome) gene homologues. Genomics 60, 57-66.
Pirottin, D., L. Grobet, et al. (2005). “Transgenic engineering of male-specific muscular hypertrophy.” Proc Natl Acad Sci USA 102(18): 6413-8.
Planchart, A., Y. You, et al. (2000). “Physical mapping of male fertility and meiotic drive quantitative trait loci in the mouse t complex using chromosome deficiencies.” Genetics 155(2): 803-12.
Ramirez-Solis, R., A. C. Davis, et al. (1993). “Gene targeting in embryonic stem cells.” Methods Enzymol 225: 855-78.
Rosen, L. L., D. C. Bullard, et al. (1990). “Molecular cloning of the t complex responder genetic locus.” Genomics 8(1): 134-40.
Rupp, R. A., L. Snider, et al. (1994). “Xenopus embryos regulate the nuclear localization of XMyoD.” Genes Dev 8(11): 1311-23.
Sambrook J., F., E. F., Maniatis, T. (1989). Molecular Cloning, A Laboratory Manual, Cold Spring Harbor Laboratory Press.
Schimenti, J., Reynolds, J. L. & Planchart (2005). Mutations in Serac1 or Synj2 cause proximal t haplotype-mediated male mouse sterility but not transmission ratio distortion. Proc Natl Acad Sci USA 102, 3342-7.
Schimenti, J. (2000). “Segregation distortion of mouse t haplotypes the molecular basis emerges.” Trends Genet 16(6): 240-3.
Schimenti, J., L. Vold, et al. (1987). “An unstable family of large DNA elements in the center of the mouse t complex.” J Mol Biol 194(4): 583-94.
Schmidt, A. and A. Hall (2002). “Guanine nucleotide exchange factors for Rho GTPases: turning on the switch.” Genes Dev 16(13): 1587-609.
Schmidt, S., S. Diriong, et al. (2002). “Identification of the first Rho-GEF inhibitor, TRIPalpha, which targets the RhoA-specific GEF domain of Trio.” FEBS Lett 523(1-3): 3542.
Self, A. J. and A. Hall (1995). “Measurement of intrinsic nucleotide exchange and GTP hydrolysis rates.” Methods Enzymol 256: 67-76.
Self, A. J. and A. Hall (1995). “Purification of recombinant Rho/Rac/G25K from Escherichia coli.” Methods Enzymol 256: 3-10.
Silver, L. M. and D. Remis (1987). “Five of the nine genetically defined regions of mouse t haplotypes are involved in transmission ratio distortion.” Genet Res 49(1): 51-6.
Van Haastert, P. J. and P. N. Devreotes (2004). “Chemotaxis: signalling the way forward.” Nat Rev Mol Cell Biol 5(8): 626-34.
Wennerberg, K. and C. J. Der (2004). “Rho-family GTPases: it's not only Rac and Rho (and I like it).” J Cell Sci 117(Pt 8): 1301-12.
Wilmut, I., A. E. Schnieke, et al. (1997). “Viable offspring derived from fetal and adult mammalian cells.” Nature 385(6619): 810-3.
Willison, K. & Ashworth A. Mammalian spermatogenic gene expression. Trends Genet. 3, 351-355 (1987).

TABLE 1
Table 1 Transmission ratio of t⁶ from males lacking or
over-expressing Tagap1.
For overexpression experiments, two independent transgenic lines
(TgH1-33 and tgH1-4) were established, the expression of the
transgenic construct was verified and both lines were bred to the
partial t-haplotype t⁶. The transmission ratio of t⁶ was
compared between transgenic and non-transgenic animals. Both
lines significantly increase the transmission ratio of t⁶.

| | Number | | | | | | |
|---|---|---|---|---|---|---|---|
| | of | Offspring | | | | | |
| | males | t⁶ | wt | total | % t⁶ | χ² | P |
| Genotype of male | | | | | | | |
| TgH1-33/0; t⁶/+ | 5 | 218 | 39 | 257 | 85 | 7.08 | 0.01 |
| t⁶/+ | 5 | 125 | 43 | 168 | 74 | | |
| TgH1-4/0; t⁶/+ | 7 | 190 | 19 | 209 | 91 | 5.17 | 0.025 |
| t⁶/+ | 5 | 200 | 39 | 239 | 84 | | |
| Combined data: | | | | | | | |
| Tg/0; t⁶/+ | 12 | 408 | 58 | 466 | 88 | 9.57 | 0.01 |
| t⁶/+ | 10 | 325 | 82 | 407 | 80 | | |
| Tagap1^{tm3Bgh}/+; t⁶/+ | 9 | 245 | 109 | 354 | 69 | 21.09 | 0.001 |
| Tagap1 +/+; t⁶/+ | 9 | 292 | 56 | 348 | 84 | | |

Abbr..:
TgH1-33, Tg(Tagap1)H1-33Bgh;
TgH1-4, Tg(Tagap1)H1-4Bgh,
wt, wild type.

An inactivated Tagap1 allele (loss-of-function) produced by gene targeting results in the complementary effect on the transmission ratio of the t-haplotype (lower part). The transmission ratio of the t-haplotype by t⁶/+ animals is strongly reduced by heterozygozity for Tagap1^{tm3Bgh}.

TABLE 2
Table 2: Transmission ratio of t^{h49} from males lacking Fgd2.
The transmission ratio of the t-haplotype by t^{h49} animals is
decreased by a loss-of-function allele of Fgd2.

| | Number | | | | | | |
|---|---|---|---|---|---|---|---|
| | of | Offspring | | | | | |
| Genotype of male | males | t^{h49} | wt | total | % t^{h49} | χ² | P |
| Fgd2 +/+; t^{h49}/+ | 7 | 150 | 169 | 319 | 47 | 8.44 | 0.01 |
| Fgd2^{tm4Bgh}/+; | 7 | 105 | 191 | 296 | 35 | | |
| t^{h49}/+ | | | | | | | |

TABLE 3
Overview of the Sequences disclosed in the Sequence listing

| | Organism/gene/allele, isoform |
|---|---|
| Coding Sequences | |
| 1 | Tagap1 mouse wildtype FVB/N |
| 2 | Tagap1 mouse wildtype BALB/c |
| 3 | Tagap1 t1 mouse |
| 4 | Tagap1 t2 mouse |
| 5 | Tagap1 t3 mouse |
| 6 | Tagap1 t4 mouse |
| 7 | Tagap homo sapiens |
| 8 | Tagap Rattus |
| 9 | Fgd2 mouse transcript variant 3 |
| 10 | Fgd2 mouse transcript variant 1 |
| 11 | Fgd2 mouse transcript variant 2 |
| 12 | Fgd2 mouse transcript variant 1 t⁶/t^{w5} |
| 13 | Tiam2 mouse wildtype |
| 14 | Tiam2 homo sapiens |
| 15 | Tcr, short 5′utr mus musculus |
| 16 | Tcr, long 5′utr mus musculus |
| 31 | Tagap1 Bos taurus |
| 32 | Tagap1 Canis familiaris |
| 33 | Tagap1 Gallus gallus |
| 34 | Fgd2 mouse transcript variant 2 t⁶/t^{w5} |
| 35 | Fgd2 Bos taurus |
| 36 | Fgd2 Canis familiaris |
| 37 | Fgd2 Rattus norvegicus |
| 38 | Fgd2 (splice variant) Rattus norvegicus |
| 47 | Tiam2 Bos taurus |
| 48 | Tiam2 Gallus gallus |
| 49 | Tiam2 Rattus norvegicus |
| 50 | Tiam2 Canis familiaris |
| 55 | Fgd2 mouse transcript variant 1a t⁶/t^{w5} |
| 56 | Tagap1 Danio rerio |
| 57 | Tagap1 Macacca mulatta |
| 58 | Tagap1 Monodelphis domestica |
| 59 | Tagap1 Xenopus tropicalis |
| 60 | Tagap1 Pan troglodytes |
| 61 | Fgd2 Macacca mulatta transcript variant 1 |
| 62 | Fgd2 Macacca mulatta transcript variant 2 |
| 63 | Fgd2 Macacca mulatta transcript variant 3 |
| 64 | Fgd2 Monodelphis domestica |
| 65 | Fgd2 Pan troglodytes |
| 66 | Fgd2 Homo sapiens |
| 67 | Tiam2 Macacca mulatta transcript variant 1 |
| 68 | Tiam2 Macacca mulatta transcript variant 2 |
| 69 | Tiam2 Monodelphis domestica transcript variant 1 |
| 70 | Tiam2 Monodelphis domestica transcript variant 2 |
| 71 | Tiam2 Monodelphis domestica transcript variant 3 |
| 72 | Tiam2 Pan troglodytes (fragment)* |
| Deduced Protein | |
| sequences | |
| 17 | Tagap1 mouse wildtype FVB/N |
| 18 | Tagap1 mouse wildtype BALB/c |
| 19 | Tagap1 t1 mouse |
| 20 | Tagap1 t2 mouse |
| 21 | Tagap1 t3 mouse |
| 22 | Tagap1 t4 mouse |
| 23 | Tagap homo sapiens |
| 24 | Tagap Rattus |
| 25 | FGD2 mouse transcript variant 3 |
| 26 | FGD2 mouse transcript variant 1 |
| 27 | FGD2 mouse transcript variant 2 |
| 28 | FGD2 mouse transcript variant 1 t⁶/t^{w5} |
| 29 | Tiam2 mouse wildtype |
| 30 | Tiam2 homo sapiens |
| 39 | Tagap1 Bos taurus |
| 40 | Tagap1 Canis familiaris |
| 41 | Tagap1 Gallus gallus |
| 42 | Fgd2 mouse transcript variant 2 t⁶/t^{w5} |
| 43 | Fgd2 Bos taurus |
| 44 | Fgd2 Canis familiaris |
| 45 | Fgd2 Rattus norvegicus |
| 46 | Fgd2 (splice variant) Rattus norvegicus |
| 51 | Tiam2 Bos taurus |
| 52 | Tiam2 Gallus gallus |
| 53 | Tiam2 Rattus norvegicus |
| 54 | Tiam2 Canis familiaris |
| 73 | Fgd2 mouse transcript variant 1a t^{6/}t^{w5} |
| 74 | Tagap1 Danio rerio |
| 75 | Tagap1 Macacca mulatta |
| 76 | Tagap1 Monodelphis domestica |
| 77 | Tagap1 Xenopus tropicalis |
| 78 | Tagap1 Pan troglodytes |
| 79 | Fgd2 Macacca mulatta transcript variant 1 |
| 80 | Fgd2 Macacca mulatta transcript variant 2 |
| 81 | Fgd2 Macacca mulatta transcript variant 3 |
| 82 | Fgd2 Monodelphis domestica |
| 83 | Fgd2 Pan troglodytes |
| 84 | Fgd2 Homo sapiens |
| 85 | Tiam2 Macacca mulatta transcript variant 1 |
| 86 | Tiam2 Macacca mulatta transcript variant 2 |
| 87 | Tiam2 Monodelphis domestica transcript variant 1 |
| 88 | Tiam2 Monodelphis domestica transcript variant 2 |
| 89 | Tiam2 Monodelphis domestica transcript variant 3 |
| 90 | Tiam2 Pan troglodytes (fragment)* |

*With the methods disclosed in the invention it is not an undue burden for the skilled person to identify and isolate the complete Tiam2 distorter gene.

TABLE 4
Oligonucleotide primer sequences and PCR conditions

| | | Number of cycles/ | |
|---|---|---|---|
| Experiment | Primer sequence | annealing temp. | Product size |
| Quantitative RT-PCR, | s: 5′-TGAAGCTCATTTTCTCCAACATCT-3′ | 40 cycles/60° C. | 71 bp |
| long transcript specific (FIG. 6e) | as: 5′-CTGCAGCTCGGGAAGGAA-3′ | | |
| | Probe: (5′-FAM, 3′-TAMRA-labeled) | | |
| | 5′-CTCCATCTATCGTTTCCACGCCCAGTT-3′ | | |
| RT-PCR, | s: 5′-GGCTGTGGTAGACCTGCTTC-3′ | 35 cycles/58° C. | 461 bp |
| long transcript specific (FIG. 6b) | as: 5′-AGACGGAGCAGGCCTAT-3′ | | |
| RT-PCR, beta Actin control | s: 5′-TGGAATCCTGTGGCATCCATGAAA-3′ | 25 cycles/58° C. | 349 bp in |
| (FIG. 6b) | as: 5′-TAAAACGCAGCTCAGTAACAGTCCG-3′ | | NM_007393 |
| PCR-amplification of template for | s: 5′-AGAATCCCGCGGTACGA-3′ | 35 cycles/58° C. | 395 bp |
| in vitro transcription of probe | as: 5′-GTCAGCTCCCGCACCT-3′ | | |
| for in situ hybridization (FIG. | | | |
| 6c) | | | |
| PCR amplification of coding region | s: 5′- GGCTAGCAGGATGGAGCGA-3′ | 38 cycles/66° C. | 2407 bp |
| of long Fgd2 transcript | as: 5′-GTGCTCTCAGGTTCTTGTGTAG-3′ | | |
| PCR amplification of probe for | 5: 5′-GGCTTGCGGCTATGTAG-3′ | 38 cycles/66° C. | 949 bp |
| northern hybridization (FIG. 6d) | as: 5′-GTGCTCTCAGGTTCTTGTGTAG-3′ | | |
| PCR amplification of long tran- | s: 5′-GGCTAGCAGGATGGAGCGA-3′ | 35 cycles/57° C. | 361 bp |
| script specific probe for northern | as: 5′-AGACGGAGCAGGCCTAT-3′ | | |
| hybridization (FIG. 6b) | | | |
| PCR amplification of left arm of | f: 5′-CTGCTTCTGGGGTAACT-3′ | 35 cycles/65° C. | 3914 bp |
| targeting construct (SpeI site in | r: 5′-ATAGGCCTGCTCCGTCT-3′ | | |
| forward primer) (FIG. 7) * | | | |
| PCR amplification of right arm of | f: 5′-AAGAATCCCGCGGTACGAACTG-3′ | 35 cycles/65° C. | 2589 bp |
| targeting construct (EcoRV site in | r: 5′-GACAACGCCCGACATCATAGAG-3′ | | |
| forward-, SalI site in reverse | | | |
| primer) (FIG. 7) | | | |
| PCR amplification of 5′-probe for | f: 5′-ACAGGTCTCACGTAGCCGAATC-3′ | 35 cycles/560 C. | 642 bp |
| detection of targeted allele | r: 5′-CGGGTGAAGCAGGTCTACCACA-3′ | | |
| (FIG. 7) | | | |
| PCR amplification of 3′-probe for | f: 5′-TGGATGCCGCTCAGTTGCTAAT-3′ | 35 cycles/56° C. | 652 bp |
| detection of targeted allele | r: 5′-TGAAACTCAGTGTGTAGACCAG-3′ | | |
| (FIG. 7) | | | |

* A 180 bp deletion at the 3′-end of the left arm occurred during construction of the targeting vector
s, sense primer; as, antisense primer, f, forward primer, r, reverse primer

1. A method for producing a transgenic non human male animal, preferably a mammal, fish, bird or insect, wherein the transgene(s) confer(s) a change in the transmission ratio of (a) genetic trait(s) to the offspring of said non human male animal, preferably mammal, fish, bird or insect to a non-Mendelian ratio, said method comprising introducing
(a) a first nucleic acid molecule encoding an expression product with a Responder function into a chromosome of a non-human germ cell, (fertilized) egg cell, embryonic cell or a cell derived therefrom, of the same species as the transgenic male to be prepared, said chromosome containing or conferring said genetic trait(s), thereby linking on said chromosome said Responder function to the genetic trait(s); and
(b) at least one second nucleic acid molecule encoding an expression product with a Distorter function into (a) chromosome(s) of a non-human germ cell, (fertilized) egg cell, embryonic cell or a cell derived of the same species as the transgenic male to be prepared, wherein said expression product with a Distorter function is a factor involved in G protein signaling,
wherein said first nucleic acid molecule encoding an expression product with Responder function and said at least one second nucleic acid molecule encoding an expression product with a Distorter function are introduced into the same or different chromosomes.

2. A method for producing a transgenic non human male animal, preferably a mammal, fish, bird or insect, wherein the transgene(s) confer(s) a change in the transmission ratio of (a) genetic trait(s) to the offspring of said non human male animal, preferably mammal, fish, bird or insect to a non-Mendelian ratio, said method comprising introducing
(a) a first nucleic acid molecule encoding an expression product with a Responder function into a chromosome of a non-human germ cell, (fertilized) egg cell, embryonic cell or a cell derived therefrom, of the same species as the transgenic male to be prepared, said chromosome containing or conferring said genetic trait(s), thereby linking on said chromosome said Responder function to the genetic trait(s); and
(b) at least one second nucleic acid molecule encoding an expression product directed against the Distorter function into (a) chromosome(s) of a non-human germ cell, (fertilized) egg cell, embryonic cell or a cell derived therefrom, of the same species as the transgenic male to be prepared, wherein said Distorter function is an expression product which is encoded by a nucleic acid molecule encoding an expression product with a Distorter function, wherein said expression product with a Distorter function is a factor involved in G protein signaling; and/or
(c) a second nucleic acid molecule for inactivation of the Distorter function by homologous recombination, wherein said second nucleic acid molecule for inactivation of the Distorter function by homologous recombination is at least partially partially identical to said nucleic acid molecule encoding an expression product with a Distorter function,
wherein said first nucleic acid molecule encoding an expression product with a Responder function and said at least one second nucleic acid molecule encoding an expression product directed against the Distorter function and/or said second nucleic acid molecule for inactivation of the Distorter function by homologous recombination are introduced into the same or different chromosomes,
thereby partially or completely inactivating the Distorter function.

3. The method of claim 1 or 2, wherein said mammal is selected from the group consisting of Mus, Rattus, Bos, Sus and Ovis.

4. The method of claim 1, wherein said genetic trait is sex.

5. The method of claim 1, wherein said chromosome is an X or Y chromosome or a corresponding sex chromosome in birds, fish or insect.

6. The method of claim 1, wherein said chromosome is an autosome.

7. The method of claim 1, wherein said factor involved in G protein signaling is a factor involved in Rho signaling.

8. The method of claim 1, wherein said first nucleic acid molecule encoding an expression product with Responder function is selected from the group consisting of
(a) a nucleic acid molecule comprising or consisting of the nucleic acid molecule as shown in SEQ ID NO: 15 or 16 or a fragment thereof;
(b) a nucleic acid molecule being an allelic variant or a homologue or orthologue of the nucleic acid molecule of (a);
(c) a nucleic acid molecule hybridizing to a nucleic acid molecule complementary to the nucleic acid molecule of (a) or (b); and
(d) a nucleic acid molecule which is related to the nucleic acid molecule of (a), (b) or (c) by the degeneration of the genetic code.

9. The method of claim 1, wherein said (at least one) second nucleic acid molecule encoding an expression product with a Distorter function is/are selected from the group consisting of
(a) a nucleic acid molecule comprising or consisting of the nucleic acid molecule as shown in any one of SEQ ID NOs:1 to 14, 31 to 38, 47 to 50 or 55 to 72 or a fragment thereof;
(b) a nucleic acid molecule being an allelic variant or a homologue or orthologue of the nucleic acid molecules of (a);
(c) a nucleic acid molecule hybridizing to a nucleic acid molecule complementary to the nucleic acid molecule of (a) or (b); and
(d) a nucleic acid molecule which is related to the nucleic acid molecule of (a), (b) or (c) by the degeneration of the genetic code.

10. The method of claim 1, wherein said at least one second nucleic acid molecule encoding an expression product with a Distorter function is/are selected from the group consisting of
(a) a nucleic acid molecule comprising or consisting of the nucleic acid molecule as shown in any one of SEQ ID NOs: 1 to 12, 31 to 38 or 55 to 66 or a fragment thereof;
(b) a nucleic acid molecule being an allelic variant or a homologue or orthologue of the nucleic acid molecule of (a);
(c) a nucleic acid molecule hybridizing to a nucleic acid molecule complementary to the nucleic acid molecule of (a) or (b); and
(d) a nucleic acid molecule which is related to the nucleic acid molecule of (a), (b) or (c) by the degeneration of the genetic code,
thereby enhancing said transmission ratio of said genetic trait(s).

11. The method of claim 1, wherein said at least one second nucleic acid molecule encoding an expression product with a Distorter function is/are selected from the group consisting of
(a) a nucleic acid molecule comprising or consisting of the nucleic acid molecule as shown in SEQ ID NO: 13 or 14, 47 to 50 or 67 to 72 or a fragment thereof;
(b) a nucleic acid molecule being an allelic variant or a homologue or orthologue of the nucleic acid molecule of (a);
(c) a nucleic acid molecule hybridizing to a nucleic acid molecule complementary to the nucleic acid molecule of (a) or (b); and
(d) a nucleic acid molecule which is related to the nucleic acid molecule of (a), (b) or (c) by the degeneration of the genetic code,
thereby reducing said transmission ratio of said genetic trait(s).

12. The method of claim 1, wherein said nucleic acid molecule encoding an expression product with a Distorter function is selected from the group consisting of
(a) a nucleic acid molecule comprising or consisting of the nucleic acid molecule as shown in any one of SEQ ID NOs 1 to 12, 31 to 38 or 55 to 66 or a fragment thereof;
(b) a nucleic acid molecule being an allelic variant or a homologue or orthologue of the nucleic acid molecule of (a);
(c) a nucleic acid molecule hybridizing to a nucleic acid molecule complementary to the nucleic acid molecule of (a) or (b); and
(d) a nucleic acid molecule which is related to the nucleic acid molecule of (a), (b) or (c) by the degeneration of the genetic code,
thereby reducing said transmission ratio of said genetic trait(s).

13. The method of claim 1, wherein said nucleic acid molecule encoding an expression product with a Distorter function is selected from the group consisting of
(a) a nucleic acid molecule comprising or consisting of the nucleic acid molecule as shown in SEQ ID NO 13 or 14, 47 to 50 or 67 to 72 or a fragment thereof;
(b) a nucleic acid molecule being an allelic variant or a homologue or orthologue of the nucleic acid molecule of (a);
(c) a nucleic acid molecule hybridizing to a nucleic acid molecule complementary to the nucleic acid molecule of (a) or (b); and
(d) a nucleic acid molecule which is related to the nucleic acid molecule of (a), (b) or (c) by the degeneration of the genetic code,
thereby enhancing said transmission ratio of said genetic trait(s).

14. The method of claim 1, further comprising crossing the transgenic non human male mammal, fish, bird or insect obtained by the method of anyone of claims 1 to 13 with a non human female mammal, fish, bird or insect and analyzing the offspring of said cross for transmission of said genetic trait(s).

15. The method of claim 1, wherein said Responder function and/or said Distorter function is the mouse-t-complex Responder/Distorter function.

16. The method of claim 2, wherein said expression product directed against the Distorter function is an aptamer, a siRNA or shRNA or miRNA, a ribozyme, or an antisense nucleic acid molecule specifically hybridizing to said nucleic acid molecules encoding a factor involved in G protein signaling, or is an antibody, an antibody fragment or derivative thereof.

17. The method of claim 1, wherein said at least one second nucleic acid molecule encoding the expression product with a Distorter function is modified, thereby further reducing or further enhancing the Distorter function activity.

18. The method of claim 1, wherein said first nucleic acid molecule encoding an expression product with a Responder function and said at least one second nucleic acid molecule encoding an expression product with Distorter function and/or said at least one second nucleic acid molecule encoding an expression product directed against the Distorter function and/or said second nucleic acid molecule for inactivation of the Distorter function by homologous recombination and a promoter controlling expression in spermatogenesis and/or spermiogenesis and/or a stop cassette are integrated in said X or Y chromosome or corresponding sex chromosome or in one of said autosomes in a reversible inactive state of expressibility.

19. A non human male or female mammal, fish, bird or insect, wherein said non human male or female mammal, fish, bird or insect is transgenic for the nucleic acid molecule encoding an expression product with a Responder function and the nucleic acid molecule encoding an expression product with a Distorter function and/or the nucleic acid molecule encoding an expression product directed against the Distorter function and/or the nucleic acid molecule for inactivation of the Distorter function by homologous recombination as defined in claim 1.

20. A pair of non human male and female animals, wherein at least one of the male and/or female is a transgenic non human mammal, fish, bird or insect as in claim 19.

21. The pair of non-human male and female animals of claim 20, wherein the nucleic acid molecule or part thereof encoding an expression product with a Responder function and/or the nucleic acid molecule or part thereof encoding an expression product with a Distorter function and/or the nucleic acid molecule or part thereof encoding an expression product directed against the Distorter function and/or the nucleic acid molecule or part thereof for inactivation of the Distorter function by homologous recombination as in claim 1 is/are flanked by recombinase recognition sites.

22. The pair of non-human male and female animals of claim 20 having further stably integrated into its genomic DNA a nucleic acid molecule encoding a site specific DNA recombinase.

23. The pair of non human male and female animals of claim 22, wherein said DNA recombinase is Cre, wherein said recognition sites are loxP sites, or flp, wherein said recognition sites are FRT sites, or Φc31, wherein said recognition sites are att sites.

24. The pair of non human male and female animals of claim 22, wherein said DNA recombinase is controlled by regulatory elements that are active prior to spermiogenesis.

25. Sperm obtainable from a male of the transgenic non-human animal, preferably mammal, fish, bird or insect as defined in claim 19.

26. Use of the sperm of claim 25 for the production of offspring.

27. Use of the nucleic acid molecule encoding an expression product with a Distorter function as defined in claim 1, for the identification of chemicals or biological compounds able to trigger the (premature) activation or inhibition of the Responder/Distorter signalling cascade.

28. Use of the nucleic acid molecule encoding an expression product with a Distorter function as defined in claim 1 for the isolation of receptor molecules and/or other members of the Responder/Distorter signaling cascade to which said expression product may bind.

29. A method for the detection of the nucleic acid molecule encoding an expression product with a Distorter function and/or the nucleic acid molecule encoding an expression product directed against the Distorter function and/or the nucleic acid molecule for inactivation of the Distorter function by homologous recombination as defined in claim 1 in the non human male or female mammal, fish, bird or insect as defined in claim 19 comprising identifying said nucleic acid molecule encoding an expression product with a Distorter function and/or said nucleic acid molecule encoding an expression product directed against the Distorter function and/or said nucleic acid molecule for inactivation of the Distorter function by homologous recombination in said non human male or female mammal, fish, bird or insect by polymerase chain reaction (PCR), gene (micro)array hybridization, single nucleotide polymorphism (SNP) analysis, and/or sequencing with primers hybridizing to said nucleic acid molecule.

30. A nucleic acid molecule encoding an expression product with a Distorter function, wherein said expression product with a Distorter function is a factor involved in G protein signaling, selected from the group consisting of:
(a) a nucleic acid molecule comprising or consisting of the nucleic acid molecule of any one of SEQ ID NOs: 3 to 6 and 12 or a fragment thereof;
(b) a nucleic acid molecule being an allelic variant or a homologue or orthologue of the nucleic acid molecule of (a);
(c) a nucleic acid molecule which hybridizes under stringent conditions to the nucleic acid molecule of (a), wherein said nucleic acid molecule encodes a polypeptide which has
(i) at the position corresponding to position 49 of SEQ ID NO: 17 an I
(ii) at the position corresponding to position 144 of SEQ ID NO: 17 an L
(iii) at the position corresponding to position 323 of SEQ ID NO: 17 a T and
(iv) which terminates after position 442;
(d) a nucleic acid molecule which hybridizes under stringent conditions to the nucleic acid molecule of (a), wherein said nucleic acid molecule encodes a polypeptide which has
(i) at the position corresponding to position 49 of SEQ ID NO: 17 an I;
(ii) at the position corresponding to position 137 of SEQ ID NO: 17 an E;
(iii) at the position corresponding to position 207 of SEQ ID NO: 17 an F;
(iv) at the position corresponding to position 301 of SEQ ID NO: 17 an M;
(v) at the position corresponding to position 323 of SEQ ID NO: 17 an T;
(vi) at the position corresponding to position 332 of SEQ ID NO: 17 a D;
(vii) at the position corresponding to position 407-413 of SEQ ID NO: 17 an internal deletion;
(viii) at the position corresponding to position 440 of SEQ ID NO: 17 an M;
(ix) at the position corresponding to position 471 of SEQ ID NO: 17 an L;
(x) at the position corresponding to position 552 of SEQ ID NO: 17 an I;
(xi) at the position corresponding to position 596 of SEQ ID NO: 17 a K;
(xii) at the position corresponding to position 607 of SEQ ID NO: 17 an R;
(xiii) at the position corresponding to position 610 of SEQ ID NO: 17 an S; and
(xiv) at the position corresponding to position 703 of SEQ ID NO: 17 a V;
(e) a nucleic acid molecule which hybridizes under stringent conditions to the nucleic acid molecule of (a), wherein said nucleic acid molecule encodes a polypeptide which has
(i) at the position corresponding to position 49 of SEQ ID NO: 17 an I;
(ii) at the position corresponding to position 54 of SEQ ID NO: 17 a G;
(iii) at the position corresponding to position 137 of SEQ ID NO: 17 an E;
(iv) at the position corresponding to position 173 of SEQ ID NO: 17 a G;
(v) at the position corresponding to position 207 of SEQ ID NO: 17 an F;
(vi) at the position corresponding to position 301 of SEQ ID NO: 17 an M;
(vii) at the position corresponding to position 323 of SEQ ID NO: 17 a T;
(viii) at the position corresponding to position 332 of SEQ ID NO: 17 a D;
(ix) at the position corresponding to position 407-413 of SEQ ID NO: 17 an internal deletion;
(x) at the position corresponding to position 440 of SEQ ID NO: 17 an M;
(xi) at the position corresponding to position 471 of SEQ ID NO: 17 an L;
(xii) at the position corresponding to position 508 of SEQ ID NO: 17 an S;
(xiii) at the position corresponding to position 552 of SEQ ID NO: 17 an I;
(xiv) at the position corresponding to position 596 of SEQ ID NO: 17 a K;
(xv) at the position corresponding to position 607 of SEQ ID NO: 17 an R;
(xvi) at the position corresponding to position 610 of SEQ ID NO: 17 an S; and
(xvii) at the position corresponding to position 703 of SEQ ID NO: 17 a V; and
(f) a nucleic acid molecule which hybridizes under stringent conditions to the nucleic acid molecule of (a), wherein said nucleic acid molecule encodes a polypeptide which has
(i) at the position corresponding to position 49 of SEQ ID NO: 17 an I;
(ii) at the position corresponding to position 137 of SEQ ID NO: 17 an E;
(iii) at the position corresponding to position 207 of SEQ ID NO: 17 an F;
(iv) at the position corresponding to position 301 of SEQ ID NO: 17 an M;
(v) at the position corresponding to position 323 of SEQ ID NO: 17 a T;
(vi) at the position corresponding to position 332 of SEQ ID NO: 17 a D;
(vii) at the position corresponding to position 407-413 of SEQ ID NO: 17 an internal deletion;
(viii) at the position corresponding to position 440 of SEQ ID NO: 17 an M;
(ix) at the position corresponding to position 471 of SEQ ID NO: 17 an L;
(x) at the position corresponding to position 530 of SEQ ID NO: 17 an E;
(xi) at the position corresponding to position 552 of SEQ ID NO: 17 an I;
(xii) at the position corresponding to position 573 of SEQ ID NO: 17 an R;
(xiii) at the position corresponding to position 596 of SEQ ID NO: 17 a K;
(xiv) at the position corresponding to position 607 of SEQ ID NO: 17 an R;
(xv) at the position corresponding to position 610 of SEQ ID NO: 17 an S; and
(xvi) at the position corresponding to position 703 of SEQ ID NO: 17 a V.

31. The nucleic acid molecule of claim 30 which is a DNA molecule.

32. The nucleic acid molecule of claim 30, wherein said expression product is an RNA or a (poly)peptide.

33. A recombinant DNA molecule comprising the nucleic acid molecule of claim 30 and a regulatory region being capable of controlling expression of said nucleic acid molecule.

34. The recombinant DNA molecule of claim 33, wherein said regulatory region is a naturally occurring regulatory region or a genetically engineered derivative thereof.

35. The recombinant DNA molecule of claim 33, wherein said regulatory region comprises or is a promoter.

36. A vector comprising the recombinant DNA molecule of claim 34.

37. The vector of claim 36 comprising a heterologous promoter.

38. The vector of claim 37, wherein the heterologous promoter is controlling gene expression in spermatogenesis and/or in spermiogenesis.

39. The vector of claim 38, wherein the heterologous promoter is the testis promoter of c-kit, ACE, Tcr or Smok.

40. A host cell or organism transformed or transfected with the nucleic acid molecule of claim 32, the recombinant DNA molecule of claim 33 or the vector of claim 36.

41. A method of recombinantly producing an expression product as defined in claim 30 comprising the steps of culturing the host cell of claim 40 under conditions to cause expression of the protein and recovering said protein from the culture.

42. An expression product encoded by the nucleic acid molecule of claim 30 or obtainable by the method of claim 41.

43. A method for the identification of a nucleic acid molecule encoding an expression product with a Distorter function, comprising the steps of
(a) isolating a nucleic acid molecule encoding a candidate expression product with a Distorter function from the mouse t-complex by means of genomic localization, wherein said nucleic acid molecule is involved in G protein signalling; and
(b) testing the nucleic acid molecule isolated in step (a) for a change of the transmission ratio of the Responder or of a genetic trait linked to a Responder in an experimental non human animal, wherein when said transmission ratio is enhanced or reduced, said nucleic acid molecule isolated in (a) is a nucleic acid molecule encoding an expression product with Distorter function.

44. A method for the identification of an expression product of a nucleic acid molecule encoding a Distorter, comprising the steps of
(a) isolating an expression product of a nucleic acid molecule encoding a candidate Distorter by means of protein-protein interaction with a known Distorter derived from the mouse t-complex; and
(b) testing the nucleic acid molecule encoding said expression product isolated in (a) for change of the transmission ratio of the Responder or of a genetic trait linked to a Responder in an experimental non human animal, wherein when said transmission ratio is enhanced or reduced, said expression product isolated in (a) is an expression product with Distorter function.

45. The method of claim 43, wherein in step (b) hypomorphic or hypermorphic alleles of said nucleic acid molecule are used for testing for change of the transmission ratio.


SEQUENCE LISTING (129 sequences)

SEQ ID NO: 1 - DNA, 2145 bp, Mouse
atgaagctgataagcagtctcgatggttcaaaaacacttaatgctaacaacatggagacattaattgaatgtcagtcagagggtgatatcaaagtgcctcccctgctgacatcatgtgagagtgaagacagcatttgccagctaactgaaattaagaagagaaagaaagtgctgtcctggccatctctcatgagaaagctctctccttcatccgacttctctgggtcattggaaccagagctgaaagtgtcgctgtttgaccaacccttgtcgatcatctgtggggagaacgacacacttcccagacccatccaggacatcctcaccatcctctgccttaaaggtccttcaacggaagggatattcaggaaagctgccagcgagaaagcccgcaaggagctgaaggaggggcttaactgtggggtctccgtgaatctgaagcagctccctgtgcacctcctagctgtggtcttcaaggacttcctccgagggatacccctgaagctactctcctgtgatctctttgaggactggatgggcgccctggagaagcccaccgaggaggacagaatcgaggccctgaagcaggttgctggtggcctcccccggcccaaccttctcctgctcaggcacttgctctacgtgctccacctcatcagcaagaacgctgaggtcaacaagatggactccagcaacctcgccatctgcatcgggcctaacatgctcactctgaagaacgaccagagcctgtccttccaggcccagaaggacctgaacaataaggttaagatcttggtggaattcctcatcgacaactgctttgagatatttggggagaacattcggacgcgttcccgcatcacttctgacgactccctggaacacactgacagttcagacgtgtcaactctgcagaatgactcggcctatgacagcaatgacccagatgtggagcccacaagtggcgcagcctctcccaacaggcaactggagggtcccactcccacaatggctggtctggatacccggggccaacgggacacctgtgagtcaagctcagagtccagcgttagcatggtagtcaggctgaaaagctccattgtccaacaagacaggcggttctctgaacccaacatgtcaccctcacgagagtgcctcgtgggcccaacatccaagcaaaagctagcaaggagtgaggacagcttcactctgtcccaggacgcctcctgttctgaaggcgatgaagctgaagatccctttacagaggaagtcttcccagcagtggacagcaaacccaagagacctgtggatttgaaaataaagaattggacccaaggtttagcatctccacagggacacataaccaaagctttctccagatcctccccaggcgaatctttgggcagctcacctgtgccttctccatcctgccccaagagaaacttcttcaccagacaccagagtttcacaacaaagacagacaaaaccaaaccccagagagaaattagaaagcactccatgtcgttttcctttgcgtctcacaagaaagtgctgccccgaacctccagcattgggtctgagaaatccaaagacttttctagagaccaactccagaaggacttgaggaaagagagccaactttctggcagaatcgtccaggaaaatgagtcagaaatccaaagccaaacatctctgggcttcagcttgtctggcacctgggccctctcagtcgacaacacgttccagttagtggatatgaggaaaccaggaagcccaccatcttacgaagaggccatttattaccagacatcaggactcacagcctacggtggccagacagttgggagtatgaggtcaagaatgttcaagccaagcacagcagtgccccctgtgccttctcaccatggaggtgacctcagtgaagggacacctggtggacacagattgtcttctgtgactgagcactggacacacagtcagactgtccatgtctctatagaaactcaggggagatctgagctacaccagttgaggacagtgtccgagtccatgcagaaggctaagctggactgtcttgggccacaacacagccacttagtctttgaggctgaccaactctgctgtgctagagaatcctacatttaa

SEQ ID NO: 2 - DNA, 2145 bp, Mouse
atgaagctgataagcagtctcgatggttcaaaaacacttaatgctaacaacatggagacattaattgaatgtcagtcagagggtgatatcaaagtgcctcccctgctgacatcatgtgagagtgaagacagcatttgccagctaactgaaattaagaagagaaagaaagtgctgtcctggccatctctcatgagaaagctctctccttcatccgacttctctgggtcattggaaccagagctgaaagtgtcgctgtttgaccaacccttgtcgatcatctgtggggagaacgacacacttcccagacccatccaggacatcctcaccatcctctgccttaaaggtccttcaacggaagggatattcaggaaagctgccagcgagaaagcccgcaaggagctgaaggaggggcttaactgtggggtctccgtgaatctgaagcagctccctgtgcacctcctagctgtggtcttcaaggacttcctccgagggatacccctgaagctactctcctgtgatctctttgaggactggatgggcgccctggagaagcccaccgaggaggacagaatcgaggccctgaagcaggttgctggtggcctcccccggcccaaccttctcctgctcaggcacttgctctacgtgctccacctcatcagcaagaacgctgaggtcaacaagatggactccagcaacctcgccatctgcatcgggcctaacatgctcactctgaagaacgaccagagcctgtccttccaggcccagaaggacctgaacaataaggttaagatcttggtggaattcctcatcgacaactgctttgagatatttggggagaacattcggacgcgttcccgcatcacttctgacgactccctggaacacactgacagttcagacgtgtcaactctgcagaatgactcggcctatgacagcaatgacccagatgtggagcccacaagtggcgcagcctctcccaacaggcaactggagggtcccactcccacaatggctggtctggatacccggggccaccgggacacctgtgagtcaagctcagagtccagcgttagcatggtagtcaggctgaaaagctccattgtccaacaagacaggcggttctctgaacccaacatgtcaccctcacgagagtgcctcgtgggcccaacatccaagcaaaagctagcaaggagtgaggacagcttcactctgtcccaggacgcctcctgttctgaaggcgatgaagctgaagatccctttacagaggaagtcttcccagcagtggacagcaaacccaagagacctgtggatttgaaaataaagaattggacccaaggtttagcatctccacagggacacataaccaaagctttctccagatcctccccaggcgaatctttgggcagctcacctgtgccttctccatcctgccccaagagaaacttcttcaccagacaccagagtttcacaacaaagacagacaaaaccaaaccccagagagaaattagaaagcactccatgtcgttttcctttgcgtctcacaagaaagtgctgccccgaacctccagcattgggtctgagaaatccaaagacttttctagagaccaactccagaaggacttgaggaaagagagccaactttctggcagaatcgtccaggaaaatgagtcagaaatccaaagccaaacatctctgggcttcagcttgtctggcacctgggccctctcagtcgacaacacgttccagttagtggatatgaggaaaccaggaagcccaccatcttacgaagaggccatttattaccagacatcaggactcacagcctacggtggccagacagttgggagtatgaggtcaagaatgttcaagccaagcacagcagtgccccctgtgccttctcaccatggaggtgacctcagtgaagggacacctggtggacacagattgtcttctgtgactgagcactggacacacagtcagactgtccatgtctctatagaaactcaggggagatctgagctacaccagttgaggacagtgtccgagtccatgcagaaggctaagctggactgtcttgggccacaacacagccacttagtctttgaggctgaccaactctgctgtgctagagaatcctacatttaa

SEQ ID NO: 3 - DNA, 1329 bp, Mouse
atgaagctgataagcagtctcgatggttcaaaaacacttaatgctaacaacatggagacattaattgaatgtcagtcagagggtgatatcaaagtgcctcccctgctgacatcatgtgagagtgaagacagcatttgccagctaattgaaattaagaagagaaagaaagtgctgtcctggccatctctcatgagaaagctctctccttcatccgacttctctgggtcattggaaccagagctgaaagtgtcgctgtttgaccaacccttgtcgatcatctgtggggagaacgacacacttcccagacccatccaggacatcctcaccatcctctgccttaaaggtccttcaacggaagggatattcaggaaagctgccagcgagaaagcccgcaaggagctgaaggaggggcttaactgtggggtctccttgaatctgaagcagctccctgtgcacctcctagctgtggtcttcaaggacttctttcgagggatacccctgaagctactctcctgtgatctctttgaggactggatgggcgccctggagaagcccaccgaggaggacagaatcgaggccctgaagcaggttgctggtggcctcccccggcccaaccttctcctgctcaggcacttgctctacgtgctccacctcatcagcaagaacgctgaggtcaacaagatggactccagcaacctcgccatctgcatcgggcctaacatgctcactctgaagaacgaccagagcctgtccttccaggcccagaaggacctgaacaataaggttaagatcttggtggaattcctcatcgacaactgctttgagatatttggggagaacattcggacgcgttcccgcatcacttctgacgactccctggaacacactgacagttcagacgtgtcaactctgcagaatgactcggcctatgacagcaatgacccagatgtggagcccacaagtggcacagcctctcccaacaggcaactggagggtcccactcccacaatggctggtctggatacccggggccaccgggacacctgtgagtcaagctcagagtccagcgttagcatggtagtcaggctgaaaagctccattgtccaacaagacaggcggttctctgaacccaacatgtcaccctcacgagagtgcctcgtgggcccaacatccaaacaaaagctaacaaggagtgaggacagcttcactctgtcccaggacgcctcctgttctgaaggcgatgaagctgaagatccctttacagaggaagtcttcccagcagtggacagcaaacccaagagacctgtggatttgaaaataaagaattga

SEQ ID NO: 4 - DNA, 2124 bp, Mouse
atgaagctgataagcagtctcgatggttcaaaaacacttaatgctaacaacatggagacattaattgaatgtcagtcagagggtgatatcaaagtgcctcccctgctgacatcatgtgagagtgaagacagcatttgccagctaattgaaattaagaagagaaaaaaagtgctgtcctggccatctctcatgagaaagctctctccttcatccgacttctctgggtcattggaaccagagctgaaagtgtcgctgtttgaccaacccttgtcgatcatctgtggggagaacgacacacttcccagacccatccaggacatcctcaccatcctctgccttaaaggtccttcaacggaagggatattcaggaaagctgccagcgagaaagcccgcaaggagctgaaggaggagcttaactgtggggtctccgtgaatctgaagcagctccctgtgcacctcctagctgtggtcttcaaggacttcctccgagggatacccctgaagctactctcctgtgatctctttgaggactggatgggcgccctggagaagcccacggaggcggacagaatcgaggccctgaagcaggttgctggcggcctcccccggcccaaccttttcctgctcaggcacttgctctacgtgctccacctcatcagcaagaacgctgaggtcaacaagatggactccagcaaccttgccatctgcatcgggcctaacatgctcactctgaagaacgaccagagcctgtccttccaggcccagaaggacctgaacaataaggttaagatcttggtggaattcctcattgacaactgctttgagatatttggggagaacattcggacgcgttcccgcatcacttctgacgactccctggaacacactgacagttcagacatgtcaactctgcagaatgactcggcctatgacagcaatgacccagatgtggagcccacaagtggcacagcctctcccaacaggcaactggaggatcccactcccacaatggctggtctggatacccggggccaccgggacacctgtgagtcaagctcagagtccagcgttagcatggtagtcaggctgaaaagctccattgtccaacaagacaggcggttctctgaacccaacatgtcaccctcacgagagtgcctcgtgggcccaacatccaagcaaaagctaacaaggagtgaggacagcttcactctgtcccaggatgaagctgaagatccctttacagaggaagtcttcccagcagtggacagcaaacccaagagacctgtggatttgaaaatgaagaattggacccaaggtttagcatctccacagggacacataaccaaagctttctccagatcctccccaggcgaatctttgggcagctcacttgtgccttctccatcctgccccaagagaaacttcttcaccagacaccagagtttcacaacaaagacagacaaaaccaaaccccagagagaaattagaaagcactccatgtcgttttcctttgcgtctcacaagaaagtgctgccccgaacctccagcattgggtctgagaaatccaaagacttttctagagaccaactccagaaggacttgaggaaagagagccaactttctggcagaatcatccaggaaaatgagtcagaaatccaaagccaaacatctctgggcttcagcttgtctggcacctgggccctctcagtggacaacacgttccagttagtggatatgaggaaaccaggaagcccaccatcttacaaagaggccatttattaccagacatcaggactcagagcctacagtggccagacagttgggagtatgaggtcaagaatgttcaagccaagcacagcggtacctcctgtgccttctcaccatggaggtgacctcagtgaagggacacctggtggacacagattgtcttctgtgactgagcactggacacacagtcagactgtccatgtctctatagaaactcaggggagatctgagctacaccagttgaggacagtgtccgagtccatgcagaaggctaagctggactgtcttgggccacaacacagccacttagtctttgaggttgaccaactctgctgtgctagagaatcctacatttaa

SEQ ID NO: 5 - DNA, 2124 bp, Mouse
atgaagctgattagcagtctcgatggttcaaaaacacttaatgctaacaacatggagacattaattgaatgtcagtcagagggtgatatcaaagtgcctcccctgctgacatcatgtgagagtgaagacagcatttgccagctaattgaaattaagaagggaaaaaaagtgctgtcctggccatctctcatgagaaagctctctccttcatccgacttctctgggtcattggaaccagagctgaaagtgtcgctgtttgaccaacccttgtcgatcatctgtggggagaacgacacacttcccagacccatccaggacatcctcaccatcctctgccttaaaggtccttcaacggaagggatattcaggaaagctgccagcgagaaagcccgcaaggagctgaaggaggagcttaactgtggggtctccgtgaatctgaagcagctccctgtgcacctcctagctgtggtcttcaaggacttcctccgagggatacccctgaagctactctcctgtggtctctttgaggactggatgggcgccctggagaagcccacggaggaggacagaatcgaggccctgaagcaggttgctggtggcctcccccggcccaaccttttcctgctcaggcacttgctctacgtgctccacctcatcagcaagaacgctgaggtcaacaagatggactccagcaaccttgccatctgcatcgggcctaacatgctcactctgaagaacgaccagagcctgtccttccaggcccagaaggacctgaacaataaggttaagatcttggtggaattcctcattgacaactgctttgagatatttggggagaacattcggacgcgttcccgcatcacttctgacgactccctggaacacactgacagttcagacatgtcaactctgcagaatgactcggcctatgacagcaatgacccagatgtggagcccacaagtggcacagcctctcccaacaggcagctggaggatcccactcccacaatggctggtctggatacccggggccaccgggacacctgtgagtcaagctcagagtccagcgttagcatggtagtcaggctgaaaagctccattgtccaacaagacaggcggttctctgaacccaacatgtcaccctcacgagagtgcctcgtgggcccaacatccaagcaaaagctaacaaggagtgaggacagcttcactctgtcccaggatgaagctgaagatccctttacagaggaagtcttcccagcagtggacagcaaacccaagagacctgtggatttgaaaatgaagaattggacccaaggtttagcatctccacagggacacataaccaaagctttctccagatcctccccaggcgaatctttgggcagctcacttgtgccttctccatcctgccccaagagaaacttcttcaccagacaccagagtttcacaacaaagacagacaaaaccaaaccccagagagaaattagaaagcactccatgtcgttttcctttgcgtctcacaagaaagtgctgccccgaacctccagcattgggtctgagaaatccaaagacttttctagagaccaactccagaaggacttgaggaaagagagccaactttctggcagaatcatccaggaaaatgagtcagaaatccaaagccaaacatctctgggcttcagcttgtctggcacctgggccctctcagtggacaacacgttccagttagtggatatgaggaaaccaggaagcccaccatcttacaaagaggccatttattaccagacatcaggactcagagcctacagtggccagacagttgggagtatgaggtcaagaatgttcaagccaagcacagcggtaccccctgtgccttctcaccatggaggtgacctcagtgaagggacacctggtggacacagattgtcttctgtgactgagcactggacacacagtcagactgtccatgtctctatagaaactcaggggagatctgagctacaccagttgaggacagtgtccgagtccatgcagaaggctaagctggactgtcttgggccacaacacagccacttagtctttgaggttgaccaactctgctgtgctagagaatcctacatttaa

SEQ ID NO: 6 - DNA, 2124 bp, Mouse
atgaagctgattagcagtctcgatggttcaaaaacacttaatgctaacaacatggagacattaattgaatgtcagtcagagggtgatatcaaagtgcctcccctgctgacatcatgtgagagtgaagacagcatttgccagctaattgaaattaagaagagaaagaaagtgctgtcctggccatctctcatgagaaagctctctccttcatccgacttctctgggtcattggaaccagagctgaaagtgtcgctgtttgaccaacccttgtcgatcatctgtggggagaacgacacacttcccagacccatccaggacatcctcaccatcctctgccttaaaggtccttcaacggaagggatattcaggaaagctgccagcgagaaagcccgcaaggagctgaaggaggagcttaactgtggggtctccgtgaatctgaagcagctccctgtgcacctcctagctgtggtcttcaaggacttcctccgagggatacccctgaagctactctcctgtgatctctttgaggactggatgggcgccctggagaagcccacggaggaggacagaatcgaggccctgaagcaggttgctggtggcctcccccggcccaaccttttcctgctcaggcacttgctctacgtgctccacctcatcagcaagaacgctgaggtcaacaagatggactccagcaaccttgccatctgcatcgggcctaacatgctcactctgaagaacgaccagagcctgtccttccaggcccagaaggacctgaacaataaggttaagatcttggtggaattcctcattgacaactgctttgagatatttggggagaacattcggacgcgttcccgcatcacttctgacgactccctggaacacactgacagttcagacatgtcaactctgcagaatgactcggcctatgacagcaatgacccagatgtggagcccacaagtggcacagcctctcccaacaggcaactggaggatcccactcccacaatggctggtctggatacccggggccaccgggacacctgtgagtcaagctcagagtccagcgttagcatggtagtcaggctgaaaagctccattgtccaacaagacaggcggttctctgaacccaacatgtcaccctcacgagagtgcctcgtgggcccaacatccaagcaaaagctaacaaggagtgaggacagcttcactctgtcccaggatgaagctgaagatccctttacagaggaagtcttcccagcagtggacagcaaacccaagagacctgtggatttgaaaatgaagaattggacccaaggtttagcatctccacagggacacataaccaaagctttctccagatcctccccaggcgaatctttgggcagctcacttgtgccttctccatcctgccccaagagaaacttcttcaccagacaccagagtttcacaacaaagacagacaaaaccaaaccccagagagaaattagaaagcactccatgtcgttttcctttgcgtctcacaagaaagtgctgccccgaacctccagcattgggtctgagaaatccgaagacttttctagagaccaactccagaaggacttgaggaaagagagccaactttctggcagaatcatccaggaaaatgagtcagaaatccaaagccaaacatctctgggcttcagcttgtctggcacccgggccctctcagtggacaacacgttccagttagtggatatgaggaaaccaggaagcccaccatcttacaaagaggccatttattaccagacatcaggactcagagcctacagtggccagacagttgggagtatgaggtcaagaatgttcaagccaagcacagcggtaccccctgtgccttctcaccatggaggtgacctcagtgaagggacacctggtggacacagattgtcttctgtgactgagcactggacacacagtcagactgtccatgtctctatagaaactcaggggagatctgagctacaccagttgaggacagtgtccgagtccatgcagaaggctaagctggactgtcttgggccacaacacagccacttagtctttgaggttgaccaactctgctgtgctagagaatcctacatttaa

SEQ ID NO: 7 - DNA, 3363 bp, Homo sapiens
gacatagctgccctaaaaggaatgaggaagcgagagctctccagtgtctggctggctccgtccgtgtgacagcccatgatgttctttccggtctctgtaatattctgaatttccacctgcccgccccttcgcttataatgcagagcatgtgaagggagaccggctcggtctctctctctcccagtggactagaaggagcagagagttatgctgtttctcccattctttacagctcaccggatgtaaaagaactctggctagagaccctccaaggacagaggcacagccacacgggagtgaaatccacccctggacagtcagccgcaatactgatgaagctgagaagcagccacaatgcttcaaaaacactaaacgccaataatatggagacactaatcgaatgtcaatcagagggtgatatcaaggaacatcccctgttggcatcatgtgagagtgaagacagtatttgccagctcattgaagttaagaagagaaagaaggtgctgtcctggccctttctcatgagaaggctctcccctgcatcagatttttctggggctttggagacagacttgaaagcatcgctatttgatcagcccttgtcaattatctgcggtgacagtgacacactccccagacccatccaggacattctcactattctatgccttaaaggcccttcaacggaagggatattcaggagagcagccaacgagaaagcccgtaaggagctgaaggaggagctcaactctggggatgcggtggatctggagaggctccccgtgcacctcctcgctgtggtctttaaggacttcctcagaagtatcccccggaagctactttcaagcgacctctttgaggagtggatgggtgctctggagatgcaggacgaggaggacagaatcgaggccctgaaacaggttgcagataagctcccccggcccaacctcctgctactcaagcacttggtctatgtgctgcacctcatcagcaagaactctgaggtgaacaggatggactccagcaatctggccatctgcattggacccaacatgctcaccctggagaatgaccagagcctgtcatttgaagcccagaaggacctgaacaacaaggtgaagacactggtggaattcctcattgataactgctttgaaatatttggggagaacattccagtgcattccagtatcacttctgatgactccctggagcacactgacagttcagatgtgtcgaccctgcagaatgactcagcctacgacagcaacgaccctgatgtggaatccaacagcagcagtggcatcagctctcccagcaggcagccccaggtgcccatggccacagctgctggcttggatagcgcgggcccacaggatgcccgagaggtcagcccagagcccattgtgagcaccgtggccaggctgaaaagctccctcgcacagcccgataggagatactcagagcccagcatgccatcctcccaggagtgcctcgagagccgggtgacaaaccaaacactaacaaagagtgaaggggacttccccgtgccccgggtaggctctcgtttggaaagtgaggaggctgaagacccatttccagaggaggtcttccctgcagtgcaaggcaaaaccaagaggccggtggacctgaagatcaagaacttggccccgggttcggtgctcccgcgggcactggttctcaaagccttctccagcagctcgctggacgcgtcctctgacagctcgcccgtggcttctccttccagtcccaaaagaaatttcttcagcagacatcagtctttcaccacaaagacagagaaaggcaagcccagccgagaaattaaaaagcactccatgtctttcacctttgcccctcacaaaaaagtgctgaccaaaaacctcagcgcgggctctgggaaatcgcaagactttaccagggaccacgtcccgaggggtgtcagaaaggaaagccagcttgccggccgaatcgtgcaggaaaatgggtgtgaaacccacaaccaaacagcccgcggcttctgcctgagaccccacgccctctcggtggatgatgtgttccagggagctgactgggagaggcctggaagcccaccctcttatgaagaggccatgcagggcccggcagccagactagtggcctccgagagccagaccgtggggagcatgacggtggggagcatgagggcgaggatgctggaggcgcactgcctcctaccccctcttccacctgctcaccacgtagaggactcaagacacaggggcagcaaagagccactccctggccacggactctctcccctgcctgagcgatggaaacagagcagaactgtccatgcttctggggactctctggggcacgtgtctggcccagggagacctgagctcctcccgctgaggaccgtctccgagtccgtgcagaggaataagcgggactgtctcgtgcgacgatgtagccagccggtctttgaggctgaccaattccaatatgccaaagaatcgtatatttaggagggaggccatacgccatgccatagcttgtgctatctgtaaatatgagacttgtaaagaactgcctgtagattgtttttaaaaggtcttgaataagctccttgagaaagttgtggaaagccctcctcagtgaggatagctacaccatggccatggcgcatcagatagtctctgtgtacctggatttgtgcaatatgtaaaaatgtatcaaatgtattatagataaggtgttaggtgcaaaggatgtctaataatccctgcacacgttttgaacttgcagtgaagtacactgctgttccttgcttcctggggcacttttctcttggttagtgtttaaaaattatcttcgcttttttaatgtggcctcaaatgtcatgccaattttcacatcttccacaaactccatttagggagaaatgtttaaatctctggtataagtttactccataccagagtaaactatatattactctatataagcagtcttgcaataactaatcaccaccatagaagaaagaaacagactgcaaggaacagagttgagtgtctggagtcatcaaaggcattaaaaactccagtaaaagctggggccgtagcaaaaatcatgaaaaacacttcaacgtgtcctttcaatcatccaattaaatgtgggtagattaatgaaaatgtattacatcaatattaactcatctatagcactttgagtatctttgtagttcatgatatcctatcctataatgtggaggtaaatgattttatatgcattgggggtcatatataaaacttcaatgtaatttcactacaataaattgccttccttatttgaaagtaaaaa

SEQ ID NO: 8 - DNA, 3027 bp, Rattus norvegicus
atggaattgttaagtgctcgcctgccccgagcaggagaaacatttgtcccaatatctgagcatcagcactggaagccagagaacgcccacaacaacggagctcagtgtttcccagagctcacgttaggccggacgcatcaaggcaacaaggtcctggacagtgagtgggtcacacagatgctaaggaccacactagcctttcggagggcactttatatcaagcttacagcatccattctagaacttcagaacaggaagaacgggaccttccatgtacttcggggtgagaattacaagttcagcagtgttgaacacagttctctggctggcatacagcaggtgttcagcaagcattcccagtctgtccaagactgttctgacgttgaaaaagttgtagcaatagttgtggctgagaaacctggaatctcattcaagaacccaccaccggctttaatggctggccccagggacttggcagccgcagagctggagcgaggagaagcaaagagacctccggaagagatccagtgcgatggggtgtcccagataacagctcctctggaatggagggaaagcaggttctatgagaatagtgtgagtgataaaacagatgaattcctgtgcccacagatccgctacaagagctccacaaggggtcgctgcaggcgccacgcagggggtcggtacatgcgcctagcaaggcccctgccactggataacaaccacctggagactcatcgtgtgaggacagtgctagtgtctattttgtccagacccctgagtcatcctgtgagggacgctgcttcacttgggctcactcaaacacagaaccaaagcctcgaggtagagaaaattccccttggactgtcagccccgaatccaatgaagttgataagcagtctcgatggtgcaaaaacgcttaatgccaacaacatggacacattaattgaatgtcagtcagagggcgatatcaaggtgcttccattgctgacgtcatgtgagagtgaagacagcatttgccagctaattgaagttaagaagagaaagaaagtgctgtcctggccatctctcatgagaaagctctctccttcaccagacttctctgggtcattggaaccagagctgaaagtgtcgctgtttgatcaacccttgtcaatcatctgtaaggagaatgacacactccctagacccatccaggacatcctcaccatcctctgccttaaaggtccttcaactgaaggaatattcaggaaagcagccagcgagaaagcccgcaaggagctgaaggaggagcttaactgtgggggctctgtgaatctgaaccagctccctgtgcacctcctggctgtggtcttcaaggacttcctccgaggaatccccctgaagctgctctcctgtgacctctttgaggactggatgggagccctggagaagcccagtgaggaggacaggatcgaggccctgaagcaggttgctgatcggctcccccggcccaacctccttctgctcaggaccttagtctacgtgctacaccttatcagcaagaacgccgaggtcaacaagatggactccagcaacctggccatctgcatcggacccaacatgctcacactgaagaatgaccagagcctgtccttccaggcccagagggacctgaacaataaggttaagatcttggtggaattcctcattgacaactgccttgaaatatttggggagaacattccgacacatccccgcatcacttctgatgactctctggaacacactgacagctcagacgtgtcgactctgcagaacgactcagcctatgacagcaatgacccggatgtagagcccgcgaatggagttgcctctccctgcaggcagttggagggtccctctaccacagtggctggcatggatacccgggggccccggaacacctgtgagtcgagctcagaatccagtgtcagcatggtagccaggctgaaaagctccattggccagcaagacaggcgtttctctgaacccaacatgccaccctcacgagagtgcttcgtgggcccgataaccaagcaaaagctaacgaggagcgaggacagcttcgttctgccccaggaagcctcctgttctgaaggcaatgaagccgaagatccctttacagaggaagtcttcccagcagttgaaggcaaacccatgagaccagtggatttgaagataaagaacttgacccaaggtttagcatctccacagggacctgcaaacaaagctttctccagcttctccgcgggggaatctttggacagctcacctgtgccttctccatcctgtcccaagagaaacttcttcaccagacaccagagtttcaccacaaagacggacaagaccaagccccagagagaaattagaaagcactccatgtcattttcctttgcgtctcacaagaaagtgctgccccggacctccagcattgggtctgagaaatccaaagacttctctagagaccagctccagaaggacttgaggaaagagagccagcttgccggcagaatcatccgggaaaatgagtccgaaatccaaagccaaacaaatctgggctccagcttgtctggaacctgggccctctcagttgataacacgttccagttcattgatgtgaggaagccaggaagcccaccatcttatgaagaggccatttattaccacacatcaggactcacagcctacagtggccagacagttgggagtatgagagcaagaatgttgaagcagagcatggcggtgccccctgtgccttctcaccatgaaggtgagctcagtgaagggatacctggtggacacagatcgtcttccgtgactgagcactggacacagagtcagactgtccatgtctctgtagaaactcgggggagatctgagctacatcgattgaggacagtgtctgagtccgtgcagagggctaagctggactaccttgggcagcaacgcagccacttggtctttgaggttgaccaactccgatgtgctaaagaatcctacatttag

SEQ ID NO: 9 - DNA, 2181 bp, Mouse
atggagcgagcctgtgagaagcaggactcagtgtgcaacctggtggctgtgtttgagaacaacaggactccgggagaagcacctggatcccatagccttgaggaccagccccatatccctgaacaccagctgtccctgtccccagagccttgggaggcaccccctgtcaaggaggccttgaagtctgaattccggccagtgagcaggacatatctgagttccctcaagaacaagctatcgagtggggcttggaggagatcctgtcagcctggggtcagcccggggccagagacacaggaacctgaggagaagagggtcgtgcgagagcttctggagacggagcaggcctatgtggctcgcctgcacctgcttgaccaggtgttcttccaggagctgctgagggaggcaggccgcagcaaggccttccctgaggacgtggtgaagctcattttctccaacatctcctccatctatcgtttccacgcccagttcttccttcccgagctgcagcggcgcgtggatgactgggcagccacgccccgcattggggatgtgatccagaagctggccccgtttctgaaaatgtacagcgagtacgtgaagaactttgagcgggccgcggaactgctggccacgtggatggacaagtctcagcccttccaggaggtggtcacccgcatccagtgcagcgaggcctccagcagcctgaccctgcagcaccacatgttggagcctgtgcaaagaatcccgcggtacgaactgctgctcaaggaatatgtgcagaagctgccagcccaggccccagacctcgaagatgcccagagagcactggacatgatcttctcagctgcacagcactccaatgcagccattgcagagatggagcggctgcagggcctgtgggatgtgtaccagcgcctgggcttggaggatgacatcgtggacccctccaacaccctgctccgagagggccctgttctcaagatctctttccgccgcagcgacccaatggaacgctacctggttttgttcaacaacatgcttctgtattgtgtaccccgagtcctccaagtgggtgcccagttccaggtgcggactcgcatcgatgtggccggcatgaaggtgcgggagctgaccgatgctgagttcccacactccttcctggtgtccggaaagcagcgcacactggagctgcaggcccggtcccgagatgaaatggtttcctggatgcaggcctgccaggcagccattgaccaggttgagaagcggagtgagaccttcaaggctgctgtccagggacctcagggggacacgcaggagcccaagccacaggtagaggagctgggtctccgagcgcctcagtgggtccgggacaagatggtgaccatgtgcatgcgctgccaggagcccttcaatgccctgactcgtcggcgccaccactgccgggcttgcggctatgtagtgtgtgccaagtgctctgactaccgtgcggagctgaaatatgacagcaacaggcccaaccgagtctgcctgacctgctacacatttctcactggaaacgtactccctcaaggcaaggaggacaagaggcggggcatcttggagaaagaggcctcagcagcacccgagcagagtctggtgtgcagcttcctgcagctcataggggacaagtgcagcaggagccttccccggagctggtgtgtgatcccccgggatgacccccttgtcctgtatgtctatgcagccccccaggacacaaaggctcacacctccatccccctactgggctatcaggtgatttcagggccccaggggacctcgggttttccagctgcaacagtcaggcgagcagtacaccttcaaggccgagtctgtggagctgcagggccgctgggtgacagctatcaagcgtgcgccagtggcacccctgaggacctgacgaagaagatgtgtctgactgaaccagcagccagctgctcatcgagagtccatgacagccttcccaggcctaccccatggttctatcacttcaccccaagctgggctacccctgacccacctgatgtctataccgagacaattcatcctgactctgtatccagcagacacaggccctttccttcaggaagataccaccaagtgtcccagctggctggagagggggcggatatccctggatca

SEQ ID NO: 10 - DNA, 1965 bp, Mouse
atggagcgagcctgtgagaagcaggactcagtgtgcaacctggtggctgtgtttgagaacaacaggactccgggagaagcacctggatcccatagccttgaggaccagccccatagccctgaacaccagctgtccctgtccccagagccttgggaggcaccccctgtcaaggaggccttgaagtctgaattccggccagtgagcaggacatatctgagttccctcaagaacaagttatcgagtggggcttggaggagatcctgtcagcctggggtcagcccggggccagagacacaggaacctgaggagaagagggtcgtgcgagagcttctggagacggagcaggcctatgtggctcgcctgcacctgcttgaccaggtgttcttccaggagctgctgagggaggcaggccgcagcaaggccttccctgaggacgtggtgaagctcattttctccaacatctcctccatctatcgtttccacgcccagttcttccttcccgagctgcagcggcgcgtggatgactgggcagccacgccccgcattggggatgtgatccagaagctggccccgtttctgaaaatgtacagcgagtacgtgaagaactttgagcgggccgcggaactgctggccacgtggatggacaagtctcagcccttccaggaggtggtcacccgcatccagtgcagcgaggcctccagcagcctgaccctgcagcaccacatgttggagcctgtgcaaagaatcccgcggtacgaactgctgctcaaggaatatgtgcagaagctgccagcccaggccccagacctcgaagatgcccagagagcactggacatgatcttctcagctgcacagcactccaatgcagccattgcagagatggagcggctgcagggcctgtgggatgtgtaccagcgcctgggcttggaggatgacatcgtggacccctccaacaccctgctccgagagggccctgttctcaagatctctttccgccgcagcgacccaatggaacgctacctggttttgttcaacaacatgcttctgtattgtgtaccccgagtcctccaagtgggtgcccagttccaggtgcggactcgcatcgatgtggccggcatgaaggtgcgggagctgaccgatgctgagttcccacactccttcctggtgtccggaaagcagcgcacactggagctgcaggcccggtcccgagatgaaatggtttcctggatgcaggcctgccaggcagccattgaccaggttgagaagcggagtgagaccttcaaggctgctgtccagggacctcagggggacacgcaggagcccaagccacaggtagaggagctgggtctccgagcgcctcagtgggtccgggacaagatggtgaccatgtgcatgcgctgccaggagcccttcaatgccctgactcgtcggcgccaccactgccgggcttgcggctatgtagtgtgtgccaagtgctctgactaccgtgcggagctgaaatatgacagcaacaggcccaaccgagtctgcctgacctgctacacatttctcactggaaacgtactccctcaaggcaaggaggacaagaggcggggcatcttggagaaagaggcctcagcagcacccgagcagagtctggtgtgcagcttcctgcagctcataggggacaagtgcagcaggagccttccccggagctggtgtgtgatcccccgggatgacccccttgtcctgtatgtctatgcagccccccaggacacaaaggctcacacctccatccccctactgggctatcaggtgatttcagggccccagggggaccctcgggttttccagctgcaacagtcaggccagcagtacaccttcaaggccgagtctgtggagctgcagggccgctgggtgacagctatcaagcgtgcggccagtggccggacccctgagggacctgacgaagaagatgtgtctgac

SEQ ID NO: 11 - DNA, 1387 bp, Mouse
atgtacagcgagtacgtgaagaactttgagcgggccgcggaactgctggccacgtggatggacaagtctcagcccttccaggaggtggtcacccgcatccagtgcagcgaggcctccagcagcctgaccctgcagcaccacatgttggagcctgtgcaaagaatcccgcggtacgaactgctgctcaaggaatatgtgcagaagctgccagcccaggccccagacctcgaagatgcccagagagcactggacatgatcttctcagctgcacagcactccaatgcagccattgcagagatggagcggctgcagggcctgtgggatgtgtaccagcgcctgggcttggaggatgacatcgtggacccctccaacaccctgctccgagagggccctgttctcaagatctctttccgccgcagcgacccaatggaacgctacctggttttgttcaacaacatgcttctgtattgtgtaccccgagtcctccaagtgggtgcccagttccaggtgcggactcgcatcgatgtggccggcatgaaggtgcgggagctgaccgatgctgagttcccacactccttcctggtgtccggaaagcagcgcacactggagctgcaggcccggtcccgagatgaaatggtttcctggatgcaggcctgccaggcagccattgaccaggttgagaagcggagtgagaccttcaaggctgctgtccagggacctcagggggacacgcaggagcccaagccacaggtagaggagctgggtctccgagcgcctcagtgggtccgggacaagatggtgaccatgtgcatgcgctgccaggagcccttcaatgccctgactcgtcggcgccaccactgccgggcttgcggctatgtagtgtgtgccaagtgctctgactaccgtgcggagctgaaatatgacagcaacaggcccaaccgagtctgcctgacctgctacacatttctcactggaaacgtactccctcaaggcaaggaggacaagaggcggggcatcttggagaaagaggcctcagcagcacccgagcagagtctggtgtgcagcttcctgcagctcataggagacaagtgcagcaggagccttccccggagttggtgtgtgatcccccgggatgacccccttgtcctgtatgtctatgcagccccccaggacacaaaggctcacacctccatccccctactgggctatcaggtgatttcagggccccagggggaccctcgggttttccagctgcaacagtcaggccagcagtacaccttcaaggccgagtctgtggagctgcagggccgctgggtgacagctatcaagcgtgcggccagtggccggacccctgagggacctgacgaagaagatgtgtctgactgac

SEQ ID NO: 12 - DNA, 1968 bp, Mouse
atggagcgagcctgtgagaagcaggactcagtgtgcaacctggtggctgtgtttgagaacaacagcaggactccgggagaagcacctggatcccatagccttgaggaccagctccatagccctgaacaccagctgtccctgtccccagagccttgggaggcaccccctgtcaaggaggccttgaagtctgaattccggccagtgagcaggacatatctgagttccctcaagaacaagctatcgagtggggcttggaggagatcctgccagcctggggtcagcccggggccagagacacaggaacctgaggagaagagggtcgtgcgagagcttctggagacggagcaggcctatgtggctcgcctgcacctgcttgaccaggtgttcttccaggagctgctgagggaggcaggccgcagcaaggccttccctgaggacgtggtgaagctcattttctccaacatctcctccatctatcgtttccacgcccagttcttccttcccgagctgcagcggcgcgtggatgactgggcagccacgccccgcattggggatgtgatccagaagctggccccgtttctgaaaatgtacagcgagtacgtgaagaactttgagcgggccgcggaactgctggccacgtggatggacaagtctcagcccttccaggaggtggtcacccgcatccagtgcagcgaggcctcgggcagcctgaccctgcagcaccacatgttggagcctgtgcaaagaatcccgcggtacgaactgctgctcaaggaatatgtgcagaagctgccagcccaggccccagacctcgaagatgcccagagagcactggacatgatcttctcagctgcacagcactccaatgcagccattgcagagatggagcggctgcagggcctgtgggatgtgtaccagcgcctgggcttggaggatgacatcgtggacccctccaacaccctgctccgagagggccctgttctcaagatctctttccgccgcagcgacccaatggaacgctacctggttttgttcaacaacatgcttctgtattgtgtaccccgagtcctccaagtgggtgcccagttccaggtgcggactcgcatcgatgtggccggcatgaaggtgcgggagctgaccgatgctgagttcccacactccttcctggtgtccggaaagcagcgcacactggagctgcaggcccggtcccgagatgaaatggtttcctggatgcaggcctgccaggcagccattgaccaggttgagaagcggagtgagaccttcaaggctgctgtccagggacctcagggggacacgcaggagcccaagccacaggtagaggagctgggtctccgagcgcctcagtgggtccgggacaagatggtgaccatgtgcatgcgttgccaggagcccttcaatgccctgactcgtcggcgccaccactgccgggcttgcggctatgtagtgtgtgccaagtgctctgactaccgtgcggagctgaaatatgacagcaacaggcccaaccgagtctgcctgacctgctacacatttctcactggaaacgtactccctcaaggcaaggaggacaagaggcggggcatcttggagaaagaggcctcagcagcacccgagcagagtctggtgtgcagcttcctgcagctcataggagacaagtgcagcaggagccttccccggagctggtgtgtgatcccccgggatgacccccttgtgctgtatgtctatgcagccccccaggacacaaaggctcacacctccatccccctactgggctatcaggtgatttcagggccccagggggaccctcgggttttccagctgcaacagtcaggccagcagtacaccttcaaggccgagtctgtggagctgcagggccgctgggtgacagctatcaagcgtgcggccagtggccggacccctgagggacctgacgaagaagatgtgtctgac

SEQ ID NO: 13 - DNA, 5756 bp, Mouse
cggagctagagaagccttgaccttcccggctcagcgagtggagaacaaagtgacggagaaaacttcgcggagtgtgccctgagtccccacggggcctcgctcaggggcgcagagccgggctcgaccggtcctctgtgttgaaacttggaccgaggatcacttcatggatttgcctcacgtacctatgtttgttaatttcctgtgtcgttcccaagatcacgtaaaaccctctgagcattattgacctccctcccagcaaaagccaggctgcagtgtaatttagaccatggctcatattgatacaactgttctcaattttggttaaaatggggaactccgagagtcaatataccttccaaggatccaagaatcatagtaatactgtcactggtgctaagcaaaagccttgctctctgaaaatacgcagcgttcatgcaaaagacgagaagtccttacatggttggactcatgggagcagcggtgcaggctacaagtccaggtccctagcccgaagctgcctttctcactttaagaatcaccagccttacgccaccagactcagtggacccacatgtaaagtctcaaagggcaccacctactctaagcacagagcaaataccccgggaaatgatttccagggcaacagtggtgctttcttacctgagaatggcttccactatgttgaccgcgagtcagaggaaagccatatcacctccaatgggcaccttctcacctgctatgggagaaaggaaagcctcgcctccactcctccaggcgaggaccacaggagccccagggtgctcatcaagaccctgggaaagctagacgggtgtttaagagtcgagttccacaacggtggcaacccccacaaggggacctccgaggaccccagtggacctgtacggctgctgagatactcccctaccttagcatcggaaacctgcccggtgcgggaaaccaggcggcattccgctgcaggctccccatccagccagcggccctctcccactgactctcgcctgcgctccagcaaaggcagctccctgagctcagagtcatcctggtatgactccccctggggcaacgctggggaggtgagcgaggtggagggctccttcctggctcccagcactccagaccccagcctccccagcagcttcccacccagtgacaccaaaaagcctttcaaccaaagctcttccctctcctccctccgggaattgtacaaagatcccaacctggggtgccgctcaccttccggcacctgcctttcttccaatgagtacatcagctctcaagtcagcctgaacaaccgagtctcctttgcgtctgacatggatgtgccctccagggtggatcacagggatcccctgcactatagttcctttactctcccctgtcgcaagtccaaagccttaactgaagatgcagctaagaaagacaccctcaaagccagaatgcggcgcttcagtgactggacaggaagcctctccaggaagaagaggaaactgcaggaacccaggtccatggagggcagtgagtactttgatagccactcagatggactgaatgcagaagggcaggtgcccgcgcagacatcttccttactgtggtcagggggctcggctcagaccctgcctcacagaagcgaatccactcacgctatcagcgtcgatcccctccgacagaacatctatgagaatttcatgcgagagctcgaaatgagcaggagcaacacagaacacgtggaaacttccacagagaccatggagtccagcagcgagtctgtcagctcactggagcagctggatctgctctttgagaaggagcagggagtggtccggaaagccgggtggctcttcttcaaaccccttgtcaccttgcagaaggagaggaaactggagctggtggctcggaggaagtggaaacaatactgggtgaccctgaaaggctgtactctgctgttttatgagacctatggaaagaattccacagagcagaatagtgccccacggtgtgccctctttgcagaggacagcatcgtgcagtctgtcccagagcatcccaagaaggaacacgtgttctgcctgagtaactcctgtggggacgtctacctattccaggccactagccagacagatctggaaaactgggtcacagccatccactcgtgcgcatccctctttgcaaagaagcacggaaaggaggacacggtgcgactgctaaagagccagaccagaagcctgcttcagaagatagacatggatagcaagatgaagaagatggcagagttgcagctgtctgtggtgagcgaccccaagaacaggaaggccatcgagaatcagatccggcaatgggagcagaatctggaaaaattccacatggacctgttccgcatgcgctgctatttggcgagcttacaaggtggggagttaccaaatcccaagagtctccttgctgccaccagccgcccctccaagctggctcttggcaggctgggcgtcctgtctgtttcgtctttccatgctctggtgtgttccagagatgattccactctcaggaaaagaacactttcccttacccagagaggaaaaagcaagaaaggcatattttcttcattgaaaggtctggacaccctagcaagaaagggtagggagaagagagcttccataactcagatgtttgattctagccacagccatggatttcttggaactcagctacctcaaaagtccactaactccaacaaggcccatgacctgcatctgtatggctccgcagtagacagcgcgctgcgagacagcatgtgggaagtccagacttatgtccacttccaggataacgaaggagttactgtgaccatcaagccagagcacagggtggaagatgttctggctttggtgtgcaagatgagacagttggaacccactcactatggtcttcagctccgaaaggtggtcgataaaagtgtggagtggtgtgtgcccgcgctgtatgaatacatgcaagagcaggtttatgatgaaatcgaagttttcccactcagtgtgtatgacgtgcagctaaccaagactggggacatgactgactttgggtttgcagtcacagttcaggtggacgaacaccagcatctcaaccggatatttatcagtgatgttctccctgacagcctggcatacggaggagggctgagaaagggcaacgaaatcacaagcttaaatggggaaccagtgtctgaccttgacatccagcagatggaggctttgttttctgagaagagcgttggcctcactctagtcgcccggcctgtgaccacaagacgaaccctgtgtgcttcctggtcagacagtgacctgttctccagggaccagaaaagtctgccgccctctcccaaccagtcccagctgctggaggaattcctggataactttagaaaaaccgccacgagtgatttcagcaatgtccctgagatcacaactggcttgaagaggagccagacagaaggcaccctggatcaagtgccccacagggaaaagatggagcagacattcctgagcgctgaccagattgcggagctctgcagggacttgaacaacacccacaccaacagtatggaagcaccaacagagagccatgacccacctcccaggcctctggctcgtcacctctcagatgcagatcgcctccggaaagtcatccaggagcttgtagacacagagaagtcttacgtgaaggatctgagctgcctctttgaactatacttggagccacttcagaatgagacctttcttacccaagatgagatggagtcactttttgggagcctgccagagatgctggagtttcaaaaggtgttcctggagacgttggaggatgcgatctccgcttcctcggactttagtgtcctggaaaccccctcacagtttcgaaaattgctgttctcccttggaggttctttcctctactatgcggatcactttaagctatacagtgggttctgtgccaaccacattaaagtacagagggttctagagcgagctaaaacggacaaggccttcaaggcttttctggatgcccgaaatcccaccaagcagcactcctccacgctggagtcctatctcatcaagcctgttcagagagtgctcaagtatcctctgcttctcaaggagctagtgtcactgactgaccatgagagtgaagaacactatcacctgacagaagcactaaaggccatggaaaaagtcgccagtcacatcaatgagatgcagaagatctacgaggattacgggatggtgtttgaccagctggtggcagagcagagtggcacagagaaggaagtgacagagctgtccatgggggaacttctgatgcactctacagtttcctggttgaatccgttcctgtctctaggaaaagccaggaaggacattgagctcacagtatttgtttttaagagagctgtcatactggtttataaagaaaactgcaagctgaaaaagaaactgccctcgaattcccggcctgctcacaactctgctgacttggatccatttaaattccgctggttgattcccatatctgcgcttcaagttagactggggaacacggcagggactgaaaataattccacgtgggagctgattcataccaagtcggaaattgaaggacggccagaaaccatctttcaactgtgctgcagtgacagcgagaacaaaaccagcattgttaaggtgattcgttctattctgagagagaacttccggcgccacataaagtgtgagctgccactggagaagacgtgtaaggaccggctagtacctcttaagaaccgagttcctgtttcagccaaattagcctcgtccaggtcgttgaagggcctcagaacatcctccagcagcgagtggcccagcgagcccagcaagggcaactcactggactcagatgagtgcagcctgagcagtggcacccagagtagcggctgccccgtagccgagagcaggcgagactctaagagcaccgagctggagaaagacgctcaggagggcctggcggagtttccagatggtcttatcaaagaaagcgacattctgagtgatgaagatgaggacttccaccaccctctgaaacagggtagccctactaaggacattgagattcagttccagagactgaaaatctctgaggaatccgacgtgcacccagttgggcagcagcctctcacagagtcaggtgaacagcccaagctggtcaggggccatttttgccccattaaacggaaagcaaacagcaccaagaggggcagaggaactttgctcaaggcgcagactcgtcaccagtccctggacagccacccagaaactgccagcattgatctaaacttggtcctggagagagaattcagtgtccagagcttaacttcagtcgtcaatgaggagggtttttatgaaacacagagccatggcaaatcatgatttgtgtagtgtgcttacaatgctcatttgttttcaaattggtggaaaagagaaactgcaggaaaactattcatgtttgagttttacgcagtatattttttccacaaaacctgtaaagatttaagttattttaatttattgtgaatccgaaaactagatttagttggtcagaatctgtaaagtactagcacctatccctttggagcagcaatcaaatgatttagtcttgtaataaaattgcattttaattaaaaaaaaaaaaaaaaaaaaaaaaaaaaaaaaaaa

SEQ ID NO: 14 - DNA, 5302 bp, Homo sapiens
gctcacttcatggactcactttgcgtgcttgttaaatgtgctgtgttgctcccaagaccatgtaaagcctactgaccactaacctccctcacagcagaaactagacgtcaggttaaaatgggcaactccgacagtcagtacacccttcaaggatctaaaaatcatagcaatactattactggtgctaagcaaattccttgctccctgaaaatacgtggcattcatgcaaaagaggaaaagtcattgcatggatggggtcacggaagcaacggagcaggttacaagtccaggtccctggcccgaagctgcctttctcactttaagagtaaccagccttacgcatcgagactcggtggccccacatgcaaggtctccagaggtgttgcctactccacgcacaggacaaatgccccagggaaggatttccagggcatcagtgctgctttctcaactgagaatggcttccactctgttggccacgagctggcagataaccacatcacctccagagactgcaacggacaccttctcaactgctacgggaggaatgagagcattgcctccaccccaccgggcgaagaccgcaagagcccccgagtgctcatcaaaacgctggggaagctggatgggtgtttaagggtcgagttccacaatggtggcaaccccagcaaagtgcctgcagaggactgcagtgagccggtgcagctgctgaggtactcacctaccttagcatcggaaacctcccctgtgcctgaagccaggagggggtccagcgccgattccctgcccagccatcgcccctctcccacggactctcgcctgcggtccagcaaaggcagctccctgagttctgagtcatcctggtacgactccccttggggcaatgctggagagctgagcgaggctgagggctccttcctggcccccggcatgcctgaccccagtctccatgccagcttcccacctggcgatgccaaaaagcctttcaaccaaagctcttccctctcctccctccgggaactgtacaaagatgccaacctggggagcctctccccctcaggtatccgcctttctgatgaatacatgggcacgcatgccagcctgagcaaccgtgtctcttttgcttccgacattgatgtgccctccagagtggcacacggggaccccatccagtacagttccttcactctcccctgtcggaagcccaaagcctttgttgaggatactgcgaagaaggactccctcaaagccaggatgcgacggatcagtgactggacgggaagcctctcaaggaagaaaaggaaactccaggagccgaggtccaaggagggcagtgactactttgacagtcgctctgatggactgaatacagatgtgcagggatcctcccaggcatctgcttttctgtggtcagggggctctactcagatcctgtctcagagaagtgaatccacacatgcgattggcagcgatcccctccggcagaacatttatgagaatttcatgcgagagttggaaatgagcaggaccaacactgagaacatagaaacatctacagaaaccgccgagtccagcagcgagtcactcagctctctggaacagctggatctgctctttgagaaggaacagggggtggtccggaaggccgggtggctcttcttcaagcccctggtcactgtgcagaaggaaaggaagcttgagctggtggcacgaaggaaatggaaacagtactgggtaacgctgaaaggatgcacgctgctgttttatgagacctatgggaagaattccatggatcagagcagtgcccctcggtgtgctctgtttgcagaagacagcatagtgcagtctgttccagagcatcccaagaaagaaaatgtgttctgcctcagcaactcctttggagatgtctaccttttccaggccaccagccagacagatctagaaaactgggtcactgctgtacactctgcttgtgcatccctttttgcaaagaagcatgggaaagaggacacgctgcggctgctgaagaaccagaccaaaaacctgcttcagaagatagacatggacagcaagatgaagaagatggcagagctgcagctgtccgtggtgagcgacccaaagaacaggaaagccatagagaaccagatccagcaatgggagcagaatcttgagaaatttcacatggatctgttcaggatgcgctgctatctggccagcctacaaggtggggagttaccgaacccaaagagtctccttgcagccgccagccgcccctccaagctggccctcggcaggctgggcatcttgtctgtttcctctttccatgctctggtatgttctagagatgactctgctctccggaaaaggacactgtcactgacccagcgagggagaaacaagaagggaatattttcttcgttaaaagggctggacacactggccagaaaaggcaaggagaagagaccttctataactcaggtcgatgaacttctgcatatatatggttcaacagtagacggtgttccccgagacaatgcatgggaaatccagacttatgtccactttcaggacaatcacggagttactgtagggatcaagccagagcacagagtagaagatattttgactttggcatgcaagatgaggcagttggaacccagccattatggcctacagcttcgaaaattagtagatgacaatgttgagtattgcatccctgcaccatatgaatatatgcaacaacaggtttatgatgaaatagaagtctttccactaaatgtttatgacgtgcagctcacgaagactgggagtgtgtgtgactttgggtttgcagttacagcgcaggtggatgagcgtcagcatctcagccggatatttataagcgacgttcttcccgatggcctggcgtatggggaagggctgagaaagggcaatgagatcatgaccttaaatggggaagctgtgtctgatcttgaccttaagcagatggaggccctgttttctgagaagagcgtcggactcactctgattgcccggcctccggacacaaaagcaaccctgtgtacatcctggtcagacagtgacctgttctccagggaccagaagagtctgctgccccctcctaaccagtcccaactgctggaggaattcctggataactttaaaaagaatacagccaatgatttcagcaacgtccctgatatcacaacaggtctgaaaaggagtcagacagatggcactctggatcaggtttcccacagggagaaaatggagcagacattcaggagtgctgagcagatcactgcactgtgcaggagttttaacgacagtcaggccaacggcatggaaggaccgcgggagaatcaggatcctcctccgaggcctctggcccgccacctgtctgatgcagaccgcctccgcaaagtcatccaggagcttgtggacacagagaagtcctacgtgaaggatttgagctgcctctttgaattatacttggagccacttcagaatgagacctttcttacccaagatgagatggagtcactttttggaagtttgccagagatgcttgagtttcagaaggtgtttctggagaccctggaggatgggatttcagcatcatctgactttaacaccctagaaaccccctcacagtttagaaaattactgttttcccttggaggctctttcctttattacgcggaccactttaaactgtacagtggattctgtgctaaccatatcaaagtacagaaggttctggagcgagctaaaactgacaaagccttcaaggcttttctggacgcccggaaccccaccaagcagcattcctccacgctggagtcctacctcatcaagccggttcagagagtgctcaagtacccgctgctgctcaaggagctggtgtccctgacggaccaggagagcgaggagcactaccacctgacggaagcactaaaggcaatggagaaagtagcgagccacatcaatgagatgcagaagatctatgaggattatgggaccgtgtttgaccagctagtagctgagcagagcggaacagagaaggaggtaacagaactttcgatgggagagcttctgatgcactctacggtttcctggttgaatccatttctgtctctaggaaaagctagaaaggaccttgagctcacagtatttgtttttaagagagccgtcatactggtttataaagaaaactgcaaactgaaaaagaaattgccctcgaattcccggcctgcacacaactctactgacttggacccatttaaattccgctggttgatccccatctccgcgcttcaagtcagactggggaatccagcagggacagaaaataattccatatgggaactgatccatacgaagtcagaaatagaaggacggccagaaaccatctttcagttgtgttgcagtgacagtgaaagcaaaaccaacattgttaaggtgattcgttctattctgagggagaacttcaggcgtcacataaagtgtgaattaccactggagaaaacgtgtaaggatcgcctggtacctcttaagaaccgagttcctgtttcggccaaattagcttcatccaggtctttaaaagtcctgaagaattcctccagcaacgagtggaccggtgagactggcaagggaaccttgctggactctgacgagggcagcttgagcagcggcacccagagcagcggctgccccacggctgagggcaggcaggactccaagagcacttctcccgggaaatacccacaccccggcttggcagattttgctgacaatctcatcaaagagagtgacatcctgagcgatgaagatgatgaccaccgtcagactgtgaagcagggcagccctactaaagacatcgaaattcagttccagagactgaggatttccgaggacccagacgttcaccccgaggctgagcagcagcctggcccggagtcgggtgagggtcagaaaggaggagagcagcccaaactggtccgggggcacttctgccccattaaacgaaaagccaacagcaccaagagggacagaggaactttgctcaaggcgcagatccgtcaccagtcccttgacagtcagtctgaaaatgccaccatcgacctaaattctgttctagagcgagaattcagtgtccagagtttaacatctgttgtcagtgaggagtgtttttatgaaacagagagccacggaaaatcatagtatgattcaatccagatatgggttaaattcctcattttacttttaaactggtggtaaagtggaaattgcaaaaaaaaaa

SEQ ID NO: 15 - DNA, 2076 bp, Mus musculus
gtttgggaggagcttgtgtgtgtgagttgtgttttaagtttatttgcgtgtgagtacctttgggtttttgtgtgtgtctgtgtgtgtttgtgtgtgtataactgtgggtgactgtaagtgcacctgtgtgtttgtacgtgagtgtgtaagactgtgtgtgtgcacaagagcgtgtgtaggtgcacgtgttgtaggtgtgagaacacctgttgtgtttaggccatcagtcagcttggtcattgtttctaaggtagcatttatactttgttacctcaagtgggctctgggagtcaacagaagtcagaaaagctcagatccaagccccctttttctgacatggagaaatttcatgctcaatatgagatgctagagactattggccagggaggctgcgcccaggtgaagctggcccgacaccgcctcacaggcacccacgtggctgtcaaagtgattgtaaagagggagtgttggttcaaccctgtcatgtctgaggcagagttactgatgatgaccgatcatccgaatatcatctctctccttcaagtcattgagaccaagaagaaagtatacctcattatggagttgtgcgagggtaaatcactttaccaacacatccaaaatgctggctacctgcaggaggatgaagcacgcccattattcaagcagctcttaagtgctatgaactactgccacaaccagggtatagttcacagggacctgacacctgacaatattatggtagaaaaagatgggaaagtgaagatcattgattttggactcggcacccaagagaagccagggcaaaaccacaacttattctgtgagatttacccatttagtactcctgaggtgctctttaacagaccctatgatatgcgcaagatcgatgtgtggggtcttggagttgtgctgtattttatggtaactggaaagattctgtttgatactgccagcgtagaaaagctgcgaaagcaaattgttgcagaaaagtgttctgttccctgtagactgtcagtagagctccaagacctgattagacttttaatgacggacatccccgaacttaggcccactgttgctgaagttatggtgcatccctgggtcacagaaggctcaggggtgttaccagatccttgtgaagaacatatacccctcaagccagaccctgcgattgcaaaagcaatgggatttatcgggttccaagctcaagacattgaagattcgttatgtcagagaaaattcaacgaaaccatggcatcttattgtctactgaaaaaacagattcttaaggaatgtgacaggccaatccgggctcagcccatgaatccatctgtgaccccactctcttcccttgttgatgctcctactttccatctcggacttcggaggacagagactgaacccacaggtctcagattatctgacaataaggaagtgcctgtctgtggcaatagtactagtaagaaaagagagagaagtttcagtgggccgggtgttctcagcaggccgattaacacaacacccacaatggaccaaacacacacccgtacttggagtggtccctgcatttactcaaatgtttgcacaatccatccaaacagcatcaatgagagtacagaaggccacatcagtacctcagcagaggataagcctgtccacagcagaggctggcccagaggcatcaagggctggactaggaagataggaaatgcaatgaggaagctctgttgctgtatcccatccaaagagacatctcacctggggcagagaagagtctgcccaaaaatttaagacacaggaaggatgtcaggagaatgagcatccagcatggcccagcctttcagaccgaaggcaagctctacctgatcctggacttcctgcggggaggtgacctcttcaccaggctttccaaagaggtgatgttcacggaggaggatgtcaagttctacctggctgagctggccttggctctagaccacctccatggcctggggatcatctacagggatctgaagccagagaatatcctcctggatgaagagggacatattaagatcacagattttggcttgagca

SEQ ID NO: 16 - DNA, 2827 bp, Mus musculus
gagaggagttggtggagttggtggagtttggtggatttggtggagttggtggtgccctttgcgatttcgttgtatctagtgagccgtgtgtggattttgtgtttgattggttcgtgtgtgagcttttgtgtgtgtgtgtgtgtgtgtgtgtgtgtgtgtgtgtgtgtgtgtgtgtagatcagtgtgtgtttgggaggagcttgtgtgtgtgagttgtgttttaagtttatttgcgtgtgagtacctttgggtttttgtgtgtgtctgtgtgtgtttgtgtgtgtataactgtgggtgactgtaagtgcacctgtgtgtttgtacgtgagtgtgtaagactgtgtgtgtgcacaagagcgtgtgtaggtgcacgtgttgtaggtgtgagaacacctgttgtgtttaggccatcagtcagcttggtcattgtttctaaggtagcatttatactttgttacctcaagtgggctctgggagtcaacagaagtcagaaaagctcagatccaagccccctttttctgacatggagaaatttcatgctcaatatgagatgctagagactattggccagggaggctgcgcccaggtgaagctggcccgacaccgcctcacaggcacccacgtggctgtcaaagtgattgtaaagagggagtgttggttcaaccctgtcatgtctgaggcagagttactgatgatgaccgatcatccgaatatcatctctctccttcaagtcattgagaccaagaagaaagtatacctcattatggagttgtgcgagggtaaatcactttaccaacacatccaaaatgctggctacctgcaggaggatgaagcacgcccattattcaagcagctcttaagtgctatgaactactgccacaaccagggtatagttcacagggacctgacacctgacaatattatggtagaaaaagatgggaaagtgaagatcattgattttggactcggcacccaagagaagccagggcaaaaccacaacttattctgtgagatttacccatttagtactcctgaggtgctctttaacagaccctatgatatgcgcaagatcgatgtgtggggtcttggagttgtgctgtattttatggtaactggaaagattctgtttgatactgccagcgtagaaaagctgcgaaagcaaattgttgcagaaaagtgttctgttccctgtagactgtcagtagagctccaagacctgattagacttttaatgacggacatccccgaacttaggcccactgttgctgaagttatggtgcatccctgggtcacagaaggctcaggggtgttaccagatccttgtgaagaacatatacccctcaagccagaccctgcgattgcaaaagcaatgggatttatcgggttccaagctcaagacattgaagattcgttatgtcagagaaaattcaacgaaaccatggcatcttattgtctactgaaaaaacagattcttaaggaatgtgacaggccaatccgggctcagcccatgaatccatctgtgaccccactctcttcccttgttgatgctcctactttccatctcggacttcggaggacagagactgaacccacaggtctcagattatctgacaataaggaagtgcctgtctgtggcaatagtactagtaagaaaagagagagaagtttcagtgggccgggtgttctcagcaggccgattaacacaacacccacaatggaccaaacacacacccgtacttggagtggtccctgcatttactcaaatgtttgcacaatccatccaaacagcatcaatgagagtacagaaggccacatcagtacctcagcagaggataagcctgtccacagcagaggctggcccagaggcatcaagggctggactaggaagataggaaatgcaatgaggaagctctgttgctgtatcccatccaaagagacatctcacctggggcagagaagagtctgcccaaaaatttaagacacaggaaggatgtcaggagaatgagcatccagcatggcccagcctttcagaccgaaggcaagctctacctgatcctggacttcctgcggggaggtgacctcttcaccaggctttccaaagaggtgatgttcacggaggaggatgtcaagttctacctggctgagctggccttggctctagaccacctccatggcctggggatcatctacagggatctgaagccagagaatatcctcctggatgaagagggacatattaagatcacagattttggcttgagcaaggaggccaccgaccatgacaagagagcctattcattttgtgggactattgaatacatggcgcccgaggtggtgaaccggcgtggacacacacagagtgccgactggtggtccttcggtgtgctcatgttcgagatgctcacagggtccctgccattccaggggaaggacaggaaggaaacaatggcccgcatcctcaaagcaaagctgggtatgccttagttcctcagtgcggaggctcagagcctgctcagggcccttttcaagcggaacccctgcaaccggctaggtaagggtccctgtgacacccccaccccaggaatgcaatgaggctgccctctagaccccccttaggaatgtgagaggccaccattctgttccccacgggatgtggaggacttcctccttatgccccaactctgaactgtatgcttttccttgctaaggttgcaggaagcagaggtaccccgacgctggggaaacactcacatgtggcctggcgcccacaggcacgtggacttatcaggattgctgaaaggcatttgaaaaaaaaaaaaaaaaaaaa

SEQ ID NO: 17 - protein, 714 aa, Mouse
MKLISSLDGSKTLNANNMETLIECQSEGDIKVPPLLTSCESEDSICQLTEIKKRKKVLSWPSLMRKLSPSSDFSGSLEPELKVSLFDQPLSIICGENDTLPRPIQDILTILCLKGPSTEGIFRKAASEKARKELKEGLNCGVSVNLKQLPVHLLAVVFKDFLRGIPLKLLSCDLFEDWMGALEKPTEEDRIEALKQVAGGLPRPNLLLLRHLLYVLHLISKNAEVNKMDSSNLAICIGPNMLTLKNDQSLSFQAQKDLNNKVKILVEFLIDNCFEIFGENIRTRSRITSDDSLEHTDSSDVSTLQNDSAYDSNDPDVEPTSGAASPNRQLEGPTPTMAGLDTRGQRDTCESSSESSVSMVVRLKSSIVQQDRRFSEPNMSPSRECLVGPTSKQKLARSEDSFTLSQDASCSEGDEAEDPFTEEVFPAVDSKPKRPVDLKIKNWTQGLASPQGHITKAFSRSSPGESLGSSPVPSPSCPKRNFFTRHQSFTTKTDKTKPQREIRKHSMSFSFASHKKVLPRTSSIGSEKSKDFSRDQLQKDLRKESQLSGRIVQENESEIQSQTSLGFSLSGTWALSVDNTFQLVDMRKPGSPPSYEEAIYYQTSGLTAYGGQTVGSMRSRMFKPSTAVPPVPSHHGGDLSEGTPGGHRLSSVTEHWTHSQTVHVSIETQGRSELHQLRTVSESMQKAKLDCLGPQHSHLVFEADQLCCARESYI

SEQ ID NO: 18 - protein, 714 aa, Mouse
MKLISSLDGSKTLNANNMETLIECQSEGDIKVPPLLTSCESEDSICQLTEIKKRKKVLSWPSLMRKLSPSSDFSGSLEPELKVSLFDQPLSIICGENDTLPRPIQDILTILCLKGPSTEGIFRKAASEKARKELKEGLNCGVSVNLKQLPVHLLAVVFKDFLRGIPLKLLSCDLFEDWMGALEKPTEEDRIEALKQVAGGLPRPNLLLLRHLLYVLHLISKNAEVNKMDSSNLAICIGPNMLTLKNDQSLSFQAQKDLNNKVKILVEFLIDNCFEIFGENIRTRSRITSDDSLEHTDSSDVSTLQNDSAYDSNDPDVEPTSGAASPNRQLEGPTPTMAGLDTRGHRDTCESSSESSVSMVVRLKSSIVQQDRRFSEPNMSPSRECLVGPTSKQKLARSEDSFTLSQDASCSEGDEAEDPFTEEVFPAVDSKPKRPVDLKIKNWTQGLASPQGHITKAFSRSSPGESLGSSPVPSPSCPKRNFFTRHQSFTTKTDKTKPQREIRKHSMSFSFASHKKVLPRTSSIGSEKSKDFSRDQLQKDLRKESQLSGRIVQENESEIQSQTSLGFSLSGTWALSVDNTFQLVDMRKPGSPPSYEEAIYYQTSGLTAYGGQTVGSMRSRMFKPSTAVPPVPSHHGGDLSEGTPGGHRLSSVTEHWTHSQTVHVSIETQGRSELHQLRTVSESMQKAKLDCLGPQHSHLVFEADQLCCARESYI

SEQ ID NO: 19 - protein, 442 aa, Mouse
MKLISSLDGSKTLNANNMETLIECQSEGDIKVPPLLTSCESEDSICQLIEIKKRKKVLSWPSLMRKLSPSSDFSGSLEPELKVSLFDQPLSIICGENDTLPRPIQDILTILCLKGPSTEGIFRKAASEKARKELKEGLNCGVSLNLKQLPVHLLAVVFKDFFRGIPLKLLSCDLFEDWMGALEKPTEEDRIEALKQVAGGLPRPNLLLLRHLLYVLHLISKNAEVNKMDSSNLAICIGPNMLTLKNDQSLSFQAQKDLNNKVKILVEFLIDNCFEIFGENIRTRSRITSDDSLEHTDSSDVSTLQNDSAYDSNDPDVEPTSGTASPNRQLEGPTPTMAGLDTRGHRDTCESSSESSVSMVVRLKSSIVQQDRRFSEPNMSPSRECLVGPTSKQKLTRSEDSFTLSQDASCSEGDEAEDPFTEEVFPAVDSKPKRPVDLKIKN

SEQ ID NO: 20 - protein, 707 aa, Mouse
MKLISSLDGSKTLNANNMETLIECQSEGDIKVPPLLTSCESEDSICQLIEIKKRKKVLSWPSLMRKLSPSSDFSGSLEPELKVSLFDQPLSIICGENDTLPRPIQDILTILCLKGPSTEGIFRKAASEKARKELKEELNCGVSVNLKQLPVHLLAVVFKDFLRGIPLKLLSCDLFEDWMGALEKPTEADRIEALKQVAGGLPRPNLFLLRHLLYVLHLISKNAEVNKMDSSNLAICIGPNMLTLKNDQSLSFQAQKDLNNKVKILVEFLIDNCFEIFGENIRTRSRITSDDSLEHTDSSDMSTLQNDSAYDSNDPDVEPTSGTASPNRQLEDPTPTMAGLDTRGHRDTCESSSESSVSMVVRLKSSIVQQDRRFSEPNMSPSRECLVGPTSKQKLTRSEDSFTLSQDEAEDPFTEEVFPAVDSKPKRPVDLKMKNWTQGLASPQGHITKAFSRSSPGESLGSSLVPSPSCPKRNFFTRHQSFTTKTDKTKPQREIRKHSMSFSFASHKKVLPRTSSIGSEKSKDFSRDQLQKDLRKESQLSGRIIQENESEIQSQTSLGFSLSGTWALSVDNTFQLVDMRKPGSPPSYKEAIYYQTSGLRAYSGQTVGSMRSRMFKPSTAVPPVPSHHGGDLSEGTPGGHRLSSVTEHWTHSQTVHVSIETQGRSELHQLRTVSESMQKAKLDCLGPQHSHLVFEVDQLCCARESYI

SEQ ID NO: 21 - protein, 707 aa, Mouse
MKLISSLDGSKTLNANNMETLIECQSEGDIKVPPLLTSCESEDSICQLIEIKKGKKVLSWPSLMRKLSPSSDFSGSLEPELKVSLFDQPLSIICGENDTLPRPIQDILTILCLKGPSTEGIFRKAASEKARKELKEELNCGVSVNLKQLPVHLLAVVFKDFLRGIPLKLLSCGLFEDWMGALEKPTEEDRIEALKQVAGGLPRPNLFLLRHLLYVLHLISKNAEVNKMDSSNLAICIGPNMLTLKNDQSLSFQAQKDLNNKVKILVEFLIDNCFEIFGENIRTRSRITSDDSLEHTDSSDMSTLQNDSAYDSNDPDVEPTSGTASPNRQLEDPTPTMAGLDTRGHRDTCESSSESSVSMVVRLKSSIVQQDRRFSEPNMSPSRECLVGPTSKQKLTRSEDSFTLSQDEAEDPFTEEVFPAVDSKPKRPVDLKMKNWTQGLASPQGHITKAFSRSSPGESLGSSLVPSPSCPKRNFFTRHQSFTTKTDKTKPQREIRKHSMSFSFASHKKVLPRTSSIGSEKSKDFSRDQLQKDLRKESQLSGRIIQENESEIQSQTSLGFSLSGTWALSVDNTFQLVDMRKPGSPPSYKEAIYYQTSGLRAYSGQTVGSMRSRMFKPSTAVPPVPSHHGGDLSEGTPGGHRLSSVTEHWTHSQTVHVSIETQGRSELHQLRTVSESMQKAKLDCLGPQHSHLVFEVDQLCCARESYI

SEQ ID NO: 22 - protein, 707 aa, Mouse
MKLISSLDGSKTLNANNMETLIECQSEGDIKVPPLLTSCESEDSICQLIEIKKRKKVLSWPSLMRKLSPSSDFSGSLEPELKVSLFDQPLSIICGENDTLPRPIQDILTILCLKGPSTEGIFRKAASEKARKELKEELNCGVSVNLKQLPVHLLAVVFKDFLRGIPLKLLSCDLFEDWMGALEKPTEEDRIEALKQVAGGLPRPNLFLLRHLLYVLHLISKNAEVNKMDSSNLAICIGPNMLTLKNDQSLSFQAQKDLNNKVKILVEFLIDNCFEIFGENIRTRSRITSDDSLEHTDSSDMSTLQNDSAYDSNDPDVEPTSGTASPNRQLEDPTPTMAGLDTRGHRDTCESSSESSVSMVVRLKSSIVQQDRRFSEPNMSPSRECLVGPTSKQKLTRSEDSFTLSQDEAEDPFTEEVFPAVDSKPKRPVDLKMKNWTQGLASPQGHITKAFSRSSPGESLGSSLVPSPSCPKRNFFTRHQSFTTKTDKTKPQREIRKHSMSFSFASHKKVLPRTSSIGSEKSEDFSRDQLQKDLRKESQLSGRIIQENESEIQSQTSLGFSLSGTRALSVDNTFQLVDMRKPGSPPSYKEAIYYQTSGLRAYSGQTVGSMRSRMFKPSTAVPPVPSHHGGDLSEGTPGGHRLSSVTEHWTHSQTVHVSIETQGRSELHQLRTVSESMQKAKLDCLGPQHSHLVFEVDQLCCARESYI

SEQ ID NO: 23 - protein, 731 aa, Homo sapiens
MKLRSSHNASKTLNANNMETLIECQSEGDIKEHPLLASCESEDSICQLIEVKKRKKVLSWPFLMRRLSPASDFSGALETDLKASLFDQPLSIICGDSDTLPRPIQDILTILCLKGPSTEGIFRRAANEKARKELKEELNSGDAVDLERLPVHLLAVVFKDFLRSIPRKLLSSDLFEEWMGALEMQDEEDRIEALKQVADKLPRPNLLLLKHLVYVLHLISKNSEVNRMDSSNLAICIGPNMLTLENDQSLSFEAQKDLNNKVKTLVEFLIDNCFEIFGENIPVHSSITSDDSLEHTDSSDVSTLQNDSAYDSNDPDVESNSSSGISSPSRQPQVPMATAAGLDSAGPQDAREVSPEPIVSTVARLKSSLAQPDRRYSEPSMPSSQECLESRVTNQTLTKSEGDFPVPRVGSRLESEEAEDPFPEEVFPAVQGKTKRPVDLKIKNLAPGSVLPRALVLKAFSSSSLDASSDSSPVASPSSPKRNFFSRHQSFTTKTEKGKPSREIKKHSMSFTFAPHKKVLTKNLSAGSGKSQDFTRDHVPRGVRKESQLAGRIVQENGCETHNQTARGFCLRPHALSVDDVFQGADWERPGSPPSYEEAMQGPAARLVASESQTVGSMTVGSMRARMLEAHCLLPPLPPAHHVEDSRHRGSKEPLPGHGLSPLPERWKQSRTVHASGDSLGHVSGPGRPELLPLRTVSESVQRNKRDCLVRRCSQPVFEADQFQYAKESYI

SEQ ID NO: 24 - protein, 1008 aa, Rattus norvegicus
MELLSARLPRAGETFVPISEHQHWKPENAHNNGAQCFPELTLGRTHQGNKVLDSEWVTQMLRTTLAFRRALYIKLTASILELQNRKNGTFHVLRGENYKFSSVEHSSLAGIQQVFSKHSQSVQDCSDVEKVVAIVVAEKPGISFKNPPPALMAGPRDLAAAELERGEAKRPPEEIQCDGVSQITAPLEWRESRFYENSVSDKTDEFLCPQIRYKSSTRGRCRRHAGGRYMRLARPLPLDNNHLETHRVRTVLVSILSRPLSHPVRDAASLGLTQTQNQSLEVEKIPLGLSAPNPMKLISSLDGAKTLNANNMDTLIECQSEGDIKVLPLLTSCESEDSICQLIEVKKRKKVLSWPSLMRKLSPSPDFSGSLEPELKVSLFDQPLSIICKENDTLPRPIQDILTILCLKGPSTEGIFRKAASEKARKELKEELNCGGSVNLNQLPVHLLAVVFKDFLRGIPLKLLSCDLFEDWMGALEKPSEEDRIEALKQVADRLPRPNLLLLRTLVYVLHLISKNAEVNKMDSSNLAICIGPNMLTLKNDQSLSFQAQRDLNNKVKILVEFLIDNCLEIFGENIPTHPRITSDDSLEHTDSSDVSTLQNDSAYDSNDPDVEPANGVASPCRQLEGPSTTVAGMDTRGPRNTCESSSESSVSMVARLKSSIGQQDRRFSEPNMPPSRECFVGPITKQKLTRSEDSFVLPQEASCSEGNEAEDPFTEEVFPAVEGKPMRPVDLKIKNLTQGLASPQGPANKAFSSFSAGESLDSSPVPSPSCPKRNFFTRHQSFTTKTDKTKPQREIRKHSMSFSFASHKKVLPRTSSIGSEKSKDFSRDQLQKDLRKESQLAGRIIRENESEIQSQTNLGSSLSGTWALSVDNTFQFIDVRKPGSPPSYEEAIYYHTSGLTAYSGQTVGSMRARMLKQSMAVPPVPSHHEGELSEGIPGGHRSSSVTEHWTQSQTVHVSVETRGRSELHRLRTVSESVQRAKLDYLGQQRSHLVFEVDQLRCAKESYI

SEQ ID NO: 25 - protein, 727 aa, Mouse
MERACEKQDSVCNLVAVFENNRTPGEAPGSHSLEDQPHIPEHQLSLSPEPWEAPPVKEALKSEFRPVSRTYLSSLKNKLSSGAWRRSCQPGVSPGPETQEPEEKRVVRELLETEQAYVARLHLLDQVFFQELLREAGRSKAFPEDVVKLIFSNISSIYRFHAQFFLPELQRRVDDWAATPRIGDVIQKLAPFLKMYSEYVKNFERAAELLATWMDKSQPFQEVVTRIQCSEASSSLTLQHHMLEPVQRIPRYELLLKEYVQKLPAQAPDLEDAQRALDMIFSAAQHSNAAIAEMERLQGLWDVYQRLGLEDDIVDPSNTLLREGPVLKISFRRSDPMERYLVLFNNMLLYCVPRVLQVGAQFQVRTRIDVAGMKVRELTDAEFPHSFLVSGKQRTLELQARSRDEMVSWMQACQAAIDQVEKRSETFKAAVQGPQGDTQEPKPQVEELGLRAPQWVRDKMVTMCMRCQEPFNALTRRRHHCRACGYVVCAKCSDYRAELKYDSNRPNRVCLTCYTFLTGNVLPQGKEDKRRGILEKEASAAPEQSLVCSFLQLIGDKCSRSLPRSWCVIPRDDPLVLYVYAAPQDTKAHTSIPLLGYQVISGPQGTSGFPAATVRRAVHLQGRVCGAAGPLGDSYQACASGTPEDLTKKMCLTEPAASCSSRVHDSLPRPTPWFYHFTPSWATPDPPDVYTETIHPDSVSSRHRPFPSGRYHQVSQLAGEGADIPGS

SEQ ID NO: 26 - protein, 655 aa, Mouse
MERACEKQDSVCNLVAVFENNRTPGEAPGSHSLEDQPHSPEHQLSLSPEPWEAPPVKEALKSEFRPVSRTYLSSLKNKLSSGAWRRSCQPGVSPGPETQEPEEKRVVRELLETEQAYVARLHLLDQVFFQELLREAGRSKAFPEDVVKLIFSNISSIYRFHAQFFLPELQRRVDDWAATPRIGDVIQKLAPFLKMYSEYVKNFERAAELLATWMDKSQPFQEVVTRIQCSEASSSLTLQHHMLEPVQRIPRYELLLKEYVQKLPAQAPDLEDAQRALDMIFSAAQHSNAAIAEMERLQGLWDVYQRLGLEDDIVDPSNTLLREGPVLKISFRRSDPMERYLVLFNNMLLYCVPRVLQVGAQFQVRTRIDVAGMKVRELTDAEFPHSFLVSGKQRTLELQARSRDEMVSWMQACQAAIDQVEKRSETFKAAVQGPQGDTQEPKPQVEELGLRAPQWVRDKMVTMCMRCQEPFNALTRRRHHCRACGYVVCAKCSDYRAELKYDSNRPNRVCLTCYTFLTGNVLPQGKEDKRRGILEKEASAAPEQSLVCSFLQLIGDKCSRSLPRSWCVIPRDDPLVLYVYAAPQDTKAHTSIPLLGYQVISGPQGDPRVFQLQQSGQQYTFKAESVELQGRWVTAIKRAASGRTPEGPDEEDVSD

SEQ ID NO: 27 - protein, 461 aa, Mouse
MYSEYVKNFERAAELLATWMDKSQPFQEVVTRIQCSEASSSLTLQHHMLEPVQRIPRYELLLKEYVQKLPAQAPDLEDAQRALDMIFSAAQHSNAAIAEMERLQGLWDVYQRLGLEDDIVDPSNTLLREGPVLKISFRRSDPMERYLVLFNNMLLYCVPRVLQVGAQFQVRTRIDVAGMKVRELTDAEFPHSFLVSGKQRTLELQARSRDEMVSWMQACQAAIDQVEKRSETFKAAVQGPQGDTQEPKPQVEELGLRAPQWVRDKMVTMCMRCQEPFNALTRRRHHCRACGYVVCAKCSDYRAELKYDSNRPNRVCLTCYTFLTGNVLPQGKEDKRRGILEKEASAAPEQSLVCSFLQLIGDKCSRSLPRSWCVIPRDDPLVLYVYAAPQDTKAHTSIPLLGYQVISGPQGDPRVFQLQQSGQQYTFKAESVELQGRWVTAIKRAASGRTPEGPDEEDVSD

SEQ ID NO: 28 - protein, 656 aa, Mouse
MERACEKQDSVCNLVAVFENNSRTPGEAPGSHSLEDQLHSPEHQLSLSPEPWEAPPVKEALKSEFRPVSRTYLSSLKNKLSSGAWRRSCQPGVSPGPETQEPEEKRVVRELLETEQAYVARLHLLDQVFFQELLREAGRSKAFPEDVVKLIFSNISSIYRFHAQFFLPELQRRVDDWAATPRIGDVIQKLAPFLKMYSEYVKNFERAAELLATWMDKSQPFQEVVTRIQCSEASGSLTLQHHMLEPVQRIPRYELLLKEYVQKLPAQAPDLEDAQRALDMIFSAAQHSNAAIAEMERLQGLWDVYQRLGLEDDIVDPSNTLLREGPVLKISFRRSDPMERYLVLFNNMLLYCVPRVLQVGAQFQVRTRIDVAGMKVRELTDAEFPHSFLVSGKQRTLELQARSRDEMVSWMQACQAAIDQVEKRSETFKAAVQGPQGDTQEPKPQVEELGLRAPQWVRDKMVTMCMRCQEPFNALTRRRHHCRACGYVVCAKCSDYRAELKYDSNRPNRVCLTCYTFLTGNVLPQGKEDKRRGILEKEASAAPEQSLVCSFLQLIGDKCSRSLPRSWCVIPRDDPLVLYVYAAPQDTKAHTSIPLLGYQVISGPQGDPRVFQLQQSGQQYTFKAESVELQGRWVTAIKRAASGRTPEGPDEEDVSD

SEQ ID NO: 29 - protein, 1714 aa, Mouse
MGNSESQYTFQGSKNHSNTVTGAKQKPCSLKIRSVHAKDEKSLHGWTHGSSGAGYKSRSLARSCLSHFKNHQPYATRLSGPTCKVSKGTTYSKHRANTPGNDFQGNSGAFLPENGFHYVDRESEESHITSNGHLLTCYGRKESLASTPPGEDHRSPRVLIKTLGKLDGCLRVEFHNGGNPHKGTSEDPSGPVRLLRYSPTLASETCPVRETRRHSAAGSPSSQRPSPTDSRLRSSKGSSLSSESSWYDSPWGNAGEVSEVEGSFLAPSTPDPSLPSSFPPSDTKKPFNQSSSLSSLRELYKDPNLGCRSPSGTCLSSNEYISSQVSLNNRVSFASDMDVPSRVDHRDPLHYSSFTLPCRKSKALTEDAAKKDTLKARMRRFSDWTGSLSRKKRKLQEPRSMEGSEYFDSHSDGLNAEGQVPAQTSSLLWSGGSAQTLPHRSESTHAISVDPLRQNIYENFMRELEMSRSNTEHVETSTETMESSSESVSSLEQLDLLFEKEQGVVRKAGWLFFKPLVTLQKERKLELVARRKWKQYWVTLKGCTLLFYETYGKNSTEQNSAPRCALFAEDSIVQSVPEHPKKEHVFCLSNSCGDVYLFQATSQTDLENWVTAIHSCASLFAKKHGKEDTVRLLKSQTRSLLQKIDMDSKMKKMAELQLSVVSDPKNRKAIENQIRQWEQNLEKFHMDLFRMRCYLASLQGGELPNPKSLLAATSRPSKLALGRLGVLSVSSFHALVCSRDDSTLRKRTLSLTQRGKSKKGIFSSLKGLDTLARKGREKRASITQMFDSSHSHGFLGTQLPQKSTNSNKAHDLHLYGSAVDSALRDSMWEVQTYVHFQDNEGVTVTIKPEHRVEDVLALVCKMRQLEPTHYGLQLRKVVDKSVEWCVPALYEYMQEQVYDEIEVFPLSVYDVQLTKTGDMTDFGFAVTVQVDEHQHLNRIFISDVLPDSLAYGGGLRKGNEITSLNGEPVSDLDIQQMEALFSEKSVGLTLVARPVTTRRTLCASWSDSDLFSRDQKSLPPSPNQSQLLEEFLDNFRKTATSDFSNVPEITTGLKRSQTEGTLDQVPHREKMEQTFLSADQIAELCRDLNNTHTNSMEAPTESHDPPPRPLARHLSDADRLRKVIQELVDTEKSYVKDLSCLFELYLEPLQNETFLTQDEMESLFGSLPEMLEFQKVFLETLEDAISASSDFSVLETPSQFRKLLFSLGGSFLYYADHFKLYSGFCANHIKVQRVLERAKTDKAFKAFLDARNPTKQHSSTLESYLIKPVQRVLKYPLLLKELVSLTDHESEEHYHLTEALKAMEKVASHINEMQKIYEDYGMVFDQLVAEQSGTEKEVTELSMGELLMHSTVSWLNPFLSLGKARKDIELTVFVFKRAVILVYKENCKLKKKLPSNSRPAHNSADLDPFKFRWLIPISALQVRLGNTAGTENNSTWELIHTKSEIEGRPETIFQLCCSDSENKTSIVKVIRSILRENFRRHIKCELPLEKTCKDRLVPLKNRVPVSAKLASSRSLKGLRTSSSSEWPSEPSKGNSLDSDECSLSSGTQSSGCPVAESRRDSKSTELEKDAQEGLAEFPDGLIKESDILSDEDEDFHHPLKQGSPTKDIEIQFQRLKISEESDVHPVGQQPLTESGEQPKLVRGHFCPIKRKANSTKRGRGTLLKAQTRHQSLDSHPETASIDLNLVLEREFSVQSLTSVVNEEGFYETQSHGKS

SEQ ID NO: 30 - protein, 1701 aa, Homo sapiens
MGNSDSQYTLQGSKNHSNTITGAKQIPCSLKIRGIHAKEEKSLHGWGHGSNGAGYKSRSLARSCLSHFKSNQPYASRLGGPTCKVSRGVAYSTHRTNAPGKDFQGISAAFSTENGFHSVGHELADNHITSRDCNGHLLNCYGRNESIASTPPGEDRKSPRVLIKTLGKLDGCLRVEFHNGGNPSKVPAEDCSEPVQLLRYSPTLASETSPVPEARRGSSADSLPSHRPSPTDSRLRSSKGSSLSSESSWYDSPWGNAGELSEAEGSFLAPGMPDPSLHASFPPGDAKKPFNQSSSLSSLRELYKDANLGSLSPSGIRLSDEYMGTHASLSNRVSFASDIDVPSRVAHGDPIQYSSFTLPCRKPKAFVEDTAKKDSLKARMRRISDWTGSLSRKKRKLQEPRSKEGSDYFDSRSDGLNTDVQGSSQASAFLWSGGSTQILSQRSESTHAIGSDPLRQNIYENFMRELEMSRTNTENIETSTETAESSSESLSSLEQLDLLFEKEQGVVRKAGWLFFKPLVTVQKERKLELVARRKWKQYWVTLKGCTLLFYETYGKNSMDQSSAPRCALFAEDSIVQSVPEHPKKENVFCLSNSFGDVYLFQATSQTDLENWVTAVHSACASLFAKKHGKEDTLRLLKNQTKNLLQKIDMDSKMKKMAELQLSVVSDPKNRKAIENQIQQWEQNLEKFHMDLFRMRCYLASLQGGELPNPKSLLAAASRPSKLALGRLGILSVSSFHALVCSRDDSALRKRTLSLTQRGRNKKGIFSSLKGLDTLARKGKEKRPSITQVDELLHIYGSTVDGVPRDNAWEIQTYVHFQDNHGVTVGIKPEHRVEDILTLACKMRQLEPSHYGLQLRKLVDDNVEYCIPAPYEYMQQQVYDEIEVFPLNVYDVQLTKTGSVCDFGFAVTAQVDERQHLSRIFISDVLPDGLAYGEGLRKGNEIMTLNGEAVSDLDLKQMEALFSEKSVGLTLIARPPDTKATLCTSWSDSDLFSRDQKSLLPPPNQSQLLEEFLDNFKKNTANDFSNVPDITTGLKRSQTDGTLDQVSHREKMEQTFRSAEQITALCRSFNDSQANGMEGPRENQDPPPRPLARHLSDADRLRKVIQELVDTEKSYVKDLSCLFELYLEPLQNETFLTQDEMESLFGSLPEMLEFQKVFLETLEDGISASSDFNTLETPSQFRKLLFSLGGSFLYYADHFKLYSGFCANHIKVQKVLERAKTDKAFKAFLDARNPTKQHSSTLESYLIKPVQRVLKYPLLLKELVSLTDQESEEHYHLTEALKAMEKVASHINEMQKIYEDYGTVFDQLVAEQSGTEKEVTELSMGELLMHSTVSWLNPFLSLGKARKDLELTVFVFKRAVILVYKENCKLKKKLPSNSRPAHNSTDLDPFKFRWLIPISALQVRLGNPAGTENNSIWELIHTKSEIEGRPETIFQLCCSDSESKTNIVKVIRSILRENFRRHIKCELPLEKTCKDRLVPLKNRVPVSAKLASSRSLKVLKNSSSNEWTGETGKGTLLDSDEGSLSSGTQSSGCPTAEGRQDSKSTSPGKYPHPGLADFADNLIKESDILSDEDDDHRQTVKQGSPTKDIEIQFQRLRISEDPDVHPEAEQQPGPESGEGQKGGEQPKLVRGHFCPIKRKANSTKRDRGTLLKAQIRHQSLDSQSENATIDLNSVLEREFSVQSLTSVVSEECFYETESHGKS

SEQ ID NO: 31 - DNA, 2148 bp, Bos taurus
atactgattctgaaaaaaattaatggacaactaactgttgatatgaataaaatggtgaatatattttgtggggaaagaggtaatatcaaggaacatcctttgttggcatcatgtgagagtgaagataatatttgccagctaattgaaatcaagaagagaaagaaggtgtctaactggctccttctcatgagaaggctttcttcttcatcagatgtttctgcagcttcggagccagaattgaagacatcccttttcgatcagcccttgtcagccatctgcagtgacaacacgcttccgggacccattcaggatattctcacgattctatgcctgaaaggcccttccactgaaggaatattcaggaaagcagccaacgagaaagcccgcaaagagctgaaggaggagctcagctccggaggtgtggtggacctgagaagcctccctgtgcacctcctggcggtggtcctcaaggacttcctcagaagtattccactgaagctcctgtcctgtgacctgtttgaggagtggatgggtgccctggccaagcagagcgaggaggaccggatcgaggccctgaaacaggttgcagataagctcccgcggcccaaccacctgctgctcaagcatctggtctccgtgctccacgtgatcagcaagaactccgaggtcaaccggatggacgccagcaatctcgccatctgcattgggcccaacgtgctgagcccggaaaacgagcacaatctatcgctggaagcccggagagacctgaacgataaggttaagacactggtggaattcctcatcgataactgctttgaaatatttggggaagactttccagcacattccagaattgcttctgatgactccctggaacacacggacagttcagacatgtcgaccctgcagaacgactcagcctacgacagcaacgaccctgaccacgacgtggagcctgcgggctccccaagctcgcagcccccagggcccccggaactggctgctggcggcgtggaacccagagccccgctgcgcccttgggagcccgtggtcaacaccacggccagactaaagggcttcctcgggcaacctgaccggaggtactcggaccccagcaccacattctccccggagtgcctcgagggcagaagagcaaacccgaaactcacgcgaagcgaggacgacttcactgccgtggctcaggcagcctcccgctttgccggcgaggaagccgaggacccgtttccagaggaggtgtttcctgcagccgaaggccgggcccagaggccccgggacctgggggagtggagcccgactcagggctccgtgtcaccgtgcgcacgggtccccaaagccccctccagcagctctctggacgctttctccgacagctcgcccctggcctctccttccagccccaaaagaaacttcttcaccagacaccagtctttcaccaaggctgagaaaagcaagcccaacagagaaattaaaaagcactccatgtcattctccttcgcctctcaccaaagagggctgaccaaaatgcgcagctttggagccacgaagtccaagggctgcccccgagaccaagagaagcgaggttccaagaaagaaagccagctcgccggccggatcgtccaagaaagctcgtcggatgcccccggccaagccgtgctgggctttaactcgggggcctacgccctctcggtggaggatgtgttccggctggtggatcagaggcaccccggccgccccccctcttacgaggaggccgtgaggctccaggcgctggagctcgccccccgcgggggccagacagtgggcagcctgagggcccgcgtgctgagcctggacgcggggctcctgcctcccctccctgcccacccccacggggactcgagaaacatccgcgggccggagcccctggacgggctccgaggggggctggggactgagacctggaggcagagctgcgcccctaaggacacggcaggacgggtgatggtccccgggacatccgagctgcagcggctgagaaccgcatccgagtcgcagcagaagggcaggcaggccgtcctggcccggcggtgtagccagcccgtgtttgacgccgagcagctccgattcgctaaggaatcctacatctag

SEQ ID NO: 32 - DNA, 2055 bp, Canis familiaris
tcaaaaacactaaatgccaataatatggagacattgattgaatgtcagtcagagggtgatatcaaggaacatcctctgttggcatcatgtgagagtgaagataacatttgccagctaattgaaattaagaagagaaagaaggtgctgtcttggccatttctcatgagaaggctttctactgtgtcagattttcccggggcttcagaaccagaattgaaagtgccactatttgatcagcccttgtcaatcatctgtggtgaggacgacacactccccagacccattcaggatattctcactattttgtgccttaaaggaccttccaccgaagggatattcaggaaagcagccaatgagaaagcccgcaaagagctaaaggaagatctcaactcgggaggcatggtagatctgaaaagtctccccgtgcacctcctggctgcaatctttaaggacttcctcagaagtatcccactgaagctactttcatgtgacctgtttgaagaatggatggacgccctggagaggcagaatgaagaggatagaattgaggccctgaaacaggttgcagagaagctcccgcggcccaacctcctgctgctcaagcacttgatctccgtgctctatctgatcagcaaaaactcggagatcaataagatggatgccagcaatttagctatctgcatcggacccaacatgctgacccgagagaatgaccaacacctgtctttcgaagcccagaaagacttgaacaacaaggttaagtcactggtggagttcctcattgataactgcttggaaatatttggggagaacattcccacacaccccagtactgcttctgatgactctctggaacacaccgacagttcagacctgtcaatgctgcagaatgactcagcctatgacagcaatgatcctgacgtggaatccagcggtgccaccagctccccaaacaggaggccccaggtgcccttggagacggctgccagctgggagtccagaggcccacagcttgcttgggagttgagcccggagcccattgtcagcaccatagccgggctgaaaaactccctcagtgaaccagacaggagctactcagagcccagcatgtcttcctcacaagagagcctggagagccagaaaacacaccaaaaactaacacggagtgaggatgacttcaccatggctcaggcaggggctcatttggaaagtgaggaggctgaagacccatttccagaggaggtgtttcctgcagttgaaggcaaaatccaaagtccacaggacctgaaggtgaagaactcaactcagggtttggtgtcactatggggactaatgcccaaagccatctccagtggctctctggatgcttcctctgacagctcacccatggcttctccttccagtcctaaaagaaatttcttcacaagacatcagtctttcacaaagacagagaaaagtaagcccaacagagaaatcaaaaaacactccatgtcattctcctttgcctctcacaaaagagtgcttaccaaaacccctagttgcgtgtctgtgaaatccaaaggctttaccagagaccaagtaaagaaaggttttaaaaaagaaagccagcttgctgggcgaatcattcaggaaaacttaagccctggaagtccgccatcttatgaagaggccattaggtgccaggcattggacctctcggcctatgggggccaaacagttggcagcatgagagctagaatgctcagccaggacactgaactaccacctctcctaccttttcgtcatggagggaattcaagaaatataggcagtgaagagccacttgatgggcacagactatctcccaggactgagagttgggaacagagcaggactgtccatgcttctgcagaaacaataggacaagtgactgttacaaggagaccagagctgtaccgcctaagaactctatctgaatcaaaacagaagagtaggctggaccacctagtgcagcaatgtagtcagccggtctttgaggctgaccaactccagtatgctaaagaatcctacatttag

SEQ ID NO: 33 - DNA, 2082 bp, Gallus gallus
atgaaagtgctaagcagctgtaatacttcgaagacactaaatgctggaaacatggagagtttgattgagtgcccatcagaggctgatgccaagaaatgccccctgttgggcccagctgacactgaggatggactttgccagttggctgatgaaatgaagaaaagaaagaaggtgatatcgtggccatttgctctgagacgcacctccaccagtggggattccccggggcagctggactctggcctcaagatcaccttgtttggccagcctctggcaattatttgtggggaagacgacacactgccccagccagtccaggatctcctagctatattgtacatgaaaggaccttccactgaaggtatattcagaaaagctgccaatgagaaagcacgcaaggagttgaaagaggatctaaacaaaggcgggaatgttgatttggaaagcaaaactgtgcatctactggcagtggttttgaaggacttcctccgaaatattccctccaaacttctgtcagatgatctgtatgacaagtggatgctcgctctggagaagccaagcaagcaagaaaaaatcgaagaactgaaagaggtggctgacaaactgcccagaccaaacctggtcttgctgaagcacttgctctctgtgctccaccgcatcagccagaatgctgacaccaacaggatggatgccaacaaccttgccatctgcgtaggcccaaacatgctgagcccagggacaggcagcatgctgccgctggaggtgcagaaggagatgaatgacaaggtgacggtgttggtggagttcctcatagaaaacagctcagaaatatttggggaggacattctatggcctgtcagcaccttggctgtggagtcgccagagcatatagacagctccacagaacacctatgtgctactcatcagaatgactctgcctatgacagcccagatccggaagctgaatgctgtacttctgagctagagcagcccaaaggaagaagcactggtttgagcagaagatatccaacatgtgtctctgccacatcactgactaattgcaaaaatgacatcaacatgatggacaggaggtactcagaaccagacctatccttccaggaccgttttgaaagcaaaataaggaaacagaagctaaacaaaagtgaggacagttttccagttcagcagaagcagctaggtctggaaaatgaggtactggacaaacggcttgcaatcttacctccacaattatcaagtgactctctatccaaaacatcctccagttgctcactagagagctctgatggctcagtcttcaccagctccccattagtttcaccctctagtcccaaaaaaaccttcttaaataggccccagtccttttgcaccaagactcatgaagactgcagcacagctaggcgagaggtcaaaaagcattctatgtcattctcttttgcaaaccaccggaaaacactaacaaaacagcagagctgggggcctggaaaacacatgggtttccagagggacagtttcacaaagaaagatgatcagttctcctgcagaattgtacaggaaaacagccctgaggacgacaaaccaccgcacgtaccatatcagcgaaggtcacgtttcaggtcagctgatgaagtgttcagagaggtagaccagaggaatcctggaagaccaccctcttatgaagaagctactaagaactgtgaggccacccacgttccctcctgcaatctcacagttcagactatgagattaaaggtgtcaaaccaggacactttgccatctgatccacgtaccagccttgcacagggcatagcctgtacagcttcgaaggacctacccagtggcagggtttctgcagtgaatgattctgatgcagaaactgaaactctcagtgttaccgttggaataaactcccgtgtgagtttacctgtgacccctggagtgtaccgattgagagccatgtctgaatcctgtcaaaagaacaaacatgagtatgtggcacggcggtgcagccagcccatttttgaggtagagcagatacagtatgctaaggaatcctatgtttaa

SEQ ID NO: 34 - DNA, 1383 bp, Mouse
atgtacagcgagtacgtgaagaactttgagcgggccgcggaactgctggccacgtggatggacaagtctcagcccttccaggaggtggtcacccgcatccagtgcagcgaggcctcgggcagcctgaccctgcagcaccacatgttggagcctgtgcaaagaatcccgcggtacgaactgctgctcaaggaatatgtgcagaagctgccagcccaggccccagacctcgaagatgcccagagagcactggacatgatcttctcagctgcacagcactccaatgcagccattgcagagatggagcggctgcagggcctgtgggatgtgtaccagcgcctgggcttggaggatgacatcgtggacccctccaacaccctgctccgagagggccctgttctcaagatctctttccgccgcagcgacccaatggaacgctacctggttttgttcaacaacatgcttctgtattgtgtaccccgagtcctccaagtgggtgcccagttccaggtgcggactcgcatcgatgtggccggcatgaaggtgcgggagctgaccgatgctgagttcccacactccttcctggtgtccggaaagcagcgcacactggagctgcaggcccggtcccgagatgaaatggtttcctggatgcaggcctgccaggcagccattgaccaggttgagaagcggagtgagaccttcaaggctgctgtccagggacctcagggggacacgcaggagcccaagccacaggtagaggagctgggtctccgagcgcctcagtgggtccgggacaagatggtgaccatgtgcatgcgttgccaggagcccttcaatgccctgactcgtcggcgccaccactgccgggcttgcggctatgtagtgtgtgccaagtgctctgactaccgtgcggagctgaaatatgacagcaacaggcccaaccgagtctgcctgacctgctacacatttctcactggaaacgtactccctcaaggcaaggaggacaagaggcggggcatcttggagaaagaggcctcagcagcacccgagcagagtctggtgtgcagcttcctgcagctcataggagacaagtgcagcaggagccttccccggagctggtgtgtgatcccccgggatgacccccttgtgctgtatgtctatgcagccccccaggacacaaaggctcacacctccatccccctactgggctatcaggtgatttcagggccccagggggaccctcgggttttccagctgcaacagtcaggccagcagtacaccttcaaggccgagtctgtggagctgcagggccgctgggtgacagctatcaagcgtgcggccagtggccggacccctgagggacctgacgaagaagatgtgtctgac

SEQ ID NO: 35 - DNA, 1971 bp, Bos taurus
atggagggggcaagtgaggaaactctgacatctgtgtccagtctggtgactgtgtttgagaatagcaggatgacaggagcagtgcccagagtccacggcctggaggccgggcatcagcagcctgggtgcagggctcctacgtctgcagggccctgggagaagcccactgtggaggaggccctggaatctgggtccaggactgtcagcaggaggtacctgagctccctgaagaacaagctgtccagcggagcttggaggaagtcttaccagcccaggacctgccctggctcggggacacaggagcctgaggagaagaagattgtccgggagctgctggagactgagcaggcctacgtggcccgcctccacctgctggaccaggtcttcttccaggagctgctgaaggaggcccgcagcagcaaggccttccctgaggacgtggtcaggctcatcttctccaacatctcctccatctaccagttccactcacagttcttcctcccagagctgcagcggcggctggacgactggactaccaccccccgcatcggtgatgtgatccagaagctggcgcccttcctgaagatgtacagtgagtatgtcaagaacttcgagcgagccatcgagctgctggccacctggacagacaaatccccacctttccaggaggttatcacccgcatccagagcagcgaggcctcagctagcctgaccctgcagcaccacatgctggaacctgtgcagagaatcccacgctacgagctgctgctcaaggagtacgtccagaagctgccaggccaggccccagacctggctgatgcccagaaagcccttgacatgatcttctcggccgctcagcactccaatgcagccatcactgaaatggagcggctgcaggaactgtgggacgtgtaccagcgcctgggcctggaggacgatatagtcgacccctccaacaccctgctccgcgagggccccgtcctcaagatctccttccgccgcagcggccccatggagcgctaccttttcttgttcaacaacatgctgctctactgtgtgcccagggtcatccaggtgggcgcccacttccaggtcaggacccgcatcgacgtggccggcatgaaggtgcgggagctgactgacgcagagtttccccactccttcctggtgtccgggaagcagcgaaccctggagctgcgagcccggtcccaggaggaaatgatctcctggttgcaggcctgccaagccgccattgaccaaattgagaagcggaatgaaaccttcaaggctgcagtccagagccctgagggagacccccaggagcaggagctgcagtccgtggagctgggcctccgggcgccccagtgggtgcgggacaagatggtaaccatgtgcatgcgctgccaggagcccttcaacgccctgacgcgccgccgccaccactgccgggcctgcggctacgtggtgtgtgccaggtgctcggactaccgggcggagctcaagtacgatgccaacaggcccaaccgagtctgcctggactgctacactttcctcaccggaaacgtactccctgaggaaaaggaggacaagagacggggcatcctggagaaaggagccgtggcggggtccgagcagagcctgatatgcagctttctgcagcttctgggggacaagtggggcaagagcgggccccggggctggtgtgtcatcccccgggatgaccccctcgtgctctacgtctatgctgcccctcaggacatgcgggcccacacctccatccccctgctgggctaccaggtgaccgctgggccccaggcggacccccgggtcttccagctgcagcagtcaggccagctctacaccttcaaagcggaaagcgaggagctgaagggccgctgggtgaaggccatggagcgggcggccagcggctggatccctggggggcccgatgacaaggacctgtcggactga

SEQ ID NO: 36 - DNA, 1994 bp, Canis familiaris
atggagggagagagtaaggagaagctggcatctgtgtccaatctggtgactgtgtttgagaacagcaggattccaggagcagcatccagtgtcagcaagctggagattgaacatcaccaccctggatgcaggtctcccccacccgcagagccattgaaggagcctaaccttggagagaccctgagggggtctgagcccaggatggtcagcaggaggtacctgagctccctgaagaacaagctgtccagtggggcctggagaaaatcttgccagcctgggaccgaccctgggccagggacacaggagcctgaggaaaagaggatcgtccaggagctactggagacagagaaggcctatgtggcacgccttcatctgctagaccaggtgttctttcaggagctactgagggaagcccgcagcagcaaggccttccctgaggatgtggtcaggctcatcttctccaacatctcctccatctaccagttccacgctcagttcttcctcccggagctgcagcggcgactggatgactggacagccaccccccgcatcggcgacgtgatccagaaactggccccattcctgaagatgtacagtgagtatgtcaagaactttgagcgagctgctgagctgctggccacctggactgacaagtctgcacccttccaggaggtgatcacccgcattcagagcagcgaggcctccggcagcctgactctgcaacaccacatgctggaacccgtgcagagaatcccacgctacgagctgctgctcaaggagtatgttcagaagctgccagcccaggccccagaccgggccgatgctcagaaagcgctcgacatgatcttctcagccgctcagcactccaacgcggccatcagtgagatggagcggctgcaggacctgtgggaggtgtaccagcgcctgggcctcgagggcgacatagtggacccctccaacaccctgctccgcgaaggccctgtcctcaagatctcctttcgccgcagcgaccccatggaacgctaccttttcctgttcaacaacatgctgctctactgtatacccaaggtcatccaggtgggcgcccaattccaggtgaggacccgcatcgacgtggctggaatgaaggtgcgggagctgacggatgcagagttcccccactccttcctggtgtccgggaagcagcgcaccctggagctgcaagcccggtcccaggaagaaatgatttcctggatgcaggcctgccaagcagccattgaccaaatcgagaagcggaatgaaaccttcaaggctgcggtccagggcccagagggagacacccaggagcaggagctgcagtccgaggagctgggcctccgggcgccgcagtgggtccgggacaagatggtgaccatgtgcatgcgctgccgggagcccttcaacgccctgcggcgccgccgccaccactgccgagcctgcggcttcgtggtgtgtgccaagtgctccgactaccgggctgagctcaaatacgatgacaaccggcccaaccgggtctgcttccactgctacacgttcctcaccggaaacgtgctcccggaggacagggaggacaggaggcggggcattctggagaaagggtccatgacagggtctgagcagagcctgatgtgcagcttccttcagctggttggggacaaatggggcaagagcagcccccggggctggtgtgtgatcccccgggacgaccccctggtcctctatgtctacgctgcccctcaggacatgcgggctcacacctccatccccctgctgggctaccaggtgactgctgggacccaggcagatccccgggtcttccagctgcaacagtcagggcagctttacaccttcaaggctgaaactgaggagctgagggaccgctgggtgaaggccatggagcgggcggccagtggctggagccccagggggcccaaggatggagacctgtctgactgacccacagccggccactctcc

SEQ ID NO: 37 - DNA, 1974 bp, Rattus norvegicus
atggagcaagcctgtgcgaagcaggactcagtgtgcaacctggtggctgtgtttgagaacagcaggtctccgggagcagcacctggaccccatagttttgaggaccagccccatagccctgaacatcagctgcccctgtccccagagccttgggaggcaccccctgccggggaggccttgacgtctgagttccggccagtcagcaggacatatctgaattccctcaagaataagctatcgagtggggcttggaggaaatcctgccaacctggggccagtccagggccggagactcaggaacctgaggagaagagggtcgtgcaggagcttctggagacggagcaggcctacgtggctcgcctgcacttgcttgaccaggccactgtgttcttccaggagctgctgagggaggcgagccgtagcaaggccttccctgaggacgtggtgaagctcatcttctccaatatctcctccatctaccgtttccacgcacagttcttcctcccggagctgcagaggcgcgtggatgactggacagccacaccacgcatcggggatgtgatccagaaactggccccctttctgaaaatgtacagcgagtacgtgaagaactttgagcgggcggcagaactgctggccacctggatggacaagtcacagcccttccaggaggtggtcacccgcatccagcgcagtgaggcctccggcagcctgaccctgcagcaccacatgttggaacctgtgcagagaatcccacggtacgaactgctgctcaaggaatatgtgcagaagctgccagcccaggccccagacctcgaagatgcccagagagcgctggacatgatcttctcagccgcacagcactcgaacgcagccattgctgagatggagcggctgcagggcctgtgggatgtgtaccagcgcctgggcctggaggatgacattgtggacccttccaacaccctgctccgagagggccctgttctcaagatctctttccgccgcagcgacccaatggagcgctacctggttttgttcaacaacatgcttctgtactgcgtgccccgggtcctccaagtgggtgcccagttccaggtgaggacccgcatcgacgtggctggcatgaaggtgcgggagctgacggacgctgagttcccgcactccttcctggtatccggcaagcagagaacgttggagctgcaggcccggtctcgagaggaaatggtttcttggatacaggcctgccaggcagctattgaccaagtcgagaagcgaagtgaaaccttcaaggctgctgtccagggacctcagggggacacacaggagcccaagccacaggcagaggagctgggtctccgagcacctcagtgggtccgagacaagatggtgaccatgtgtatgcgctgccaggagcccttcaatgccctgactcgtcggcgtcaccactgccgggcttgtggctacgtagtgtgtgcaaagtgttctgactaccgtgcggagctgaaatatgacggcaacaggcccaaccgagtctgcctgacctgctacacgttcctcaccggaaacctgctccctgacagcaaggaggacaagaggcgaggcatcttggagaaagagacctccgcagggcccgaccagagtgtggtgtgcagtttcctgcagctcatgggggacaagagtatcaggagcattccccggagctggtgtgtgatcccccgggatgacccgcttgtgctgtatgtctatgcagccccccaggacatgaaggctcacacctccatccccttgctgggttatcaggtgacttcagggccccagggggaccctcgggttttccagctgcaacagtcaggccagcagtacaccttcaaggctgagtctgtggagctgcagggccgctgggtgagagctatcaagcgtgcggccagcggctggacccccgagggacctgacgaagaagacatgtctgactga

SEQ ID NO: 38 - DNA, 1740 bp, Rattus norvegicus
atggagcaagcctgtgcgaagcaggactcagtgtgcaacctggtggctgtgtttgagaacagcaggaacctgaggagaagagggtcgtgcaggagcttctggagacggagcaggcctacgtggctcgcctgcacttgcttgaccaggccagtgttcttccaggagctgctgagggaggcgagccgtagcaaggccttccctgaggacgtggtgaagctcatcttctccaatatctcctccatctaccgtttccacgcacagttcttcctcccggagctgcagaggcgcgtggatgactggacagccacaccacgcatcggggatgtgatccagaaactggccccctttctgaaaatgtacagcgagtacgtgaagaactttgagcgggcggcagaactgctggccacctggatggacaagtcacagcccttccaggaggtggtcacccgcatccagcgcagtgaggcctccggcagcctgaccctgcagcaccacatgttggaacctgtgcagagaatcccacggtacgaactgctgctcaaggaatatgtgcagaagctgccagcccaggccccagacctcgaagatgcccagagagcgctggacatgatcttctcagccgcacagcactcgaacgcagccattgctgagatggagcggctgcagggcctgtgggatgtgtaccagcgcctgggcctggaggatgacattgtggacccttccaacaccctgctccgagagggccctgttctcaagatctctttccgccgcagcgacccaatggagcgctacctggttttgttcaacaacatgcttctgtactgcgtgccccgggtcctccaagtgggtgcccagttccaggtgaggacccgcatcgacgtggctggcatgaaggtgcgggagctgacggacgctgagttcccgcactccttcctggtatccggcaagcagagaacgttggagctgcaggcccggtctcgagaggaaatggtttcttggatacaggcctgccaggcagctattgaccaagtcgagaagcgaagtgaaaccttcaaggctgctgtccagggacctcagggggacacacaggagcccaagccacaggcagaggagctgggtctccgagcacctcagtgggtccgagacaagatggtgaccatgtgtatgcgctgccaggagcccttcaatgccctgactcgtcggcgtcaccactgccgggcttgtggctacgtagtgtgtgcaaagtgttctgactaccgtgcggagctgaaatatgacggcaacaggcccaaccgagtctgcctgacctgctacacgttcctcaccggaaacctgctccctgacagcaaggaggacaagaggcgaggcatcttggagaaagagacctccgcagggcccgaccagagtgtggtgtgcagtttcctgcagctcatgggggacaagagtatcaggagcattccccggagctggtgtgtgatcccccgggatgacccgcttgtgctgtatgtctatgcagccccccaggacatgaaggctcacacctccatccccttgctgggttatcaggtgacttcagggccccagggggaccctcgggttttccagctgcaacagtcaggccagcagtacaccttcaaggctgagtctgtggagctgcagggccgctgggtgagagctatcaagcgtgcggccagcggctggacccccgagggacctgacgaagaagacatgtctgactga

SEQ ID NO: 39 - protein, 715 aa, Bos taurus
ILILKKINGQLTVDMNKMVNIFCGERGNIKEHPLLASCESEDNICQLIEIKKRKKVSNWLLLMRRLSSSSDVSAASEPELKTSLFDQPLSAICSDNTLPGPIQDILTILCLKGPSTEGIFRKAANEKARKELKEELSSGGVVDLRSLPVHLLAVVLKDFLRSIPLKLLSCDLFEEWMGALAKQSEEDRIEALKQVADKLPRPNHLLLKHLVSVLHVISKNSEVNRMDASNLAICIGPNVLSPENEHNLSLEARRDLNDKVKTLVEFLIDNCFEIFGEDFPAHSRIASDDSLEHTDSSDMSTLQNDSAYDSNDPDHDVEPAGSPSSQPPGPPELAAGGVEPRAPLRPWEPVVNTTARLKGFLGQPDRRYSDPSTTFSPECLEGRRANPKLTRSEDDFTAVAQAASRFAGEEAEDPFPEEVFPAAEGRAQRPRDLGEWSPTQGSVSPCARVPKAPSSSSLDAFSDSSPLASPSSPKRNFFTRHQSFTKAEKSKPNREIKKHSMSFSFASHQRGLTKMRSFGATKSKGCPRDQEKRGSKKESQLAGRIVQESSSDAPGQAVLGFNSGAYALSVEDVFRLVDQRHPGRPPSYEEAVRLQALELAPRGGQTVGSLRARVLSLDAGLLPPLPAHPHGDSRNIRGPEPLDGLRGGLGTETWRQSCAPKDTAGRVMVPGTSELQRLRTASESQQKGRQAVLARRCSQPVFDAEQLRFAKESYI

SEQ ID NO: 40 - protein, 684 aa, Canis familiaris
SKTLNANNMETLIECQSEGDIKEHPLLASCESEDNICQLIEIKKRKKVLSWPFLMRRLSTVSDFPGASEPELKVPLFDQPLSIICGEDDTLPRPIQDILTILCLKGPSTEGIFRKAANEKARKELKEDLNSGGMVDLKSLPVHLLAAIFKDFLRSIPLKLLSCDLFEEWMDALERQNEEDRIEALKQVAEKLPRPNLLLLKHLISVLYLISKNSEINKMDASNLAICIGPNMLTRENDQHLSFEAQKDLNNKVKSLVEFLIDNCLEIFGENIPTHPSTASDDSLEHTDSSDLSMLQNDSAYDSNDPDVESSGATSSPNRRPQVPLETAASWESRGPQLAWELSPEPIVSTIAGLKNSLSEPDRSYSEPSMSSSQESLESQKTHQKLTRSEDDFTMAQAGAHLESEEAEDPFPEEVFPAVEGKIQSPQDLKVKNSTQGLVSLWGLMPKAISSGSLDASSDSSPMASPSSPKRNFFTRHQSFTKTEKSKPNREIKKHSMSFSFASHKRVLTKTPSCVSVKSKGFTRDQVKKGFKKESQLAGRIIQENLSPGSPPSYEEAIRCQALDLSAYGGQTVGSMRARMLSQDTELPPLLPFRHGGNSRNIGSEEPLDGHRLSPRTESWEQSRTVHASAETIGQVTVTRRPELYRLRTLSESKQKSRLDHLVQQCSQPVFEADQLQYAKESYI

SEQ ID NO: 41 - protein, 693 aa, Gallus gallus
MKVLSSCNTSKTLNAGNMESLIECPSEADAKKCPLLGPADTEDGLCQLADEMKKRKKVISWPFALRRTSTSGDSPGQLDSGLKITLFGQPLAIICGEDDTLPQPVQDLLAILYMKGPSTEGIFRKAANEKARKELKEDLNKGGNVDLESKTVHLLAVVLKDFLRNIPSKLLSDDLYDKWMLALEKPSKQEKIEELKEVADKLPRPNLVLLKHLLSVLHRISQNADTNRMDANNLAICVGPNMLSPGTGSMLPLEVQKEMNDKVTVLVEFLIENSSEIFGEDILWPVSTLAVESPEHIDSSTEHLCATHQNDSAYDSPDPEAECCTSELEQPKGRSTGLSRRYPTCVSATSLTNCKNDINMMDRRYSEPDLSFQDRFESKIRKQKLNKSEDSFPVQQKQLGLENEVLDKRLAILPPQLSSDSLSKTSSSCSLESSDGSVFTSSPLVSPSSPKKTFLNRPQSFCTKTHEDCSTARREVKKHSMSFSFANHRKTLTKQQSWGPGKHMGFQRDSFTKKDDQFSCRIVQENSPEDDKPPHVPYQRRSRFRSADEVFREVDQRNPGRPPSYEEATKNCEATHVPSCNLTVQTMRLKVSNQDTLPSDPRTSLAQGIACTASKDLPSGRVSAVNDSDAETETLSVTVGINSRVSLPVTPGVYRLRAMSESCQKNKHEYVARRCSQPIFEVEQIQYAKESYV

SEQ ID NO: 42 - protein, 461 aa, Mouse
MYSEYVKNFERAAELLATWMDKSQPFQEVVTRIQCSEASGSLTLQHHMLEPVQRIPRYELLLKEYVQKLPAQAPDLEDAQRALDMIFSAAQHSNAAIAEMERLQGLWDVYQRLGLEDDIVDPSNTLLREGPVLKISFRRSDPMERYLVLFNNMLLYCVPRVLQVGAQFQVRTRIDVAGMKVRELTDAEFPHSFLVSGKQRTLELQARSRDEMVSWMQACQAAIDQVEKRSETFKAAVQGPQGDTQEPKPQVEELGLRAPQWVRDKMVTMCMRCQEPFNALTRRRHHCRACGYVVCAKCSDYRAELKYDSNRPNRVCLTCYTFLTGNVLPQGKEDKRRGILEKEASAAPEQSLVCSFLQLIGDKCSRSLPRSWCVIPRDDPLVLYVYAAPQDTKAHTSIPLLGYQVISGPQGDPRVFQLQQSGQQYTFKAESVELQGRWVTAIKRAASGRTPEGPDEEDVSD

SEQ ID NO: 43 - protein, 656 aa, Bos taurus
MEGASEETLTSVSSLVTVFENSRMTGAVPRVHGLEAGHQQPGCRAPTSAGPWEKPTVEEALESGSRTVSRRYLSSLKNKLSSGAWRKSYQPRTCPGSGTQEPEEKKIVRELLETEQAYVARLHLLDQVFFQELLKEARSSKAFPEDVVRLIFSNISSIYQFHSQFFLPELQRRLDDWTTTPRIGDVIQKLAPFLKMYSEYVKNFERAIELLATWTDKSPPFQEVITRIQSSEASASLTLQHHMLEPVQRIPRYELLLKEYVQKLPGQAPDLADAQKALDMIFSAAQHSNAAITEMERLQELWDVYQRLGLEDDIVDPSNTLLREGPVLKISFRRSGPMERYLFLFNNMLLYCVPRVIQVGAHFQVRTRIDVAGMKVRELTDAEFPHSFLVSGKQRTLELRARSQEEMISWLQACQAAIDQIEKRNETFKAAVQSPEGDPQEQELQSVELGLRAPQWVRDKMVTMCMRCQEPFNALTRRRHHCRACGYVVCARCSDYRAELKYDANRPNRVCLDCYTFLTGNVLPEEKEDKRRGILEKGAVAGSEQSLICSFLQLLGDKWGKSGPRGWCVIPRDDPLVLYVYAAPQDMRAHTSIPLLGYQVTAGPQADPRVFQLQQSGQLYTFKAESEELKGRWVKAMERAASGWIPGGPDDKDLSD

SEQ ID NO: 44 - protein, 657 aa, Canis familiaris
MEGESKEKLASVSNLVTVFENSRIPGAASSVSKLEIEHHHPGCRSPPPAEPLKEPNLGETLRGSEPRMVSRRYLSSLKNKLSSGAWRKSCQPGTDPGPGTQEPEEKRIVQELLETEKAYVARLHLLDQVFFQELLREARSSKAFPEDVVRLIFSNISSIYQFHAQFFLPELQRRLDDWTATPRIGDVIQKLAPFLKMYSEYVKNFERAAELLATWTDKSAPFQEVITRIQSSEASGSLTLQHHMLEPVQRIPRYELLLKEYVQKLPAQAPDRADAQKALDMIFSAAQHSNAAISEMERLQDLWEVYQRLGLEGDIVDPSNTLLREGPVLKISFRRSDPMERYLFLFNNMLLYCIPKVIQVGAQFQVRTRIDVAGMKVRELTDAEFPHSFLVSGKQRTLELQARSQEEMISWMQACQAAIDQIEKRNETFKAAVQGPEGDTQEQELQSEELGLRAPQWVRDKMVTMCMRCREPFNALRRRRHHCRACGFVVCAKCSDYRAELKYDDNRPNRVCFHCYTFLTGNVLPEDREDRRRGILEKGSMTGSEQSLMCSFLQLVGDKWGKSSPRGWCVIPRDDPLVLYVYAAPQDMRAHTSIPLLGYQVTAGTQADPRVFQLQQSGQLYTFKAETEELRDRWVKAMERAASGWSPRGPKDGDLSD

SEQ ID NO: 45 - protein, 657 aa, Rattus norvegicus
MEQACAKQDSVCNLVAVFENSRSPGAAPGPHSFEDQPHSPEHQLPLSPEPWEAPPAGEALTSEFRPVSRTYLNSLKNKLSSGAWRKSCQPGASPGPETQEPEEKRVVQELLETEQAYVARLHLLDQATVFFQELLREASRSKAFPEDVVKLIFSNISSIYRFHAQFFLPELQRRVDDWTATPRIGDVIQKLAPFLKMYSEYVKNFERAAELLATWMDKSQPFQEVVTRIQRSEASGSLTLQHHMLEPVQRIPRYELLLKEYVQKLPAQAPDLEDAQRALDMIFSAAQHSNAAIAEMERLQGLWDVYQRLGLEDDIVDPSNTLLREGPVLKISFRRSDPMERYLVLFNNMLLYCVPRVLQVGAQFQVRTRIDVAGMKVRELTDAEFPHSFLVSGKQRTLELQARSREEMVSWIQACQAAIDQVEKRSETFKAAVQGPQGDTQEPKPQAEELGLRAPQWVRDKMVTMCMRCQEPFNALTRRRHHCRACGYVVCAKCSDYRAELKYDGNRPNRVCLTCYTFLTGNLLPDSKEDKRRGILEKETSAGPDQSVVCSFLQLMGDKSIRSIPRSWCVIPRDDPLVLYVYAAPQDMKAHTSIPLLGYQVTSGPQGDPRVFQLQQSGQQYTFKAESVELQGRWVRAIKRAASGWTPEGPDEEDMSD

SEQ ID NO: 46 - protein, 579 aa, Rattus norvegicus
MEQACAKQDSVCNLVAVFENSRNLRRRGSCRSFWRRSRPTWLACTCLTRPVFFQELLREASRSKAFPEDVVKLIFSNISSIYRFHAQFFLPELQRRVDDWTATPRIGDVIQKLAPFLKMYSEYVKNFERAAELLATWMDKSQPFQEVVTRIQRSEASGSLTLQHHMLEPVQRIPRYELLLKEYVQKLPAQAPDLEDAQRALDMIFSAAQHSNAAIAEMERLQGLWDVYQRLGLEDDIVDPSNTLLREGPVLKISFRRSDPMERYLVLFNNMLLYCVPRVLQVGAQFQVRTRIDVAGMKVRELTDAEFPHSFLVSGKQRTLELQARSREEMVSWIQACQAAIDQVEKRSETFKAAVQGPQGDTQEPKPQAEELGLRAPQWVRDKMVTMCMRCQEPFNALTRRRHHCRACGYVVCAKCSDYRAELKYDGNRPNRVCLTCYTFLTGNLLPDSKEDKRRGILEKETSAGPDQSVVCSFLQLMGDKSIRSIPRSWCVIPRDDPLVLYVYAAPQDMKAHTSIPLLGYQVTSGPQGDPRVFQLQQSGQQYTFKAESVELQGRWVRAIKRAASGWTPEGPDEEDMSD

SEQ ID NO: 47 - DNA, 5121 bp, Bos taurus
ctcttactgacactgctgttcttaattcaggtgacgatgggaaactccgagagtcagtacaccctacaaggacctaaaaatcatagcaatactattactggtgctaaacaaaagccttgctccctgaaaatccgtggccttcatgccaaggatgacaagtcattgcacggatggggtcatggaagcagcggagcggggtacaagtccaggtcgttggcccgaagctgcctttctcactttaagagcaatcagccttacgcatcccgaccagggggccccacgtgcaaagcctccaaaggcagtgcctacgccaagcataggacaggtgccgcagggtcagatttccagggcaccgatgccgctttctcgcccgagaacggcttccactatgtcggacgcccgcccgaggagaaccactcggcttcgagggactgccgcaatgggcaccttctcaactgctacggcgggaacgagagcatcgcatccaccccgcctggcgaggaccgcaagagcccccgggtgctcatcaagacgctgggcaagctggacgggtgcctgcgggtagagttccacagcggcgccgcgcccccgggggcctccccatcgggcggcccggtgcagctgctgcgctactcggccggttccgcgcccagccccagcgcctccccggcagccgccgcgcgcccccgctccagcaagggcagctcgctcagctccgagtcctcctggtacgactcgccctggggcccggccggcgaggtcagtgaggctgagggctccttcgtggcgccagacacgcctgagcccggcctccacactggcttcccgaccagggatgctccaaagcctttcagccaaagcgcctccctctcatccctccgggacccctaccccaacgcctcccttgggagcctggcccccgcggccctgcggctttccgatgactacatgggcacccgcgccagcctcagtgcccgcgtctccttcgcctccgacatggacgtgccggcccgcgtggagcgcggggagcccgggcagttcgcgtccttcaccctcccttgccgcaagtccagggccctgggcgaggagtcctccaagaaggacaccctgaaagccaggatgcgccgcatcagcgactggacgggaagcctctccagaaagaaaagaagactccaggagccgaggtccaaggagggcagtgactactttgacagccgttcggatgggctgaacgtggacacgcaagggccctcccaagggtctgtgtccctgtggtcagggggctctgctcagatcctgtcccatagaagtgaatctgctcatgcgatcggcagcgaccccctccagcagaacatttatgagaatttcatgcgagagctggagatgagcaggaccaacacggagaacctcgagacgtccacggagacggccgagtccagcagcgagtcgctcagctctttggagcaactggacctgctctttgagaaggaacaaggggtggtgcgcaaagccgggtggctcttcttcaaacccctcgtcactctgcagaaggaaaggaagctggaactggtggcccggaggaaatggaagcagtattgggtgacacttaaaggatgcacgctgctgttttacgagacctacgggaagaattctatggatcagagcagtgccccccgctgtgccctctttgcagaagacagcatagtgcagtctgtcccggaacatcccaagaaggaaaatgtcttctgcctcagcaactccttcggggatgtctacctattccaggccaccagccagacagacctggagaactgggtcaccgccatccactcggcctgcgcatccctcttcgcaaagaaacacggcaaagaggacactgtccggctgctgaagaaccagaccagaaacctcctccagaagatagacatggacagcaagatgaagaagatggcagagttgcagctgtccgtggtgagcgacccaaagaacaggaaggccatagagaaccagaatagggccttaaaaaattggcaagagattctcctggacacccaacttggagttttcaggtccttatgtgttgtcaaatgtttaggtcagggagaggagctgcccttgaccgccctccccacccctccatcagggacctactatgctccgataattctcagggtcgctctttatctcacacactgttttgtgtgttctagagacgactctgcccttcggaaaagaactctctcgctgactcagcggggaagacacaagaaggggttgttttcttcattaaaaggcctggacacacttgcaagaaaagggaaggagaaaagaccgtctataactcagatattcgattccagtggcagccatggattttccggaactcagctacctcaaagctccaataactccagtgaggtggatgacctgctacacttatacgggtcagcagtggatggcgttccccgagacggcacgtgggagagccaggttgacgttcactttccggatcgtcaagtcgtgactgtgatggtcaagccggaaaccagggtagaagatgttctgactctggcctgcaagatgaggcagttggaacccagccattatggcctacaacttcggaagttagtggacgagaacgttgagtactgtattcctgcaccctacgaatatatgcaggaccaggtttatgatgagatagaagtctttccactaagcgtgtatgatgtgcagctgacaaagaccgggggtgtgtctgacttcgggtttgcagtgactgctcaggtggacgagcaccagcatctcagccgcatctttataagtgacgtcctccctgacggcctggcgtatggggaagggttgagaaagggcaatgaaatcatgaccttaaatggggaagccgtgtctgatcttgacctcaagcagatggaggctctgttttctgagaagagtgtcgggctcactctgattgccaggccaccagacacaaaagcgactctgtgttcttcctggtcagacagtgacctgttctcccgggaccagaagggtctgctgccccctcctaaccagtcccaacttctggaggaattcctggatcactttaaaaaggatacggcaaatgatttcagcaacgtccccgatgtcacaactggcctgaaaaggagtcagactgatggcacactggatcaggtttcccacagggagaagaaggagcagacattcaggagtgctgagcagatcactgcgctgtgtaggaattttaacgacacccaggccaacggcatggaagggccaagggagggtcaggaccctgctccaaggccactggctcgccacctctctgatgcagatcgcctccggaaagtcatccaggagctcatggacacagagaagtcctatgtgaaggatctgagctgcctcttcgacttatatttggagccacttcagagtgagaccatcctggtcttcactcagatggagtcactttttggaagcttgccagagatgcttgaatttcaaaaggtgtttctcgagaccctggaggatgggatttcagcgtcgtctgactttaacatacttgaaaccccctcgcagttcagaaaactgctgttttccctcggaggctctttcctttattatgcggaccactttaaactgtatagcggattctgtgctaatcatattaaagtacagaaggttctagagcgagctaaaactgataaagcctttaaggcttttctggatgcccggaaccccaccaagcagcactcctccacgctggagtcttacctcatcaagccagttcagagggtgctcaagtacccgctgctgctcaaggagctggtgtccctgacggaccacgagagcgaggagcactatcatctgacggaagcactaaaggcaatggaaaaagtagccagccacatcaatgagatgcagaagatatatgaagattatgggaccgtatttgaccagcttgtagcagagcagagtggaacagagaaggaggtcacagaactttcaatgggggagcttctgatgcactctacagtgtcctggttgaatccatttctgtctctaggaaaagccagaaaggaccttgaactcacagtatttgtttttaagagagctgtcatattggtttataaagaaaactgcaaactgaaaaagaaattgccctcaaattcccggcctgcacacagcaatgctgacctggatccgtttaagttccgctggttgatccccatatccgcgcttcaagtcagactgggaaatacagcagggacagaaaataattccttatgggagctgatccacaccaagtcagaaatagaaggacggccagagaccatctttcagttgtgctgcagtgacaatgaaagcaaaaccaacatcgtcaaggtgattcgatctattctgagggaaaatttcaggcgtcacataaagtgtgaattacccctggagaaaacatgtaaggatcgcctggtaccccttaagaaccgcgttcctgtctcagccaaattagcttcatccaggtccttaaaagtccttaagaattcctccagcagcgagtggccgggggagccgggcaagggcagctccctggactcggacgagggcagcctgagcagcagcacccagagcagcggctgcccccctgccggaagggggcaggacacccagcagcacccccacgcgggcctggccgacttctccgacagtctcatcaaagagagcgacattctgagcgacgacgaggacgactacccccaggctcggaggcagggcagccccaccaaagacatcgagatccagttccagagactgaggatctctgaggaccccgatgctcacccggctgaagagccgcccagcaaggatgggcagcccaagctggtgcgggggcacttctgcgccatcaaacgcaaggccaacagcaccaagcgggacaggggcaccctgctgaaggcgcagacgcgtcaccagtccctggacagtcatcctggaaacgccaaccttgatctcagctctgtccttgagcgagaattcagcgtccagagcttaacatcggttgtcaacgaagagtgcttttatgaaacagagagccatggaaagtcctag

SEQ ID NO: 48 - DNA, 5188 bp, Gallus gallus
atgggaaattcagaaagccaatacagtcttcagggatcaaaaaatcatgctgctgcttcaactggttccaagcagaagccttgctctctaaaaattcgcagcattcatgctaaagatgaaaagtcttgctccctgcatggatggggacataccaatagtggctcaaactacaagtcaaggtctcttgctagaagctgcctttcacacttcaagagtagccaaccttattcagctagactcagtgacactgtggtgaaggcctctaaaagtaacgtccttgccaaacacaggacacacacctcaggggactactgtccaggaaataatgcagtgtttttgcctgataatggtttccactatattggccttcaagctggaagtaatcatgctgcaccccgagattgcaatgggcacattttaaaatgctatggaaagaatgagagtcttgcatcaacctctccatcagaggacaggaggagtccaaaagtactcattaaaacactggggaagctggatggttgcttgagagtcgaattccacaacagtagcaacagcaaagtaccaactgaggagtccagtgggccagtccagctgctgaggtattcacctaccttggaatctaagtcaaataacctgctagatgtcaggaggaactccagcgcagactgttcttcaaaccatcgtctgtcacctactgattcaaggcttcgatctagtaagggaagctccctaagctccgagtcttcatggtatgactctctctggggaaatgctggggatatcagtgagttggatggtccatatttgactaggagcactccagatacaagcattcatgccagtttcccagcaagtgacaagaaatccttcaatcaaagttcatctctgtcctcacttcgagatctctataaggatacaaatttggaaggcactcctccacctgggatcaggctgtctgatgagtatattgacactcatggtagcctaagcaaccgtgtatcatttgcttcagacattgatgttccctccagggtagagcagggaagtcctgctcattattcctcttacactctcccatgtagaaagtccaagccactcgctgaggatgcatccaagaaggacacattaaaaagccgcatgaggcgcatcagcgattggacaggaagtctctcgaggaagaaaaggaagttgcaggaacccaaatgtaaagatgggagcgaatactttgacagcagaatggacaacttcagcacagacacgctggcaccatcacagcagtctactttgttgtggtcacccagctccagtcacatcctgtcccagagaagtgagtccaccaatgcagtcagcagtgatgccctgaggcagaacatttatgaaaacttcatgcgggagctggagatgagcaggacaaacctggagaacactgagacctcatcagaaacggaggactccagcagtgagtctctcagctccttggagcaactggatttgttgtacgagaaggagcaaggagtggtgcgcaaagcagggtggctgttcttcaaaccactggtgaccctgcagaaagagaagaagctggagctggtcacacggcgaaagtggaagcagtactgggtaacactaaaaggttgtactctgctcttctatgagacctatggaaggaattcaatggagcagagtagtttgcctcgatacgccctgtttgctgaagacagtatagtccagtctgttccagaacatcccaagaaagaaaacgtgttctgtctcagcaattcttttggagatgtctacctatttcaggcaacaagtcagacagatctggaaaactgggttactgccatacattcagcctgtgcttccctctttgcaaaaaagcttgggaaggaggacacagttcggctactgaaaaatcagaccaagagtctcttccagaagattgatatggacagcaagatgaaaaagatggcagagttgcagctctcaattgttagcgatccaaaaaacaggaaggcgatagagaatcagatccagcaatgggaacagaacctggaaaaatttaacatggaccttttccgaatgcgatgttacctagccagtttgcaaggtggggagcttccgaacccaaagagccttttggctgctgccagtcgtccttcaaaattggcactgggaaggctcggcattttctcagtgtcatccttccatgcactgatctgctccagggatgaagctgctctcaggaagcgtaccctgtctctgtctcaaagagtccgaaataagaagggtttgttttcttcactaaaaggactggacacactggcaagaaaaggaaaagagaagcgaccttccataacgcagatttttgactccactggaggacatggatttgccggtgtccagaattcagccaactctgctgagcaagttgatgaatttttgaatgtctactgctcagtaccggacagcatccagaaggagaatgcttgggaaacacaaacgtatgttcacttctgtgacggtcaaggagtagctttaactctgaaaccagaccacagggtggaagatgttctgtctctggcatgcaagatgaaacaactggagccaaggaactacggtctacaactcagaagattggttgatgaaaatactgaatactgtgctcctgaaccatacgaatatatagtagaccaggtgtatgatgaaatagaaatctgcccattaaatgtttatcacattcatcttacaaagactgaaaatataacagattttggttttgctgtcacagctcaagttgatgaaaatcagcatctcacacacatatttgtaagtgatgttcttcctgatgggcttgcatacagggaagggctacgagtaggcaatgagatcctgagcataaatggagagtctgtgtctgatcttgacctcaggcagatggagctattgttttcagagagaagcgtaatgctcactttgagaatgagccactgtgggaatcagcaacccttgtgtgcatcctggtcatcagatggtgacatttccagggttccaaaaagtttgttgccgcctccaaaccagtcacagctcctggaagagtttttagataacttcagaaaaaacacagaaaatgatgcagattttaataacgtgcctgatgtcacatccagcttgaaaaggagcagtactgatggcactctggaccaagtaccacacagggagaagactgatccacctttcaggagtgcagagcagatcagtgcgttgtgccgcaacttccaagaggtccagacgagcagcatggaagggcagaaggacaaccaggatccgcctccacgaccactggctcgccacctttctgatgcagacagattgaggaaagtcatccaagaacttatggatactgagaaatcttatgtcaaggacttgagttgcctctttgagctatacttggagcctcttcaaaatgaaaccttccttacacaagatgagatggagtccttgttcggcagtctgccagaaatgctggattttcagaaggtgtttttggagacccttgaagatggaatatcttcttcctcggactttaacacactggagacaccgtctcagttccggaaattgctgttttccctgggaggatcgtttctgtattatgctgaccacttcaaactgtacagtggcttttgtgccaaccacatcaaagttcagaaagttcttgagagagccaaaacagacagtgcatttaaggccttcttggatgctcgcaatcctacaaagcaacactcctctacgctggagtcgtatctcataaagcctgttcagagagtgctgaaatatcctctgcttttgaaagagctggtgtccctgacagacaacgagagtgaggagcactaccatttgacagaagcgctgaaggcaatggaaaaagtagcaagtcacatcaatgagatgcagaagatatatgaagattatggcactgtatttgatcaactggttgcagatcaaagtggaacagagaaggaggtgactgaactttccatgggagaacttctgatgcactctacagtttcctggctaaatcccttcccatcactgggcaaagcaagaaaagaccttgaacttacagtgtttgcctttaagaagagggctgtaatactggtgtataaagagaactacaaactgaaaaagaaaatgcctactaatgttcgtgctgcccataattatggtgacttggatccatttaagtttcgctggctgattcctctatctgctcttcaagttcggcttgggaacacagcaggcacaggaacagagaacagctgtatctgggaactgattcacacaaagtcagaactggaaggcaggccagaaaccatttttcagttatgcagcagcgactgcgagaacaagactaacatcgtgaaggtgatccgttctatcttgcgggagaatttcagacgtcacataaaatgtgagctgcctctggacaaaacctgtaaagatcgcctcattccactgaagaaccgtgtgcctgcgacagctaaactggcttccaccaggtccttgaaggtactgaagaattcacccagcagtgagtggaacggtgaccaggggaaaggcaccttccaggactctgatgactgcagcctgagcagcagcacccagagcagcagctgcaacaccacagagagcatacaggagcccaaaacttcatcccccgatcaacacgtacagagctgttcctccgacttttccaatgctcttgttaaagaatccgatattcttagtgatgatgatgatgacgactatcagagcctaaagaagggcagccctactaaagacattgaaattcagttccagcggctgaagatttcagaggaacccagtactgactctgaacgagatcaagctgcagaaaaggaggaaggagatggtttcaagatgggagaacatccaaagctgatacgtggccatttctgcccagtgaagcgaaaagtaaacagtacgaagcgtaacaggggaactttaacggcaatgcaggaacgtcaccagtctcttgacagccactctgatgctgcaaacttggacctgaactctattttggagagggaatttagcgtccagagtttaacatctgtagttaacgaggactgtttttatgaagctgtggagagacatggaaagtcct

SEQ ID NO: 49 - DNA, 5163 bp, Rattus norvegicus
gttaagatggggaactccgagagtcaatataccttccaaggatccaagaatcaaagcaatactgtcactggtgctaagcagaagccgtgctctctgaagatacgcagcattcatgcaaaagatgagaagtccttgcatggttgggctcacgggagcggcggagcaggctacaagtccagatccctagccaggagctgcctttctcactttaagagtcaccagccttacgccaccagactcaatggacccacgtgtaaagtctcaaagggcactgcctactctaagcacagagcaaatgccccaggaaatgattttcagggcaacaatggtgctttcttacccgagaatggcttccactatgttgggcgcgagtcaggggagagccacatcacctccagggactgcaatggacaccttctcacctgctatgggagacacgacagcattgcctccactcctccaggtgaggaccgcaggagccccagggtgctcatcaagacactggggaagctagatgggtgtttaagagttgagttccacaatggcggcaacccccacaaagggccctcagaggaccccagtgaacctgtacagctgctaagatactcccctacgttagcagcggaaacctgcccggtgccggaaagtaggcggctttcgggtgcaggctcctcagccagccagcgcccttctcccactgactcttgcctgcgctccagtaaaggcagctccctgagctcggagtcctcctggtatgactccccctggggcaacgcaggggaggcgagtgaagtggacggcaccttcctggctcccagtgctctagaccccagcctccccagcagcttcccacccagtgacaccaaaaagcctttcaaccaaagctcttccctctcctctctccgggaactgtacaaagatgccaacctggggtgccgctcaccttccggcacctgcctttcttccaacgagtacattagctcccaagtcggcctgaacaaccgagtctcctttgcttctgacatggatgtgccctccagggtggatcacagggacaccatgcagtacagctcctttactctcccgtgtcgcaagtccaaagccttaactgaggatgcagctaagaaagacaccctcaaagccagaatgcggcgcttcagtgactggacaggaagcctttccaggaagaagaggaaattgcaggaacccagatccacggagggcagtgagtacttcgatagccactcggatggactgaatgcagatgtgcaggggcccacgcagacatctgccttactgtggtcagggggctcagctcagaccctgcctcacagaagcgaatccactcacgcaatcggcagcgatcccctccaacagaacatctacgagaatttcatgcgagagctggaaatgagcaggaccaacatggagaacgcggaaacgtccacagagactgtggagtccagcagcgagtccctcagctcgctggagcagctggatctgctctttgagaaggagcagggggtggtccgaaaagctgggtggctcttcttcaaaccccttgtcaccttgcagaaggagaggaaactggagctggtggctcggaggaagtggaaacagtactgggtgaccctgaaaggctgtactctgatgttttatgagacctacggaaagaattccacagaccagaatagcgccccacggtgtgccctctttgcagaggacagcattgtgcagtctgtcccagagcatcccaagaaggagcacgtgttctgcctgagtaactcctatggagatgtctaccttttccaggccactagccagacggatctggaaaactggatcactgccatacactcggcgtgcgcatccctctttgcaaagaagcatgggaaagaagacacggtgcggctgctgaagagccagaccagaggcctgcttcagaagatagacatggacagcaagatgaagaagatggcagagctgcagctgtctgtggtgagcgaccccaagaacaggaaggccatcgaaaaccagatccggcaatgggagcagaacctggaaaaattccacatggacctgttccgcatgcgctgctatttggcaagcttacaaggtggggagttaccaaaccccaagagtctccttgcagccaccagccgcccctccaagctggctcttggcagactgggcgtcctgtctgtttcatctttccatgctctggtatgttccagagacgattctgctctcaggaaaagaacactttcctttacccagagagcaaaaagcaagaaaggcctattttcttcattgaaaggtctggacactctagcaagaaaagggagggagaagagagcttctataactcagatgtttgattcgagccacagccatggatatcttggaactcaactacctcaaaactccactaactccagtgaggtccatgacctgcatctgtatggctcctcagcagacagcactctccgagacagcacgtgggaagtacagacttatgtccacttccaggataacgaaggagtcactgtgatcatcaagccagagcacagggtggaagatgttctggctttggcatgcaagatgaggcacttggaacccactcgctatggtcttcagctccgaaaggtggtggatgaaagtgtggagtggtgtgtgcccgcgttgtatgagtacatgcaggagcaggcaagttgtgtttatgatgaaattgaagtcttcccactcagtgtgtatgacgtgcagctaaccaagacgggggacatgactgactttgggtttgcagtcacagcccaggtggacgagcaccagcatctcagccggatatttatcagcgatgttctccctgacagcctggcgtatggaggaggactgagaaagggcaatgaaatcaggagcttaaatggggaagcagtgtctgaccttgacctcaagcagatggaggctttgttttctgagaagagcgttggactcactctagtcgcccggcctgcagacacaagagcaagcctgtgtacttcctgctcagacagtgacctgttctccagggaccagaaaagtctgctgccctctcccaaccagtgccaactgctggaggaattcctggataactttaaaaaaacctccacaaacggtaaggctgatgtacccagacctttgtctggctgccttagacagatggaaacgcttggtgttccggataaggtgcaccatgacctgccctatcttgtcacctctcagagtgctgagcagattgtggagctctgcagaggcaccaccagtatggaagcaccgacagacagccgcgacccgcctcccaggcctctggctcgtcacctctcagatgcagaccgcctccgaaaagtcatccaggagcttgtagacaccgagaagtcttacgtgaaggatctgagctgcctctttgaactgtacttggagccccttcagaacgagacctttcttacccaagatgagatggagtcactttttgggagcttgccagagatgctggaatttcaaaaggtgttcctggagactctggaggatgggatctctgcttcctcagactttagtgtcctggaaaccccctcacagtttcggaaattgctgttctctctcggaggctctttcctctactacgcagatcactttaaactgtacagtggattctgcgccaaccacattaaagtacagaaggtcctagagcgagctaaaaccgacagagctttcaaggcttttctggacgcgcggaaccccacgaagcagcactcctccacgctggagtcatatctcatcaagcctgttcagagagtgctcaagtatcctctgctcctcaaggagctcgtgtcactgactgaccatgagagcgaagaacactatcacctgacagaagctctaaaggccatggaaaaagtagccagtcacatcaatgagatgcagaagatctacgaggactatgggacggtgtttgaccagctggtggcagagcagagtggcacagagaaggaggtaacagagctgtccatgggggaacttctgatgcactctacagtttcctggttgaatccgttcctgtctctgggaaaagccaggaaggacattgagctcacagtgtttgtttttaagagagctgtcatactggtttataaagaaaactgcaagctgaaaaagaaactgccctccaattcccggcctgctcacaactctgctgacttggacccatttaaattccgctggttgatccccatatctgcgcttcaagttagactggggaacacggcagggactgaaaacaattccacctgggagctgattcataccaagtcagaaattgaaggacggccagagaccatctttcaactgtgctgcagtgacagcgagagcaaaaccagcatcgttaaggtgatccgttctattctgagagagaacttcaggcgccacataaagtgtgagttaccaatggagaagacatgtaaggaccggctggtacctcttaagaaccgagttcctgtttcagccaaattagcctcatccaggtctttgaaggtcctcagaacatcctccagcagcgagtggcccagcgaccccagcaagggcaactcactggactcggatgagtgcagcctgagcagtggcacccagagcagtggctgcccagtagctgagagcagacaagactgtaagagcaccgtgctggagaaagacgctcatgaggtcctggcagagtttccagatggtctcatcaaagaaagcgacattctaagtgatgaagaggaggacttccaccaccctctgaaacagggcagccctactaaggacattgagcttcagttccagagactgcgaatctctgaggaacccgatatgcactcaactgggcagcagccgcctctcacagggccgggtgaacagcccaagctggtcaggggccacttttgccccattaagcgtaaagcaaacagcactaagaggggccgaggaactttgctcaaggcgcagacacgtcaccagtccctggacagccacccggaaaccgccagcatcgatctaaatttggttctggagagagaattcagtgtccagagcttaacttcagttgttaatgaggagtgtttttatgaaacacagagccatggcaaatcctag

SEQ ID NO: 50 - DNA, 3298 bp, Canis familiaris
atgcgggagttggaaatgagtaggactaatacggagaatgtagaaacgtccacagacactgcagactccagcagcgggtcactcagctctttggagcaacttgatctgctctttgagaaggaacaaggggtggtccgaaaagctggatggctgttcttcaaacccctcgtcactttgcagaaggaaaggaagctcgaattggtggcccggaggaagtggaaacagtactgggtaaccctgaaaggatgcaccctgctattctacgagacctatgggaagaattccatggatcagagtaatgcccctcgctgtgccttgtttgcagaagacagcatagtgcagtctgtcccagaacatcccaagaaggaaaacgtgttctgcctcagcaactcctttggagatgtctaccttttccaggccacaagccagacagatctggaaaattgggtcactgccatacattcagcttgcgcatccctttttgcaaagaagcatgggaaagaggacacggttcggctactgaagaaccagaccaaaaaccttgttcagaagatagatatggacagcaagatgaagaagatggcagagttacagctgtcggtggtgagcgacccaaagaacaggaaagccatagaaaaccagatccagcaatgggagcagaacctggagaaatttcacatggatctgttcaggatgcgttgctatctggccagcctacaaggcggggagctgccaaaccccaagagcctcctcgctgccgccagccgcccctccaagctggccctcggcaggctgggcatcttgtcagtttcttctttccatgctctagtttgttctagagatgactctgctttccggaaaagaacgctctcactgacccagcgaggaagaaacaagaaggggatattttcttcattaaaagggctggacactctggcaagaaaagggaaggagaaaagaccttctgtaactcaggcgagttcctcagtggatgaacttctgcatatatatggttcaacagccgatggtgttccccgagacaacacatgggaagcccagacttacgttcattttcaggataatcaaggagttactgtaatgatcaagccagaacacagagtagaagatattttgactcttgcatgcaagatgaggcagctggaacccagccactatggcctacaactccgaaaattagtagatgaaaacactgagtactgtatccctgcaccatatgaatacatgcaagaacaggcaagtgtttatgatgaaatagaaatctttccgctgagtgtttacgatgtacaacttaccaagactggcagtgtgtctgattttgggtttgcagtgacagcgcaggtggacgagcatcagcatctcagccgaatatttataagtgatgttctccctgacggcttggcatatggagaagggttgaggaagggcaatgaaatcatgaccttaaatggggaagctgtgtctgatcttgatctcaagcagatggaagccctgttttctgagaagagcgttgggctcaccctgattgcccggccaccagacagaaagagcaccttgtgttcttcctggtcagacagtgacctgttctcaagggaccagaagagtctacttccccccccaaaccagtcccagcttctggaggagttcctggataacttcaaaaagaatacaaccaatgatttcagcaatgtccctgatgtcacaagtggcctgaaaaggagtcagactgatggcgcactggcccgagttccccgtgaggaggacacagcacagacgctcgggagtgcagagcagatcaccgtgctgtgtaggagtttcaacgacacgcagagtaacggcatggaagggcctagggggactcgggacccacctccgaggccactggctcgccacctctctgatgcagatcgcctccggaaagtcatccaggagctgatggacacggagaagtcctacgtgaaggatctgagctgcctctttgaattatacctggagccacttcaaaatgagacctttctcacccaagatgagatggagtcactttttggaagtttgccagagatgcttgaatttcaaaaggtgtttctagagactctggaggatgggctttcagcatcatctgactttaatatcctcgagactccttcacagttcagaaaattactgttttcccttggaggctctttcctttattacgcggaccattttaaactgtacagcggattctgtgctaatcatattaaagtacagaaggttctagagcgagctaaaactgataaagccttcaaggcttttctggatgcccggaatcctaccaagcagcattcctccacactggagtcctacctcatcaagccagttcagagagtgctcaagtaccctctgctgctcaaggagctcgtgtcgctgacggaccacgagagtgaggagcactatcacctgacagaagcactaaaggcaatggaaaaagtagcgagccacatcaatgagatgcagaagatctacgaggattatgggactgtgtttgaccagctagtggcagagcaaagtggaacagagaaggagcctgtctctgtggtctccatgtcttctttatctcctgttatgatacaggtaacagaactttccatgggggaacttctgatgcactctgcagtttcctggttgaatccatttctgtctctaggaaaagccagaaaggaccttgagctcacagtgtttgtttttaaaagagctgtcatattggtttataaagaaaactgcaaactgaaaaagaaactgccctcgaattcccggcctgcacatggctctgccgatttggacccatttaaatttcgctggttgatccccatatccgcacttcaagtcagactggggaatacagcagggacagaaaataattccatatgggaactgatccatacgaagtcagaaatagaaggacggccagaaaccatcttccaactgtgctgcagtgacaatgaaagcaagaccaacatcgtcaaggtgattcggtctattctgagggagaacttcaggcgtcacatcaagtgtgagttacccctggagaagacctgtaaggatcgcctggttccccttaagaaccgagttcctgtttcagccaagttagcttcctccaggtccttaaaagtcctcaaaacttcctccagcagcgagtggcccagcgagccgggcaaggccagctccctggactctgacgagtgcagcctgagcagcagcacgcagagcagtggctgccacccaggtggaagccggcagg

SEQ ID NO: 51 - protein, 1706 aa, Bos taurus
LLLTLLFLIQVTMGNSESQYTLQGPKNHSNTITGAKQKPCSLKIRGLHAKDDKSLHGWGHGSSGAGYKSRSLARSCLSHFKSNQPYASRPGGPTCKASKGSAYAKHRTGAAGSDFQGTDAAFSPENGFHYVGRPPEENHSASRDCRNGHLLNCYGGNESIASTPPGEDRKSPRVLIKTLGKLDGCLRVEFHSGAAPPGASPSGGPVQLLRYSAGSAPSPSASPAAAARPRSSKGSSLSSESSWYDSPWGPAGEVSEAEGSFVAPDTPEPGLHTGFPTRDAPKPFSQSASLSSLRDPYPNASLGSLAPAALRLSDDYMGTRASLSARVSFASDMDVPARVERGEPGQFASFTLPCRKSRALGEESSKKDTLKARMRRISDWTGSLSRKKRRLQEPRSKEGSDYFDSRSDGLNVDTQGPSQGSVSLWSGGSAQILSHRSESAHAIGSDPLQQNIYENFMRELEMSRTNTENLETSTETAESSSESLSSLEQLDLLFEKEQGVVRKAGWLFFKPLVTLQKERKLELVARRKWKQYWVTLKGCTLLFYETYGKNSMDQSSAPRCALFAEDSIVQSVPEHPKKENVFCLSNSFGDVYLFQATSQTDLENWVTAIHSACASLFAKKHGKEDTVRLLKNQTRNLLQKIDMDSKMKKMAELQLSVVSDPKNRKAIENQNRALKNWQEILLDTQLGVFRSLCVVKCLGQGEELPLTALPTPPSGTYYAPIILRVALYLTHCFVCSRDDSALRKRTLSLTQRGRHKKGLFSSLKGLDTLARKGKEKRPSITQIFDSSGSHGFSGTQLPQSSNNSSEVDDLLHLYGSAVDGVPRDGTWESQVDVHFPDRQVVTVMVKPETRVEDVLTLACKMRQLEPSHYGLQLRKLVDENVEYCIPAPYEYMQDQVYDEIEVFPLSVYDVQLTKTGGVSDFGFAVTAQVDEHQHLSRIFISDVLPDGLAYGEGLRKGNEIMTLNGEAVSDLDLKQMEALFSEKSVGLTLIARPPDTKATLCSSWSDSDLFSRDQKGLLPPPNQSQLLEEFLDHFKKDTANDFSNVPDVTTGLKRSQTDGTLDQVSHREKKEQTFRSAEQITALCRNFNDTQANGMEGPREGQDPAPRPLARHLSDADRLRKVIQELMDTEKSYVKDLSCLFDLYLEPLQSETILVFTQMESLFGSLPEMLEFQKVFLETLEDGISASSDFNILETPSQFRKLLFSLGGSFLYYADHFKLYSGFCANHIKVQKVLERAKTDKAFKAFLDARNPTKQHSSTLESYLIKPVQRVLKYPLLLKELVSLTDHESEEHYHLTEALKAMEKVASHINEMQKIYEDYGTVFDQLVAEQSGTEKEVTELSMGELLMHSTVSWLNPFLSLGKARKDLELTVFVFKRAVILVYKENCKLKKKLPSNSRPAHSNADLDPFKFRWLIPISALQVRLGNTAGTENNSLWELIHTKSEIEGRPETIFQLCCSDNESKTNIVKVIRSILRENFRRHIKCELPLEKTCKDRLVPLKNRVPVSAKLASSRSLKVLKNSSSSEWPGEPGKGSSLDSDEGSLSSSTQSSGCPPAGRGQDTQQHPHAGLADFSDSLIKESDILSDDEDDYPQARRQGSPTKDIEIQFQRLRISEDPDAHPAEEPPSKDGQPKLVRGHFCAIKRKANSTKRDRGTLLKAQTRHQSLDSHPGNANLDLSSVLEREFSVQSLTSVVNEECFYETESHGKS

SEQ ID NO: 52 - protein, 1729 aa, Gallus gallus
MGNSESQYSLQGSKNHAAASTGSKQKPCSLKIRSIHAKDEKSCSLHGWGHTNSGSNYKSRSLARSCLSHFKSSQPYSARLSDTVVKASKSNVLAKHRTHTSGDYCPGNNAVFLPDNGFHYIGLQAGSNHAAPRDCNGHILKCYGKNESLASTSPSEDRRSPKVLIKTLGKLDGCLRVEFHNSSNSKVPTEESSGPVQLLRYSPTLESKSNNLLDVRRNSSADCSSNHRLSPTDSRLRSSKGSSLSSESSWYDSLWGNAGDISELDGPYLTRSTPDTSIHASFPASDKKSFNQSSSLSSLRDLYKDTNLEGTPPPGIRLSDEYIDTHGSLSNRVSFASDIDVPSRVEQGSPAHYSSYTLPCRKSKPLAEDASKKDTLKSRMRRISDWTGSLSRKKRKLQEPKCKDGSEYFDSRMDNFSTDTLAPSQQSTLLWSPSSSHILSQRSESTNAVSSDALRQNIYENFMRELEMSRTNLENTETSSETEDSSSESLSSLEQLDLLYEKEQGVVRKAGWLFFKPLVTLQKEKKLELVTRRKWKQYWVTLKGCTLLFYETYGRNSMEQSSLPRYALFAEDSIVQSVPEHPKKENVFCLSNSFGDVYLFQATSQTDLENWVTAIHSACASLFAKKLGKEDTVRLLKNQTKSLFQKIDMDSKMKKMAELQLSIVSDPKNRKAIENQIQQWEQNLEKFNMDLFRMRCYLASLQGGELPNPKSLLAAASRPSKLALGRLGIFSVSSFHALICSRDEAALRKRTLSLSQRVRNKKGLFSSLKGLDTLARKGKEKRPSITQIFDSTGGHGFAGVQNSANSAEQVDEFLNVYCSVPDSIQKENAWETQTYVHFCDGQGVALTLKPDHRVEDVLSLACKMKQLEPRNYGLQLRRLVDENTEYCAPEPYEYIVDQVYDEIEICPLNVYHIHLTKTENITDFGFAVTAQVDENQHLTHIFVSDVLPDGLAYREGLRVGNEILSINGESVSDLDLRQMELLFSERSVMLTLRMSHCGNQQPLCASWSSDGDISRVPKSLLPPPNQSQLLEEFLDNFRKNTENDADFNNVPDVTSSLKRSSTDGTLDQVPHREKTDPPFRSAEQISALCRNFQEVQTSSMEGQKDNQDPPPRPLARHLSDADRLRKVIQELMDTEKSYVKDLSCLFELYLEPLQNETFLTQDEMESLFGSLPEMLDFQKVFLETLEDGISSSSDFNTLETPSQFRKLLFSLGGSFLYYADHFKLYSGFCANHIKVQKVLERAKTDSAFKAFLDARNPTKQHSSTLESYLIKPVQRVLKYPLLLKELVSLTDNESEEHYHLTEALKAMEKVASHINEMQKIYEDYGTVFDQLVADQSGTEKEVTELSMGELLMHSTVSWLNPFPSLGKARKDLELTVFAFKKRAVILVYKENYKLKKKMPTNVRAAHNYGDLDPFKFRWLIPLSALQVRLGNTAGTGTENSCIWELIHTKSELEGRPETIFQLCSSDCENKTNIVKVIRSILRENFRRHIKCELPLDKTCKDRLIPLKNRVPATAKLASTRSLKVLKNSPSSEWNGDQGKGTFQDSDDCSLSSSTQSSSCNTTESIQEPKTSSPDQHVQSCSSDFSNALVKESDILSDDDDDDYQSLKKGSPTKDIEIQFQRLKISEEPSTDSERDQAAEKEEGDGFKMGEHPKLIRGHFCPVKRKVNSTKRNRGTLTAMQERHQSLDSHSDAANLDLNSILEREFSVQSLTSVVNEDCFYEAVERHGKS

SEQ ID NO: 53 - protein, 1720 aa, Rattus norvegicus
VKMGNSESQYTFQGSKNQSNTVTGAKQKPCSLKIRSIHAKDEKSLHGWAHGSGGAGYKSRSLARSCLSHFKSHQPYATRLNGPTCKVSKGTAYSKHRANAPGNDFQGNNGAFLPENGFHYVGRESGESHITSRDCNGHLLTCYGRHDSIASTPPGEDRRSPRVLIKTLGKLDGCLRVEFHNGGNPHKGPSEDPSEPVQLLRYSPTLAAETCPVPESRRLSGAGSSASQRPSPTDSCLRSSKGSSLSSESSWYDSPWGNAGEASEVDGTFLAPSALDPSLPSSFPPSDTKKPFNQSSSLSSLRELYKDANLGCRSPSGTCLSSNEYISSQVGLNNRVSFASDMDVPSRVDHRDTMQYSSFTLPCRKSKALTEDAAKKDTLKARMRRFSDWTGSLSRKKRKLQEPRSTEGSEYFDSHSDGLNADVQGPTQTSALLWSGGSAQTLPHRSESTHAIGSDPLQQNIYENFMRELEMSRTNMENAETSTETVESSSESLSSLEQLDLLFEKEQGVVRKAGWLFFKPLVTLQKERKLELVARRKWKQYWVTLKGCTLMFYETYGKNSTDQNSAPRCALFAEDSIVQSVPEHPKKEHVFCLSNSYGDVYLFQATSQTDLENWITAIHSACASLFAKKHGKEDTVRLLKSQTRGLLQKIDMDSKMKKMAELQLSVVSDPKNRKAIENQIRQWEQNLEKFHMDLFRMRCYLASLQGGELPNPKSLLAATSRPSKLALGRLGVLSVSSFHALVCSRDDSALRKRTLSFTQRAKSKKGLFSSLKGLDTLARKGREKRASITQMFDSSHSHGYLGTQLPQNSTNSSEVHDLHLYGSSADSTLRDSTWEVQTYVHFQDNEGVTVIIKPEHRVEDVLALACKMRHLEPTRYGLQLRKVVDESVEWCVPALYEYMQEQASCVYDEIEVFPLSVYDVQLTKTGDMTDFGFAVTAQVDEHQHLSRIFISDVLPDSLAYGGGLRKGNEIRSLNGEAVSDLDLKQMEALFSEKSVGLTLVARPADTRASLCTSCSDSDLFSRDQKSLLPSPNQCQLLEEFLDNFKKTSTNGKADVPRPLSGCLRQMETLGVPDKVHHDLPYLVTSQSAEQIVELCRGTTSMEAPTDSRDPPPRPLARHLSDADRLRKVIQELVDTEKSYVKDLSCLFELYLEPLQNETFLTQDEMESLFGSLPEMLEFQKVFLETLEDGISASSDFSVLETPSQFRKLLFSLGGSFLYYADHFKLYSGFCANHIKVQKVLERAKTDRAFKAFLDARNPTKQHSSTLESYLIKPVQRVLKYPLLLKELVSLTDHESEEHYHLTEALKAMEKVASHINEMQKIYEDYGTVFDQLVAEQSGTEKEVTELSMGELLMHSTVSWLNPFLSLGKARKDIELTVFVFKRAVILVYKENCKLKKKLPSNSRPAHNSADLDPFKFRWLIPISALQVRLGNTAGTENNSTWELIHTKSEIEGRPETIFQLCCSDSESKTSIVKVIRSILRENFRRHIKCELPMEKTCKDRLVPLKNRVPVSAKLASSRSLKVLRTSSSSEWPSDPSKGNSLDSDECSLSSGTQSSGCPVAESRQDCKSTVLEKDAHEVLAEFPDGLIKESDILSDEEEDFHHPLKQGSPTKDIELQFQRLRISEEPDMHSTGQQPPLTGPGEQPKLVRGHFCPIKRKANSTKRGRGTLLKAQTRHQSLDSHPETASIDLNLVLEREFSVQSLTSVVNEECFYETQSHGKS

SEQ ID NO: 54 - protein, 933 aa, Canis familiaris
MRELEMSRTNTENVETSTDTADSSSGSLSSLEQLDLLFEKEQGVVRKAGWLFFKPLVTLQKERKLELVARRKWKQYWVTLKGCTLLFYETYGKNSMDQSNAPRCALFAEDSIVQSVPEHPKKENVFCLSNSFGDVYLFQATSQTDLENWVTAIHSACASLFAKKHGKEDTVRLLKNQTKNLVQKIDMDSKMKKMAELQLSVVSDPKNRKAIENQIQQWEQNLEKFHMDLFRMRCYLASLQGGELPNPKSLLAAASRPSKLALGRLGILSVSSFHALVCSRDDSAFRKRTLSLTQRGRNKKGIFSSLKGLDTLARKGKEKRPSVTQASSSVDELLHIYGSTADGVPRDNTWEAQTYVHFQDNQGVTVMIKPEHRVEDILTLACKMRQLEPSHYGLQLRKLVDENTEYCIPAPYEYMQEQASVYDEIEIFPLSVYDVQLTKTGSVSDFGFAVTAQVDEHQHLSRIFISDVLPDGLAYGEGLRKGNEIMTLNGEAVSDLDLKQMEALFSEKSVGLTLIARPPDRKSTLCSSWSDSDLFSRDQKSLLPPPNQSQLLEEFLDNFKKNTTNDFSNVPDVTSGLKRSQTDGALARVPREEDTAQTLGSAEQITVLCRSFNDTQSNGMEGPRGTRDPPPRPLARHLSDADRLRKVIQELMDTEKSYVKDLSCLFELYLEPLQNETFLTQDEMESLFGSLPEMLEFQKVFLETLEDGLSASSDFNILETPSQFRKLLFSLGGSFLYYADHFKLYSGFCANHIKVQKVLERAKTDKAFKAFLDARNPTKQHSSTLESYLIKPVQRVLKYPLLLKELVSLTDHESEEHYHLTEALKAMEKVASHINEMQKIYEDYGTVFDQLVAEQSGTEKEPVSVVSMSSLSPVMIQVTELSMGELLMHSAVSWLNPFLSLGKARKDLELTVFVFKRAVILVYKENCKLKKKL

SEQ ID NO: 55 - DNA, 1965 bp, Mouse
atggagcgagcctgtgagaagcaggactcagtgtgcaacctggtggctgtgtttgagaacaacaggactccgggagaagcacctggatcccatagccttgaggaccagctccatagccctgaacaccagctgtccctgtccccagagccttgggaggcaccccctgtcaaggaggccttgaagtctgaattccggccagtgagcaggacatatctgagttccctcaagaacaagctatcgagtggggcttggaggagatcctgccagcctggggtcagcccggggccagagacacaggaacctgaggagaagagggtcgtgcgagagcttctggagacggagcaggcctatgtggctcgcctgcacctgcttgaccaggtgttcttccaggagctgctgagggaggcaggccgcagcaaggccttccctgaggacgtggtgaagctcattttctccaacatctcctccatctatcgtttccacgcccagttcttccttcccgagctgcagcggcgcgtggatgactgggcagccacgccccgcattggggatgtgatccagaagctggccccgtttctgaaaatgtacagcgagtacgtgaagaactttgagcgggccgcggaactgctggccacgtggatggacaagtctcagcccttccaggaggtggtcacccgcatccagtgcagcgaggcctcgggcagcctgaccctgcagcaccacatgttggagcctgtgcaaagaatcccgcggtacgaactgctgctcaaggaatatgtgcagaagctgccagcccaggccccagacctcgaagatgcccagagagcactggacatgatcttctcagctgcacagcactccaatgcagccattgcagagatggagcggctgcagggcctgtgggatgtgtaccagcgcctgggcttggaggatgacatcgtggacccctccaacaccctgctccgagagggccctgttctcaagatctctttccgccgcagcgacccaatggaacgctacctggttttgttcaacaacatgcttctgtattgtgtaccccgagtcctccaagtgggtgcccagttccaggtgcggactcgcatcgatgtggccggcatgaaggtgcgggagctgaccgatgctgagttcccacactccttcctggtgtccggaaagcagcgcacactggagctgcaggcccggtcccgagatgaaatggtttcctggatgcaggcctgccaggcagccattgaccaggttgagaagcggagtgagaccttcaaggctgctgtccagggacctcagggggacacgcaggagcccaagccacaggtagaggagctgggtctccgagcgcctcagtgggtccgggacaagatggtgaccatgtgcatgcgttgccaggagcccttcaatgccctgactcgtcggcgccaccactgccgggcttgcggctatgtagtgtgtgccaagtgctctgactaccgtgcggagctgaaatatgacagcaacaggcccaaccgagtctgcctgacctgctacacatttctcactggaaacgtactccctcaaggcaaggaggacaagaggcggggcatcttggagaaagaggcctcagcagcacccgagcagagtctggtgtgcagcttcctgcagctcataggagacaagtgcagcaggagccttccccggagctggtgtgtgatcccccgggatgacccccttgtgctgtatgtctatgcagccccccaggacacaaaggctcacacctccatccccctactgggctatcaggtgatttcagggccccagggggaccctcgggttttccagctgcaacagtcaggccagcagtacaccttcaaggccgagtctgtggagctgcagggccgctgggtgacagctatcaagcgtgcggccagtggccggacccctgagggacctgacgaagaagatgtgtctgac

SEQ ID NO: 56 - DNA, 3484 bp, Danio rerio
caacatcatcatcatcacgcgaaatacgactactgtggattactaactcaagccatgcttttttctgcattgcagcttgcctgagctgagggatcactggttacagactttacacaggaaaactgtggaggcaaggttgttagcgggcagcacttccccaccccccagcgtcctcatgaaggtgctgagcggcaataccacgaataaaacgctaagtggagatgggatggactctgacttacttgttgatagcaacggcaaaaactccgccctttcaaaagagttgcataaccaggaggcacatgcacagcagccgattggggaaaacggaggcaaaaagtcctttaaaatcccatttaagctgaagagaagctccacaagcccaagtgttactgctcatcctgagtccagtacaaagaacttgctgttcggacgaaatctccaagatgacactgcgctcccaaaaccaattgctgaaatactcctgttgttgttcaggaaggggccagttactgagggggtctttagagtctcatgtaacagcaaaaacctgcatactctcagaaatcagctcaattctggggctgaggtggacatggatgccctgcctgtgacactcttggttggtttgctgaagatttttctgagggaactgcctggaggtttgctgatgtctgagcattatgaaagctggattggtgctctggagaaagagcggacagaggaggtgcaaagtgagctgagaggaatggccgagaagttgcccaaagcaaacagtctcctcctgcagcacctgttgtgcctttttcaccacatcagccagcgatcagaaaccaacaaaatggatgccaagaatctagctgtgtgcattgctccaaccttgctgcatcgtgacagccagcccctagacgtagatgttgtggaaaaggtcacaaaacttatacagttcctgactgagaattgctgtgagatatttggacatgatatcctgacgttgtttggggatctagaagatcggaaggccactgacaagtcggattcagcctccttgatgtcccctgatatatctttcgaagtccaccaacatgactctgcatacgacagcacagatcctgatgcggattgtgactgtgtggaagctgagagctatgaagaaggcatgatggcacatgggagcctaggcctaagcaaaaagggacgctctgacattcagtcttgttcctctgatgctatatttgacacattcaccaaaactttcagccgacgaagctctgagccctccattttatctactctccctataacgggtttgagagagcttgctcgcagccatgatgacttttcaacagagaaggaacactttgacaatcagccgcttaaaaagcagaactcagatgactctttcctgcttcccaaccgctctgagaacagaagatccctcaaaaaactggctgggagttttaacatggacttgcctatcattgtgtcttctccaacatctaagattggttcctgtccatcattctgttcctcagatagcagctcctcaaatcactcagagcagtcaaaaactccttcacctctgccatcccctgcaaaccctcgcaaaacccagtccaccaggcatgcatcgttcatgattaaatctagacataaccatgctcaaggcgaccaagaagttacccgccgatccctctcaatgagagccaagagtctcggaaactttacatttaacagaagcagcctgaagaaaggggactctcagaaggaagtggtcttcccatgtgaaactctccaggaggactcgcaaaatgagacagagaacccagatgaacttgttcgccgtcgacgacctctttctgccattgaagtcttccagcaggtggacagccggatgccatgcagccccccgtcctacgaacaggcacttcagactggagttcaccaggccccaccacaatacagagaaatgacagtccaacacgccagagaactaggcaagaaatctcgtcccatatccatgaacgacaacctactagacatttacaaagtaaacgagtccacagaatgcttagaaacattcacagagagcattcaattagataaaccgcagctggtgacattccggcagagggccatgtccgagtcggtatcccagttgaaacatgagaaagtgtcacgcaggtgtagtcagcctgtttttgaggagttctcctatgctaaggaatcttatgtgtaagtgattctttcttaattgcaatattaaacgccttattaaaggatcagttcacccacatttttagttatgctattaattatttaccatcatgtcattttaactcctcttcagaacacaaatgaaatattttagatcaaatctgagagcttcctcatcctccgtaggcagcaatggtcctgagttaaaagtcttgaaaaagaaccaaaaacattgtcaaagcattccatatgaccttagtggttcaactgtaatcatacgaagctacaagaacactattgtgcaccaaaaacatgtcatattgttcgtagtaaatgtccaccctgatctaatgtagtagacatagccaaaaggtttttacagtttttatgtgcacaaaagtgttcatggagcttcgaatgactacagttgaaccactgaagtcacataaaatgctttgacaactttgttggacttttatggacttttgggtactgttgccatctataaaggatgagggagctctcagattttgtctaaaataccttaatttatgttctgacaatgaacgaagggatttaagtgatatgagggagagtaattaataacagaattctcatgtttgggatgaactaatcctttaatattttaaagtgaagtgtgcgatttttgtgcaaaaaaatcctcaaggaaatctgcaactgtttcccaaactctttccatcggtccaatagtccaaccctaaactcacttcgttggttaacccactgttgctatgtcaaaataaggtactcatactttgctgcattggttgccactatctttccctaccaatcgcttgcttgttaatatgctgtaaaatgtgagtttgtttgattattaatacttttcaagcatcacactgagcactcctgcaaacgtaatgtggaatgtgattaaataagattaatttgactgcattctaataccaaacaggactttaagtttatttgagataggataacctattttaatatagtgaaactgcacactttagctttaaaataccttgctaagctgaactgtagtgacacattaaatgtgttacactgatctcatatgctaatattgaatcttgccagtatgaagctatgtaaatagttacagtaattaaattgtacaacataaaaaatagtctgcaattttgatgtagtatgtacagctgtaaaatacaatatttctgtataatttactctttatttcactacaataaaaatctttaaggccaca

SEQ ID NO: 57 - DNA, 3364 bp, Macacca mulatta
gacagagctgccctaaaaggaatgaggaagtgagagctctccagtgtctggctggcttcgtctgtgtgacagcccatgatgttctttccggtctctgtaatattctgaatttccacctgcccgccccctcgcttataatgcagagcatgtgaagggagaccggctcagtctccctctctcccagtggactagaagcagcagagtgtcatgctctttctcccattatttacagctcaccggatgtaaaagaactctggctagagaccctccgaggacagagccacagccgcacaggagtgaaattcactcccggacagtcagtcccaatactgatgaagctgagaagcagccacaatgcttcaaaaacactaaatgccagtaatatggagacgctaatcgaatgtcaatcagagggtgacatcaaggaacatcccctgttggcatcatgtgagagtgaagacagtatttgccagctcattgaagttaagaagagaaagaaggtgctgtcctggccctttctcatgagaaggctctcccctgcatcagatttttctggggctttggagacagacttgaaagcatcactatttgatcagcccttgtcaattatctgtggtggcagcgacacactccccagacccatccaggacattctcactattctatgccttaaaggcccttcaacggaagggatattcaggagagcagccaacgagaaagcccgcaaggagctgaaggaggagctcaactctggggatgcggtggatctggagagcctccccgtgcacctcctcgctgtggtctttaaggacttcctcagaagtatcccccggaagctactttcaagcgacctctttgaggagtggatgggcactctggagatgcaggacgaggaggacagaatcgaggccctgaaacaggttgcagataagctcccccagcccaacctcctgctgctcaagcacttggtctatgtgctacacctcatcagcaagaactctgaggtcaacaggatggactccagcaacctggccatctgcattggacccaacatgctcaccctggagaatgaccagtgtctgtcatttgaagcccagaaggacctgaacaacaaggtgaagacactggtggaattcctcattgataactgctttgaaatatttggggagaacattccagtacattccagtatcacttctgttgactccctggagcacactgacagttcagatgtgtcgaccctgcagaatgactcagcctacgacagcaacgatcctgatgtggaatccaacagcagcagtggcatcagctctcccagcaggcagccccaggtgcccatggccacagctgctggctttgatagtgggggcccacaggatgcccgagaggtcagcccagagcccattgtgagcaccgtggccaggctgaaaagctccctcgcacagcccgataggaggtactcagagcccagcatgccgtcctcccaggagtgccttgagagccgggtgacaaaccaaacactaacaaagagtgaaggggacttccccgtgccccgggtaggctctcgtttggaaagggaggaggctgaagacccatttccagaggaggtcttccctgcagtgcaaggcaaaaccaagaggccggtggacctgaagatcaagaacttgaccccgggttcggtgctcccaagggcactggttcccaaagccttctccagcagctcgctggacgcgtcctctgacagctcacccgtggcttctccttccagtcccaaaagaaatttcttcagcagacatcagtctttcaccacaaagacagaaaaaggcaagcccagcagagaaattaaaaagcactccatgtctttctcctttgcccctcacaaaaaagtgctgaccaaaaacctcagcgcagggtctgggaaatcgcaagactttaccagggaccacgtcccaaggggtgttagaaaggaaagccagcttgctggccgaatcgtgcaggaaaatgggtctgaaacccacaatcaaacaacccgcgccttctgcctgagaccccacgccctctcggtggatgatgtgttccagggagctgactgggagaggcctggaagcccaccctcttatgaagaggccatgcagggcccggcagcccgactcgcggcctacgggagccagaccgtggggagcatgactgtggggagcatgagggcgaggatgctggaggtggactccctcctaccccctcttccacctgctcaccacacaggagactcaagacacagggacagcaaagagccactccctggccacggaccctctcccctgcctgagcgatggatacagagcagaactgtccatgcttcttgggactctgtggggcacgtgtctggcccagggagacctgagctcctccggctgaggaccgtctccgagtccgtgcagaggaataagcgggactgtctcatgcgacgatgtagccagccggtctttgaggctgaccagttccaacatgccaaagaatcgtatatttaggagggaggccatacaccatgccacagcttgtgctatctgtaaatatgagacttgtaaagaactgcctttagattgtggttttaaaggtcttgaataagctcctttagaacgctgtgcaaagccctcctcagtgaggatagctacaccatggccatggtgcatcagatagtctgtgtgtacccggatttgtgcaatatgtaacaaatgtataaaatgtattatagataaggtgttaggtgcaaagaatgtctaataatccctgcatacgtttgtggatttgcagtgaagtacattgctgttccttgcttcctggggcacttttctcttggctagtgtttgaaaattatctttgcttttataatgtggcctcaaatgtcatgccaattttcacattttccacaaactccatttagggagaagtgtttaaacccccggtaagtttactctataccagagtaaactatatattactccatataagcagccttgcaataactaatcaccaccatagacgaaagaaacagactgcaaggaacagagctgagtgtctggagccatcaaaggcattaaaaactccagcaaaagcagaggtcatagcaaaaatcatgaaaaacacttcaacctgtcctttcaatcatccaattaaatttgggtaaattaatgaaaatgtattatatcaatattaactcatctacagcactttgagtttctttgtaattcatgatgtcctatcctatagtgtggaggtaaatgattttatatgcattgggggtcatatataaaacttcaatgtaatttcactacaataaattgccttccttatttgaaagtaaaaa

SEQ ID NO: 58 - DNA, 2175 bp, Monodelphis domestica
atgaaggtgttaagtggctgcaatgcttccaaaacactaaatgccagtgacatggaaactttaattgaatgtcagccagagggcaatgtgaagcaacaccacttgctggcacaatgtgatagtgaagatgggatttgccacctgattgaaaacaagaagagaaaggtgatagcctggccttttcccatgagaagaacagcttcttcttcagattcttctggatctttaacaccagaatggaaggcttccctgtttgaccagcccttgtcagcaatctgcactgatgaagatatccttccccaaccaattcaggaaattcttaatattcttcaaaacaaaggcccttcaactgaaggaatattcagaaaggctgccaatgaaaaggcccggaaagaacttaaggaagagctcaactctgggggaatggtcaatttggaaaccaaatctgttcacctactggctgcagttttaaaggatttcctcaggagtatcccactcaaactactttcatcagatctatttgaagaatggatgacagccttagaaaggccaaatgaggatgacaaaattgagagcttgaaacaggttgtaaagaaactaccaaggacaaatatcctgttgctcaaacatttggtctatgtgctttacaacatcagtaagaattcggatgtcagcaagatggactcaagcaatcttgccatttgtattggacccaacatgctgtccctcaacactgaccaaagcctatcttttgatgctcagaaggagttgaacaacaagattaaaactttggtggaatttttcattgataactgctttgaaatatttgaagaggacatccctggacattcccccaggttcaattctgatgattcactggaacacatcaacaattcagatatgtcaactctgcaaaatgactcagcctatgagagtacagatgctgacacagaatgcagcaacagttttggctctcaaaacaagccgtccctagacactgcaggactgtccagtgacttggacaacagaaagcatctgtatgagtcaagaccagggtctgttgtctcttacaccaacttactaaaaagctcccttagcactcaagaaaggaggtactcagagcctagcacaccatccacgaaagactgccttgaaagcaggatcaccagccacaaattgactaaaagtgaggacagcttcgctgtcccccaggcaagttcctgttttgggggtcaagaaataaaagaatcatttccagaggaatgtttccctagtctacaacacaggaaacaaaagtcattaggtcttcaaataaaggaaggaactttatgcacagaatcacttgttgagccttcccctaaaacctcctcctgtggttccctggatagttcctctgatagctcagtctttgccaactcgccagtggtttctccttccagtccaaaaaggaactttttcaccaggcatcaatcctttacaacaaagactgctggggcggacagtaagctgacccgagagagaaaaaagcactccatgtcattctcttttgctactcataagaaggtgccaaacaaaactgcaaattggcgctttcccagagaccagggcaagaaagacctcaaaaaggagagccaactcactggcaggattgtccaagaagtctgtgttggagctatagatgactaccaaccaactccagaatgtgggagttcaaggtcacatcttctttcagttgaagaagtatttcaacttgtagatcagaaaaaccctggaagccccccatcctatgaggaggcagttcaatattgccggctttccaaaattcctccttatgagagccggacagtccaaagtatgagagacactatgcaaagccagaattctagactgccatctctttcacttttggactatggtgagcatgccaagaatacacacggcaaagaaacactcaacagagacagtgcatcatctgtgggtgaaacttgggtacagaatggagcttccaatgtggctatggaaaggaaaggacaaataccaaaatctgaggttcgccggttaagggtcttgtctgaatccttgctaaagaataaacaggactgtgtcattagaaggtgtagtcagccaatctttgaggttgaccaaatccaatatgccaaggaatcctatgtttag

SEQ ID NO: 59 - DNA, 2109 bp, Xenopus tropicalis
atgaaggtgctcacaggctgcaatgcttttaaagcaatcaataccagcaacatggaagacttaatagaatgcccaacagaggcagatgccaagaaataccagttattggcagcaatgataagtgaagatggcatgtgccatgtaattgaaaacaacaaaaaaagaaaggctgtcatatcctggccattcacattcagaagaagctcaacactatcagaaacttcagttccacctgaactaaaagcaactttgtttgaccaaccactttctattgtctgtgaagaggacgcccttcccaaaccaatacttgaaatccttactattctgtgccagcagggcccatccactgagggcattttccgtaaagctgccaacgagaaagcacgcaaagaactcaaggaggatctcaatagtggaaagacggtggatttaaaaagcaagcatgtgcatttattggctgtggttctcaaggtaaatatattgtctacgcaaaaagaaatgatcatcaataacaactttttcttttttcttaacaacttcattagtgatgagcgaatatgttctgtttcacgtgttgcggataaactgcccagacctaactggattctactgcaacatttaatctgcgtcctctatcacatcagcaaagcctcaactttaaacaagatggattcaaacaatctagctgtctgtattggcccaaacatgctccaacctcaccatgactataatctttccctagaagctcagaagcaggccaatgacagggttatttccctagttgagttcttcatagacaattgctttgatctgtttggtcaaaatgtatcacagtgtctaagtacctcaaaggaggagttgctggaggacactgatgtctcagaaatcccttttcaacaaaatgactcagcatatgacagcacagatcctgaatatgagggacataatagtaccatcaccagctgtcaaaaagacttggggattaacagtgggagagaggttctaaatagctcaatggatcaggatataaatctgactcaatcttctataaacttgttgaaagtcagcaatatggacaggaggaagtcagaaccacacatattcccctcacaggataccaaggtggtcgcaggcagaaaactgaccagaagccatgatgatgtcactgttcgtaaaagtggagaacctttaactaaggaggacctatcagagcaagtgtcagaaatagccttatataagaaaaagatgccaaaaggtctgacagttaataccagttattcagaagatgtgttagatgatgtactgcagaatgctctttccagttgttcactagagagttgcttttcagactgttctgtgtttaccagctcaccgcttgcttctcccataagtccaaagaacaactgtttgataagacatcaatcatgttcttcgaagagtggaattagaaatgacattaaattatcaagcagagaaataaaaaagcattcaaagtcattttcctatgtgaatcaaaagaaaaagttggcaaagacacagagctgggggcctgaaggacaaaatccaggcttacagaggtacatgtttaacagcagtctcagaaatagacatcaatatgaaaatccacaggctaaatgttttcagcagcctgctgttgttaggcttagaaggccacaatctgcccgcaaaatgtcagttgatgaggtgttccgaatagtagaccagagaaaccctggcaaacctccatcatatgaggaagccattcataaaaatgttccaccctttaaaggtatgacagtccaaactgtgagagctactgtttcaaataatgaatattcatcttcccatgttgtcagtgatacccccatggacagagaatgtagaactcatgcagagaaacagacaacaataataaatgaagggccaacagaaaaaaactgtgccgaggaaatgcaacagctctgtcagtttggaaaaactaaaagtgttgttatacgaactatgtctgagtctgttcagaaacacaagcatgaaaccttaagtcggagatgcagccagccttttgagctttatgatcagattcagtatgccaaggaatcctatgtttag

SEQ ID NO: 60 - DNA, 3354 bp, Pan troglodytes
gacatagctgccctaaaaggaatgaggaagcgagagctctccagtgtctggctggctccgtccgtgtgacagcccatgatgttctttccggtctctgtaatattctgaatttccacctgcccgccccctcgcttataatgcagagcatgtgaagggagaccggctcagtctctctctctcccagtggactagaaggagcagagtgttacgctgtttctcccattctttacagctcaccggatgtaaaagaactctggctagagaccctccaaggacagaggcacagccacacgggagtgaaatccacccctggacagtcagccgcaatactgatgaagctgagaagcagccacaatgcttcaaaaacactaaacgccaataatatggagactctaatcgaatgtcaatcagagggtgatatcaaggaacatcccctgttggcatcatgtgagagtgaagacagtatttgccagctcattgaagttaagaagagaaagaaggtgctgtcctggccctttctcatgagaaggctctcccctgcatcagatttttctggggctttggagacagacttgaaagcatcgctatttgatcagcccttgtcaattatctgcggtgacagtgacacactccccagacccatccaggacattctcactattctatgccttaaaggcccttcaacggaagggatattcaggagagcagccaacgagaaagcccgtaaggagctgaaggaggagctcaactctggggatgcggtggatctggagaggctccccgtgcacctccttgctgtggtctttaaggacttcctcagaagtataccccggaagctactttcaagcgacctctttgaggagtggatgggtgctctggagatgcaggacgaggaggacagaatcgaggccctgaaacaggttgcagataagctcccccagcccaacctcctgctactcaagcacttggtctatgtgctgcacctcatcagcaagaactctgaggtgaacaggatggactccagcaatctggccatctgcattggacccaacatgctcaccctggagaatgaccagagcctgtcatttgaagcccagaaggacctgaacaacaaggtgaagacactggtggaattcctcattgataactgctttgaaatatttggggagaacattccagtgcattccagtatcacttctgatgactccctggagcacactgacagttcagatgtgtccaccctgcagaatgactcagcctacgacagcaacgaccctgatgtggaatccaacagcagcagtggcatcagctctcccagcaggcagccccaggtgcccatggccacagctgctggcttggatagcgcgggcccacaggatgcccgagaggtcagcccagagcccattgtgagcaccgtggccaggctgaaaagctccctcgcacagcccgataggagatactcagagcccagcatgccatcctcccaggagtgcctcgagagccgggtgacaaaccaaacactaacaaagagtgaaggggacttccccgtgccccgggtaggctctcgtttggaaagtgaggaggctgaagacccatttccagaggaggtcttccctgcagtgcaaggcaaaaccaagaggccggtggacctgaagatcaagaacttggccccgggttcggtgctcccgcgggcactggttctcaaagccttctccagcagctcgctggacgcgtcctctgacagctcgcccgtggcttctccttccagtcccaaaagaaatttcttcagcagacatcagtctttcaccacaaagacggagaaaggcaagcccagccgagaaattaaaaagcactccatgtctttctccttcgcccctcacaaaaaagtgctgaccaaaaacctcagcgcgggctctgggaaatcgcaagactttaccagggaccacgtcccgaggggtgtcagaaaggaaagccagcttgccggccgaatcgtgcaggaaaatgggtgtgaaacccacaaccaaacagcccgcggcttctgcctgagaccccacgccctctcggtggatgatgtgttccagggagctgactgggagaggcctggaagcccaccctcttatgaagaggccatgcagggcccggcagccagactagtggcctacgagagccagaccgtggggagcatgacggtggggagcatgagggcgaggatgctggaggcgcactgcctcctaccccctcttccacctgctcaccacgtagaggactcaagacacaggggcagcaaagagccactccctggacacggactctctcccctgcctgagcgatggaaacagagcagaactgtccatgcttctggggactctctggggcacgtgtctggcccagggagacctgagctcctcccgctgaggactgtctccgagtccatgcagaggaataagcgggactgtctcgtgcgacgatgtagccagccggtctttgaggctgaccaattccaatatgccaaagaatcgtatatttaggagggacgccatacgccatgccatagcttgtgctatctgtaaatatgagacttgtaaagaactgcctgtagattgcttttaaaaggtcttgaataagctccttgagaaagttgtggaaagccctcctcagtgaggatagctacaccatggccatggcgcatcagatagtctctgtgtacctggatttgtgcaatatgtaaaaatgtatcaaatgtattatagataaggtgttaggtgcaaaggatgtctaataatccctgcacacgtttgtgaacttgcagtgaagtacactgctgttccttgcttcctggggcacttttctcttggttggtgtttaaaaattatcttcgcttttttaatgtggcctcaaatgtcatgccaattttcacattttccacaaactccatttagggagaaatgtttaaatctctggtataagtttactccataccagagtaaactatatattactctatataagcagccttgcaataactaatcaccaccatagaagaaagaaacagactgcaaggaacagagttgagtgtctggagtcatcaaaggcattaaaaactccagtaaaagctggggccatagcagaaatcatgaaaaacacttcaacatgtcctttcaatcatccagttaaatttgggtagattaatgaaaatgtattacatcaatattaactcatctatagcactttgagtttctttgtagttcatgatatcctatcctataatgtggaggtaaatgattttatatgcattgggggtcacatataaaacttcaatgtaatttcactacaataaattgccttccttatttg

SEQ ID NO: 61 - DNA, 2480 bp, Macacca mulatta
agaacagattcacgggtgatttagcctatctgtcccaggccaaggtggctgcatgtgctagctggaggcctctctctctgcttcaagggtagctgagatccaccccggaaaccggcaggatgaaggaggcaagtgaggagaagctggcgtctgtgtccaacctggtcactgtgtttgagaatagcaggactccagaagcagcacccagaggccacaggctagaggacgcgcatcaccaccctgagtgcaggcctcccaggtccccaggaccgtgggagaagctgaatgttggggaggccatggggtctgagcccaggacagtcagcgggaggtacctgaactccctgaagaacaagctgtccagtggagcctggaggaaatcttgccagcctgtgaccctctcaggatcggggatgcaggagccagagaagaagatcgtccaggagctgctggagacagagcaggcctacgtggcacgcctccacctgctagaccaggccatggtgtttttccaggagctgctgagggcggcccgcaggagcaaggccttccccgaggacgtggtcagggtcatcttctccaacatctcctccatctatcagttccattctcagttcttcctcccagagctgcagcggcgcctggatgactggacagccaacccccgcatcggcgacgtgatacagaagctggcccccttcctgaagatgtacggtgagtatgtcaagaactttgagcgagcggctgagctgctggccacctggaccgagaagtctccactcttccaggaggttctcactcgcatccagagcggtgaggcttcgggcagcctgaccctgcagcaccacatgctggaaccagtgcagagaattccacgctacgagctgctgctcaaggagtacgtccagaagctgccagcccaggccccagaccgggccgatgctcagaaagccctggacatgatcttctcagctgcccagcactccaatgcagccatcactgagatggagcggctgcaggacctgtgggaggtgtaccagcgcctgggcctcgaggacgacatagtagacccttctaacaccctgctccgtgagggcccggtcttcaagatctccttccgccgcaacgatcccatggagcgctaccttttcttgttcaacaacatgctgctctactgcgtgcccagggtgatccaggtgggtgcccagttccaggtgaggacccgcatcgacgtggccgggatgaaggtgcgggagctgatggacgctgagttcccccactccttcctggtgtctgggaagcagcgcactctggagctgcaagcccggtcccaggaggaaaagatttcctggatgcaggccttccaagcagctgttgaccaaattgagaagcggaatgaaaccttcaaggcggcggcccaggggcctgagggagacacccaggagcaggagctgcagtctgaggagctgggcctccgagcaccacagtgggtccgggacaaaatggtgaccatgtgcatgcgctgccaggagcccttcaacgctctgacgcgccgtcgccaccactgccgggcctgcggctatgtggtgtgtgccaggtgctccgactaccgggccgagctgaaatataacgacaataggcaaaaccgagtctgcctccactgctacacgttcctcactggaaacgtgctccctgaggccaaggaggacaagaggcggggcatcctggagaaagggtcctcagccacgcctgaccagagcctgatgtgcagcttcctgcagctcatcggggacaagtggggcaagagtggcccccggggctggtgtgtgatccctcgagatgacccccttgtgctctatgtctacgctgcccctcaggacatgagggctcacacctccatccccctgctgggctaccaggtgaccgttgggccccagggggaccctcgggtcttccagctacagcagtcaggccaactctacactttcaaggccgagacggaggagctgaagggccgctgggtgaaggccatggagcgggcggccagtggatggagtcccagctggcccaacgatggggacctgtccgactgagccactgccagccactctcctgcccacctctccccaccctgaacccagctcctgccacagactgaccctgtggcctcaggacccactgccccaaggggtgctttcacagaattgattcagccatctgcgctcaggccatgtgtcccgatctgggatgcagaaaatatgggtccattcctttctagaaaggggacaaccaagtgtctcagtttgccttgcggggagggggctcctgggccatgggacttccagtgctgaaactgggaaagccccaggtaaccccggactggtggtaccagagtgtggttttcaaccggggctgctcatttgtgtctcccggggagcttttaaagagtactggtgaaaaacacatagtaaattaatttt

SEQ ID NO: 62 - DNA, 2471 bp, Macacca mulatta
acagattcacgggtgatttagcctatctgtcccaggccaaggtggctgcatgtgctagctggaggcctctctctctgcttcaagggtagctgagatccaccccggaaaccggcaggatgaaggaggcaagtgaggagaagctggcgtctgtgtccaacctggtcactgtgtttgagaatagcaggactccagaagcagcacccagaggccacaggctagaggacgcgcatcaccaccctgagtgcaggcctcccaggtccccaggaccgtgggagaagctgaatgttggggaggccatggggtctgagcccaggacagtcagcgggaggtacctgaactccctgaagaacaagctgtccagtggagcctggaggaaatcttgccagcctgtgaccctctcaggatcggggatgcaggagccagagaagaagatcgtccaggagctgctggagacagagcaggcctacgtggcacgcctccacctgctagaccaggtgtttttccaggagctgctgagggcggcccgcaggagcaaggccttccccgaggacgtggtcagggtcatcttctccaacatctcctccatctatcagttccattctcagttcttcctcccagagctgcagcggcgcctggatgactggacagccaacccccgcatcggcgacgtgatacagaagctggcccccttcctgaagatgtacggtgagtatgtcaagaactttgagcgagcggctgagctgctggccacctggaccgagaagtctccactcttccaggaggttctcactcgcatccagagcggtgaggcttcgggcagcctgaccctgcagcaccacatgctggaaccagtgcagagaattccacgctacgagctgctgctcaaggagtacgtccagaagctgccagcccaggccccagaccgggccgatgctcagaaagccctggacatgatcttctcagctgcccagcactccaatgcagccatcactgagatggagcggctgcaggacctgtgggaggtgtaccagcgcctgggcctcgaggacgacatagtagacccttctaacaccctgctccgtgagggcccggtcttcaagatctccttccgccgcaacgatcccatggagcgctaccttttcttgttcaacaacatgctgctctactgcgtgcccagggtgatccaggtgggtgcccagttccaggtgaggacccgcatcgacgtggccgggatgaaggtgcgggagctgatggacgctgagttcccccactccttcctggtgtctgggaagcagcgcactctggagctgcaagcccggtcccaggaggaaaagatttcctggatgcaggccttccaagcagctgttgaccaaattgagaagcggaatgaaaccttcaaggcggcggcccaggggcctgagggagacacccaggagcaggagctgcagtctgaggagctgggcctccgagcaccacagtgggtccgggacaaaatggtgaccatgtgcatgcgctgccaggagcccttcaacgctctgacgcgccgtcgccaccactgccgggcctgcggctatgtggtgtgtgccaggtgctccgactaccgggccgagctgaaatataacgacaataggcaaaaccgagtctgcctccactgctacacgttcctcactggaaacgtgctccctgaggccaaggaggacaagaggcggggcatcctggagaaagggtcctcagccacgcctgaccagagcctgatgtgcagcttcctgcagctcatcggggacaagtggggcaagagtggcccccggggctggtgtgtgatccctcgagatgacccccttgtgctctatgtctacgctgcccctcaggacatgagggctcacacctccatccccctgctgggctaccaggtgaccgttgggccccagggggaccctcgggtcttccagctacagcagtcaggccaactctacactttcaaggccgagacggaggagctgaagggccgctgggtgaaggccatggagcgggcggccagtggatggagtcccagctggcccaacgatggggacctgtccgactgagccactgccagccactctcctgcccacctctccccaccctgaacccagctcctgccacagactgaccctgtggcctcaggacccactgccccaaggggtgctttcacagaattgattcagccatctgcgctcaggccatgtgtcccgatctgggatgcagaaaatatgggtccattcctttctagaaaggggacaaccaagtgtctcagtttgccttgcggggagggggctcctgggccatgggacttccagtgctgaaactgggaaagccccaggtaaccccggactggtggtaccagagtgtggttttcaaccggggctgctcatttgtgtctcccggggagcttttaaagagtactggtgaaaaacacatagtaaattaatttt

SEQ ID NO: 63 - DNA, 2043 bp, Macacca mulatta
atgtttccaaagaaagccaggcacccagggttccctgcactggagatttgcaccaggcaaccatccacaccagggacttgctcgtgttttccctgttctccagggaggaaaccctcaggtctgtctcttctcctcaggactccagaagcagcacccagaggccacaggctagaggacgcgcatcaccaccctgagtgcaggcctcccaggtccccaggaccgtgggagaagctgaatgttggggaggccatggggtctgagcccaggacagtcagcgggaggtacctgaactccctgaagaacaagctgtccagtggagcctggaggaaatcttgccagcctgtgaccctctcaggatcggggatgcaggagccagagaagaagatcgtccaggagctgctggagacagagcaggcctacgtggcacgcctccacctgctagaccaggccatggtgtttttccaggagctgctgagggcggcccgcaggagcaaggccttccccgaggacgtggtcagggtcatcttctccaacatctcctccatctatcagttccattctcagttcttcctcccagagctgcagcggcgcctggatgactggacagccaacccccgcatcggcgacgtgatacagaagctggcccccttcctgaagatgtacggtgagtatgtcaagaactttgagcgagcggctgagctgctggccacctggaccgagaagtctccactcttccaggaggttctcactcgcatccagagcggtgaggcttcgggcagcctgaccctgcagcaccacatgctggaaccagtgcagagaattccacgctacgagctgctgctcaaggagtacgtccagaagctgccagcccaggccccagaccgggccgatgctcagaaagccctggacatgatcttctcagctgcccagcactccaatgcagccatcactgagatggagcggctgcaggacctgtgggaggtgtaccagcgcctgggcctcgaggacgacatagtagacccttctaacaccctgctccgtgagggcccggtcttcaagatctccttccgccgcaacgatcccatggagcgctaccttttcttgttcaacaacatgctgctctactgcgtgcccagggtgatccaggtgggtgcccagttccaggtgaggacccgcatcgacgtggccgggatgaaggtgcgggagctgatggacgctgagttcccccactccttcctggtgtctgggaagcagcgcactctggagctgcaagcccggtcccaggaggaaaagatttcctggatgcaggccttccaagcagctgttgaccaaattgagaagcggaatgaaaccttcaaggcggcggcccaggggcctgagggagacacccaggagcaggagctgcagtctgaggagctgggcctccgagcaccacagtgggtccgggacaaaatggtgaccatgtgcatgcgctgccaggagcccttcaacgctctgacgcgccgtcgccaccactgccgggcctgcggctatgtggtgtgtgccaggtgctccgactaccgggccgagctgaaatataacgacaataggcaaaaccgagtctgcctccactgctacacgttcctcactggaaacgtgctccctgaggccaaggaggacaagaggcggggcatcctggagaaagggtcctcagccacgcctgaccagagcctgatgtgcagcttcctgcagctcatcggggacaagtggggcaagagtggcccccggggctggtgtgtgatccctcgagatgacccccttgtgctctatgtctacgctgcccctcaggacatgagggctcacacctccatccccctgctgggctaccaggtgaccgttgggccccagggggaccctcgggtcttccagctacagcagtcaggccaactctacactttcaaggccgagacggaggagctgaagggccgctgggtgaaggccatggagcgggcggccagtggatggagtcccagctggcccaacgatggggacctgtccgactga

SEQ ID NO: 64 - DNA, 1890 bp, Monodelphis domestica
agcaactccatctcccagatgccccagactctgatggagccccattttgataggactccaggaccctgggaaagggacagtctggaagatagaccaggacctggccactgggcagtcagcaccaggctactctcctctctaagaaataagatctccaacagcggctggaggaagtcctgtgaatttccattctctaaccccaatctgccaccccacttgcaggaaccagaagagaatagaattgtcagggagctcctggagacagagcaggcctatgtgtcccgcctctaccttctggatcaggtcttctttgaggagctactaaaggaggctcgaaacagcaaggctttctctgaagacattgtcaagctcatcttttctaacatttcctccattcaccaattccactcccagttcttcctcccagagcttcagaggcgtgtggatgaatgggttgttgtcccccgcattggtgatgtgatccagaaactggcaccctttctgaagatgtacagtgaatacgtgaagaactttaagcgggcagcagagctgctggtcatctggacagagaagtgcccacctttccaagaggtcatcactcgaatccagaacagtgacatctctgacagcctcaccctgcaacaccacatgctggagcccgtgcagagaatccctcgctatgagctgttgctgaaggagtacgtccagaagctgccccctgatgccccagaccgggctgatgcccagaaagctttggacatgatcttctctgcagctcagcattctaatgcagccatcactgaaatggagcgtctgcagaacctgtgggatgtgtatcagcgtcttggccttgaagatgatattgtggatccctctaatgaattattgcaggagggtcccatccagaagatctccttcagacatagtagcaccatggagcgctacttattcctgttcaacaatatgctgctgtactgtgtgcctaaggtgattcaggttggggctcagttccaggtgaggacccgaattgatgtggcggggatgaaggtccgggaactgaatgatgtggaatttcctcactcgttcttggtgtcagggaaacagagaaccctggaactacaagcccagtctcaggaagaaatgaacacctggatccaggcctgccaacgggccatcgatctgattgggaggcggcacggaaccttcaaggctgctgtccaggggacggagggagactctgaggagcaccaactgaagtctgaagagctgggaatccgagctccccagtgggtccgggacaagatggtgaccatgtgtatgcgttgcaagacacccttcaatgccctcaccagaagacgacaccactgcagagcctgtagctatgtggtgtgtgctaagtgctccgattaccgagctaggttgcaatatgatgacaatcggctccaccgggtctgcctgcagtgttacgttttcctcactgggaacctactgccagaggacaaggaagagaagaagaaagggatcctggagaaagagtcttcgaaggtatcagaacagagcgtgatgtgcagcttcctgcaactcctgggagacaagtgggcgaagggcagcagccgaggctggtgtgtcatccccagagatgaccccctggcactttacgtctatgctgccccacaggacatgaaagctcacacctccattccactgttggggtaccaagtgaccacaggttccttggccgatccccgggcattccagatacagcagtcgggtctggtctactcattcagggcagaatctgaggagctaaaggggcaatggatgaaggccatagaacgagcatccagaggtcagagcttcactgggccagaaggagagctgtcagactga

SEQ ID NO: 65 - DNA, 2467 bp, Pan troglodytes
acagattcatgggtgatttagcctatctgtcccaggccagggcggctgagtgtgctggctggaggcctctctctctgcttcgagggtagctgagatccaccccggaaaccggcaggatgaagggggcaagtgaggagaagctggcatctgtgtccaacctggtcactgtgtttgagaatagcaggaccccagaagcagcacccagaggccacaggctagaggacgtgcatcaccgccctgagtgcaggcctcccgagtccccaggaccacgggagaagacgaatgtcggggaggccgtggggtctgagcccaggacagtcagcaggaggtacctgaactccctgaagaacaagctgtccagcgaagcctggaggaaatcttgccagcctgtgaccctctcaggatcggggacgcaggagccagagaagaagatcgtccaggagctgctggagacagagcaggcctatgtggcgcgcctccacctgctagaccaggtgtttttccaggagctgctgaagacagcccgcagcagcaaggccttcccagaggatgtggtcagggtcatcttctccaacatctcctccatctatcagttccattctcagttcttcctcccagagctgcagcggcgcctggacgactggacagctaacccccgcatcggtgacgtgatccagaagctggcccccttcctgaagatgtacagtgagtatgtcaagaactttgagcgagcggctgagctgctggccacctggaccgacaagtctccactcttccaggaggttctcactcgcatccagagcagcgaggcttcgggcagcctgaccctgcagcaccacatgctggaaccagtgcagagaattccacgttacgagctgctgctcaaggagtacatccagaagctgccagcccaggccccagaccaggccgatgcccagaaagccctggacatgatcttctcagctgcccagcactccaatgcagccatcactgagatggagcggctgcaggacctgtgggaggtgtaccagcgcctgggcctcgaggacgacatagtagacccctctaacaccctgctccgtgagggcccggtcctcaagatctccttccgccgcaacgaccccatggagcgctaccttttcttgttcaacaacatgctgctctactgtgtgcccagggtgatccaggtgggcgcccagttccaggtgaggacccgcatcgatgtggccgggatgaaggtgcgggagctgatggatgctgagtttccccactccttcctggtgtccgggaagcagcgcaccctggagctgcaagcccggtcccaggaggaaatgatttcctggatgcaggccttccaagcagccattgaccaaatcgagaagcggaatgaaaccttcaaggctgcggcccaggggcctgagggagacacccaggagcaggagctgcagtctgaggagctgggcctccgggcaccgcagtgggtccgggacaagatggtgaccatgtgcatgcgctgccaggagcccttcaacgctctgacgcgccgtcgccaccactgccgggcctgcggctatgtggtgtgtgccaggtgctcccactaccgggccgaactgaaatacgacgacaacaggcccaaccgagtctgcctccactgctacgcattcctcactggaaatgtgctgcctgaggccaaggaggacaagaggcggggcatcctggagaaagggtcctcagccacgcctgaccagagcctgatgtgcagcttcctgcagctcatcggggacaagtggggcaagagcggcccccggggctggtgtgtgatccctcgggatgaccccctcgtgctctatgtctacgctgcccctcaggacatgagggctcacacctccatccccctgctgggctaccaggtgactgttgggccccagggggaccctcgggtcttccagttacagcagtcaggccagctctacaccttcaaggccgagacggaggagctgaagggccgctgggtgaaggccatggagcgggcggccagtggctggagccccagctggcccaacgacggggacctgtccgactgagccactgccagccgctctcctgcccacctctccccaccctgaacccagctcctgccacagactgaccctgtggcctcagtgacccactgccccaagtggtgctttcagagaattgattcagccatctgcgcccaggccacgtgtcccaatctgggattagaaaatatgggtccattcctttctagaaaggggacaaccaagtgtctcagtttgccttgtggggagggggctcctgggccttgggacttccagtgctaaaactaggaaagccccaggtaaccccggactggtggtcaccatagtgtggtttttcatttgtatctcctggggagcttttaaagagtactggtgaaaaacacatagtaaattaattttaaaaatgt

SEQ ID NO: 66 - DNA, 2401 bp, Homo sapiens
tctttcttcactctgaagccaagagccgaccttctgagccctcaagaaagatcagaacagattcatgggtgatttagcctatctgtcccaggccagcgtggctgagtgtgctggctggaggcctctctctctgcttcgagggtagctgagatccaccccggaaaccggcaggatgaagggggcaagtgaggagaagctggcatctgtgtccaacctggtcactgtgtttgagaatagcaggaccccagaagcagcacccagaggccagaggctagaggacgtgcatcaccgccctgagtgcaggcctcccgagtccccaggaccacgggagaagacgaatgtcggggaggccgtggggtctgagcccaggacagtcagcaggaggtacctgaactccctgaagaacaagctgtccagcgaagcctggaggaaatcttgccagcctgtgaccctctcaggatcggggacgcaggagccagagaagaagatcgtccaggagctgctggagacagagcaggcctatgtggcgcgcctccacctgctagaccaggccatgagtgacctgtcgtggcggctacaggtgtttttccaggagctgctgaagacagcccgcagcagcaaggccttcccagaggatgtggtcagggtcatcttctccaacatctcctccatctatcagttccattctcagttcttcctcccagagctgcagcggcgcctggacgactggacagctaacccccgcatcggtgacgtgatccagaagctggcccccttcctgaagatgtacagtgagtatgtcaagaactttgagcgagcggctgagctgctggccacctggaccgacaagtctccactcttccaggaggttctcactcgcatccagagcagcgaggcttcgggcagcctgaccctgcagcaccacatgctggaaccagtgcagagaattccacgttacgagctgctgctcaaggagtacatccagaagctgccagcccaggccccagaccaggccgatgcccagaaagccctggacatgatcttctcagctgcccagcactccaatgcagccatcactgagatggagcggctgcaggacctgtgggaggtgtaccagcgcctgggcctcgaggacgacatagtagacccctctaacaccctgctccgtgagggcccggtcctcaagatctccttccgccgcaacgaccccatggagcgctaccttttcttgttcaacaacatgctgctctactgtgtgcccagggtgatccaggtgggcgcccagttccaggtgaggacccgcatcgatgtggccgggatgaagatgcaccggaatttcttcaaccagtcttctgctgagagacacttagattgttttcagttgtcagcagctgcaaatgaggctgcagtgaatagcctggtgcgggagctgatggatgctgagtttccccactccttcctggtgtccgggaagcagcgcaccctggagctgcaagcccggtcccaggaggaaatgatttcctggatgcaggccttccaagcagccattgaccaaatcgagaagcggaatgaaaccttcaaggctgcggcccaggggcctgagggagacatccaggagcaggagctgcagtctgaggagctgggcctccgggcaccgcagtgggtccgggacaagatggtgaccatgtgcatgcgctgccaggagcccttcaacgctctgacgcgccgtcgccaccactgccgggcctgcggctatgtggtgtgtgccaggtgctccgactaccgggccgaactgaaatacgacgacaacaggcccaaccgagtctgcctccactgctacgcattcctcactggaaatgtgctgcctgaggccaaggaggacaagaggcggggcatcctggagaaagggtcctcagccacgcctgaccagagcctgatgtgcagcttcctgcagctcatcggggacaagtggggcaagagcggcccccggggctggtgtgtgatccctcgggatgaccccctcgtgctctatgtctatgctgcccctcaggacatgagggctcacacctccatccccctgctgggctaccaggtgactgttgggccccagggggaccctcgggtcttccagctacagcagtcaggccagctctacaccttcaaggccgagacggaggagctgaagggccgctgggtgaaggccatggagcgggcggccagtggctggagccccagctggcccaacgatggggacctgtccgactgagccactgccagccgctctcctgcccacctctccccaccctgaacccagctcctgccacagactgaccctgtggcctcagtgacccactgccccaagtggtgctttcagagaattgattcagccatctgcgcccag

SEQ ID NO: 67 - DNA, 5248 bp, Macacca mulatta
atgggcaactccgagagtcagtacacccttcaaggatctaaaaatcatagcaatactatcactggtgctaagcaaattccttgctccctgaaaatacgtgggattcatgcaaaagaggaaaagtcattgcatggatgggttcacggaagcagcggagcaggttacaagtccaggtccctggcccgaagctgcctttctcactttaagagtaaccagccttacgcatccagactcggtggctccacgtgcaaggtctccagaggcgttgcctactccacgcacaggacaaatgccccagggaaggatttccagggcatcagtgctgctttctcaactgagaacggcttccactctgttggccatgagccggcagataaccacatcacctccagagactgcaatggacaccttctcaactgctacgggaggaatgagagcgttgcctccaccccgccgggcgaagaccgcaagagccccagagtgctcatcaaaacgctggggaagctggatgggtgtttaagggtcgagttccacaatggtggcaaccccagcaaagtgcctgcagaggactccagtgagccagtgcagctgctgaggtactcacctaccttagcatcagaaacctccccagtgcctgaagccaggagggggtccagcgccgattccctgcccagccatcgcccctctcccacggactctcgcctgcggtccagcaaaggcagctccctgagttctgagtcatcctggtacgactccccttggggcaacgctggagagctgagcgaggctgagggctccttcctggcccccggcatgcctgaccccagcctccatgccagcttcccacctggtgatgccaaaaagcctttcaaccaaagctcttccctctcctcccttcgggaactgtacaaagatgccaacttggggagcctgtccccctcaggtatccgtctttctgatgaatacatgggcacgcatgccagcctgagcaaccgcgtctcttttgcctctgacattgatgtgccctccagggtggcacacagggaccccgtccagtacagttccttcactctcccctgtcggaagcccaaagccttagttgaggatactgcaaagaaggactccctcaaagccaggatgcgacggatcagtgactggacgggaagcctctcaaggaagaaaaggaaactccaggagccgaggtccaaggagggcagtgactactttgacagtcgctctgatggactgaatacagaagtgcaggggccctcccaggcgtctgcttttctgtggtcagggggctctgctcaaatcctgtctcagagaagtgaatccacgcatgcaattggcagcgatcccctccgacagaacatttatgagaatttcatgcgagagttggaaatgagcaggaccaacactgagaacatagaaatgtctacagaaaccgccgagtccagcagcgagtcgctcagctctctggaacagctggatctgctctttgagaaggaacagggggtggtccggaaagccgggtggctcttcttcaagcccctggtcactgtgcagaaggaaaggaagctcgagctggtggcacgaaggaaatggaaacagtactgggtaacgctgaaaggatgcacgctgctgttttatgagacctatgggaagaattccatggatcagagcagtgcccctcggtgtgctctgtttgcagaagacagcatagtgcaggctgttccagaacatcccaagaaagaaaacgtgttctgcctcagcaactcctttggagacgtctaccttttccaggccaccagccagacagatctagaaaactgggtcaccgccgtacactctgcttgtgcatccctttttgcaaagaagcatgggaaagaggacacactgcggctactgaagaaccagaccaaaaacctgcttcagaagatagacatggacagcaagatgaagaagatggcagagctgcagctgtccattgtgagcgacccaaagaacaggaaagccatagagaaccagatccagcaatgggagcagaatcttgagaaatttcacatggatctgttcaggatgcgctgctatctggccagcctacaaggcggagagttaccgaaccccaagagtctccttgcagccgccagccgcccctccaagctggccctcggcaggctgggcatcttgtctgtttcctctttccatgctctggtatgttctagagatgactctgctctccggaaaaggacactgtcactgacccagcgagggagaaacaagaagggaatattttcttcgttaaaagggctggacacactggccagaaaagggaaggagaagagaccttctataactcagatatttgatttaagtggcagccatggattttctggaactcggctacctcaaaactccaataactccagtgaggtcgacgaacttctgcatatgtatggttcaacagtagacagtgttccccgagacaatgcgtgggaaatccagacttatgtccactttcaggataatcatggagttactgtagggatcaagccagagcacagagtagaagatattttgactctggcatgcaagatgaggcaattggaacccagccattatggcctacaacttcgaaaattagtagatgacaacgttgagtactacatccctgcaccatatgaatatatgcaagaacaggtttatgatgaaatagaagtctttccactaaatgtttatgatgtgcagctcacgaagactgggagtgtgtgtgactttgggtttgcagttacagcgcaggtggatgagcatcagcatctcagccggatatttgtaagcgacgttcttcccgatggcctggcgtatggggaagggctgagaaagggcaatgagatcatgaccttaaatggggaagctgtgtctgatcttgacctcaagcagatggaggctctattttctgagaagagcgtcggactcactctgattgcccggcctccggacacaaaagccaccctgtgtacatcctggtcagacagtgacctgttctccagggaccagaagagtctgctaccccctcctaaccagtctcaactgctggaggaattcctggataactttaaaaagaatacagccaatgatttcagcaacgtccctgatatcacaacaggtctgaaaaggagtcagacagatggcactctggatcaggtttcccacagggagaaaatggagcagacattcaggagtgctgagcagatcactgcactgtgcaggagttttaacgacactcaggccaacggcatggaaggaccgcgggagagtcaggatcctcctccgaggcctctggcccgccacctctctgatgcagaccgcctccgcaaagtcatccaggagcttgtggacacagagaagtcctacgtgaaggatctgagctgcctctttgagttatacttggagccacttcagaatgagacctttcttactcaagatgagatggagtcactttttggaagtttgccagagatgcttgagtttcagaaggtgtttctggagaccctggaggatgggatttcagcatcatctgactttaacaccctagaaaccccctcacagtttagaaaattactgttttcccttggaggctctttcctttattacgcggaccactttaaactgtacagtggattctgtgctaaccatatcaaagtacagaaggttctggagcgagctaaaactgacaaagccttcaaggcttttctggacgcccggaaccccaccaagcagcattcctccacgctggagtcctacctcatcaagccggttcagagagtgctcaagtacccgctgctgctcaaggagctggtgtccctgacggaccaggagagcgaggaacattaccacctgacggaagcactaaaggcaatggagaaagtagcgagccacatcaacgagatgcagaagatctatgaggattatgggaccgtgtttgaccagctagtagctgagcagagcggaaccgagaaggaggtaacagaactttcgatgggagagcttctgatgcactctacggtttcctggctgaatccatttctgtctctaggaaaagctagaaaggaccttgagctcacagtatttgtttttaagagagccgtcatactggtttataaagaaaactgcaaactgaaaaagaaattgccctcgaattcccggcctgcacacaactctgctgacttggacccatttaaattccgctggttgatccccatctccgcgcttcaagtcagactggggaatccagcagggacagaaaataattccatatgggaactgatccatacgaagtcagaaatagaaggacggccagaaaccatctttcagttgtgttgcagtgacagtgaaagcaaaaccaacattgttaaggtgattcgttctatcctgagggaaaacttcaggcgtcacataaagtgtgaattaccactggagaaaacatgtaaggatcgcctggtacctcttaagaaccgagttcctgtttcagctaaattagcttcatccaggtctttaaaagtcctgaagaattcctccagcaacgagtggaccggtgagactggcaagggaacctcgctggactcagacgagggcagcttgagcagcggcacccagagcagcggctgccccacggccgagggcaggcaggactccaagactgcttctcccgggaaatacccacaccctggcttggcagattttgcggacaatctcatcaaagagagtgacatcctgagcgatgaagatgatgaccaccatcaaactctgaagcggggcagccctactaaagacatcgaaattcagttccagagactgaggatttctgaggacccagacgttcaccccgaggctgagcagcagcctggcccggaggcgggcgagggccagaaaggaggagagcagcccaaactggtccgggggcacttctgccccatcaaacgaaaagccaacagcaccaagagggacagaggaactttgctcaaggcacagctccgtcaccagtcccttgacagtcaatctgaaaatgccaccatcgatctaaattctgttctagagcgagaattcagtgtccagagtttaacatctgttgtcaatgaggagtgtttttatgaaacagagagccatggaaaatcatagtacgattcaatccagatatgggttaaattcctcattaaatcttttaaactggtggtaaagtggaaattgc

SEQ ID NO: 68 - DNA, 6386 bp, Macacca mulatta
atgggcaactccgagagtcagtacacccttcaaggatctaaaaatcatagcaatactatcactggtgctaagcaaattccttgctccctgaaaatacgtgggattcatgcaaaagaggaaaagtcattgcatggatgggttcacggaagcagcggagcaggttacaagtccaggtccctggcccgaagctgcctttctcactttaagagtaaccagccttacgcatccagactcggtggctccacgtgcaaggtctccagaggcgttgcctactccacgcacaggacaaatgccccagggaaggatttccagggcatcagtgctgctttctcaactgagaacggcttccactctgttggccatgagccggcagataaccacatcacctccagagactgcaatggacaccttctcaactgctacgggaggaatgagagcgttgcctccaccccgccgggcgaagaccgcaagagccccagagtgctcatcaaaacgctggggaagctggatgggtgtttaagggtcgagttccacaatggtggcaaccccagcaaagtgcctgcagaggactccagtgagccagtgcagctgctgaggtactcacctaccttagcatcagaaacctccccagtgcctgaagccaggagggggtccagcgccgattccctgcccagccatcgcccctctcccacggactctcgcctgcggtccagcaaaggcagctccctgagttctgagtcatcctggtacgactccccttggggcaacgctggagagctgagcgaggctgagggctccttcctggcccccggcatgcctgaccccagcctccatgccagcttcccacctggtgatgccaaaaagcctttcaaccaaagctcttccctctcctcccttcgggaactgtacaaagatgccaacttggggagcctgtccccctcaggtatccgtctttctgatgaatacatgggcacgcatgccagcctgagcaaccgcgtctcttttgcctctgacattgatgtgccctccagggtggcacacagggaccccgtccagtacagttccttcactctcccctgtcggaagcccaaagccttagttgaggatactgcaaagaaggactccctcaaagccaggatgcgacggatcagtgactggacgggaagcctctcaaggaagaaaaggaaactccaggagccgaggtccaaggagggcagtgactactttgacagtcgctctgatggactgaatacagaagtgcaggggccctcccaggcgtctgcttttctgtggtcagggggctctgctcaaatcctgtctcagagaagtgaatccacgcatgcaattggcagcgatcccctccgacagaacatttatgagaatttcatgcgagagttggaaatgagcaggaccaacactgagaacatagaaatgtctacagaaaccgccgagtccagcagcgagtcgctcagctctctggaacagctggatctgctctttgagaaggaacagggggtggtccggaaagccgggtggctcttcttcaagcccctggtcactgtgcagaaggaaaggaagctcgagctggtggcacgaaggaaatggaaacagtactgggtaacgctgaaaggatgcacgctgctgttttatgagacctatgggaagaattccatggatcagagcagtgcccctcggtgtgctctgtttgcagaagacagcatagtgcaggctgttccagaacatcccaagaaagaaaacgtgttctgcctcagcaactcctttggagacgtctaccttttccaggccaccagccagacagatctagaaaactgggtcaccgccgtacactctgcttgtgcatccctttttgcaaagaagcatgggaaagaggacacactgcggctactgaagaaccagaccaaaaacctgcttcagaagatagacatggacagcaagatgaagaagatggcagagctgcagctgtccattgtgagcgacccaaagaacaggaaagccatagagaaccagatccagcaatgggagcagaatcttgagaaatttcacatggatctgttcaggatgcgctgctatctggccagcctacaaggcggagagttaccgaaccccaagagtctccttgcagccgccagccgcccctccaagctggccctcggcaggctgggcatcttgtctgtttcctctttccatgctctggtatgttctagagatgactctgctctccggaaaaggacactgtcactgacccagcgagggagaaacaagaagggaatattttcttcgttaaaagggctggacacactggccagaaaagggaaggagaagagaccttctataactcaggtcgacgaacttctgcatatgtatggttcaacagtagacagtgttccccgagacaatgcgtgggaaatccagacttatgtccactttcaggataatcatggagttactgtagggatcaagccagagcacagagtagaagatattttgactctggcatgcaagatgaggcaattggaacccagccattatggcctacaacttcgaaaattagtagatgacaacgttgagtactacatccctgcaccatatgaatatatgcaagaacaggtttatgatgaaatagaagtctttccactaaatgtttatgatgtgcagctcacgaagactgggagtgtgtgtgactttgggtttgcagttacagcgcaggtggatgagcatcagcatctcagccggatatttgtaagcgacgttcttcccgatggcctggcgtatggggaagggctgagaaagggcaatgagatcatgaccttaaatggggaagctgtgtctgatcttgacctcaagcagatggaggctctattttctgagaagagcgtcggactcactctgattgcccggcctccggacacaaaagccaccctgtgtacatcctggtcagacagtgacctgttctccagggaccagaagagtctgctaccccctcctaaccagtctcaactgctggaggaattcctggataactttaaaaagaatacagccaatgatttcagcaacgtccctgatatcacaacaggtctgaaaaggagtcagacagatggcactctggatcaggtttcccacagggagaaaatggagcagacattcaggagtgctgagcagatcactgcactgtgcaggagttttaacgacactcaggccaacggcatggaaggaccgcgggagagtcaggatcctcctccgaggcctctggcccgccacctctctgatgcagaccgcctccgcaaagtcatccaggagcttgtggacacagagaagtcctacgtgaaggatctgagctgcctctttgagttatacttggagccacttcagaatgagacctttcttactcaagatgagatggagtcactttttggaagtttgccagagatgcttgagtttcagaaggtgtttctggagaccctggaggatgggatttcagcatcatctgactttaacaccctagaaaccccctcacagtttagaaaattactgttttcccttggaggctctttcctttattacgcggaccactttaaactgtacagtggattctgtgctaaccatatcaaagtacagaaggttctggagcgagctaaaactgacaaagccttcaaggcttttctggacgcccggaaccccaccaagcagcattcctccacgctggagtcctacctcatcaagccggttcagagagtgctcaagtacccgctgctgctcaaggagctggtgtccctgacggaccaggagagcgaggaacattaccacctgacggaagcactaaaggcaatggagaaagtagcgagccacatcaacgagatgcagaagatctatgaggattatgggaccgtgtttgaccagctagtagctgagcagagcggaaccgagaaggaggtaacagaactttcgatgggagagcttctgatgcactctacggtttcctggctgaatccatttctgtctctaggaaaagctagaaaggaccttgagctcacagtatttgtttttaagagagccgtcatactggtttataaagaaaactgcaaactgaaaaagaaattgccctcgaattcccggcctgcacacaactctgctgacttggacccatttaaattccgctggttgatccccatctccgcgcttcaagtcagactggggaatccagcagggacagaaaataattccatatgggaactgatccatacgaagtcagaaatagaaggacggccagaaaccatctttcagttgtgttgcagtgacagtgaaagcaaaaccaacattgttaaggtgattcgttctatcctgagggaaaacttcaggcgtcacataaagtgtgaattaccactggagaaaacatgtaaggatcgcctggtacctcttaagaaccgagttcctgtttcagctaaattagcttcatccaggtctttaaaagtcctgaagaattcctccagcaacgagtggaccggtgagactggcaagggaacctcgctggactcagacgagggcagcttgagcagcggcacccagagcagcggctgccccacggccgagggcaggcaggactccaagactgcttctcccgggaaatacccacaccctggcttggcagattttgcggacaatctcatcaaagagagtgacatcctgagcgatgaagatgatgaccaccatcaaactctgaagcggggcagccctactaaagacatcgaaattcagttccagagactgaggatttctgaggacccagacgttcaccccgaggctgagcagcagcctggcccggaggcgggcgagggccagaaaggaggagagcagcccaaactggtccgggggcacttctgccccatcaaacgaaaagccaacagcaccaagagggacagaggaactttgctcaaggcacagctccgtcaccagtcccttgacagtcaatctgaaaatgccaccatcgatctaaattctgttctagagcgagaattcagtgtccagagtttaacatctgttgtcaatgaggagtgtttttatgaaacagagagccatggaaaatcatagtacgattcaatccagatatgggttaaattcctcattaaatcttttaaactggtggtaaagtggaaattgcaaaaaaactattcattcctgggttttgtgcagtatacattttcccacgaaatggttgtaaagatttaagttattttaatttattgtggatcagaaacctagaagaaactggtcagaatctgtaaattacttagtttatatccactttgagcaggtatcaaatgatttagaatccttaaaattgccttctaattattttaagttatgtggaaaaagtaaggctggggaagttgtgattaatagttttcaaagggtcattttttaaaatcctctgggcattttctttcagctgtcagtttttgctttatttaaagcatatttaagttattttaatgtggtttaggggcaaaatgtgcagatacttcatttttgtaagattgtaatagatgctgtttatactaaacctgttgtatctatacagtatatattaaaagaaagcttgtactgtaccttatttgatgatatttattttctctgccaagctgtatagtaaaaggaaagtaagtcacatctggtcattggcatttttattgtcattctgtaaagacaaaagggtacccatataagaagctccacgcagtgcaaatcgacatctggtaggctgctcgcccccaggcagcagctagagtctgtaactcttcgcgtcatcctgtttttctttctttttgatttttctttgcctgagttcttctctgaaattatatgcaaagagttgtgggtcttcatcacacattcttctgtatacctcacagaggcttctaaagtgtgagatggagagctggcgaggccgaagagtagggtctatgtctgccaactctaacagcctgcccgtgttttccaagcgctgcgcttcagggaataacattctgagccctcgatggcagtatttccttcggaactgaaatacattctgaaccactttttccaccagcttgaacggctgctctatcttgggctgtatcaagggagtgaagtgcaccacgcccacgtccaccttcattgtaagcaaacatattatcattctgtggcatgatatgtggcatagcgtgatcaatcagctcatccttgttaaacaggaggatgggctgtcaacagcctgttttcataaaccgactcttccatgaacttcagtgtcatctctaggcatggaagatggtacattctggatttgcaaatgacatggagaaatcagctggctgcacctgttctct

SEQ ID NO: 69 - DNA, 5193 bp, Monodelphis domesticus
gttacgatgggaaactctgagagtcaatacagtctccaaggatctaaaaatcatagtagttcttctactggtgctaaacagaagccatgtcccctgaaaatccgcagcatccatgcaaaagatgaaaagtcttgctcattgcatgggtggggtcatggaaacagtgggacaaattacaagtctcgctccctggccaggagctgcctttctcactttaagagtaaccagccttattcatccagactcgatccgatggccaaagtctccaaaggcagtgcccacacaaagcacaaagggaatgtgtcaggaaactatttccaagggaagaattctgccttcttgtctgaaaatggctttcattatattagtcatgaaccagcagataaccacatcacctccagagactgcaatggacaccttctgaactgttatgggaaaaatgaaagtcttgcatcaacaccaccagctgaagacagaaagagccccaaggttctaattaaaacactagggaaactggatggatgtttaagggttgagttccacaatagcagcaacaacaaagtgcctacagaggactccactggaccagtccagcttttgagatattcccctaccttagagtctgaaacttccaatctaactgaaatcaggagaaattctagcaccgattattctgccagccattgcctttctcctactgattcccggctgagatctagcaaaggcagctcttttagctccgagtcttcctggtatgactccccttggggaaatgctggagacatcaatgagttggagggaccctacctgaccagaagcactccagataccagcatccactccagttttccatccgatgatgccaaaaagcctttcaaccaaagttcatctctttcttccctccgggaactgtacaaagatgccaccttagaaagtcttccaccctcgggcattagattttctgatgaatacattggcacacatgccagtttaaatagtagagtttcatttgtctcagacattgatgtgccctcaagagcagagcagagaggccctgtacagtactgttcttatacccttccctgtcggaagtccaaacccttaacagaagatacttccaaaaaggacacgttaaaaactagaatgaggcgaatcagtgattggacaggaagtctctcaagaaagaaaaggaaactgcaggagcccaaatccaaggatggatatgaccatttggaaaaccgaacagatggtctcagtgcagaagtgctggtcccttctcagctgtctagtttactgtggccaggtggatctggccagaccctgcctcagaggagtgagtctactaatgcaattagcagcgatcccttgcgacagaacatctatgaaaatttcatgcgggagttggagatgagtaggactcatgtggataacacagaaacatctacagacactgcagattctagtagtgattcacttagctctttagagcaactggacctgctctttgagaaggaacaaggagtggtccggaaagcagggtggctctttttcaagcctctggtcactcttcagaaagagaagaagctagagcttgtggcacggaggaaatggaaacagtactgggtcactcttaaaggctgtaccctgctgttctatgagacatatgggagaaattccatggatcaaagtagttcacctagatgtgctctgtttgcggaagatagcgtagtgcaatctgttcctgaacatcccaagaaggaaaatgtattctgcctcagtaactcctttggagatgtctacctttttcaggccactagccagactgacctggaaaactgggtgactgccatccattctgcttgtgcttctctttttgcaaagaagcatgggaaagaagacacagtccggttgttgaagaatcagaccaaaaatctactccaaaagattgacatggatagcaagatgaaaaaaatggctgagttgcagctctctgtggttagtgaccctaagaacagaaaagccatagaaaatcagatccagcagtgggagcagaatctagaaaaatttcacatggatctcttcaggatgcgctgctatttggctagtctacaaggtggagagttaccaaatcccaagagtctccttgctgccacaagtcgtccttctaaactggcactgggcagattgggcatcttatcagtttcttccttccatgctttgatatgttccagagatgaatcagctctgaggaaacgaacattatcattgactcagcaaggtcaaagtaagaagggattgttttcatcactgaaaggactggatactttggcaagaaaagggaaagacaaaaggccatccataactcagatttttgattcaagtggcaatcatggatttcccggaactcagctgcctcagagctccaataattccagtgaggtggatgaatttcaacatatttatagttctgcatcagacagtggtccccgagaaaacatttgggaaagccagacttatgttcattttcaggataatcaaggagttactctaatcatcaaaccagaacatagagtggaagatattttgtctctatcatgcaagatgaagcaactagaccccagctactatggcctacaacttagaagactggttgatgacaactttgagtattcaattccagctctttatgaatacatgcaagaacaggcaattgtttatgatgaaatagaaatcttccctctcagtatttatcatgtacttcttactaagactgggaacataacagattttgggtttgcagttacagctcaagttgatgaacaccagcacctcagccgaatatttataagtgatgttctccctgatggcctggcatatggggaagggttgagaacaggcaatgagatcttgactataaatggggaagtcatttctgaccttgaccttaggcagatggagtcattattttctgagagaagtgtgaggctcaccctgagaaccaattcttcagatacgaagcgaactttgggtacctcctggtcagacagcgacctttccagggaccagaaaaacctgctcccccctcccaaccaatcacagcttctggaagagtttttggataatcttaagaagaacacagcaaatgatttcagcaatgttcctgatgttacagctggtttgaaaagaagtcaaactgatggtaccttggaccaagttccccacagggagaaaactgaacatacattcaggagtgctgaacaaattgctgctctgtgtaggagctttaacgaggttcagccaagcacagacagcatggaagggcccaaagaggcacaggagccacccccaaggccattggctcggcacctctctgacgcagatcgactgagaaaagttatccaggaactcatggacacggagaagtcctatgtcaaggatctgacctgcctttttgaattatacttagaaccacttcagaatgagacatttcttacccaagatgaaatggaatctctttttggaagtctgccagaaatgctggagtttcaaaaggtgtttttggagaccctggaagatggcatttcttcatcctctgacttcaacatacttgaaactccctcccagttcagaaaattactcttctcccttggaggctctttcctttattatacagaccactttaaattgtacagtggattttgtgccaatcatatcaaagtacaaaaagttctagagagagcgaaaacagataaagcatttaaggcgttcttggatgctcgaaaccctaccaagcagcatgcttccaccttggagtcatacctcatcaagcctgttcagagagttctgaagtaccctctgttgctcaaagagttggtgtcactgacagacaatgaaagcgaggaacattatcacctgacagaagcactaaaggcaatggaaaaggtagcaagtcatatcaatgaaatgcagaaaatctatgaagattatggcactgtgtttgatcagctggtagcagagcagagtgggacagagaaggaggttacagaactttccatgggggagctcctgcttcactctacagtttcctggctgaatccctttctgtcccttggaaaagcaaggaaggatctcgagcttaccgtgtttgtttttaagagagccgtcatactggtttataaagaaaactgcaaactgaagaagaaattgccttccagttcccgacctacacatgttcacggtgacttggaccccttcaaattccggtggctaattcctttatctgctcttcaagtcagactgggaaatgcagcagggacagaaaataattctatctgggagctgatccacaccaagtcggagatagaagggcggccggagacaacctttcagttatgttgcagtgactgtgaaagtaaaaccaacatcgtgaaggtgatccgttccatcctgagggagaatttcagacgtcacataaagagtgaattgccactagagaagacgtgtaaagatcgcttgataccactcaaaaatcgcattcctgtctcagccaaattagcttcatcaaggtccttaagggtgctaaagaattctcccagcaacgagtggaacagtgatactggaaaaggaaacttactggattctgatgaatgtagcctgagtagtagcacgcaaagcagtggctgtcacactactgaaagtcggcaggaatccaaggattcgtcacctgagaagtacccacagacatgttcatctgatttttcagacagtcttattaaagagtctgatattttgagtgatgaagatgatgattttcatcaaactctgaaaaaaggcagccctacaaaagacattgaaattcagttccaaaggctgaagatctccgaggactctgagggtgacagcactgccgatcaacagcccagaacagaggttggaaacaatcttaacagtgtagcgcatccaaagctggtgcgtggacatttctgtcccattaagcggaaagcaaacagtaccaaaagggatagggggactctgttaacactgcaagcacgccatcagtctcttgacagtaaatctgaaaatgctaacattgatttaaattccatactagagagggaattcagtgtccagagtttaacatctgtcgttaatgaggagtgcttttatgagacagacagccatggaaaatcatag

SEQ ID NO: 70 - DNA, 5121 bp, Monodelphis domesticus
gttacgatgggaaactctgagagtcaatacagtctccaaggatctaaaaatcatagtagttcttctactggtgctaaacagaagccatgtcccctgaaaatccgcagcatccatgcaaaagatgaaaagtcttgctcattgcatgggtggggtcatggaaacagtgggacaaattacaagtctcgctccctggccaggagctgcctttctcactttaagagtaaccagccttattcatccagactcgatccgatggccaaagtctccaaaggcagtgcccacacaaagcacaaagggaatgtgtcaggaaactatttccaagggaagaattctgccttcttgtctgaaaatggctttcattatattagtcatgaaccagcagataaccacatcacctccagagactgcaatggacaccttctgaactgttatgggaaaaatgaaagtcttgcatcaacaccaccagctgaagacagaaagagccccaaggttctaattaaaacactagggaaactggatggatgtttaagggttgagttccacaatagcagcaacaacaaagtgcctacagaggactccactggaccagtccagcttttgagatattcccctaccttagagtctgaaacttccaatctaactgaaatcaggagaaattctagcaccgattattctgccagccattgcctttctcctactgattcccggctgagatctagcaaaggcagctcttttagctccgagtcttcctggtatgactccccttggggaaatgctggagacatcaatgagttggagggaccctacctgaccagaagcactccagataccagcatccactccagttttccatccgatgatgccaaaaagcctttcaaccaaagttcatctctttcttccctccgggaactgtacaaagatgccaccttagaaagtcttccaccctcgggcattagattttctgatgaatacattggcacacatgccagtttaaatagtagagtttcatttgtctcagacattgatgtgccctcaagagcagagcagagaggccctgtacagtactgttcttatacccttccctgtcggaagtccaaacccttaacagaagatacttccaaaaaggacacgttaaaaactagaatgaggcgaatcagtgattggacaggaagtctctcaagaaagaaaaggaaactgcaggagcccaaatccaaggatggatatgaccatttggaaaaccgaacagatggtctcagtgcagaagtgctggtcccttctcagctgtctagtttactgtggccaggtggatctggccagaccctgcctcagaggagtgagtctactaatgcaattagcagcgatcccttgcgacagaacatctatgaaaatttcatgcgggagttggagatgagtaggactcatgtggataacacagaaacatctacagacactgcagattctagtagtgattcacttagctctttagagcaactggacctgctctttgagaaggaacaaggagtggtccggaaagcagggtggctctttttcaagcctctggtcactcttcagaaagagaagaagctagagcttgtggcacggaggaaatggaaacagtactgggtcactcttaaaggctgtaccctgctgttctatgagacatatgggagaaattccatggatcaaagtagttcacctagatgtgctctgtttgcggaagatagcgtagtgcaatctgttcctgaacatcccaagaaggaaaatgtattctgcctcagtaactcctttggagatgtctacctttttcaggccactagccagactgacctggaaaactgggtgactgccatccattctgcttgtgcttctctttttgcaaagaagcatgggaaagaagacacagtccggttgttgaagaatcagaccaaaaatctactccaaaagattgacatggatagcaagatgaaaaaaatggctgagttgcagctctctgtggttagtgaccctaagaacagaaaagccatagaaaatcagatccagcagtgggagcagaatctagaaaaatttcacatggatctcttcaggatgcgctgctatttggctagtctacaaggtggagagttaccaaatcccaagagtctccttgctgccacaagtcgtccttctaaactggcactgggcagattgggcatcttatcagtttcttccttccatgctttgatatgttccagagatgaatcagctctgaggaaacgaacattatcattgactcagcaaggtcaaagtaagaagggattgttttcatcactgaaaggactggatactttggcaagaaaagggaaagacaaaaggccatccataactcaggtggatgaatttcaacatatttatagttctgcatcagacagtggtccccgagaaaacatttgggaaagccagacttatgttcattttcaggataatcaaggagttactctaatcatcaaaccagaacatagagtggaagatattttgtctctatcatgcaagatgaagcaactagaccccagctactatggcctacaacttagaagactggttgatgacaactttgagtattcaattccagctctttatgaatacatgcaagaacaggcaattgtttatgatgaaatagaaatcttccctctcagtatttatcatgtacttcttactaagactgggaacataacagattttgggtttgcagttacagctcaagttgatgaacaccagcacctcagccgaatatttataagtgatgttctccctgatggcctggcatatggggaagggttgagaacaggcaatgagatcttgactataaatggggaagtcatttctgaccttgaccttaggcagatggagtcattattttctgagagaagtgtgaggctcaccctgagaaccaattcttcagatacgaagcgaactttgggtacctcctggtcagacagcgacctttccagggaccagaaaaacctgctcccccctcccaaccaatcacagcttctggaagagtttttggataatcttaagaagaacacagcaaatgatttcagcaatgttcctgatgttacagctggtttgaaaagaagtcaaactgatggtaccttggaccaagttccccacagggagaaaactgaacatacattcaggagtgctgaacaaattgctgctctgtgtaggagctttaacgaggttcagccaagcacagacagcatggaagggcccaaagaggcacaggagccacccccaaggccattggctcggcacctctctgacgcagatcgactgagaaaagttatccaggaactcatggacacggagaagtcctatgtcaaggatctgacctgcctttttgaattatacttagaaccacttcagaatgagacatttcttacccaagatgaaatggaatctctttttggaagtctgccagaaatgctggagtttcaaaaggtgtttttggagaccctggaagatggcatttcttcatcctctgacttcaacatacttgaaactccctcccagttcagaaaattactcttctcccttggaggctctttcctttattatacagaccactttaaattgtacagtggattttgtgccaatcatatcaaagtacaaaaagttctagagagagcgaaaacagataaagcatttaaggcgttcttggatgctcgaaaccctaccaagcagcatgcttccaccttggagtcatacctcatcaagcctgttcagagagttctgaagtaccctctgttgctcaaagagttggtgtcactgacagacaatgaaagcgaggaacattatcacctgacagaagcactaaaggcaatggaaaaggtagcaagtcatatcaatgaaatgcagaaaatctatgaagattatggcactgtgtttgatcagctggtagcagagcagagtgggacagagaaggaggttacagaactttccatgggggagctcctgcttcactctacagtttcctggctgaatccctttctgtcccttggaaaagcaaggaaggatctcgagcttaccgtgtttgtttttaagagagccgtcatactggtttataaagaaaactgcaaactgaagaagaaattgccttccagttcccgacctacacatgttcacggtgacttggaccccttcaaattccggtggctaattcctttatctgctcttcaagtcagactgggaaatgcagcagggacagaaaataattctatctgggagctgatccacaccaagtcggagatagaagggcggccggagacaacctttcagttatgttgcagtgactgtgaaagtaaaaccaacatcgtgaaggtgatccgttccatcctgagggagaatttcagacgtcacataaagagtgaattgccactagagaagacgtgtaaagatcgcttgataccactcaaaaatcgcattcctgtctcagccaaattagcttcatcaaggtccttaagggtgctaaagaattctcccagcaacgagtggaacagtgatactggaaaaggaaacttactggattctgatgaatgtagcctgagtagtagcacgcaaagcagtggctgtcacactactgaaagtcggcaggaatccaaggattcgtcacctgagaagtacccacagacatgttcatctgatttttcagacagtcttattaaagagtctgatattttgagtgatgaagatgatgattttcatcaaactctgaaaaaaggcagccctacaaaagacattgaaattcagttccaaaggctgaagatctccgaggactctgagggtgacagcactgccgatcaacagcccagaacagaggttggaaacaatcttaacagtgtagcgcatccaaagctggtgcgtggacatttctgtcccattaagcggaaagcaaacagtaccaaaagggatagggggactctgttaacactgcaagcacgccatcagtctcttgacagtaaatctgaaaatgctaacattgatttaaattccatactagagagggaattcagtgtccagagtttaacatctgtcgttaatgaggagtgcttttatgagacagacagccatggaaaatcatag

SEQ ID NO: 71 - DNA, 5193 bp, Monodelphis domesticus
gttacgatgggaaactctgagagtcaatacagtctccaaggatctaaaaatcatagtagttcttctactggtgctaaacagaagccatgtcccctgaaaatccgcagcatccatgcaaaagatgaaaagtcttgctcattgcatgggtggggtcatggaaacagtgggacaaattacaagtctcgctccctggccaggagctgcctttctcactttaagagtaaccagccttattcatccagactcgatccgatggccaaagtctccaaaggcagtgcccacacaaagcacaaagggaatgtgtcaggaaactatttccaagggaagaattctgccttcttgtctgaaaatggctttcattatattagtcatgaaccagcagataaccacatcacctccagagactgcaatggacaccttctgaactgttatgggaaaaatgaaagtcttgcatcaacaccaccagctgaagacagaaagagccccaaggttctaattaaaacactagggaaactggatggatgtttaagggttgagttccacaatagcagcaacaacaaagtgcctacagaggactccactggaccagtccagcttttgagatattcccctaccttagagtctgaaacttccaatctaactgaaatcaggagaaattctagcaccgattattctgccagccattgcctttctcctactgattcccggctgagatctagcaaaggcagctcttttagctccgagtcttcctggtatgactccccttggggaaatgctggagacatcaatgagttggagggaccctacctgaccagaagcactccagataccagcatccactccagttttccatccgatgatgccaaaaagcctttcaaccaaagttcatctctttcttccctccgggaactgtacaaagatgccaccttagaaagtcttccaccctcgggcattagattttctgatgaatacattggcacacatgccagtttaaatagtagagtttcatttgtctcagacattgatgtgccctcaagagcagagcagagaggccctgtacagtactgttcttatacccttccctgtcggaagtccaaacccttaacagaagatacttccaaaaaggacacgttaaaaactagaatgaggcgaatcagtgattggacaggaagtctctcaagaaagaaaaggaaactgcaggagcccaaatccaaggatggatatgaccatttggaaaaccgaacagatggtctcagtgcagaagtgctggtcccttctcagctgtctagtttactgtggccaggtggatctggccagaccctgcctcagaggagtgagtctactaatgcaattagcagcgatcccttgcgacagaacatctatgaaaatttcatgcgggagttggagatgagtaggactcatgtggataacacagaaacatctacagacactgcagattctagtagtgattcacttagctctttagagcaactggacctgctctttgagaaggaacaaggagtggtccggaaagcagggtggctctttttcaagcctctggtcactcttcagaaagagaagaagctagagcttgtggcacggaggaaatggaaacagtactgggtcactcttaaaggctgtaccctgctgttctatgagacatatgggagaaattccatggatcaaagtagttcacctagatgtgctctgtttgcggaagatagcgtagtgcaatctgttcctgaacatcccaagaaggaaaatgtattctgcctcagtaactcctttggagatgtctacctttttcaggccactagccagactgacctggaaaactgggtgactgccatccattctgcttgtgcttctctttttgcaaagaagcatgggaaagaagacacagtccggttgttgaagaatcagaccaaaaatctactccaaaagattgacatggatagcaagatgaaaaaaatggctgagttgcagctctctgtggttagtgaccctaagaacagaaaagccatagaaaatcagatccagcagtgggagcagaatctagaaaaatttcacatggatctcttcaggatgcgctgctatttggctagtctacaaggtggagagttaccaaatcccaagagtctccttgctgccacaagtcgtccttctaaactggcactgggcagattgggcatcttatcagtttcttccttccatgctttgatatgttccagagatgaatcagctctgaggaaacgaacattatcattgactcagcaaggtcaaagtaagaagggattgttttcatcactgaaaggactggatactttggcaagaaaagggaaagacaaaaggccatccataactcagatttttgattcaagtggcaatcatggatttcccggaactcagctgcctcagagctccaataattccagtgaggtggatgaatttcaacatatttatagttctgcatcagacagtggtccccgagaaaacatttgggaaagccagacttatgttcattttcaggataatcaaggagttactctaatcatcaaaccagaacatagagtggaagatattttgtctctatcatgcaagatgaagcaactagaccccagctactatggcctacaacttagaagactggttgatgacaactttgagtattcaattccagctctttatgaatacatgcaagaacaggcaaaagtttatgatgaaatagaaatcttccctctcagtatttatcatgtacttcttactaagactgggaacataacagattttgggtttgcagttacagctcaagttgatgaacaccagcacctcagccgaatatttataagtgatgttctccctgatggcctggcatatggggaagggttgagaacaggcaatgagatcttgactataaatggggaagtcatttctgaccttgaccttaggcagatggagtcattattttctgagagaagtgtgaggctcaccctgagaaccaattcttcagatacgaagcgaactttgggtacctcctggtcagacagcgacctttccagggaccagaaaaacctgctcccccctcccaaccaatcacagcttctggaagagtttttggataatcttaagaagaacacagcaaatgatttcagcaatgttcctgatgttacagctggtttgaaaagaagtcaaactgatggtaccttggaccaagttccccacagggagaaaactgaacatacattcaggagtgctgaacaaattgctgctctgtgtaggagctttaacgaggttcagccaagcacagacagcatggaagggcccaaagaggcacaggagccacccccaaggccattggctcggcacctctctgacgcagatcgactgagaaaagttatccaggaactcatggacacggagaagtcctatgtcaaggatctgacctgcctttttgaattatacttagaaccacttcagaatgagacatttcttacccaagatgaaatggaatctctttttggaagtctgccagaaatgctggagtttcaaaaggtgtttttggagaccctggaagatggcatttcttcatcctctgacttcaacatacttgaaactccctcccagttcagaaaattactcttctcccttggaggctctttcctttattatacagaccactttaaattgtacagtggattttgtgccaatcatatcaaagtacaaaaagttctagagagagcgaaaacagataaagcatttaaggcgttcttggatgctcgaaaccctaccaagcagcatgcttccaccttggagtcatacctcatcaagcctgttcagagagttctgaagtaccctctgttgctcaaagagttggtgtcactgacagacaatgaaagcgaggaacattatcacctgacagaagcactaaaggcaatggaaaaggtagcaagtcatatcaatgaaatgcagaaaatctatgaagattatggcactgtgtttgatcagctggtagcagagcagagtgggacagagaaggaggttacagaactttccatgggggagctcctgcttcactctacagtttcctggctgaatccctttctgtcccttggaaaagcaaggaaggatctcgagcttaccgtgtttgtttttaagagagccgtcatactggtttataaagaaaactgcaaactgaagaagaaattgccttccagttcccgacctacacatgttcacggtgacttggaccccttcaaattccggtggctaattcctttatctgctcttcaagtcagactgggaaatgcagcagggacagaaaataattctatctgggagctgatccacaccaagtcggagatagaagggcggccggagacaacctttcagttatgttgcagtgactgtgaaagtaaaaccaacatcgtgaaggtgatccgttccatcctgagggagaatttcagacgtcacataaagagtgaattgccactagagaagacgtgtaaagatcgcttgataccactcaaaaatcgcattcctgtctcagccaaattagcttcatcaaggtccttaagggtgctaaagaattctcccagcaacgagtggaacagtgatactggaaaaggaaacttactggattctgatgaatgtagcctgagtagtagcacgcaaagcagtggctgtcacactactgaaagtcggcaggaatccaaggattcgtcacctgagaagtacccacagacatgttcatctgatttttcagacagtcttattaaagagtctgatattttgagtgatgaagatgatgattttcatcaaactctgaaaaaaggcagccctacaaaagacattgaaattcagttccaaaggctgaagatctccgaggactctgagggtgacagcactgccgatcaacagcccagaacagaggttggaaacaatcttaacagtgtagcgcatccaaagctggtgcgtggacatttctgtcccattaagcggaaagcaaacagtaccaaaagggatagggggactctgttaacactgcaagcacgccatcagtctcttgacagtaaatctgaaaatgctaacattgatttaaattccatactagagagggaattcagtgtccagagtttaacatctgtcgttaatgaggagtgcttttatgagacagacagccatggaaaatcatag

SEQ ID NO: 72 - DNA, 3251 bp, Pan troglodytes
agtgctgagcagatcactgcactgtgcaggagttttaacgacagtcaggccaacggcatggaaggaccgcgggagagtcaggatcctcctccgaggcctctggcccgccacctgtctgatgcagaccgcctccgcaaagtcatccaggagcttgtggacacagagaagtcctacgtgaaggatttgagctgcctctttgaattatacttggagccacttcagaatgagacctttcttacccaagatgagatggagtcactttttggaagtttgccagagatgcttgagtttcagaaggtgtttctggagaccctggaggatgggatttcagcatcatctgactttaacaccctagaaaccccctcacagtttagaaaattactgttttcccttggaggctctttcctttattacgcggaccactttaaactgtacagtggattctgtgctaaccatatcaaagtacagaaggttctggagcgagctaaaactgacaaagccttcaaggcttttctggacacccggaaccccaccaagcagcattcctccacgctggagtcctacctcatcaagccggttcagagagtgctcaagtacccactgctgctcaaggagctggtgtccctgacggaccaggagagcgaggagcactaccacctgacggaagcactaaaggcaatggagaaagtagcgagccacatcaatgagatgcagaagatctatgaggattatgggaccgtgtttgaccagctagtagctgagcagagcggaacagagaaggaggtaacagaactttcgatgggagagcttctgatgcactctacggtttcctggttgaatccatttctgtctctaggaaaagctagaaaggaccttgagctcacagtatttgtttttaagagagccgtcatactggtttataaagaaaactgcaaactgaaaaagaaattggcctcgaattcccggcctgcacacaactctactgacttggacccatttaaattccgctggttgatccccatctccgcgcttcaagtcagactggggaatccagcagggacagaaaataattccatatgggaactgatccatacgaagtcagaaatagaaggacggccagaaaccatctttcagttgtgttgtagtgacagtgaaagcaaaaccaacattgttaaggtgattcgttctattctgagggaaaacttcaggcgtcacataaagtgtgaattaccactggagaaaacgtgtaaggatcgcctggtacctcttaagaaccgagttcctgtttcggccaaattagcttcatccaggtctttaaaagtcctgaagaattcctccagcaacgagtggaccggtgagactggcaagggaaccttgctggactctgacgagggcagcttgagcagcagcacccagagcagcggctgccccacggctgagggcaggcaggactccaagagcacttctcccgggaaatacccacaccccggcttggcagattttgctgacaatctcatcaaagagagtgacatcctgagcgatgaagatgatgaccaccgtcagactgtgaagcagggcagccctactaaagacatcgaaattcagttccagagactgaggatttccgaggacccagacgttcaccccgaggctgagcagcagcctggcccggagtcgggtgagggtcagaaaggaggagagcagcccaaactggtccgggggcacttctgccccattaaacgaaaagccaacagcaccaagagggacagaggaactttgctcaaggcgcagatccgtcaccagtcccttgacagtcagtctgaaaatgccaccatcgacctaaattctgttctagagcgagaattcagtgtccagagtttaacatctgttgtcagtgaggagtgtttttatgaaacagagagccacggaaaatcatagtatgattcaatccagatatgggttaaattcctcattttacttttaaactggtggtaaagtggaaattgcaaaaaaaaccaaaaaacaaaaaactgttcattcctgggttttgtgcagtatacattttcccacataatggttgtaaagatttaagttattttaatttattgtggatcagaaacctagatgaaactggtcagaatctgtaaattacttagtttatatccactttgagcaggtatcaaatgatttaggatccttaaaattacattctaataattattttaagttatgtggaaaaagtaaggctggggaagttgtgattaatagttttcaaagggccatttttttaaatcctctgggcattttctttcagctgttagtttttgctttatttaaagcatatttaagttattttaatgtggtttaggggcaaaatgtgcagatacttcatttttgtaagattgtaatagatgctgtttatactaaacatgtcatatctatacagtatatattaaaagaaagcttgtactgtatctatcttatttgatgatatttattttctctgccaagctgtatagtaaaaggaaaataagtaagtcacatctggtcattggcatttgtatcgtcattctgtaaagaaagacaaaagagtacctatataagaagctccacgtagtgcaaatcgacatctggtaggctgctcgcccccaggcagcagctagagtctgtaattctctgcgtcatcctcttctttttcttcatttttgctttttcttcgcttgagttcttctctgaaattatatgcaaagagttgtgggtcttcatcacacatttttctgtatacctcacagaggctcttaaagtgtgagatggagagctggcggggccgaagagtagggtctatgtctgccaactctaacagcctgcccgtgctttccaagcgctgcgcttcagggaataacattctgagccctcgatggcagtatttccttcggaactgaaatacattctgaaccactttttccaccagcttgaatggctgctctatcttgggctgtatcaagggagtgaagtgcaccacgcccacgtccaccttcgttgtaagcaaacatattatcattctgtggcatgatatgtggcatagtgtgatcaatcaactcatccttgtaaaacaggaggatgggctgtcaacagcctgttttcataaacagacctttccacgtactttggtttcatctctaggcatggaagatggtacattctggatttgcaaatgacatggagaaatcagctggctgcacctgttctct

SEQ ID NO: 73 - protein, 655 aa, Mouse
MERACEKQDSVCNLVAVFENNRTPGEAPGSHSLEDQLHSPEHQLSLSPEPWEAPPVKEALKSEFRPVSRTYLSSLKNKLSSGAWRRSCQPGVSPGPETQEPEEKRVVRELLETEQAYVARLHLLDQVFFQELLREAGRSKAFPEDVVKLIFSNISSIYRFHAQFFLPELQRRVDDWAATPRIGDVIQKLAPFLKMYSEYVKNFERAAELLATWMDKSQPFQEVVTRIQCSEASGSLTLQHHMLEPVQRIPRYELLLKEYVQKLPAQAPDLEDAQRALDMIFSAAQHSNAAIAEMERLQGLWDVYQRLGLEDDIVDPSNTLLREGPVLKISFRRSDPMERYLVLFNNMLLYCVPRVLQVGAQFQVRTRIDVAGMKVRELTDAEFPHSFLVSGKQRTLELQARSRDEMVSWMQACQAAIDQVEKRSETFKAAVQGPQGDTQEPKPQVEELGLRAPQWVRDKMVTMCMRCQEPFNALTRRRHHCRACGYVVCAKCSDYRAELKYDSNRPNRVCLTCYTFLTGNVLPQGKEDKRRGILEKEASAAPEQSLVCSFLQLIGDKCSRSLPRSWCVIPRDDPLVLYVYAAPQDTKAHTSIPLLGYQVISGPQGDPRVFQLQQSGQQYTFKAESVELQGRWVTAIKRAASGRTPEGPDEEDVSD

SEQ ID NO: 74 - protein, 687 aa, Danio rerio
MKVLSGNTTNKTLSGDGMDSDLLVDSNGKNSALSKELHNQEAHAQQPIGENGGKKSFKIPFKLKRSSTSPSVTAHPESSTKNLLFGRNLQDDTALPKPIAEILLLLFRKGPVTEGVFRVSCNSKNLHTLRNQLNSGAEVDMDALPVTLLVGLLKIFLRELPGGLLMSEHYESWIGALEKERTEEVQSELRGMAEKLPKANSLLLQHLLCLFHHISQRSETNKMDAKNLAVCIAPTLLHRDSQPLDVDVVEKVTKLIQFLTENCCEIFGHDILTLFGDLEDRKATDKSDSASLMSPDISFEVHQHDSAYDSTDPDADCDCVEAESYEEGMMAHGSLGLSKKGRSDIQSCSSDAIFDTFTKTFSRRSSEPSILSTLPITGLRELARSHDDFSTEKEHFDNQPLKKQNSDDSFLLPNRSENRRSLKKLAGSFNMDLPIIVSSPTSKIGSCPSFCSSDSSSSNHSEQSKTPSPLPSPANPRKTQSTRHASFMIKSRHNHAQGDQEVTRRSLSMRAKSLGNFTFNRSSLKKGDSQKEVVFPCETLQEDSQNETENPDELVRRRRPLSAIEVFQQVDSRMPCSPPSYEQALQTGVHQAPPQYREMTVQHARELGKKSRPISMNDNLLDIYKVNESTECLETFTESIQLDKPQLVTFRQRAMSESVSQLKHEKVSRRCSQPVFEEFSYAKESYV

SEQ ID NO: 75 - protein, 731 aa, Macaca mulatta
MKLRSSHNASKTLNASNMETLIECQSEGDIKEHPLLASCESEDSICQLIEVKKRKKVLSWPFLMRRLSPASDFSGALETDLKASLFDQPLSIICGGSDTLPRPIQDILTILCLKGPSTEGIFRRAANEKARKELKEELNSGDAVDLESLPVHLLAVVFKDFLRSIPRKLLSSDLFEEWMGTLEMQDEEDRIEALKQVADKLPQPNLLLLKHLVYVLHLISKNSEVNRMDSSNLAICIGPNMLTLENDQCLSFEAQKDLNNKVKTLVEFLIDNCFEIFGENIPVHSSITSVDSLEHTDSSDVSTLQNDSAYDSNDPDVESNSSSGISSPSRQPQVPMATAAGFDSGGPQDAREVSPEPIVSTVARLKSSLAQPDRRYSEPSMPSSQECLESRVTNQTLTKSEGDFPVPRVGSRLEREEAEDPFPEEVFPAVQGKTKRPVDLKIKNLTPGSVLPRALVPKAFSSSSLDASSDSSPVASPSSPKRNFFSRHQSFTTKTEKGKPSREIKKHSMSFSFAPHKKVLTKNLSAGSGKSQDFTRDHVPRGVRKESQLAGRIVQENGSETHNQTTRAFCLRPHALSVDDVFQGADWERPGSPPSYEEAMQGPAARLAAYGSQTVGSMTVGSMRARMLEVDSLLPPLPPAHHTGDSRHRDSKEPLPGHGPSPLPERWIQSRTVHASWDSVGHVSGPGRPELLRLRTVSESVQRNKRDCLMRRCSQPVFEADQFQHAKESYI

SEQ ID NO: 76 - protein, 724 aa, Monodelphis domestica
MKVLSGCNASKTLNASDMETLIECQPEGNVKQHHLLAQCDSEDGICHLIENKKRKVIAWPFPMRRTASSSDSSGSLTPEWKASLFDQPLSAICTDEDILPQPIQEILNILQNKGPSTEGIFRKAANEKARKELKEELNSGGMVNLETKSVHLLAAVLKDFLRSIPLKLLSSDLFEEWMTALERPNEDDKIESLKQVVKKLPRTNILLLKHLVYVLYNISKNSDVSKMDSSNLAICIGPNMLSLNTDQSLSFDAQKELNNKIKTLVEFFIDNCFEIFEEDIPGHSPRFNSDDSLEHINNSDMSTLQNDSAYESTDADTECSNSFGSQNKPSLDTAGLSSDLDNRKHLYESRPGSVVSYTNLLKSSLSTQERRYSEPSTPSTKDCLESRITSHKLTKSEDSFAVPQASSCFGGQEIKESFPEECFPSLQHRKQKSLGLQIKEGTLCTESLVEPSPKTSSCGSLDSSSDSSVFANSPVVSPSSPKRNFFTRHQSFTTKTAGADSKLTRERKKHSMSFSFATHKKVPNKTANWRFPRDQGKKDLKKESQLTGRIVQEVCVGAIDDYQPTPECGSSRSHLLSVEEVFQLVDQKNPGSPPSYEEAVQYCRLSKIPPYESRTVQSMRDTMQSQNSRLPSLSLLDYGEHAKNTHGKETLNRDSASSVGETWVQNGASNVAMERKGQIPKSEVRRLRVLSESLLKNKQDCVIRRCSQPIFEVDQIQYAKESYV

SEQ ID NO: 77 - protein, 702 aa, Xenopus tropicalis
MKVLTGCNAFKAINTSNMEDLIECPTEADAKKYQLLAAMISEDGMCHVIENNKKRKAVISWPFTFRRSSTLSETSVPPELKATLFDQPLSIVCEEDALPKPILEILTILCQQGPSTEGIFRKAANEKARKELKEDLNSGKTVDLKSKHVHLLAVVLKVNILSTQKEMIINNNFFFFLNNFISDERICSVSRVADKLPRPNWILLQHLICVLYHISKASTLNKMDSNNLAVCIGPNMLQPHHDYNLSLEAQKQANDRVISLVEFFIDNCFDLFGQNVSQCLSTSKEELLEDTDVSEIPFQQNDSAYDSTDPEYEGHNSTITSCQKDLGINSGREVLNSSMDQDINLTQSSINLLKVSNMDRRKSEPHIFPSQDTKVVAGRKLTRSHDDVTVRKSGEPLTKEDLSEQVSEIALYKKKMPKGLTVNTSYSEDVLDDVLQNALSSCSLESCFSDCSVFTSSPLASPISPKNNCLIRHQSCSSKSGIRNDIKLSSREIKKHSKSFSYVNQKKKLAKTQSWGPEGQNPGLQRYMFNSSLRNRHQYENPQAKCFQQPAVVRLRRPQSARKMSVDEVFRIVDQRNPGKPPSYEEAIHKNVPPFKGMTVQTVRATVSNNEYSSSHVVSDTPMDRECRTHAEKQTTIINEGPTEKNCAEEMQQLCQFGKTKSVVIRTMSESVQKHKHETLSRRCSQPFELYDQIQYAKESYV

SEQ ID NO: 78 - protein, 731 aa, Pan troglodytes
MKLRSSHNASKTLNANNMETLIECQSEGDIKEHPLLASCESEDSICQLIEVKKRKKVLSWPFLMRRLSPASDFSGALETDLKASLFDQPLSIICGDSDTLPRPIQDILTILCLKGPSTEGIFRRAANEKARKELKEELNSGDAVDLERLPVHLLAVVFKDFLRSIPRKLLSSDLFEEWMGALEMQDEEDRIEALKQVADKLPQPNLLLLKHLVYVLHLISKNSEVNRMDSSNLAICIGPNMLTLENDQSLSFEAQKDLNNKVKTLVEFLIDNCFEIFGENIPVHSSITSDDSLEHTDSSDVSTLQNDSAYDSNDPDVESNSSSGISSPSRQPQVPMATAAGLDSAGPQDAREVSPEPIVSTVARLKSSLAQPDRRYSEPSMPSSQECLESRVTNQTLTKSEGDFPVPRVGSRLESEEAEDPFPEEVFPAVQGKTKRPVDLKIKNLAPGSVLPRALVLKAFSSSSLDASSDSSPVASPSSPKRNFFSRHQSFTTKTEKGKPSREIKKHSMSFSFAPHKKVLTKNLSAGSGKSQDFTRDHVPRGVRKESQLAGRIVQENGCETHNQTARGFCLRPHALSVDDVFQGADWERPGSPPSYEEAMQGPAARLVAYESQTVGSMTVGSMRARMLEAHCLLPPLPPAHHVEDSRHRGSKEPLPGHGLSPLPERWKQSRTVHASGDSLGHVSGPGRPELLPLRTVSESMQRNKRDCLVRRCSQPVFEADQFQYAKESYI

SEQ ID NO: 79 - protein, 657 aa, Macacca mulatta
MKEASEEKLASVSNLVTVFENSRTPEAAPRGHRLEDAHHHPECRPPRSPGPWEKLNVGEAMGSEPRTVSGRYLNSLKNKLSSGAWRKSCQPVTLSGSGMQEPEKKIVQELLETEQAYVARLHLLDQAMVFFQELLRAARRSKAFPEDVVRVIFSNISSIYQFHSQFFLPELQRRLDDWTANPRIGDVIQKLAPFLKMYGEYVKNFERAAELLATWTEKSPLFQEVLTRIQSGEASGSLTLQHHMLEPVQRIPRYELLLKEYVQKLPAQAPDRADAQKALDMIFSAAQHSNAAITEMERLQDLWEVYQRLGLEDDIVDPSNTLLREGPVFKISFRRNDPMERYLFLFNNMLLYCVPRVIQVGAQFQVRTRIDVAGMKVRELMDAEFPHSFLVSGKQRTLELQARSQEEKISWMQAFQAAVDQIEKRNETFKAAAQGPEGDTQEQELQSEELGLRAPQWVRDKMVTMCMRCQEPFNALTRRRHHCRACGYVVCARCSDYRAELKYNDNRQNRVCLHCYTFLTGNVLPEAKEDKRRGILEKGSSATPDQSLMCSFLQLIGDKWGKSGPRGWCVIPRDDPLVLYVYAAPQDMRAHTSIPLLGYQVTVGPQGDPRVFQLQQSGQLYTFKAETEELKGRWVKAMERAASGWSPSWPNDGDLSD

SEQ ID NO: 80 - protein, 657 aa, Macacca mulatta; identical to SEQ ID NO: 79

SEQ ID NO: 81 - protein, 680 aa, Macaca mulatta
MFPKKARHPGFPALEICTRQPSTPGTCSCFPCSPGRKPSGLSLLLRTPEAAPRGHRLEDAHHHPECRPPRSPGPWEKLNVGEAMGSEPRTVSGRYLNSLKNKLSSGAWRKSCQPVTLSGSGMQEPEKKIVQELLETEQAYVARLHLLDQAMVFFQELLRAARRSKAFPEDVVRVIFSNISSIYQFHSQFFLPELQRRLDDWTANPRIGDVIQKLAPFLKMYGEYVKNFERAAELLATWTEKSPLFQEVLTRIQSGEASGSLTLQHHMLEPVQRIPRYELLLKEYVQKLPAQAPDRADAQKALDMIFSAAQHSNAAITEMERLQDLWEVYQRLGLEDDIVDPSNTLLREGPVFKISFRRNDPMERYLFLFNNMLLYCVPRVIQVGAQFQVRTRIDVAGMKVRELMDAEFPHSFLVSGKQRTLELQARSQEEKISWMQAFQAAVDQIEKRNETFKAAAQGPEGDTQEQELQSEELGLRAPQWVRDKMVTMCMRCQEPFNALTRRRHHCRACGYVVCARCSDYRAELKYNDNRQNRVCLHCYTFLTGNVLPEAKEDKRRGILEKGSSATPDQSLMCSFLQLIGDKWGKSGPRGWCVIPRDDPLVLYVYAAPQDMRAHTSIPLLGYQVTVGPQGDPRVFQLQQSGQLYTFKAETEELKGRWVKAMERAASGWSPSWPNDGDLSD

SEQ ID NO: 82 - protein, 629 aa, Monodelphis domestica
SNSISQMPQTLMEPHFDRTPGPWERDSLEDRPGPGHWAVSTRLLSSLRNKISNSGWRKSCEFPFSNPNLPPHLQEPEENRIVRELLETEQAYVSRLYLLDQVFFEELLKEARNSKAFSEDIVKLIFSNISSIHQFHSQFFLPELQRRVDEWVVVPRIGDVIQKLAPFLKMYSEYVKNFKRAAELLVIWTEKCPPFQEVITRIQNSDISDSLTLQHHMLEPVQRIPRYELLLKEYVQKLPPDAPDRADAQKALDMIFSAAQHSNAAITEMERLQNLWDVYQRLGLEDDIVDPSNELLQEGPIQKISFRHSSTMERYLFLFNNMLLYCVPKVIQVGAQFQVRTRIDVAGMKVRELNDVEFPHSFLVSGKQRTLELQAQSQEEMNTWIQACQRAIDLIGRRHGTFKAAVQGTEGDSEEHQLKSEELGIRAPQWVRDKMVTMCMRCKTPFNALTRRRHHCRACSYVVCAKCSDYRARLQYDDNRLHRVCLQCYVFLTGNLLPEDKEEKKKGILEKESSKVSEQSVMCSFLQLLGDKWAKGSSRGWCVIPRDDPLALYVYAAPQDMKAHTSIPLLGYQVTTGSLADPRAFQIQQSGLVYSFRAESEELKGQWMKAIERASRGQSFTGPEGELSD

SEQ ID NO: 83 - protein, 655 aa, Pan troglodytes
MKGASEEKLASVSNLVTVFENSRTPEAAPRGHRLEDVHHRPECRPPESPGPREKTNVGEAVGSEPRTVSRRYLNSLKNKLSSEAWRKSCQPVTLSGSGTQEPEKKIVQELLETEQAYVARLHLLDQVFFQELLKTARSSKAFPEDVVRVIFSNISSIYQFHSQFFLPELQRRLDDWTANPRIGDVIQKLAPFLKMYSEYVKNFERAAELLATWTDKSPLFQEVLTRIQSSEASGSLTLQHHMLEPVQRIPRYELLLKEYIQKLPAQAPDQADAQKALDMIFSAAQHSNAAITEMERLQDLWEVYQRLGLEDDIVDPSNTLLREGPVLKISFRRNDPMERYLFLFNNMLLYCVPRVIQVGAQFQVRTRIDVAGMKVRELMDAEFPHSFLVSGKQRTLELQARSQEEMISWMQAFQAAIDQIEKRNETFKAAAQGPEGDTQEQELQSEELGLRAPQWVRDKMVTMCMRCQEPFNALTRRRHHCRACGYVVCARCSHYRAELKYDDNRPNRVCLHCYAFLTGNVLPEAKEDKRRGILEKGSSATPDQSLMCSFLQLIGDKWGKSGPRGWCVIPRDDPLVLYVYAAPQDMRAHTSIPLLGYQVTVGPQGDPRVFQLQQSGQLYTFKAETEELKGRWVKAMERAASGWSPSWPNDGDLSD

SEQ ID NO: 84 - protein, 697 aa, Homo sapiens
MKGASEEKLASVSNLVTVFENSRTPEAAPRGQRLEDVHHRPECRPPESPGPREKTNVGEAVGSEPRTVSRRYLNSLKNKLSSEAWRKSCQPVTLSGSGTQEPEKKIVQELLETEQAYVARLHLLDQAMSDLSWRLQVFFQELLKTARSSKAFPEDVVRVIFSNISSIYQFHSQFFLPELQRRLDDWTANPRIGDVIQKLAPFLKMYSEYVKNFERAAELLATWTDKSPLFQEVLTRIQSSEASGSLTLQHHMLEPVQRIPRYELLLKEYIQKLPAQAPDQADAQKALDMIFSAAQHSNAAITEMERLQDLWEVYQRLGLEDDIVDPSNTLLREGPVLKISFRRNDPMERYLFLFNNMLLYCVPRVIQVGAQFQVRTRIDVAGMKMHRNFFNQSSAERHLDCFQLSAAANEAAVNSLVRELMDAEFPHSFLVSGKQRTLELQARSQEEMISWMQAFQAAIDQIEKRNETFKAAAQGPEGDIQEQELQSEELGLRAPQWVRDKMVTMCMRCQEPFNALTRRRHHCRACGYVVCARCSDYRAELKYDDNRPNRVCLHCYAFLTGNVLPEAKEDKRRGILEKGSSATPDQSLMCSFLQLIGDKWGKSGPRGWCVIPRDDPLVLYVYAAPQDMRAHTSIPLLGYQVTVGPQGDPRVFQLQQSGQLYTFKAETEELKGRWVKAMERAASGWSPSWPNDGDLSD

SEQ ID NO: 85 - protein, 1725 aa, Macacca mulatta
MGNSESQYTLQGSKNHSNTITGAKQIPCSLKIRGIHAKEEKSLHGWVHGSSGAGYKSRSLARSCLSHFKSNQPYASRLGGSTCKVSRGVAYSTHRTNAPGKDFQGISAAFSTENGFHSVGHEPADNHITSRDCNGHLLNCYGRNESVASTPPGEDRKSPRVLIKTLGKLDGCLRVEFHNGGNPSKVPAEDSSEPVQLLRYSPTLASETSPVPEARRGSSADSLPSHRPSPTDSRLRSSKGSSLSSESSWYDSPWGNAGELSEAEGSFLAPGMPDPSLHASFPPGDAKKPFNQSSSLSSLRELYKDANLGSLSPSGIRLSDEYMGTHASLSNRVSFASDIDVPSRVAHRDPVQYSSFTLPCRKPKALVEDTAKKDSLKARMRRISDWTGSLSRKKRKLQEPRSKEGSDYFDSRSDGLNTEVQGPSQASAFLWSGGSAQILSQRSESTHAIGSDPLRQNIYENFMRELEMSRTNTENIEMSTETAESSSESLSSLEQLDLLFEKEQGVVRKAGWLFFKPLVTVQKERKLELVARRKWKQYWVTLKGCTLLFYETYGKNSMDQSSAPRCALFAEDSIVQAVPEHPKKENVFCLSNSFGDVYLFQATSQTDLENWVTAVHSACASLFAKKHGKEDTLRLLKNQTKNLLQKIDMDSKMKKMAELQLSIVSDPKNRKAIENQIQQWEQNLEKFHMDLFRMRCYLASLQGGELPNPKSLLAAASRPSKLALGRLGILSVSSFHALVCSRDDSALRKRTLSLTQRGRNKKGIFSSLKGLDTLARKGKEKRPSITQIFDLSGSHGFSGTRLPQNSNNSSEVDELLHMYGSTVDSVPRDNAWEIQTYVHFQDNHGVTVGIKPEHRVEDILTLACKMRQLEPSHYGLQLRKLVDDNVEYYIPAPYEYMQEQVYDEIEVFPLNVYDVQLTKTGSVCDFGFAVTAQVDEHQHLSRIFVSDVLPDGLAYGEGLRKGNEIMTLNGEAVSDLDLKQMEALFSEKSVGLTLIARPPDTKATLCTSWSDSDLFSRDQKSLLPPPNQSQLLEEFLDNFKKNTANDFSNVPDITTGLKRSQTDGTLDQVSHREKMEQTFRSAEQITALCRSFNDTQANGMEGPRESQDPPPRPLARHLSDADRLRKVIQELVDTEKSYVKDLSCLFELYLEPLQNETFLTQDEMESLFGSLPEMLEFQKVFLETLEDGISASSDFNTLETPSQFRKLLFSLGGSFLYYADHFKLYSGFCANHIKVQKVLERAKTDKAFKAFLDARNPTKQHSSTLESYLIKPVQRVLKYPLLLKELVSLTDQESEEHYHLTEALKAMEKVASHINEMQKIYEDYGTVFDQLVAEQSGTEKEVTELSMGELLMHSTVSWLNPFLSLGKARKDLELTVFVFKRAVILVYKENCKLKKKLPSNSRPAHNSADLDPFKFRWLIPISALQVRLGNPAGTENNSIWELIHTKSEIEGRPETIFQLCCSDSESKTNIVKVIRSILRENFRRHIKCELPLEKTCKDRLVPLKNRVPVSAKLASSRSLKVLKNSSSNEWTGETGKGTSLDSDEGSLSSGTQSSGCPTAEGRQDSKTASPGKYPHPGLADFADNLIKESDILSDEDDDHHQTLKRGSPTKDIEIQFQRLRISEDPDVHPEAEQQPGPEAGEGQKGGEQPKLVRGHFCPIKRKANSTKRDRGTLLKAQLRHQSLDSQSENATIDLNSVLEREFSVQSLTSVVNEECFYETESHGKS

SEQ ID NO: 86 - protein, 1701 aa, Macacca mulatta
MGNSESQYTLQGSKNHSNTITGAKQIPCSLKIRGIHAKEEKSLHGWVHGSSGAGYKSRSLARSCLSHFKSNQPYASRLGGSTCKVSRGVAYSTHRTNAPGKDFQGISAAFSTENGFHSVGHEPADNHITSRDCNGHLLNCYGRNESVASTPPGEDRKSPRVLIKTLGKLDGCLRVEFHNGGNPSKVPAEDSSEPVQLLRYSPTLASETSPVPEARRGSSADSLPSHRPSPTDSRLRSSKGSSLSSESSWYDSPWGNAGELSEAEGSFLAPGMPDPSLHASFPPGDAKKPFNQSSSLSSLRELYKDANLGSLSPSGIRLSDEYMGTHASLSNRVSFASDIDVPSRVAHRDPVQYSSFTLPCRKPKALVEDTAKKDSLKARMRRISDWTGSLSRKKRKLQEPRSKEGSDYFDSRSDGLNTEVQGPSQASAFLWSGGSAQILSQRSESTHAIGSDPLRQNIYENFMRELEMSRTNTENIEMSTETAESSSESLSSLEQLDLLFEKEQGVVRKAGWLFFKPLVTVQKERKLELVARRKWKQYWVTLKGCTLLFYETYGKNSMDQSSAPRCALFAEDSIVQAVPEHPKKENVFCLSNSFGDVYLFQATSQTDLENWVTAVHSACASLFAKKHGKEDTLRLLKNQTKNLLQKIDMDSKMKKMAELQLSIVSDPKNRKAIENQIQQWEQNLEKFHMDLFRMRCYLASLQGGELPNPKSLLAAASRPSKLALGRLGILSVSSFHALVCSRDDSALRKRTLSLTQRGRNKKGIFSSLKGLDTLARKGKEKRPSITQVDELLHMYGSTVDSVPRDNAWEIQTYVHFQDNHGVTVGIKPEHRVEDILTLACKMRQLEPSHYGLQLRKLVDDNVEYYIPAPYEYMQEQVYDEIEVFPLNVYDVQLTKTGSVCDFGFAVTAQVDEHQHLSRIFVSDVLPDGLAYGEGLRKGNEIMTLNGEAVSDLDLKQMEALFSEKSVGLTLIARPPDTKATLCTSWSDSDLFSRDQKSLLPPPNQSQLLEEFLDNFKKNTANDFSNVPDITTGLKRSQTDGTLDQVSHREKMEQTFRSAEQITALCRSFNDTQANGMEGPRESQDPPPRPLARHLSDADRLRKVIQELVDTEKSYVKDLSCLFELYLEPLQNETFLTQDEMESLFGSLPEMLEFQKVFLETLEDGISASSDFNTLETPSQFRKLLFSLGGSFLYYADHFKLYSGFCANHIKVQKVLERAKTDKAFKAFLDARNPTKQHSSTLESYLIKPVQRVLKYPLLLKELVSLTDQESEEHYHLTEALKAMEKVASHINEMQKIYEDYGTVFDQLVAEQSGTEKEVTELSMGELLMHSTVSWLNPFLSLGKARKDLELTVFVFKRAVILVYKENCKLKKKLPSNSRPAHNSADLDPFKFRWLIPISALQVRLGNPAGTENNSIWELIHTKSEIEGRPETIFQLCCSDSESKTNIVKVIRSILRENFRRHIKCELPLEKTCKDRLVPLKNRVPVSAKLASSRSLKVLKNSSSNEWTGETGKGTSLDSDEGSLSSGTQSSGCPTAEGRQDSKTASPGKYPHPGLADFADNLIKESDILSDEDDDHHQTLKRGSPTKDIEIQFQRLRISEDPDVHPEAEQQPGPEAGEGQKGGEQPKLVRGHFCPIKRKANSTKRDRGTLLKAQLRHQSLDSQSENATIDLNSVLEREFSVQSLTSVVNEECFYETESHGKS

SEQ ID NO: 87 - protein, 1730 aa, Monodelphis domesticus
VTMGNSESQYSLQGSKNHSSSSTGAKQKPCPLKIRSIHAKDEKSCSLHGWGHGNSGTNYKSRSLARSCLSHFKSNQPYSSRLDPMAKVSKGSAHTKHKGNVSGNYFQGKNSAFLSENGFHYISHEPADNHITSRDCNGHLLNCYGKNESLASTPPAEDRKSPKVLIKTLGKLDGCLRVEFHNSSNNKVPTEDSTGPVQLLRYSPTLESETSNLTEIRRNSSTDYSASHCLSPTDSRLRSSKGSSFSSESSWYDSPWGNAGDINELEGPYLTRSTPDTSIHSSFPSDDAKKPFNQSSSLSSLRELYKDATLESLPPSGIRFSDEYIGTHASLNSRVSFVSDIDVPSRAEQRGPVQYCSYTLPCRKSKPLTEDTSKKDTLKTRMRRISDWTGSLSRKKRKLQEPKSKDGYDHLENRTDGLSAEVLVPSQLSSLLWPGGSGQTLPQRSESTNAISSDPLRQNIYENFMRELEMSRTHVDNTETSTDTADSSSDSLSSLEQLDLLFEKEQGVVRKAGWLFFKPLVTLQKEKKLELVARRKWKQYWVTLKGCTLLFYETYGRNSMDQSSSPRCALFAEDSVVQSVPEHPKKENVFCLSNSFGDVYLFQATSQTDLENWVTAIHSACASLFAKKHGKEDTVRLLKNQTKNLLQKIDMDSKMKKMAELQLSVVSDPKNRKAIENQIQQWEQNLEKFHMDLFRMRCYLASLQGGELPNPKSLLAATSRPSKLALGRLGILSVSSFHALICSRDESALRKRTLSLTQQGQSKKGLFSSLKGLDTLARKGKDKRPSITQIFDSSGNHGFPGTQLPQSSNNSSEVDEFQHIYSSASDSGPRENIWESQTYVHFQDNQGVTLIIKPEHRVEDILSLSCKMKQLDPSYYGLQLRRLVDDNFEYSIPALYEYMQEQAIVYDEIEIFPLSIYHVLLTKTGNITDFGFAVTAQVDEHQHLSRIFISDVLPDGLAYGEGLRTGNEILTINGEVISDLDLRQMESLFSERSVRLTLRTNSSDTKRTLGTSWSDSDLSRDQKNLLPPPNQSQLLEEFLDNLKKNTANDFSNVPDVTAGLKRSQTDGTLDQVPHREKTEHTFRSAEQIAALCRSFNEVQPSTDSMEGPKEAQEPPPRPLARHLSDADRLRKVIQELMDTEKSYVKDLTCLFELYLEPLQNETFLTQDEMESLFGSLPEMLEFQKVFLETLEDGISSSSDFNILETPSQFRKLLFSLGGSFLYYTDHFKLYSGFCANHIKVQKVLERAKTDKAFKAFLDARNPTKQHASTLESYLIKPVQRVLKYPLLLKELVSLTDNESEEHYHLTEALKAMEKVASHINEMQKIYEDYGTVFDQLVAEQSGTEKEVTELSMGELLLHSTVSWLNPFLSLGKARKDLELTVFVFKRAVILVYKENCKLKKKLPSSSRPTHVHGDLDPFKFRWLIPLSALQVRLGNAAGTENNSIWELIHTKSEIEGRPETTFQLCCSDCESKTNIVKVIRSILRENFRRHIKSELPLEKTCKDRLIPLKNRIPVSAKLASSRSLRVLKNSPSNEWNSDTGKGNLLDSDECSLSSSTQSSGCHTTESRQESKDSSPEKYPQTCSSDFSDSLIKESDILSDEDDDFHQTLKKGSPTKDIEIQFQRLKISEDSEGDSTADQQPRTEVGNNLNSVAHPKLVRGHFCPIKRKANSTKRDRGTLLTLQARHQSLDSKSENANIDLNSILEREFSVQSLTSVVNEECFYETDSHGKS

SEQ ID NO: 88 - protein, 1706 aa, Monodelphis domestica
VTMGNSESQYSLQGSKNHSSSSTGAKQKPCPLKIRSIHAKDEKSCSLHGWGHGNSGTNYKSRSLARSCLSHFKSNQPYSSRLDPMAKVSKGSAHTKHKGNVSGNYFQGKNSAFLSENGFHYISHEPADNHITSRDCNGHLLNCYGKNESLASTPPAEDRKSPKVLIKTLGKLDGCLRVEFHNSSNNKVPTEDSTGPVQLLRYSPTLESETSNLTEIRRNSSTDYSASHCLSPTDSRLRSSKGSSFSSESSWYDSPWGNAGDINELEGPYLTRSTPDTSIHSSFPSDDAKKPFNQSSSLSSLRELYKDATLESLPPSGIRFSDEYIGTHASLNSRVSFVSDIDVPSRAEQRGPVQYCSYTLPCRKSKPLTEDTSKKDTLKTRMRRISDWTGSLSRKKRKLQEPKSKDGYDHLENRTDGLSAEVLVPSQLSSLLWPGGSGQTLPQRSESTNAISSDPLRQNIYENFMRELEMSRTHVDNTETSTDTADSSSDSLSSLEQLDLLFEKEQGVVRKAGWLFFKPLVTLQKEKKLELVARRKWKQYWVTLKGCTLLFYETYGRNSMDQSSSPRCALFAEDSVVQSVPEHPKKENVFCLSNSFGDVYLFQATSQTDLENWVTAIHSACASLFAKKHGKEDTVRLLKNQTKNLLQKIDMDSKMKKMAELQLSVVSDPKNRKAIENQIQQWEQNLEKFHMDLFRMRCYLASLQGGELPNPKSLLAATSRPSKLALGRLGILSVSSFHALICSRDESALRKRTLSLTQQGQSKKGLFSSLKGLDTLARKGKDKRPSITQVDEFQHIYSSASDSGPRENIWESQTYVHFQDNQGVTLIIKPEHRVEDILSLSCKMKQLDPSYYGLQLRRLVDDNFEYSIPALYEYMQEQAIVYDEIEIFPLSIYHVLLTKTGNITDFGFAVTAQVDEHQHLSRIFISDVLPDGLAYGEGLRTGNEILTINGEVISDLDLRQMESLFSERSVRLTLRTNSSDTKRTLGTSWSDSDLSRDQKNLLPPPNQSQLLEEFLDNLKKNTANDFSNVPDVTAGLKRSQTDGTLDQVPHREKTEHTFRSAEQIAALCRSFNEVQPSTDSMEGPKEAQEPPPRPLARHLSDADRLRKVIQELMDTEKSYVKDLTCLFELYLEPLQNETFLTQDEMESLFGSLPEMLEFQKVFLETLEDGISSSSDFNILETPSQFRKLLFSLGGSFLYYTDHFKLYSGFCANHIKVQKVLERAKTDKAFKAFLDARNPTKQHASTLESYLIKPVQRVLKYPLLLKELVSLTDNESEEHYHLTEALKAMEKVASHINEMQKIYEDYGTVFDQLVAEQSGTEKEVTELSMGELLLHSTVSWLNPFLSLGKARKDLELTVFVFKRAVILVYKENCKLKKKLPSSSRPTHVHGDLDPFKFRWLIPLSALQVRLGNAAGTENNSIWELIHTKSEIEGRPETTFQLCCSDCESKTNIVKVIRSILRENFRRHIKSELPLEKTCKDRLIPLKNRIPVSAKLASSRSLRVLKNSPSNEWNSDTGKGNLLDSDECSLSSSTQSSGCHTTESRQESKDSSPEKYPQTCSSDFSDSLIKESDILSDEDDDFHQTLKKGSPTKDIEIQFQRLKISEDSEGDSTADQQPRTEVGNNLNSVAHPKLVRGHFCPIKRKANSTKRDRGTLLTLQARHQSLDSKSENANIDLNSILEREFSVQSLTSVVNEECFYETDSHGKS

SEQ ID NO: 89 - protein, 1730 aa, Monodelphis domestica
VTMGNSESQYSLQGSKNHSSSSTGAKQKPCPLKIRSIHAKDEKSCSLHGWGHGNSGTNYKSRSLARSCLSHFKSNQPYSSRLDPMAKVSKGSAHTKHKGNVSGNYFQGKNSAFLSENGFHYISHEPADNHITSRDCNGHLLNCYGKNESLASTPPAEDRKSPKVLIKTLGKLDGCLRVEFHNSSNNKVPTEDSTGPVQLLRYSPTLESETSNLTEIRRNSSTDYSASHCLSPTDSRLRSSKGSSFSSESSWYDSPWGNAGDINELEGPYLTRSTPDTSIHSSFPSDDAKKPFNQSSSLSSLRELYKDATLESLPPSGIRFSDEYIGTHASLNSRVSFVSDIDVPSRAEQRGPVQYCSYTLPCRKSKPLTEDTSKKDTLKTRMRRISDWTGSLSRKKRKLQEPKSKDGYDHLENRTDGLSAEVLVPSQLSSLLWPGGSGQTLPQRSESTNAISSDPLRQNIYENFMRELEMSRTHVDNTETSTDTADSSSDSLSSLEQLDLLFEKEQGVVRKAGWLFFKPLVTLQKEKKLELVARRKWKQYWVTLKGCTLLFYETYGRNSMDQSSSPRCALFAEDSVVQSVPEHPKKENVFCLSNSFGDVYLFQATSQTDLENWVTAIHSACASLFAKKHGKEDTVRLLKNQTKNLLQKIDMDSKMKKMAELQLSVVSDPKNRKAIENQIQQWEQNLEKFHMDLFRMRCYLASLQGGELPNPKSLLAATSRPSKLALGRLGILSVSSFHALICSRDESALRKRTLSLTQQGQSKKGLFSSLKGLDTLARKGKDKRPSITQIFDSSGNHGFPGTQLPQSSNNSSEVDEFQHIYSSASDSGPRENIWESQTYVHFQDNQGVTLIIKPEHRVEDILSLSCKMKQLDPSYYGLQLRRLVDDNFEYSIPALYEYMQEQAKVYDEIEIFPLSIYHVLLTKTGNITDFGFAVTAQVDEHQHLSRIFISDVLPDGLAYGEGLRTGNEILTINGEVISDLDLRQMESLFSERSVRLTLRTNSSDTKRTLGTSWSDSDLSRDQKNLLPPPNQSQLLEEFLDNLKKNTANDFSNVPDVTAGLKRSQTDGTLDQVPHREKTEHTFRSAEQIAALCRSFNEVQPSTDSMEGPKEAQEPPPRPLARHLSDADRLRKVIQELMDTEKSYVKDLTCLFELYLEPLQNETFLTQDEMESLFGSLPEMLEFQKVFLETLEDGISSSSDFNILETPSQFRKLLFSLGGSFLYYTDHFKLYSGFCANHIKVQKVLERAKTDKAFKAFLDARNPTKQHASTLESYLIKPVQRVLKYPLLLKELVSLTDNESEEHYHLTEALKAMEKVASHINEMQKIYEDYGTVFDQLVAEQSGTEKEVTELSMGELLLHSTVSWLNPFLSLGKARKDLELTVFVFKRAVILVYKENCKLKKKLPSSSRPTHVHGDLDPFKFRWLIPLSALQVRLGNAAGTENNSIWELIHTKSEIEGRPETTFQLCCSDCESKTNIVKVIRSILRENFRRHIKSELPLEKTCKDRLIPLKNRIPVSAKLASSRSLRVLKNSPSNEWNSDTGKGNLLDSDECSLSSSTQSSGCHTTESRQESKDSSPEKYPQTCSSDFSDSLIKESDILSDEDDDFHQTLKKGSPTKDIEIQFQRLKISEDSEGDSTADQQPRTEVGNNLNSVAHPKLVRGHFCPIKRKANSTKRDRGTLLTLQARHQSLDSKSENANIDLNSILEREFSVQSLTSVVNEECFYETDSHGKS

SEQ ID NO: 90 - protein, 644 aa, Pan troglodytes
SAEQITALCRSFNDSQANGMEGPRESQDPPPRPLARHLSDADRLRKVIQELVDTEKSYVKDLSCLFELYLEPLQNETFLTQDEMESLFGSLPEMLEFQKVFLETLEDGISASSDFNTLETPSQFRKLLFSLGGSFLYYADHFKLYSGFCANHIKVQKVLERAKTDKAFKAFLDTRNPTKQHSSTLESYLIKPVQRVLKYPLLLKELVSLTDQESEEHYHLTEALKAMEKVASHINEMQKIYEDYGTVFDQLVAEQSGTEKEVTELSMGELLMHSTVSWLNPFLSLGKARKDLELTVFVFKRAVILVYKENCKLKKKLASNSRPAHNSTDLDPFKFRWLIPISALQVRLGNPAGTENNSIWELIHTKSEIEGRPETIFQLCCSDSESKTNIVKVIRSILRENFRRHIKCELPLEKTCKDRLVPLKNRVPVSAKLASSRSLKVLKNSSSNEWTGETGKGTLLDSDEGSLSSSTQSSGCPTAGRQDSKSTSPGKYPHPGLADFADNLIKESDILSDEDDDHRQTVKQGSPTKDIEIQFQRLRISEDPDVHPEAEQQPGPESGEGQKGGEQPKLVRGHFCPIKRKANSTKRDRGTLLKAQIRHQSLDSQSENATIDLNSVLEREFSVQSLTSVVSEECFYETESHGKS

SEQ ID NO: 91 - DNA, 24 bp, Artificial Sequence
  Description of Artificial Sequence Sense primer
gactcctagggtcagagtgtcatg

SEQ ID NO: 92 - DNA, 22 bp, Artificial Sequence
  Description of Artificial Sequence Antisense primer
tgggctccacatctgggtcatt

SEQ ID NO: 93 - DNA, 30 bp, Artificial Sequence
  Description of Artificial Sequence Primer
atcctctgccttaaaggtccttcaacggaa

SEQ ID NO: 94 - DNA, 20 bp, Artificial Sequence
  Description of Artificial Sequence Sense primer
ccagacccatccaggacatc

SEQ ID NO: 95 - DNA, 21 bp, Artificial Sequence
  Description of Artificial Sequence Antisense primer
ctggcagctttcctgaatatc

SEQ ID NO: 96 - DNA, 18 bp, Artificial Sequence
  Description of Artificial Sequence Sense primer
agggcccttggggtcagg

SEQ ID NO: 97 - DNA, 24 bp, Artificial Sequence
  Description of Artificial Sequence Antisense primer
ctgtcagtctccattccaatgaag

SEQ ID NO: 98 - DNA, 22 bp, Artificial Sequence
  Description of Artificial Sequence Antisense primer
cagttagctggcaaatgctgtc

SEQ ID NO: 99 - DNA, 23 bp, Artificial Sequence
  Description of Artificial Sequence Primer
actagtctgcttctggggtaact

SEQ ID NO: 100 - DNA, 17 bp, Artificial Sequence
  Description of Artificial Sequence Primer
ataggcctgctccgtct

SEQ ID NO: 101 - DNA, 28 bp, Artificial Sequence
  Description of Artificial Sequence Primer
gatatcaagaatcccgcggtacgaactg

SEQ ID NO: 102 - DNA, 28 bp, Artificial Sequence
  Description of Artificial Sequence Primer
gtcgacgacaacgcccgacatcatagag

SEQ ID NO: 103 - DNA, 22 bp, Artificial Sequence
  Description of Artificial Sequence Primer
acaggtctcacgtagccgaatc

SEQ ID NO: 104 - DNA, 22 bp, Artificial Sequence
  Description of Artificial Sequence Primer
cgggtgaagcaggtctaccaca

SEQ ID NO: 105 - DNA, 22 bp, Artificial Sequence
  Description of Artificial Sequence Primer
tggatgccgctcagttgctaat

SEQ ID NO: 106 - DNA, 22 bp, Artificial Sequence
  Description of Artificial Sequence Primer
tgaaactcagtgtgtagaccag

SEQ ID NO: 107 - DNA, 24 bp, Artificial Sequence
  Description of Artificial Sequence Oligonucleotide primer sequences
tgaagctcattttctccaacatct

SEQ ID NO: 108 - DNA, 18 bp, Artificial Sequence
  Description of Artificial Sequence Oligonucleotide primer sequences
ctgcagctcgggaaggaa

SEQ ID NO: 109 - DNA, 27 bp, Artificial Sequence
  Description of Artificial Sequence Oligonucleotide primer sequences
ctccatctatcgtttccacgcccagtt

SEQ ID NO: 110 - DNA, 20 bp, Artificial Sequence
  Description of Artificial Sequence Oligonucleotide primer sequences
ggctgtggtagacctgcttc

SEQ ID NO: 111 - DNA, 17 bp, Artificial Sequence
  Description of Artificial Sequence Oligonucleotide primer sequences
agacggagcaggcctat

SEQ ID NO: 112 - DNA, 24 bp, Artificial Sequence
  Description of Artificial Sequence Oligonucleotide primer sequences
tggaatcctgtggcatccatgaaa

SEQ ID NO: 113 - DNA, 25 bp, Artificial Sequence
  Description of Artificial Sequence Oligonucleotide primer sequences
taaaacgcagctcagtaacagtccg

SEQ ID NO: 114 - DNA, 17 bp, Artificial Sequence
  Description of Artificial Sequence Oligonucleotide primer sequences
agaatcccgcggtacga

SEQ ID NO: 115 - DNA, 16 bp, Artificial Sequence
  Description of Artificial Sequence Oligonucleotide primer sequences
gtcagctcccgcacct

SEQ ID NO: 116 - DNA, 19 bp, Artificial Sequence
  Description of Artificial Sequence Oligonucleotide primer sequences
ggctagcaggatggagcga

SEQ ID NO: 117 - DNA, 22 bp, Artificial Sequence
  Description of Artificial Sequence Oligonucleotide primer sequences
gtgctctcaggttcttgtgtag

SEQ ID NO: 118 - DNA, 17 bp, Artificial Sequence
  Description of Artificial Sequence Oligonucleotide primer sequences
ggcttgcggctatgtag

SEQ ID NO: 119 - DNA, 22 bp, Artificial Sequence
  Description of Artificial Sequence Oligonucleotide primer sequences
gtgctctcaggttcttgtgtag

SEQ ID NO: 120 - DNA, 19 bp, Artificial Sequence
  Description of Artificial Sequence Oligonucleotide primer sequences
ggctagcaggatggagcga

SEQ ID NO: 121 - DNA, 17 bp, Artificial Sequence
  Description of Artificial Sequence Oligonucleotide primer sequences
agacggagcaggcctat

SEQ ID NO: 122 - DNA, 17 bp, Artificial Sequence
  Description of Artificial Sequence Oligonucleotide primer sequences
ctgcttctggggtaact

SEQ ID NO: 123 - DNA, 17 bp, Artificial Sequence
  Description of Artificial Sequence Oligonucleotide primer sequences
ataggcctgctccgtct

SEQ ID NO: 124 - DNA, 22 bp, Artificial Sequence
  Description of Artificial Sequence Oligonucleotide primer sequences
aagaatcccgcggtacgaactg

SEQ ID NO: 125 - DNA, 22 bp, Artificial Sequence
  Description of Artificial Sequence Oligonucleotide primer sequences
gacaacgcccgacatcatagag

SEQ ID NO: 126 - DNA, 22 bp, Artificial Sequence
  Description of Artificial Sequence Oligonucleotide primer sequences
acaggtctcacgtagccgaatc

SEQ ID NO: 127 - DNA, 22 bp, Artificial Sequence
  Description of Artificial Sequence Oligonucleotide primer sequences
cgggtgaagcaggtctaccaca

SEQ ID NO: 128 - DNA, 22 bp, Artificial Sequence
  Description of Artificial Sequence Oligonucleotide primer sequences
tggatgccgctcagttgctaat

SEQ ID NO: 129 - DNA, 22 bp, Artificial Sequence
  Description of Artificial Sequence Oligonucleotide primer sequences
tgaaactcagtgtgtagaccag